(19) United States
(12) Patent Application Publication
(10) Pub. No.: US 2026/0258012 A1
(43) Pub. Date: Sep. 3, 2026

(54) TYROSINE KINASE INHIBITORS

(71) Applicant: TicTwo Therapeutics, Inc., Philadelphia, PA (US)

(72) Inventors: Ryan MOSLIN, Princeton, NJ (US); Naresh VEMULA, Vaudreuil-Dorion (CA); Curtis COLWELL, Orléans (CA); Thomas PINTER, Montreal (CA); Jean-François FOURNIER, Mont-Royal (CA); Samuel BLAIS, Montreal (CA); Hiu Fung Kevin LEE, Montreal (CA); David TONG, Dollard-des-Ormeaux (CA); Karthik DEVARAJ, Montreal (CA); Michael NEEB, Paoli, PA (US)

(21) Appl. No.: 19/545,555

(22) Filed: Feb. 20, 2026

Related U.S. Application Data
(60) Provisional application No. 63/761,385, filed on Feb. 21, 2025.
(60) Provisional application No. 63/853,889, filed on Jul. 30, 2025.
(60) Provisional application No. 63/902,281, filed on Oct. 20, 2025.

(51) Int. Cl. C07D 401/12; A61K 31/444; A61K 31/497; A61K 31/501; A61K 31/506; C07B 59/00; C07D 213/74; C07D 213/82; C07D 401/14; C07D 403/12; C07D 405/14; C07D 491/107

(52) U.S. Cl. CPC C07D 401/12; A61K 31/444; A61K 31/497; A61K 31/501; A61K 31/506; C07B 59/002; C07D 213/74; C07D 213/82; C07D 401/14; C07D 403/12; C07D 405/14; C07D 491/107

(57) ABSTRACT

Provided herein are compounds of Formula (I), or a pharmaceutically acceptable salt, deuterated form, or stereoisomer thereof; wherein R¹, R², R³, R⁴, Y³, Y², Y¹, Y, Z, U, X¹, and X² are defined herein, and methods of making such compounds. Also provided herein are pharmaceutical compositions comprising a compound of Formula (I) or a pharmaceutically acceptable salt, deuterated form, or stereoisomer thereof.

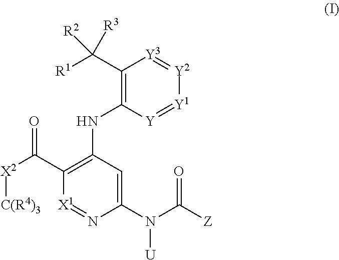

### CROSS REFERENCE TO RELATED APPLICATIONS

This application claims the benefit of U.S. Provisional Application No. 63/761,385, filed on Feb. 21, 2025, U.S. Provisional Application No. 63/853,889, filed on Jul. 30, 2025, and U.S. Provisional Application No. 63/902,281, filed on Oct. 20, 2025, the disclosures of each of which are incorporated by reference herein in its entirety.

### BACKGROUND

Janus kinase (JAK) is a family of non-receptor tyrosine kinases that transduce cytokine-mediated signals via the JAK-STAT pathway. The JAK-STAT signaling pathway is critical in immune regulation and is involved in various allergic reactions, autoimmune diseases, inflammation responses, and cancers. The mammalian JAK family consists of four members, JAK1, JAK2, JAK3, and Tyrosine kinase 2 (TYK2). TYK2 has been shown to be critical in regulating the signal transduction cascade downstream of receptors for IL-12, IL-23 and type I interferons. TYK2 mediates the receptor-induced phosphorylation of members of the STAT family of transcription factors, an essential signal that leads to the dimerization of STAT proteins and the transcription of STAT-dependent pro-inflammatory genes.

There remains a need for inhibitors of TYK2, including TYK2 selective inhibitors as well as compositions comprising same.

### SUMMARY

The present disclosure relates to compounds and methods of making such compounds useful for inhibition (e.g., allosteric inhibition) of non-receptor tyrosine-protein kinase 2, also known as Tyrosine kinase 2 (TYK2), as well as pharmaceutical compositions comprising such compounds.

In embodiments, compounds of the present disclosure are selective for TYK2 over other JAKs, for example selective for TYK2 over JAK1. In embodiments, the compounds of the present disclosure selectively inhibit the function of cytokines such as IFNα e.g., by acting on TYK2 to mediate signal transduction. In embodiments, the compounds of the present disclosure penetrate the blood brain barrier. In embodiments, compounds of the present disclosure are effective in treating TYK2 mediated diseases or disorders. For example, in embodiments, compounds of the present disclosure are effective in treating inflammatory and autoimmune diseases, neurodegenerative and neuroinflammatory diseases, or cancer (e.g., lung cancer, breast cancer, liver cancer, hematological malignancy such as leukemia, or sarcoma).

The present disclosure also provides processes and intermediates for making the compounds of the present invention.

In embodiments, the present disclosure provides a compound of Formula (I):

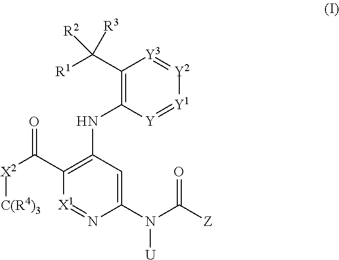

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl; R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, —OH, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl), or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂, NH, or C(R⁸)₂NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —NR^{A}R^{B}, —C₁₋₆ alkyl, —OC₁₋₆ alkyl, —C₁₋₆ alkenyl, —C₁₋₆ alkynyl, —C₃₋₆ cycloalkyl, heterocyclyl, or aryl, wherein the, —C₁₋₆ alkyl, —OC₁₋₆ alkyl, —C₁₋₆ alkenyl, —C₁₋₆ alkynyl, —C₃₋₆ cycloalkyl, heterocyclyl, or aryl are each optionally substituted with -halo, —C₁₋₆ alkyl, —C₁₋₆ haloalkyl, —OC₁₋₆ alkyl, —OC₃₋₆ cycloalkyl, or —CN;
R^{A} and R^{B} are each independently —H, —C₁-C₆ alkyl, cycloalkyl, or heterocyclyl; U is H;
Z is —OR^{Z}, —C₁₋₆alkyl, —(CH₂)₀₋₃—(C₃₋₆ cycloalkyl), or —(CH₂)₀₋₃—(C₃-C₄ heterocyclyl), and wherein the —C₁₋₆alkyl, —C₃₋₆ cycloalkyl or —C₃-C₄ heterocyclyl is optionally substituted with 1 or more substituents each independently selected from the group consisting of -halo, —C₁₋₆ alkyl, —C₁₋₆haloalkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.

In embodiments, the present disclosure provides a compound of Formula (I):

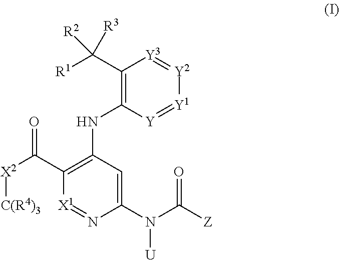

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —NR^{A}R^{B}, —C₁₋₆ alkyl, —OC₁₋₆ alkyl, —C₁₋₆ alkenyl, —C₁₋₆ alkynyl, —C₃₋₆ cycloalkyl, heterocyclyl, or aryl, wherein the, —C₁₋₆ alkyl, —OC₁₋₆ alkyl, —C₁₋₆ alkenyl, —C₁₋₆ alkynyl, —C₃₋₆ cycloalkyl, heterocyclyl, or aryl are each optionally substituted with -halo, —C₁₋₆ alkyl, —C₁₋₆ haloalkyl, —OC₁₋₆ alkyl, —OC₃₋₆ cycloalkyl, or —CN;
R^{A} and R^{B} are each independently —H, —C₁-C₆ alkyl, cycloalkyl, or heterocyclyl;
U is H;
Z is —OR^{Z}, —C₁₋₆alkyl, —(CH₂)₀₋₃—(C₃₋₆ cycloalkyl), or —(CH₂)₀₋₃—(C₃-C₄ heterocyclyl), and wherein the —C₁₋₆alkyl, —C₃₋₆ cycloalkyl or —C₃-C₄ heterocyclyl is optionally substituted with 1 or more substituents each independently selected from the group consisting of -halo, —C₁₋₆ alkyl, —C₁₋₆haloalkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide

In embodiments the present disclosure provides a compound of Formula (I):

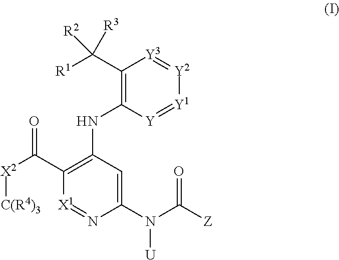

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z} or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.

In embodiments the present disclosure provides a compound of Formula (I):

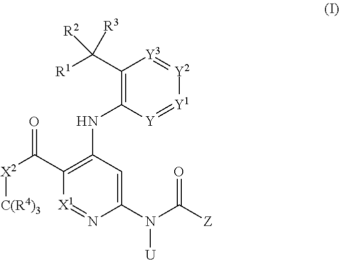

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z} or —(CH₂)₀₋₃—(C₃₋₆ cycloalkyl), or —(CH₂)₀₋₃—(C₃₋₄ heterocyclyl), wherein the —C₃₋₆ cycloalkyl is optionally substituted with 1 or more substituents each independently selected from the group consisting of -halo, —C₁₋₆ alkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.

In embodiments the present disclosure provides a compound of Formula (I):

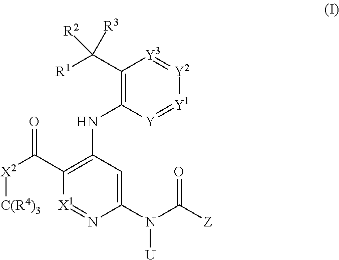

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z}, —NR⁹R¹⁰, or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl;
R⁹ and R¹⁰ are each independently —H or —C₁₋₆ alkyl; or
U and one of R⁹ or R¹⁰ are taken together to form a 5-7 membered heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.

In embodiments, the present disclosure provides a compound having a structure of Formula (IA):

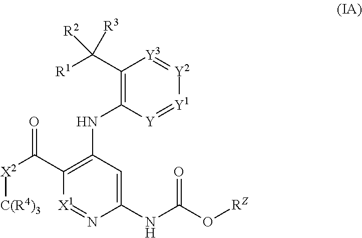

or a pharmaceutically acceptable salt, deuterated form, or stereoisomer thereof;
wherein R¹, R², R³, R⁴, Y³, Y², Y¹, Y, R^{Z}, X¹, and X² are defined herein.

In embodiments, the present disclosure provides a compound having a structure of Formula (IB):

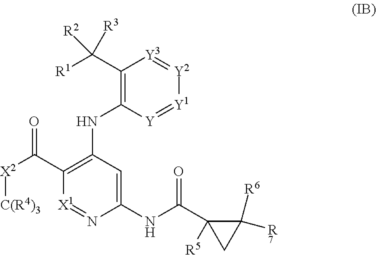

or a pharmaceutically acceptable salt, deuterated form, or stereoisomer thereof;
wherein R¹, R², R³, R⁴, R⁵, R⁶, R⁷, Y³, Y², Y¹, Y, X², and X² are defined herein.

In embodiments, the present disclosure provides a compound having a structure of Formula (IIA):

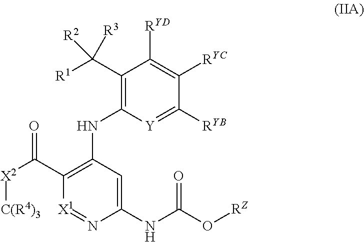

or a pharmaceutically acceptable salt, deuterated form, or stereoisomer thereof;
wherein R¹, R², R³, R⁴, R^{YD}, R^{YC}, R^{YB}, Y, R^{Z}, X¹, and X² are defined herein.

In embodiments, the present disclosure provides a compound having a structure of Formula (IIB):

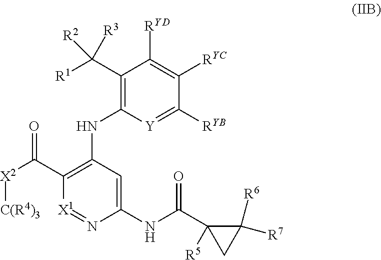

or a pharmaceutically acceptable salt, deuterated form, or stereoisomer thereof;
wherein R¹, R², R³, R⁴, R⁵, R⁶, R⁷, R^{YD}, R^{YC}, R^{YB}, X¹, and X² are defined herein.

In embodiments provided herein is a pharmaceutical composition comprising a compound of the present disclosure, or a pharmaceutically acceptable salt, deuterated form, or stereoisomer thereof, and a pharmaceutically acceptable carrier.

### BRIEF DESCRIPTION OF THE FIGURES

FIG. 1A shows inhibition of IFNα-stimulated ISG production in the blood by brain-penetrant Compound 12 compared to peripherally restricted inhibitor deucravacitinib.

FIG. 1B shows inhibition of IFNα-stimulated ISG production in the brain by brain-penetrant Compound 12 compared to peripherally restricted inhibitor deucravacitinib.

FIG. 2A shows inhibition of IFNα-stimulated ISG production in the blood by brain-penetrant Compound 20.

FIG. 2B shows inhibition of IFNα-stimulated ISG production in the brain by brain-penetrant Compound 20.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

Listed below are definitions of various terms used in the specification and claims to describe the present disclosure.

Unless defined otherwise, all technical and scientific terms used in this disclosure have the same meanings as commonly understood by one of ordinary skill in the art to which this disclosure belongs.

The term “about” when immediately preceding a numerical value means a range encompassing said numerical value plus or minus an acceptable amount of variation in the art (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. The pharmaceutically acceptable salts of the present disclosure can be synthesized from the parent compound which contains a basic or acidic moiety by conventional chemical methods. Lists of suitable salts are found in Remington's Pharmaceutical Sciences, 18th Edition, Mack Publishing Company, Easton, Pa. (1990), the disclosure of which is hereby incorporated by reference.

Compounds described herein may also comprise one or more isotopic substitutions. Isotopes include those atoms having the same atomic number but different mass numbers. By way of general example and without limitation, isotopes of hydrogen include ²H and ³H. Isotopes of carbon include ¹¹C, ¹³C and ¹⁴C. Isotopically enriched compounds of the disclosure can be prepared, for example, by conventional techniques known to those skilled in the art or by processes analogous to those described in the schemes and examples herein using appropriate isotopically enriched reagents and/or intermediates.

All stereoisomers of the compounds of the present disclosure are contemplated, either in admixture or in pure or substantially pure form. Stereoisomers may include compounds which are optical isomers through possession of one or more chiral atoms, as well as compounds which are optical isomers by virtue of limited rotation about one or more bonds (atropisomers). Compounds of the present disclosure and salts thereof may exist in their tautomeric form, in which hydrogen atoms are transposed to other parts of the molecules and the chemical bonds between the atoms of the molecules are consequently rearranged. It should be understood that the all tautomeric forms, insofar as they may exist, are included within the present disclosure.

In embodiments, the compounds of the present disclosure are provided as a mixture of diastereomers. In embodiments, a diastereomer of a compound of the present disclosure is provided substantially free of other possible diastereomer(s). Additionally, compounds of the present disclosure may have trans- and cis-isomers.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Halo” or “halogen” refers to fluoro, chloro, bromo, or iodo.

“Cyano” refers to the —CN radical.

“Hydroxy” or “hydroxyl” refers to the —OH radical.

“Oxo” refers to the ═O substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethenylene, propenylene, butenylene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes Cs alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkynyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems (when fused with a saturated or partially unsaturated ring, the aryl is bonded through an aromatic ring atom). In embodiments, the aryl is a 6-10 membered aryl. Examples of aryl groups include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged or spirocyclic ring systems, having from three to twenty carbon atoms (e.g., C₃-C₁₂, C₃-C₁₀, C₃-C₈, or C₃-C₆), and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, spirocyclic cycloalkyl such as spiro[2.2]pentanyl, spiro[2.3]hexyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, e.g., having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, e.g., having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Haloalkyl” refers to an alkyl radical, as defined above, that is substituted by one or more (e.g., 1, 2, 3, 4, 5, or 6) halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, partially unsaturated heterocyclyls and fully unsaturated heterocyclyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered aromatic ring system comprising hydrogen atoms, one to nineteen carbon atoms, one to six heteroatoms selected from the group consisting of nitrogen, oxygen and sulfur, which is attached to the rest of the molecule by a single bond. For purposes of this disclosure, the heteroaryl can be a monocyclic, bicyclic, tricyclic or tetracyclic ring system; and the nitrogen, carbon or sulfur atoms in the heteroaryl can be optionally oxidized; the nitrogen atom can be optionally quaternized. Heteroaryl groups can also be fused or bridged with carbocyclic or heterocyclic rings, which are not aromatic so as to form a multicyclic system, wherein the heteroaryl is attached to the rest of the molecule by a single bond to an aromatic atom. In some embodiments, the heteroaryl is a 5- to 10-membered heteroaryl. In some embodiments, the heteroaryl is a 5- to 6-membered monocyclic heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“The term “substituted” used herein means any of the groups described herein (e.g., alkyl, alkenyl, alkynyl, aryl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl group. In embodiments, “substituted” includes any of the above groups in which one or more hydrogen atoms are replaced with halo, nitro, —CN, —C₁-C₆alkyl, NR^{A}R^{B}, C₁-C₆-haloalkyl, —C₁-C₆-alkyl-NR^{A}R^{B}, —C₁-C₆-alkyl-OH, —C(═O)R^{D}, —C(═O)N(R^{E})R^{F}, —C(═O)—OR^{D}, —N(R^{G})C(═O)R^{D}, —N(R^{G})C(═O)N(R^{E})R^{F}, —N(R^{G})C(═O)OR^{D}, —N(R^{G})S(═O)R^{D}, —N(R^{G})S(═O)₂R^{D}, —N═S(═O)(R^{E})R^{F}, —OR^{D}, —O(C═O)R^{D}, —O(C═O)N(R^{E})R^{F}, —O(C═O)OR^{D}, —C₂-C₆ alkenyl, or —C₂-C₆ alkynyl; wherein R^{A} and R^{B} are each independently —H, —C₁-C₆ alkyl, cycloalkyl, aryl, heteroaryl, —C(O)R^{C}, —C(O)OR^{C}, —S(O)R^{C}, or —S(O)₂R^{C}; R^{C} is H, —C₁-C₆ alkyl, aryl, cycloalkyl, or heterocyclyl; and R^{D}, R^{E}, R^{F}, R^{G}, are each independently selected from —H, C₁-C₆-alkyl-, C₁-C₆-haloalkyl-, —C₁-C₆-alkyl-OH, cycloalkyl, C₂-C₆-alkenyl-, heterocyclyl, aryl, or heteroaryl. In addition, each of the foregoing substituents can also be optionally substituted with one or more of the above substituents.

The present disclosure relates to compounds and methods of making such compounds useful for inhibiting non-receptor tyrosine-protein kinase 2, also known as Tyrosine kinase 2 (TYK2), as well as pharmaceutical compositions comprising such compounds.

In embodiments, compounds of the present disclosure are selective for TYK2 over other JAKs, for example selective for TYK2 over JAK1. In embodiments, the compounds of the present disclosure selectively inhibit the function of cytokines such as IFNα e.g., by acting on TYK2 to mediate signal transduction. In embodiments, the compounds of the present disclosure penetrate the blood brain barrier. In embodiments, compounds of the present disclosure are effective in treating TYK2 mediated diseases or disorders. For example, in embodiments, compounds of the present disclosure are effective in treating inflammatory and autoimmune diseases, neurodegenerative and neuroinflammatory diseases, or cancer (e.g., lung cancer, breast cancer, liver cancer, hematological malignancy such as leukemia, or sarcoma).

The present disclosure also provides processes and intermediates for making the compounds of the present invention.

### Compounds

In embodiments, the present disclosure provides a compound of Formula (I):

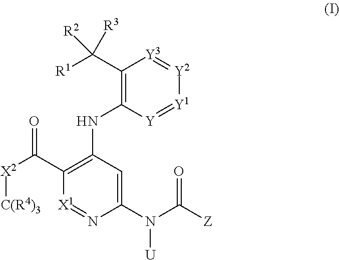

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, —OH, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl), or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂, NH, or C(R⁸)₂NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —NR^{A}R^{B}, —C₁₋₆ alkyl, —OC₁₋₆ alkyl, —C₁₋₆ alkenyl, —C₁₋₆ alkynyl, —C₃₋₆ cycloalkyl, heterocyclyl, or aryl, wherein the, —C₁₋₆ alkyl, —OC₁₋₆ alkyl, —C₁₋₆ alkenyl, —C₁₋₆ alkynyl, —C₃₋₆ cycloalkyl, heterocyclyl, or aryl are each optionally substituted with -halo, —C₁₋₆ alkyl, —C₁₋₆ haloalkyl, —OC₁₋₆ alkyl, —OC₃₋₆ cycloalkyl, or —CN;
R^{A} and R^{B} are each independently —H, —C₁-C₆ alkyl, cycloalkyl, or heterocyclyl;
U is H;
Z is —OR^{Z}, —C₁₋₆alkyl, —(CH₂)₀₋₃—(C₃₋₆ cycloalkyl), or —(CH₂)₀₋₃—(C₃-C₄ heterocyclyl), and wherein the —C₁₋₆alkyl, —C₃₋₆ cycloalkyl or —C₃-C₄ heterocyclyl is optionally substituted with 1 or more substituents each independently selected from the group consisting of -halo, —C₁₋₆ alkyl, —C₁₋₆haloalkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.

In embodiments, the present disclosure provides a compound of Formula (I):

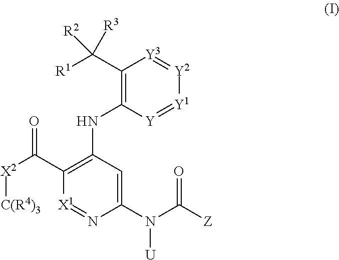

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —NR^{A}R^{B}, —C₁₋₆ alkyl, —OC₁₋₆ alkyl, —C₁₋₆ alkenyl, —C₁₋₆ alkynyl, —C₃₋₆ cycloalkyl, heterocyclyl, or aryl, wherein the, —C₁₋₆ alkyl, —OC₁₋₆ alkyl, —C₁₋₆ alkenyl, —C₁₋₆ alkynyl, —C₃₋₆ cycloalkyl, heterocyclyl, or aryl are each optionally substituted with -halo, —C₁₋₆ alkyl, —C₁₋₆ haloalkyl, —OC₁₋₆ alkyl, —OC₃₋₆ cycloalkyl, or —CN;
R^{A} and R^{B} are each independently —H, —C₁-C₆ alkyl, cycloalkyl, or heterocyclyl;
U is H;
Z is —OR^{Z}, —C₁₋₆alkyl, —(CH₂)₀₋₃—(C₃₋₆ cycloalkyl), or —(CH₂)₀₋₃—(C₃-C₄ heterocyclyl), and wherein the —C₁₋₆alkyl, —C₃₋₆ cycloalkyl or —C₃-C₄ heterocyclyl is optionally substituted with 1 or more substituents each independently selected from the group consisting of -halo, —C₁₋₆ alkyl, —C₁₋₆haloalkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.

In embodiments the present disclosure provides a compound of Formula (I):

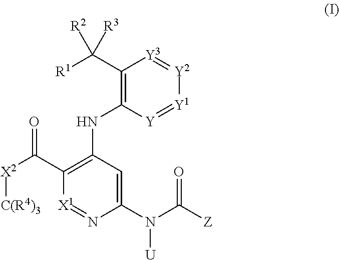

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z} or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl.

In embodiments the present disclosure provides a compound of Formula (I):

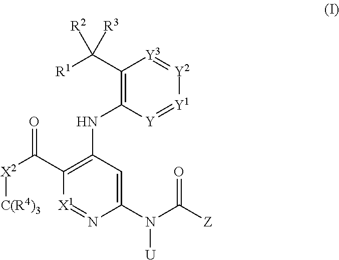

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z} or —(CH₂)₀₋₃—(C₃₋₆ cycloalkyl), or —(CH₂)₀₋₃—(C₃₋₄ heterocyclyl), and wherein the —C₃₋₆ cycloalkyl is optionally substituted with 1 or more substituents each independently selected from the group consisting of -halo, —C₁₋₆ alkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl.

In embodiments the present disclosure provides a compound of Formula (I):

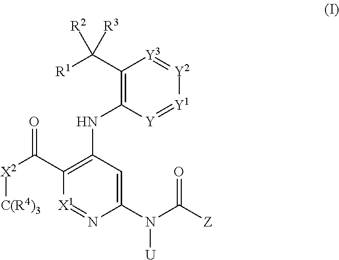

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl);
R² is —H, -halo, —C₁₋₆ alkyl, —C₁₋₆ alkenyl, —C₁₋₆ alkynyl, —C₃₋₆ cycloalkyl, or heterocyclyl; or
R¹ and R² are taken together to form a carbocyclyl or a saturated heterocyclyl;
R³ is —H, -halo, —C₁₋₆ alkyl, —O—C₁₋₆ alkyl, or —CN;
each R⁴ is independently —H or -D;
X¹ is CH or N;
X² is CH₂ or NH;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z} or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl.

In embodiments the present disclosure provides a compound of Formula (I):

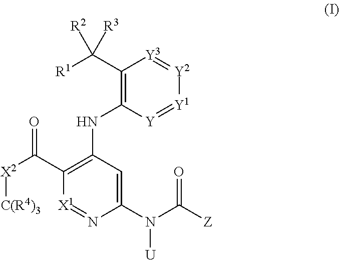

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z}, —NR⁹R¹⁰, or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl;
R⁹ and R¹⁰ are each independently —H or —C₁₋₆ alkyl; or
U and one of R⁹ or R¹⁰ are taken together to form a 5-7 membered heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.

In embodiments, the compound (e.g., of Formula (I) or (IB)) is not:

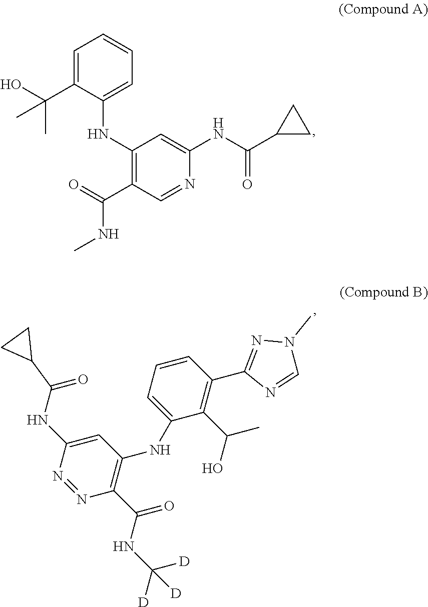 [SMILES: CNC(=O)c1cnc(NC(=O)C2CC2)cc1Nc1ccccc1C(C)(C)O ; [2H]C([2H])([2H])NC(=O)c1nnc(NC(=O)C2CC2)cc1Nc1cccc(-c2ncn(C)n2)c1C(C)O]

or a pharmaceutically acceptable salt, deuterated form, or stereoisomer thereof. In embodiments, the compound of Formula (I) or (IB) is not Compound A, Compound B or a pharmaceutically acceptable salt or deuterated form thereof. In embodiments, the compound of Formula (I) or (IB) is not Compound A, Compound B, or a pharmaceutically acceptable salt thereof. In embodiments, the compound of Formula (I) or (IB) is not Compound A or Compound B.

In embodiments of the compounds of Formula (I), Z is —OR^{Z} or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl.

In embodiments of the compounds of Formula (I), Z is —OR^{Z}, —C₃₋₆ cycloalkyl, or —C₃-C₄heterocyclyl, wherein the —C₃₋₆ cycloalkyl or —C₃₋₄ heterocyclyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl.

In embodiments of the compounds of Formula (I), Z is —OR^{Z} or —C₃₋₆ cycloalkyl, wherein the —C₃₋₆ cycloalkyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents are taken together to form a carbocyclyl.

In embodiments of the compounds of Formula (I), Z is —OR^{Z} or cyclopropyl, wherein the cyclopropyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents are taken together to form a carbocyclyl.

In embodiments of the compounds of Formula (I), Z is —OR^{Z} or cyclopropyl, cyclobutyl, bicyclopentyl, or spirohexyl, or oxetanyl, wherein the cyclopropyl, cyclobutyl, bicyclopentyl, or spirohexyl, or oxetanyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN, or two substituents are taken together to form a carbocyclyl.

In embodiments of the compounds of Formula (I), the bicyclyopentyl is bicyclo[1.1.1]pentane. embodiments of the compounds of Formula (I), the spirohexyl is spiro[2.3]hexyl.

In embodiments of the compounds of Formula (I), Z is —OR^{Z}, —NR⁹R¹⁰, or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl.

In embodiments of the compounds of Formula (I), Z is —OR^{Z} or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents on the same atom are taken together to form a C₃₋₆ cycloalkyl.

In embodiments of the compounds of Formula (I), Z is —OR^{Z} or —C₃₋₆ cycloalkyl, wherein the —C₃₋₆ cycloalkyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents on the same atom are taken together to form a —C₃₋₆ cycloalkyl.

In embodiments of the compounds of Formula (I), Z is —OR^{Z} or cyclopropyl, wherein the cyclopropyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents on the same atom are taken together to form a —C₃₋₆ cycloalkyl.

In embodiments of the compounds of Formula (I), Z is —OR^{Z}, cyclopropyl, cyclobutyl, bicyclo[1.1.1]pentane, spiro[2.3]hexane, or oxetanyl, wherein the cyclopropyl, cyclobutyl, bicyclo[1.1.1]pentane, or spiro[2.3]hexane, or oxetanyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN, or two substituents on the same atom are taken together to form a —C₃₋₆ cycloalkyl.

In embodiments of the compounds of Formula (I), Z is —OR^{Z} or cyclopropyl, wherein the cyclopropyl is optionally substituted with 1, 2, or 3 substituents each independently selected from the group consisting of -halo, —C₁₋₆ alkyl, —OC₁₋₆ alkyl, or —OC₃₋₆ cycloalkyl.

In embodiments of the compounds of Formula (I), Z is —OR^{Z}.

In embodiments of the compounds of Formula (I), Z is —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl.

In embodiments of the compounds of Formula (I), Z is —C₃₋₆ cycloalkyl, wherein the —C₃₋₆ cycloalkyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents are taken together to form a carbocyclyl.

In embodiments of the compounds of Formula (I), Z is —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents on the same atom are taken together to form a —C₃₋₆ cycloalkyl.

In embodiments of the compounds of Formula (I), Z is —C₃₋₆ cycloalkyl, wherein the —C₃₋₆ cycloalkyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents on the same atom are taken together to form a —C₃₋₆ cycloalkyl.

In embodiments of the compounds of Formula (I), Z is cyclopropyl, cyclobutyl, bicyclo[1.1.1]pentane, or spiro[2.3]hexane, or oxetanyl, wherein the cyclopropyl, cyclobutyl, bicyclo[1.1.1]pentane, or spiro[2.3]hexane, or oxetanyl is optionally substituted with 1, 2, or 3 substituents each independently selected from the group consisting of -halo, —C₁₋₆ alkyl, —OC₁₋₆ alkyl, or —OC₃₋₆ cycloalkyl.

In embodiments of the compounds of Formula (I), Z is cyclopropyl, cyclobutyl, bicyclo[1.1.1]pentane, or spiro[2.3]hexane, or oxetanyl, wherein the cyclopropyl, cyclobutyl, bicyclo[1.1.1]pentane, or spiro[2.3]hexane, or oxetanyl is optionally substituted with 1, 2, or 3 substituents each independently selected from the group consisting of -halo, —C₁₋₆ alkyl, —OC₁₋₆ alkyl, or —OC₃₋₆ cycloalkyl.

In embodiments of the compounds of Formula (I), Z is unsubstituted cyclopropyl.

In embodiments of the compounds of Formula (I), U is H; or U and Z are taken together to form a heterocyclyl optionally substituted with 1 or more substituents independently selected from the group consisting of -halo, —C₁₋₆ alkyl, or —OC₁₋₆ alkyl.

In embodiments of the compounds of Formula (I), U is H.

In embodiments of the compounds of Formula (I), U and Z are taken together to form a heterocyclyl optionally substituted with 1 or more substituents independently selected from the group consisting of -halo, —C₁₋₆ alkyl, or —OC₁₋₆ alkyl.

In embodiments of the compounds of Formula (I), U and Z are taken together to form a 5-8 membered heterocyclyl, wherein the 5-8 membered heterocyclyl is optionally substituted with 1, 2, or 3 substituents independently selected from the group consisting of -halo, —C₁₋₆ alkyl, or —OC₁₋₆ alkyl.

In embodiments of the compounds of Formula (I), U and Z are taken together to form a 5-membered saturated heterocyclyl, wherein the 5-membered saturated heterocyclyl is optionally substituted with —C₁₋₆ alkyl.

In embodiments of the compounds of Formula (I), U and Z are taken together to form a 5-membered saturated heterocyclyl, wherein the 5-membered saturated heterocyclyl is optionally substituted with —C₁₋₃ alkyl.

In embodiments of the compounds of Formula (I), the 5-membered saturated heterocyclyl is optionally substituted with CH₃, CH₂CH₃, CH₂CH₂CH₃, or CH(CH₃)₂.

In embodiments of the compounds of Formula (I), Z is —NR⁹R¹⁰.

In embodiments of the compounds of Formula (I), Z is —NR⁹R¹⁰, wherein one of R⁹ and R¹⁰ is —H or —C₁₋₆ alkyl; and the other R⁹ or R¹⁰ is taken together with U to form a 5-7 membered heterocyclyl optionally substituted with 1 or more substituents independently selected from the group consisting of -halo, —C₁₋₆ alkyl, and —OC₁₋₆ alkyl.

In embodiments of the compounds of Formula (I), R⁹ and R¹⁰ are each independently —H or —C₁₋₆ alkyl or one of R⁹ and R¹⁰ is —H or —C₁₋₆ alkyl. and the other of R⁹ or R¹⁰ is taken together with U to form a 5-7 membered heterocyclyl optionally substituted with —C₁₋₆ alkyl.

In embodiments of the compounds of Formula (I), R⁹ is —H or —C₁₋₆ alkyl; and R¹⁰ and U are taken together to form a 5-7 membered heterocyclyl optionally substituted with 1 or more substituents independently selected from the group consisting of -halo, —C₁₋₆ alkyl, or —OC₁₋₆ alkyl.

In embodiments of the compounds of Formula (I), R⁹ is —H or —C₁₋₆ alkyl; and R¹⁰ and U are taken together to form a 5 membered heterocyclyl.

In embodiments, the compounds of Formula (I) is a compound having a structure of Formula (IA):

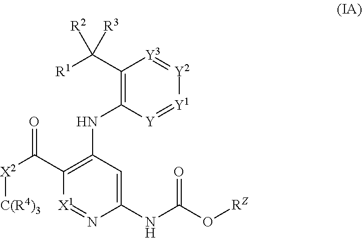

or a pharmaceutically acceptable salt, deuterated form, or stereoisomer thereof;
wherein:
   R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
   R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
   or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
   R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
   each R⁴ is independently —H or -D;
   X¹ is CH or N;
   X² is C(R⁸)₂ or NH;
   each R⁸ is independently H, halo, —C₁₋₆ alkyl, —C₁₋₆ haloalkyl, —O—C₁₋₆ alkyl, or —CN;
   Y is N or CR^{YA};
   Y¹ is N or CR^{YB};
   Y² is N or CR^{YC}; and
   Y³ is N or CR^{YD};
   R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl; and
   R^{Z} is —C₁₋₆ alkyl, or —C₃₋₆ cycloalkyl.

In embodiments, the compound of Formula (I) is a compound having a structure of Formula (IB):

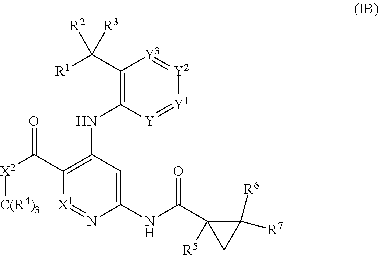

or a pharmaceutically acceptable salt, deuterated form, or stereoisomer thereof;
wherein:
   R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
   R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
   or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
   R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
   each R⁴ is independently —H or -D;
   X¹ is CH or N;
   X² is C(R⁸)₂ or NH;
   each R⁸ is independently H, halo, —C₁₋₆ alkyl, —C₁₋₆ haloalkyl, —O—C₁₋₆ alkyl, or —CN;
   Y is N or CR^{YA};
   Y¹ is N or CR^{YB};
   Y² is N or CR^{YC}; and
   Y³ is N or CR^{YD};
   R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl; and
   R⁵, R⁶ and R⁷ are each independently selected from the group consisting of —H, —CN, -halo, —C₁₋₆ alkyl, —C₁₋₆ haloalkyl, —OC₁₋₆ alkyl, or —OC₃₋₆ cycloalkyl, or R⁶ and R⁷ are taken together to form a carbocyclyl.

In embodiments of the compounds of Formula (IB), the compound is not:
3-pyridinecarboxamide, 6-[(cyclopropylcarbonyl)amino]-4-[[2-(1-hydroxy-1-methylethyl)phenyl]amino]-N-methyl- (shown below), or a pharmaceutically acceptable salt, deuterated form, or stereoisomer thereof

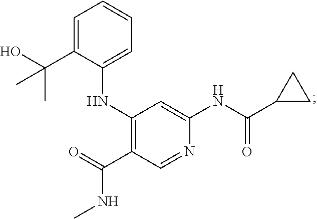 [SMILES: CNC(=O)c1cnc(NC(=O)C2CC2)cc1Nc1ccccc1C(C)(C)O]

6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide (shown below), or a pharmaceutically acceptable salt, deuterated form, or stereoisomer thereof

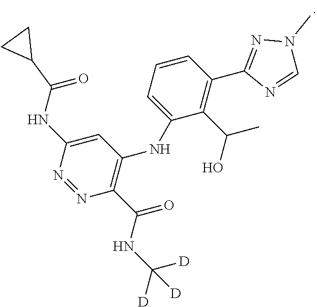 [SMILES: [2H]C([2H])([2H])NC(=O)c1nnc(NC(=O)C2CC2)cc1Nc1cccc(-c2ncn(C)n2)c1C(C)O]

In embodiments of the compounds of Formula (I), (IA), or (IB):
Y is N or CR^{YA};
Y¹ is N or CR^{YB};
Y² is N or CR^{YC}; and
Y³ is N or CR^{YD}.

In embodiments of the compounds of Formula (I), (IA), or (IB):
Y is N or CR^{YA};
Y¹ is N or CR^{YB};
Y² is N or CR^{YC}; and
Y³ is CR^{YD}.

In embodiments of the compounds of Formula (I), (IA), or (IB), Y, Y¹, and Y² are not all CH.

In embodiments of the compounds of Formula (I), (IA), or (IB), at least one of Y, Y¹, Y², and Y³ is N.

In embodiments of the compounds of Formula (I), (IA), or (IB), one or two of Y¹, Y², and Y³ is N.

In embodiments of the compounds of Formula (I), (IA), or (IB):
Y is N;
Y¹ is CR^{YB};
Y² is CR^{YC}; and
Y³ is CR^{YD}.

In embodiments of the compounds of Formula (I), (IA), or (IB), Y is N. In embodiments of the compounds of Formula (I), (IA), or (IB), Y is CR^{YA}.

In embodiments of the compounds of Formula (I), (IA), or (IB), Y¹ is N. In embodiments of the compounds of Formula (I), (IA), or (IB), Y¹ is CR^{YB}.

In embodiments of the compounds of Formula (I), (IA), or (IB), Y² is N. In embodiments of the compounds of Formula (I), (IA), or (IB), Y² is CR^{YC}.

In embodiments of the compounds of Formula (I), (IA), or (IB), Y³ is N. In embodiments of the compounds of Formula (I), (IA), or (IB), Y³ is CR^{YD}.

In embodiments of the compounds of Formula (I), (IA), or (IB), Y is N and Y² is N.

In embodiments of the compounds of Formula (I), (IA), or (IB), Y is N and Y³ is N.

In embodiments of the compounds of Formula (I), (IA), or (IB), Y is N and Y² is CR^{YC} wherein R^{YC} is CH₃, —CN, F, or Cl; or wherein Y is N and Y³ is CR^{YD} wherein R^{YD} is CH₃, —CN, F, or Cl

In embodiments of the compounds of Formula (I) or (IA), the compound is of Formula (IIA):

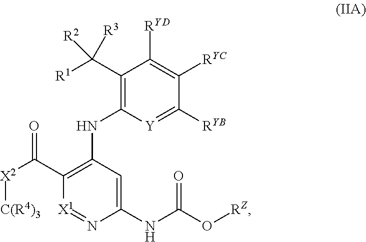

or a pharmaceutically acceptable salt, deuterated form, or stereoisomer thereof;
wherein:
   R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
   R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
   or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
   R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
   each R⁴ is independently —H or -D;
   X¹ is CH or N;
   X² is C(R⁸)₂ or NH;
   each R⁸ is independently H, halo, —C₁₋₆ alkyl, —C₁₋₆ haloalkyl, —O—C₁₋₆ alkyl, or —CN;
   Y is N or CR^{YA};
   R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
   R^{Z} is —C₁₋₆ alkyl, or —C₃₋₆ cycloalkyl.

In embodiments of the compounds of Formula (IIA), Y is N.

In embodiments of the compounds of Formula (I) or (IB), the compound is of Formula (IIB):

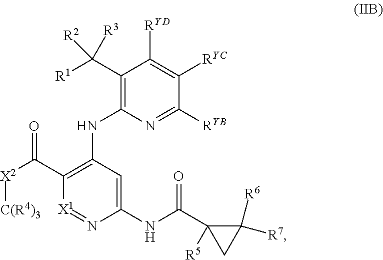

or a pharmaceutically acceptable salt, deuterated form, or stereoisomer thereof;
wherein:
   R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
   R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
   or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
   R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
   each R⁴ is independently —H or -D;
   X¹ is CH or N;
   X² is C(R⁸)₂ or NH;
   each R⁸ is independently H, halo, —C₁₋₆ alkyl, —C₁₋₆ haloalkyl, —O—C₁₋₆ alkyl, or —CN;
   R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl; and
   R⁵, R⁶ and R⁷ are each independently selected from the group consisting of —H, —CN, -halo, —C₁₋₆ alkyl, —C₁₋₆ haloalkyl, —OC₁₋₆ alkyl, or —OC₃₋₆ cycloalkyl, or R⁶ and R⁷ are taken together to form a carbocyclyl.

In embodiments of the compounds of Formula (I), (IA), or (IB), the compound is of Formula (IIA) or (IIB):

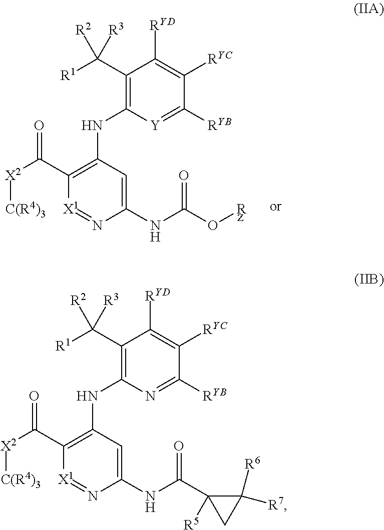

or a pharmaceutically acceptable salt, deuterated form, or stereoisomer thereof.

In embodiments of the compounds of Formula (I), (IA), or (IIA), R^{Z} is —C₁₋₆ alkyl, or —C₃₋₆ cycloalkyl.

In embodiments of the compounds of Formula (I), (IA), or (IIA), R^{Z} is —C₁₋₆ alkyl.

In embodiments of the compounds of Formula (I), (IA), or (IIA), R^{Z} is —CH₃.

In embodiments of the compounds of Formula (I), (IA), or (IIA), R^{Z} is —C₃₋₆ cycloalkyl.

In embodiments of the compounds of Formula (IB) or (IIB), R⁵, R⁶ and R⁷ are each independently selected from the group consisting of —H, -halo, —C₁₋₆ alkyl, —OC₁₋₆ alkyl, or —OC₃₋₆ cycloalkyl, or R⁶ and R⁷ are taken together to form a carbocyclyl.

In embodiments of the compounds of Formula (IB) or (IIB), R⁵, R⁶ and R⁷ are each independently selected from the group consisting of —H, -halo, —C₁₋₆ alkyl, —OC₁₋₆ alkyl, or —OC₃₋₆ cycloalkyl, or R⁶ and R⁷ are taken together to form a C₃₋₆cycloalkyl.

In embodiments of the compounds of Formula (IB) or (IIB), R⁵, R⁶ and R⁷ are each independently selected from the group consisting of —H, -halo, —CN, —C₁₋₆ alkyl, —C₁₋₆ haloalkyl, —OC₁₋₆ alkyl, or —OC₃₋₆ cycloalkyl, or R⁶ and R⁷ are taken together to form a carbocyclyl.

In embodiments of the compounds of Formula (IB) or (IIB), R⁶ and R⁷ are each independently selected from the group consisting of —H, -halo, —CN, —C₁₋₆ alkyl, or —C₁₋₆ haloalkyl, or R⁶ and R⁷ are taken together to form a C₃₋₆cycloalkyl.

In embodiments of the compounds of Formula (IB) or (IIB), R⁵ is —H, —C₁₋₆ alkyl, —CN, or halo.

In embodiments of the compounds of Formula (IB) or (IIB), R⁵ is —H or halo.

In embodiments of the compounds of Formula (IB) or (IIB), R⁶ and R⁷ are each independently selected from the group consisting of —H, -halo, —C₁₋₆ alkyl, or —OC₁₋₆ alkyl, or R⁶ and R⁷ are taken together to form a C₃₋₆carbocyclyl.

In embodiments of the compounds of Formula (IB) or (IIB), R⁶ and R⁷ are each independently selected from the group consisting of —H, -halo, —C₁₋₆ alkyl, or —OC₁₋₆ alkyl, or R⁶ and R⁷ are taken together to form a C₃₋₆cycloalkyl.

In embodiments of the compounds of Formula (IB) or (IIB), R⁶ and R⁷ are each independently selected from the group consisting of -halo, —C₁₋₆ alkyl, or —OC₁₋₆ alkyl, or R⁶ and R⁷ are taken together to form a C₃₋₆carbocyclyl.

In embodiments of the compounds of Formula (IB) or (IIB), R⁶ and R⁷ are each independently selected from the group consisting of -halo, —C₁₋₆ alkyl, or —OC₁₋₆ alkyl, or R⁶ and R⁷ are taken together to form a C₃₋₆cycloalkyl.

In embodiments of the compounds of Formula (IB) or (IIB), R⁶ and R⁷ are each independently selected from the group consisting of —H, -halo, —C₁₋₆ alkyl, or —OC₁₋₆ alkyl, or R⁶ and R⁷ are taken together to form a cyclopropyl or cyclobutyl.

In embodiments of the compounds of Formula (IB) or (IIB), R⁶ and R⁷ are each independently selected from the group consisting of —H, -halo, —C₁₋₆ alkyl, or —OC₁₋₆ alkyl.

In embodiments of the compounds of Formula (IB) or (IIB), R⁵, R⁶ and R⁷ are H.

In embodiments of the compounds of Formula (I), (IA), (IB), or (IIA), R^{YA}, R^{YB}, and R^{YC} are each independently —H, —CN, -halo, —N(C₁₋₆ alkyl)(saturated heterocyclyl), —C₁₋₆ alkyl, —C₁₋₆ alkenyl, —C₁₋₆ alkynyl, —OC₁₋₆ alkyl, —C₃₋₆ cycloalkyl, saturated heterocyclyl, aryl, or heteroaryl, wherein the —C₁₋₆ alkyl, —C₁₋₆ alkenyl, —C₁₋₆ alkynyl, —OC₁₋₆ alkyl, —C₃₋₆ cycloalkyl, saturated heterocyclyl, aryl, or heteroaryl are optionally substituted with —F, —C₁₋₆ alkyl, or —OC₁₋₆ alkyl and R^{YD} is —H, —CN, -halo, —C₁₋₆ alkyl, —C₁₋₆ alkenyl, —C₁₋₆ alkynyl, —C₃₋₆ cycloalkyl, a saturated heterocyclyl, or aryl.

In embodiments of the compounds of Formula (I), (IA), (IB), or (IIA), R^{YB}, R^{YC}, and R^{YD} are each independently —H, —N(C₁₋₆ alkyl)(saturated heterocyclyl), —C₁₋₆ alkyl, —OC₁₋₆ alkyl, —CN, halo, saturated heterocyclyl, or heteroaryl, wherein the —C₁₋₆ alkyl, —OC₁₋₆ alkyl, saturated heterocyclyl, or heteroaryl are optionally substituted with —F, —C₁₋₆ alkyl, or —OC₁₋₆ alkyl.

In embodiments of the compounds of Formula (I), (IA), (IB), or (IIA), R^{YB} and R^{YD} are —H, —F —CN, or —CH₃ and R^{YC} is —H, —CN, —F, —OCHF₂, —OCF₃, —OCH₃, —CH₃, —Cl;

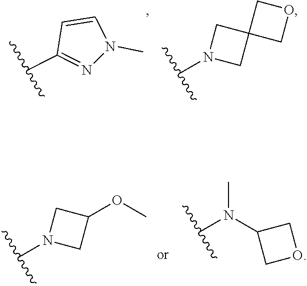

In embodiments of the compounds of Formula (I), (IA), (IB), or (IIA), R^{YB} and R^{YD} are —H, —F —CN, or —CH₃ and R^{YC} is —H, —CN, —F, —OCHF₂, —OCF₃, —OCH₃, —CH₃, or —C₁.

In embodiments of the compounds of Formula (I), (IA), (IB), or (IIA), R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl.

In embodiments of the compounds of Formula (I), (IA), (IB), or (IIA), R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, saturated heterocyclyl, aryl, or heteroaryl.

In embodiments of the compounds of Formula (I), (IA), (IB), or (IIA), R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, saturated heterocyclyl, or aryl.

In embodiments of the compounds of Formula (I), (IA), (IB), or (IIA), R^{YA}, R^{YB}, and R^{YC} are each independently —H, —CN, -halo, —C₁₋₆ alkyl, —C₁₋₆ alkenyl, —C₁₋₆ alkynyl, —C₃₋₆ cycloalkyl, saturated heterocyclyl, aryl, or heteroaryl and R^{YD} is —H, —CN, -halo, —C₁₋₆ alkyl, —C₁₋₆ alkenyl, —C₁₋₆ alkynyl, —C₃₋₆ cycloalkyl, a saturated heterocyclyl, or aryl.

In embodiments of the compounds of Formula (IIB), R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl.

In embodiments of the compounds of Formula (IIB), R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, saturated heterocyclyl, aryl, or heteroaryl.

In embodiments of the compounds of Formula (IIB), R^{YB}, and R^{YC} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, saturated heterocyclyl, aryl, or heteroaryl and R^{YD} is —H, —CN, -halo, —C₁₋₆ alkyl, —C₁₋₆ alkenyl, —C₁₋₆ alkynyl, —C₃₋₆ cycloalkyl, a saturated heterocyclyl, or aryl.

In embodiments of the compounds of Formula (I), (IA), (IB), (IIA), or (IIB), R^{YB}, R^{YC}, and R^{YD} are each independently —H, —C₁₋₆ alkyl, —OC₁₋₆ alkyl, —CN, or halo.

In embodiments of the compounds of Formula (I), (IA), (IB), (IIA), or (IIB), R^{YB} and R^{YD} are —H and R^{YC} is —H, —C₁₋₆ alkyl, —OC₁₋₆ alkyl, —CN, or —F.

In embodiments of the compounds of Formula (I), (IA), (IB), (IIA), or (IIB), R^{YB}, R^{YC}, and R^{YD} are —H.

In embodiments of the compounds of Formula (I), (IA), (IB), (IIA), or (IIB), two or three of R^{YB}, R^{YC}, and R^{YD} are —H.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); or
R¹ and R² are taken together to form a carbocyclyl or saturated or partially unsaturated heterocyclyl.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); or
R¹ and R² are taken together to form a C₃₋₆ cycloalkyl or saturated or partially unsaturated heterocyclyl.

In embodiments of the compounds of Formula (I), (IA), (IB), (IIA), or (IIB), at least one of R¹, R², and R³ is not —H. In embodiments of the compounds of Formula (I), (IA), (IB), (IIA), or (IIB), at least one of R¹, R², and R³ is -halo, —C₁₋₆ alkyl, —O—C₁₋₆ alkyl, or —CN.

In embodiments of the compounds of Formula (I), (IA), (IB), (IIA), or (IIB), R¹, R², and R³ is not —OH. In embodiments of the compounds of Formula (I), (IA), (IB), (IIA), or (IIB), when two of R¹, R², and R³ are —C₁₋₆ alkyl, the other is not —OH.

In embodiments of the compounds of Formula (I), (IA), (IB), (IIA), or (IIB), when R, and R² are taken together to form a cyclopropyl, R³ is not H.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl; or
R¹ and R² are taken together to form a carbocyclyl or saturated or partially unsaturated heterocyclyl, wherein the carbocyclyl or saturated or partially unsaturated heterocyclyl are optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or -CO₂alkyl.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl; or
R¹ and R² are taken together to form a C₃₋₆ cycloalkyl or saturated or partially unsaturated heterocyclyl, wherein the C₃₋₆ cycloalkyl or saturated or partially unsaturated heterocyclyl are optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or -CO₂alkyl.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more -halo, —CN, —OC₁₋₆ alkyl, —OC₁₋₆ haloalkyl, or —CO₂alkyl.

In embodiments of the compounds of Formula (I), (IA), (IB), (IIA), or (IIB), R¹ and R² are taken together to form a carbocyclyl or saturated or partially unsaturated heterocyclyl, wherein the carbocyclyl or saturated or partially unsaturated heterocyclyl are each independently optionally substituted with 1 or more -halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl.

In embodiments of the compounds of Formula (I), (IA), (IB), (IIA), or (IIB), R¹ and R² are taken together to form a C₃₋₆cycloalkyl or saturated or partially unsaturated heterocyclyl, wherein the C₃₋₆cycloalkyl or saturated or partially unsaturated heterocyclyl are each independently optionally substituted with 1 or more -halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl.

In embodiments of the compounds of Formula (I), (IA), (IB), (IIA), or (IIB),
R¹ is —C₁₋₆ alkyl or —C₃₋₆ cycloalkyl optionally substituted with 1 or more halo;
R² is —H, —C₁₋₆ alkyl, or -halo; or
R¹ and R² are taken together to form a carbocyclyl or saturated heterocyclyl, wherein the carbocyclyl or saturated heterocyclyl are optionally substituted with 1 or more halo.

In embodiments of the compounds of Formula (I), (IA), (IB), (IIA), or (IIB),
R¹ is —C₁₋₆ alkyl or —C₃₋₆ cycloalkyl optionally substituted with 1 or more halo;
R² is —C₁₋₆ alkyl or -halo; or
R¹ and R² are taken together to form a carbocyclyl or saturated heterocyclyl, wherein the carbocyclyl or saturated heterocyclyl are optionally substituted with 1 or more halo.

In embodiments of the compounds of Formula (I), (IA), (IB), (IIA), or (IIB):
R¹ is —H, -halo, —CN, —C₁₋₆ alkyl, —C₃₋₆ cycloalkyl, —OC₁₋₆ alkyl, —OC₃₋₆ cycloalkyl, or —O-saturated heterocyclyl;
R² is —H, -halo, —C₁₋₆ alkyl, —C₃₋₆ cycloalkyl, or a saturated heterocyclyl; or
R¹ and R² are taken together to form a carbocyclyl or saturated heterocyclyl.

In embodiments of the compounds of Formula (I), (IA), (IB), (IIA), or (IIB):
R¹ is —H, -halo, —CN, —C₁₋₆ alkyl, —C₃₋₆ cycloalkyl, —OC₁₋₆ alkyl, —OC₃₋₆ cycloalkyl, or —O-saturated heterocyclyl;
R² is —H, -halo, —C₁₋₆ alkyl, —C₃₋₆ cycloalkyl, or a saturated heterocyclyl; or
R¹ and R² are taken together to form a —C₃₋₆ cycloalkyl or saturated heterocyclyl.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O heterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more -halo, —CN, —OC₁₋₆ alkyl, —OC₁₋₆ haloalkyl, or —CO₂alkyl;
R² is —H, -halo, —C₁₋₆ alkyl, —C₁₋₆ alkenyl, —C₁₋₆ alkynyl, —C₃₋₆ cycloalkyl, or heterocyclyl; wherein the —C₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl; or
R¹ and R² are taken together to form a carbocyclyl or saturated or partially unsaturated heterocyclyl, wherein the carbocyclyl or saturated or partially unsaturated heterocyclyl are optionally substituted with 1 or more -halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or -CO₂alkyl.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl;
R² is —H, -halo, —C₁₋₆ alkyl, —C₁₋₆ alkenyl, —C₁₋₆ alkynyl, —C₃₋₆ cycloalkyl, or a saturated heterocyclyl; wherein the —C₁₋₆ alkyl, —C₁₋₆ alkenyl, —C₁₋₆ alkynyl, —C₃₋₆ cycloalkyl, or a saturated heterocyclyl are each independently optionally substituted with 1 or more halo, —CN, —OC₁₋₆ alkyl, —OC₁₋₆ haloalkyl, or —CO₂alkyl; or
R¹ and R² are taken together to form a carbocyclyl or saturated heterocyclyl, wherein the carbocyclyl or saturated heterocyclyl are optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or -CO₂alkyl.

In embodiments of the compounds of Formula (I), (IA), (IB), (IIA), or (IIB), R¹ is —H, -halo, —CN, —C₁₋₆ alkyl, —C₃₋₆ cycloalkyl, —OC₁₋₆ alkyl, —OC₃₋₆ cycloalkyl, or —O-saturated heterocyclyl; wherein the —C₁₋₆ alkyl, —C₃₋₆ cycloalkyl, —OC₁₋₆ alkyl, —OC₃₋₆ cycloalkyl, or —O-saturated heterocyclyl, are each independently optionally substituted with 1 or more halo, —CN, —OC₁₋₆ alkyl, —OC₁₋₆ haloalkyl, or —CO₂alkyl;
R² is —H, -halo, —C₁₋₆ alkyl, —C₃₋₆ cycloalkyl, or a saturated heterocyclyl; wherein the —C₁₋₆ alkyl, —C₃₋₆ cycloalkyl, or a saturated heterocyclyl are each independently optionally substituted with 1 or more halo, —CN, —OC₁₋₆ alkyl, —OC₁₋₆ haloalkyl, or —CO₂alkyl; or
R¹ and R² are taken together to form a carbocyclyl or saturated heterocyclyl, wherein the carbocyclyl or saturated heterocyclyl are optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or -CO₂alkyl.

In embodiments of the compounds of Formula (I), (IA), (IB), (IIA), or (IIB), R¹ is —H, -halo, —OH, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, or —O-saturated heterocyclyl;
R² is —H, -halo, —C₁₋₆ alkyl, —C₁₋₆ alkenyl, —C₁₋₆ alkynyl, —C₃₋₆ cycloalkyl, or saturated heterocyclyl; or
R¹ and R² are taken together to form a carbocyclyl or saturated heterocyclyl.

In embodiments of the compounds of Formula (I), (IA), (IB), (IIA), or (IIB),
R¹ is —H, -halo, —OH, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, or —O-saturated heterocyclyl;
R² is —H, -halo, —C₁₋₆ alkyl, —C₁₋₆ alkenyl, —C₁₋₆ alkynyl, —C₃₋₆ cycloalkyl, or saturated heterocyclyl; or
R¹ and R² are taken together to form a —C₃₋₆ cycloalkyl or saturated heterocyclyl.

In embodiments of the compounds of Formula (I), (IA), (IB), (IIA), or (IIB),
R¹ is —C₁₋₆ alkyl;
R² is —H or -halo; or
R¹ and R² are taken together to form a carbocyclyl or saturated heterocyclyl.

In embodiments of the compounds of Formula (I), (IA), (IB), (IIA), or (IIB),
R¹ is —C₁₋₆ alkyl;
R² is —H or -halo; or
R¹ and R² are taken together to form a —C₃₋₆ cycloalkyl or saturated heterocyclyl.

In embodiments of the compounds of Formula (I), (IA), (IB), (IIA), or (IIB),
R¹ is —C₁₋₆ alkyl;
R² is —H, —C₁₋₆ alkyl, or -halo; or
R¹ and R² are taken together to form a carbocyclyl or saturated heterocyclyl.

In embodiments of the compounds of Formula (I), (IA), (IB), (IIA), or (IIB),
R¹ is —C₁₋₆ alkyl;
R² is —H, —C₁₋₆ alkyl, or -halo; or
R¹ and R² are taken together to form a —C₃₋₆ cycloalkyl or saturated heterocyclyl.

In embodiments of the compounds of Formula (I), (IA), (IB), (IIA), or (IIB),
R¹ is —CH₃;
R² is —H, —CH₃, —F; or
R¹ and R² are taken together to form a cyclopropyl, spiropentyl, or oxetanyl.

In embodiments of the compounds of Formula (I), (IA), (IB), (IIA), or (IIB),
R¹ is —CH₃;
R² is —H or —F; or
R¹ and R² are taken together to form a cyclopropyl, spiropentyl, or oxetanyl.

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl).

In embodiments of the compounds of Formula (I), (IA), (IB), (IIA), or (IIB), R¹ is —H, -halo, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl.

In embodiments of the compounds of Formula (I), (IA), (IB), (IIA), or (IIB), R¹ is C₁₋₆ alkyl.

In embodiments of the compounds of Formula (I), (IA), (IB), (IIA), or (IIB), R¹ is —CH₃.

In embodiments of the compounds of Formula (I), (IA), (IB), (IIA), or (IIB), R¹ and R² are taken together to form a carbocyclyl or saturated or partially unsaturated heterocyclyl.

In embodiments of the compounds of Formula (I), (IA), (IB), (IIA), or (IIB), R¹ and R² are taken together to form a —C₃₋₆ cycloalkyl or saturated or partially unsaturated heterocyclyl.

In embodiments of the compounds of Formula (I), (IA), (IB), (IIA), or (IIB), R¹ and R² are taken together to form a —C₃₋₆ cycloalkyl or saturated 4-7 membered saturated heterocyclyl.

In embodiments of the compounds of Formula (I), (IA), (IB), (IIA), or (IIB), R¹ and R² are taken together to form a carbocyclyl or saturated heterocyclyl, wherein the carbocyclyl or saturated heterocyclyl are optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl.

In embodiments of Formula (I), (IA), (IB), (IIA), or (IIB), R¹ and R² are taken together to form a C₆₋₈ bicyclic cycloalkyl.

In embodiments of the compounds of Formula (I), (IA), (IB), (IIA), or (IIB), R¹ and R² are taken together to form a —C₃₋₆ cycloalkyl or saturated heterocyclyl, wherein the —C₃₋₆ cycloalkyl or saturated heterocyclyl are optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or -CO₂alkyl.

In embodiments of the compounds of Formula (I), (IA), (IB), (IIA), or (IIB), R¹ and R² are taken together to form a carbocyclyl.

In embodiments of the compounds of Formula (I), (IA), (IB), (IIA), or (IIB), R¹ and R² are taken together to form a saturated heterocyclyl.

In embodiments of the compounds of Formula (I), (IA), (IB), (IIA), or (IIB), R¹ and R² are taken together to form a —C₃₋₆ cycloalkyl.

In embodiments of the compounds of Formula (I), (IA), (IB), (IIA), or (IIB), R¹ and R² are taken together to form a cyclopropyl optionally substituted with 1 or 2 —F, oxetanyl, cyclobutyl,

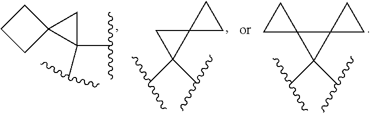

In embodiments of the compounds of Formula (I), (IA), (IB), (IIA), or (IIB), R¹ and R² are taken together to form a cyclopropyl or

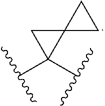 [SMILES: *C1(*)CC12CC2]

In embodiments of the compounds of Formula (I), (IA), (IB), (IIA), or (IIB), R¹ and R² are taken together to form a cyclopropyl or oxetanyl.

In embodiments of the compounds of Formula (I), (IA), (IB), (IIA), or (IIB), R¹ and R² are taken together to form a cyclopropyl.

In embodiments, the carbocycle or heterocycle formed by R¹ and R² is substituted with —C₁₋₆ alkyl-OC₁₋₆ alkyl. In embodiments, the carbocycle or heterocycle formed by R¹ and R² is substituted with —C₁₋₃ alkyl-OC₁₋₃ alkyl. In embodiments, the carbocycle or heterocycle formed by R¹ and R² is substituted with —CH₂OCH₃.

In embodiments, R¹ and R² are taken together to form a cyclopropyl, wherein the carbocycle is substituted by two substituents which come together to form a cyclopropyl or cyclobutyl ring.

In embodiments of the compounds of Formula (I), (IA), (IB), (IIA), or (IIB):
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; or
R¹ and R² are taken together to form a carbocyclyl or saturated or partially unsaturated heterocyclyl.

In embodiments of the compounds of Formula (I), (IA), (IB), (IIA), or (IIB), R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl; or
R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl.

In embodiments of the compounds of Formula (I), (IA), (IB), (IIA), or (IIB), R² is —H, -halo, —C₁₋₆ alkyl, —C₁₋₆ alkenyl, —C₁₋₆ alkynyl, —C₃₋₆ cycloalkyl, or a saturated heterocyclyl; wherein the —C₁₋₆ alkyl, —C₁₋₆ alkenyl, —C₁₋₆ alkynyl, —C₃₋₆ cycloalkyl, or a saturated heterocyclyl are each independently optionally substituted with 1 or more halo, —CN, —OC₁₋₆ alkyl, —OC₁₋₆ haloalkyl, or —CO₂alkyl; or
R¹ and R² are taken together to form a carbocyclyl or saturated heterocyclyl, wherein the carbocyclyl or saturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or -CO₂alkyl.

In embodiments of the compounds of Formula (I), (IA), (IB), (IIA), or (IIB), R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl.

In embodiments of the compounds of Formula (I), (IA), (IB), (IIA), or (IIB), R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl.

In embodiments of the compounds of Formula (I), (IA), (IB), (IIA), or (IIB), R² is —H, -halo, —C₁₋₆ alkyl, —C₁₋₆ alkenyl, —C₁₋₆ alkynyl, —C₃₋₆ cycloalkyl, or a saturated heterocyclyl; wherein the —C₁₋₆ alkyl, —C₁₋₆ alkenyl, —C₁₋₆ alkynyl, —C₃₋₆ cycloalkyl, or a saturated heterocyclyl are each independently optionally substituted with 1 or more halo, —CN, —OC₁₋₆ alkyl, —OC₁₋₆ haloalkyl, or —CO₂alkyl.

In embodiments of the compounds of Formula (I), (IA), (IB), (IIA), or (IIB), R² is —H, —C₁₋₆ alkyl or -halo.

In embodiments of the compounds of Formula (I), (IA), (IB), (IIA), or (IIB), R² is —C₁₋₆ alkyl or -halo.

In embodiments of the compounds of Formula (I), (IA), (IB), (IIA), or (IIB), R² is —H, —CH₃, or -halo.

In embodiments of the compounds of Formula (I), (IA), (IB), (IIA), or (IIB), R² is —H or -halo.

In embodiments of the compounds of Formula (I), (IA), (IB), (IIA), or (IIB), R² is —H.

In embodiments of the compounds of Formula (I), (IA), (IB), (IIA), or (IIB), R² is —C₁₋₆ alkyl.

In embodiments of the compounds of Formula (I), (IA), (IB), (IIA), or (IIB), R² is —CH₃.

In embodiments of the compounds of Formula (I), (IA), (IB), (IIA), or (IIB), R² is halo.

In embodiments of the compounds of Formula (I), (IA), (IB), (IIA), or (IIB), R² is F.

In embodiments of the compounds of Formula (I), (IA), (IB), (IIA), or (IIB), R³ is —H, -halo, —C₁₋₆ alkyl, or —CN; wherein the —C₁₋₆ alkyl is independently optionally substituted with 1 or more halo.

In embodiments of the compounds of Formula (I), (IA), (IB), (IIA), or (IIB), R³ is -halo, —C₁₋₆ alkyl, or —CN; wherein the —C₁₋₆ alkyl is independently optionally substituted with 1 or more halo.

In embodiments of the compounds of Formula (I), (IA), (IB), (IIA), or (IIB), R³ is -halo or —C₁₋₆ alkyl; wherein the —C₁₋₆ alkyl is independently optionally substituted with 1 or more halo.

In embodiments of the compounds of Formula (I), (IA), (IB), (IIA), or (IIB), R³ is —H, -halo, —C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or -CO₂alkyl.

In embodiments of the compounds of Formula (I), (IA), (IB), (IIA), or (IIB), R³ is —H, -halo, —C₁₋₆ alkyl, —C₁₋₆ haloalkyl, —O—C₁₋₆ alkyl, or CN, wherein the —C₁₋₆ alkyl is optionally substituted with 1 or more OC₁₋₆ alkyl.

In embodiments of the compounds of Formula (I), (IA), (IB), (IIA), or (IIB), R³ is —H, -halo, —C₁₋₆ alkyl, —C₁₋₆ haloalkyl, —O—C₁₋₆ alkyl, or CN.

In embodiments of the compounds of Formula (I), (IA), (IB), (IIA), or (IIB), R³ is —H, -halo, —C₁₋₆ haloalkyl, —O—C₁₋₆ alkyl, or CN.

In embodiments of the compounds of Formula (I), (IA), (IB), (IIA), or (IIB), R³ is -halo, —C₁₋₆ haloalkyl, —O—C₁₋₆ alkyl, or CN.

In embodiments of the compounds of Formula (I), (IA), (IB), (IIA), or (IIB), R³ is C₁₋₆ haloalkyl, —OC₁₋₆ alkyl, or CN.

In embodiments of the compounds of Formula (I), (IA), (IB), (IIA), or (IIB), R³ is C₁₋₃ haloalkyl, —OC₁₋₃ alkyl, or CN.

In embodiments of the compounds of Formula (I), (IA), (IB), (IIA), or (IIB), R³ is halo, —C₁₋₆ alkyl, —C₁₋₆ haloalkyl or CN.

In embodiments of the compounds of Formula (I), (IA), (IB), (IIA), or (IIB), R³ is —H, —F, —Cl, —CH₃, —CH₂F, —CHF₂, —CF₃, —OCH₃, —OCH₂CH₃, —CH₂CH₃, —CH₂OCH₃ or CN.

In embodiments of the compounds of Formula (I), (IA), (IB), (IIA), or (IIB), R³ is —H, —F, —CH₃, —CF₃, —OCH₃, —OCH₂CH₃, or CN.

In embodiments of the compounds of Formula (I), (IA), (IB), (IIA), or (IIB), R³ is —F, —CH₃, —CF₃, —OCH₃, —OCH₂CH₃, or CN.

In embodiments of the compounds of Formula (I), (IA), (IB), (IIA), or (IIB), —CH₃, —CF₃, —OCH₃, —OCH₂CH₃, or CN.

In embodiments of the compounds of Formula (I), (IA), (IB), (IIA), or (IIB), R³ is —H or halo.

In embodiments, R³ is F or Cl. In embodiments, R³ is F.

In embodiments of the compounds of Formula (I), (IA), (IB), (IIA), or (IIB), R¹ and R² are taken together to form a carbocyclyl, and R³ is -halo, —C₁₋₆ alkyl, —C₁₋₆ haloalkyl, —O—C₁₋₆ alkyl, or CN. In embodiments of the compounds of Formula (I), (IA), (IB), (IIA), or (IIB), R¹ and R² are taken together to form a carbocyclyl, and R³ is -halo or —O—C₁₋₆ alkyl.

In embodiments of the compounds of Formula (I), (IA), (IB), (IIA), or (IIB), R¹ and R² are taken together to form a cyclopropyl, and R³ is —C₁₋₆ alkyl, —C₁₋₆ haloalkyl, —O—C₁₋₆ alkyl, or CN. In embodiments, R³ is —C₁₋₃ alkyl, —C₁₋₃ haloalkyl, —O—C₁₋₃ alkyl, or CN. In embodiments, R³ is —CH₃—CF₃, —O—CH₃, or CN.

In embodiments of the compounds of Formula (I), (IA), (IB), (IIA), or (IIB), X² is C(R⁸)₂, NH, or C(R⁸)₂NH.

In embodiments of the compounds of Formula (I), (IA), (IB), (IIA), or (IIB), X² is C(R⁸)₂ or NH.

In embodiments of the compounds of Formula (I), (IA), (IB), (IIA), or (IIB), each R⁸ is independently H, halo, —C₁₋₆ alkyl, —C₁₋₆ haloalkyl, —O—C₁₋₆ alkyl, or —CN.

In embodiments of the compounds of Formula (I), (IA), (IB), (IIA), or (IIB), X² is C(R⁸)₂.

In embodiments of the compounds of Formula (I), (IA), (IB), (IIA), or (IIB), X² is C(R⁸)₂NH.

In embodiments of the compounds of Formula (I), (IA), (IB), (IIA), or (IIB), X² is CHR⁸.

In embodiments of the compounds of Formula (I), (IA), (IB), (IIA), or (IIB), X² is CH₂.

In embodiments of the compounds of Formula (I), (IA), (IB), (IIA), or (IIB), X² is NH.

In embodiments of the compounds of Formula (I), (IA), (IB), (IIA), or (IIB), each R⁴ is independently —H or -D.

In embodiments of the compounds of Formula (I), (IA), (IB), (IIA), or (IIB), two R⁴ are H and one R⁴ is D.

In embodiments of the compounds of Formula (I), (IA), (IB), (IIA), or (IIB), two R⁴ are D and one R⁴ is H.

In embodiments of the compounds of Formula (I), (IA), (IB), (IIA), or (IIB), each R⁴ is —H.

In embodiments of the compounds of Formula (I), (IA), (IB), (IIA), or (IIB), each R⁴ is -D.

In embodiments of the compounds of Formula (I), (IA), (IB), (IIA), or (IIB), X¹ is CH or N.

In embodiments of the compounds of Formula (I), (IA), (IB), (IIA), or (IIB), X¹ is CH.

In embodiments of the compounds of Formula (I), (IA), (IB), (IIA), or (IIB), X¹ is N.

In embodiments of Formula (I), (IA), (IB), (IIA), or (IIB): R¹ and R² are taken together to form a carbocyclyl; and R³ is halo, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl). In embodiments, the carbocyclyl is cyclopropyl, spiropentyl, or oxetanyl. In embodiments, each R⁴ is D. In embodiments, X¹ is N. In embodiments, X² is NH. In embodiments, Y is N. In embodiments, Y¹ is CH. In embodiments, In embodiments, Y² is CH. In embodiments, Y³ is CH. In embodiments, Y¹, Y², Y³ are each CH. In embodiments, U is H. In embodiments, Z is cyclopropyl.

In embodiments of Formula (I), (IA), (IB), (IIA), or (IIB):
R¹ and R² are independently —C₁₋₃ alkyl or fluoro, or R¹ and R² are taken together to form a cyclopropyl;
R³ is —C₁₋₃ alkyl or fluoro, wherein the —C₁₋₃ alkyl is optionally substituted with 1 or more fluoro;
R⁴ is D;
X¹ is N;
X² is NH;
Y is N;
Y¹, Y², and Y³ are each CH;
U is H; and
Z is cyclopropyl.

In embodiments of Formula (I), (IA), (IB), (IIA), or (IIB):
R¹ and R² are independently —CH₃ or fluoro, or R¹ and R² are taken together to form a cyclopropyl;
R³ is —CH₃, —CF₃, or fluoro;
R⁴ is D;
X¹ is N;
X² is NH;
Y is N;
Y¹, Y², and Y³ are each CH;
U is H; and
Z is cyclopropyl.

In some embodiments, provided herein is one or more compounds selected from Table 1, or a pharmaceutically acceptable salt, deuterated form, or stereoisomer thereof,
wherein:

In some embodiments, provided herein is one or more compounds selected from Table 1, or a pharmaceutically acceptable salt or deuterated form thereof.

In some embodiments, provided herein is one or more compounds selected from Table 1, or a pharmaceutically acceptable salt thereof.

In some embodiments, provided herein is one or more compounds selected from Table 1.

TABLE 1
Compounds

| Cmpd No. | Structure |
|---|---|
| 1 | 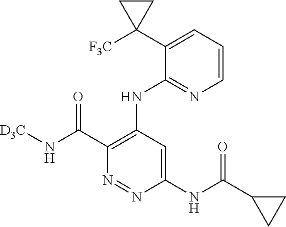 [SMILES: [2H]C([2H])([2H])NC(=O)c1nnc(NC(=O)C2CC2)cc1Nc1ncccc1C1(C(F)(F)F)CC1] |
| 2 | 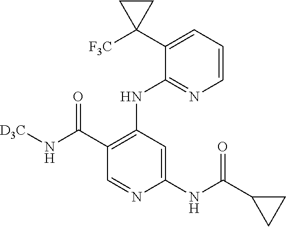 [SMILES: [2H]C([2H])([2H])NC(=O)c1cnc(NC(=O)C2CC2)cc1Nc1ncccc1C1(C(F)(F)F)CC1] |
| 3 | 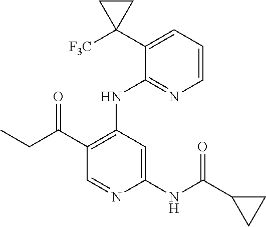 [SMILES: CCC(=O)c1cnc(NC(=O)C2CC2)cc1Nc1ncccc1C1(C(F)(F)F)CC1] |
| 4 | 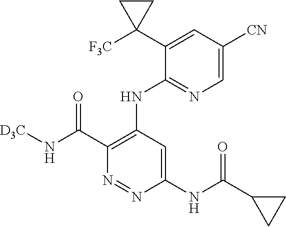 [SMILES: [2H]C([2H])([2H])NC(=O)c1nnc(NC(=O)C2CC2)cc1Nc1ncc(C#N)cc1C1(C(F)(F)F)CC1] |
| 5 | 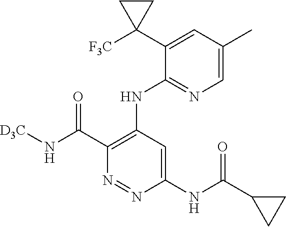 [SMILES: [2H]C([2H])([2H])NC(=O)c1nnc(NC(=O)C2CC2)cc1Nc1ncc(C)cc1C1(C(F)(F)F)CC1] |
| 6 | 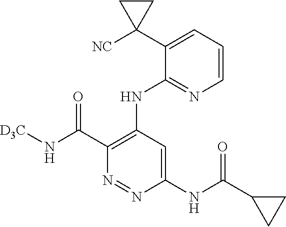 [SMILES: [2H]C([2H])([2H])NC(=O)c1nnc(NC(=O)C2CC2)cc1Nc1ncccc1C1(C#N)CC1] |
| 7 | 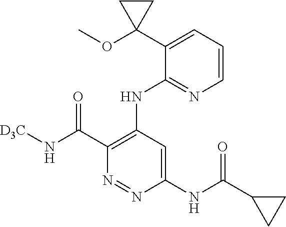 [SMILES: [2H]C([2H])([2H])NC(=O)c1nnc(NC(=O)C2CC2)cc1Nc1ncccc1C1(OC)CC1] |
| 8 | 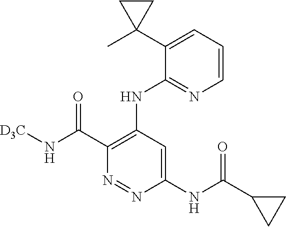 [SMILES: [2H]C([2H])([2H])NC(=O)c1nnc(NC(=O)C2CC2)cc1Nc1ncccc1C1(C)CC1] |
| 9 | 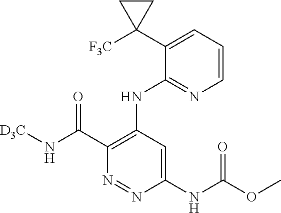 [SMILES: [2H]C([2H])([2H])NC(=O)c1nnc(NC(=O)OC)cc1Nc1ncccc1C1(C(F)(F)F)CC1] |
| 10 | 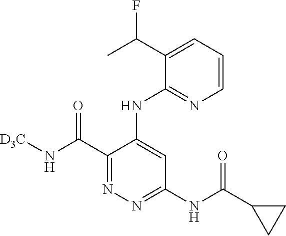 [SMILES: [2H]C([2H])([2H])NC(=O)c1nnc(NC(=O)C2CC2)cc1Nc1ncccc1C(C)F] |
| 11 | 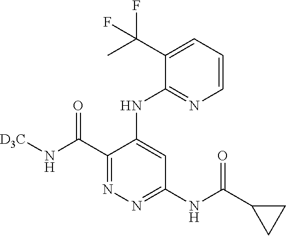 [SMILES: [2H]C([2H])([2H])NC(=O)c1nnc(NC(=O)C2CC2)cc1Nc1ncccc1C(C)(F)F] |
| 12 | 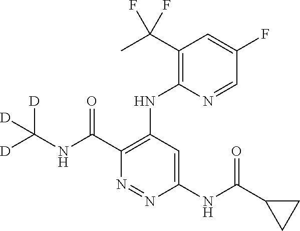 [SMILES: [2H]C([2H])([2H])NC(=O)c1nnc(NC(=O)C2CC2)cc1Nc1ncc(F)cc1C(C)(F)F] |
| 13 | 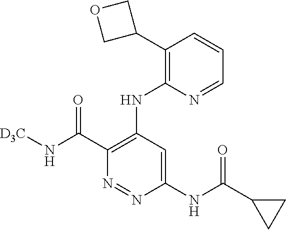 [SMILES: [2H]C([2H])([2H])NC(=O)c1nnc(NC(=O)C2CC2)cc1Nc1ncccc1C1COC1] |
| 14 | 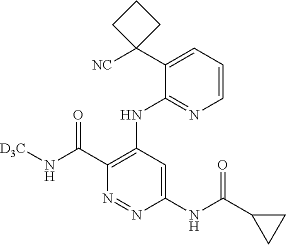 [SMILES: [2H]C([2H])([2H])NC(=O)c1nnc(NC(=O)C2CC2)cc1Nc1ncccc1C1(C#N)CCC1] |
| 15 | 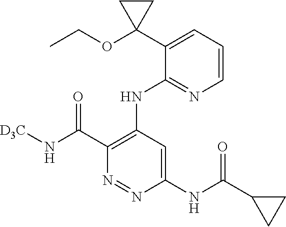 [SMILES: [2H]C([2H])([2H])NC(=O)c1nnc(NC(=O)C2CC2)cc1Nc1ncccc1C1(OCC)CC1] |
| 16 | 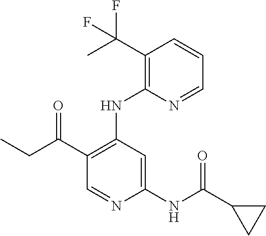 [SMILES: CCC(=O)c1cnc(NC(=O)C2CC2)cc1Nc1ncccc1C(C)(F)F] |
| 17 | 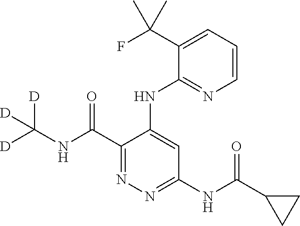 [SMILES: [2H]C([2H])([2H])NC(=O)c1nnc(NC(=O)C2CC2)cc1Nc1ncccc1C(C)(C)F] |
| 18 | 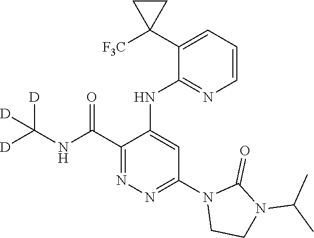 [SMILES: [2H]C([2H])([2H])NC(=O)c1nnc(N2CCN(C(C)C)C2=O)cc1Nc1ncccc1C1(C(F)(F)F)CC1] |
| 19 | 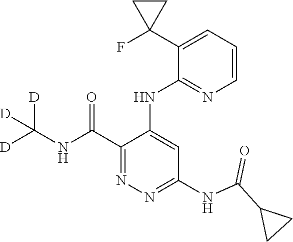 [SMILES: [2H]C([2H])([2H])NC(=O)c1nnc(NC(=O)C2CC2)cc1Nc1ncccc1C1(F)CC1] |
| 20 | 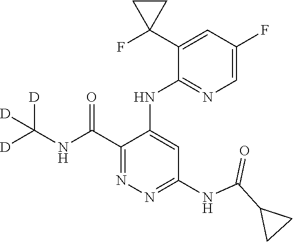 [SMILES: [2H]C([2H])([2H])NC(=O)c1nnc(NC(=O)C2CC2)cc1Nc1ncc(F)cc1C1(F)CC1] |
| 21 | 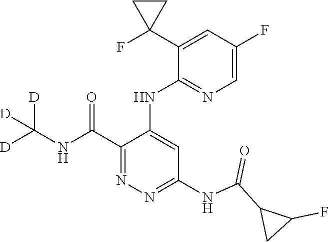 [SMILES: [2H]C([2H])([2H])NC(=O)c1nnc(NC(=O)C2CC2F)cc1Nc1ncc(F)cc1C1(F)CC1] |
| 21A | 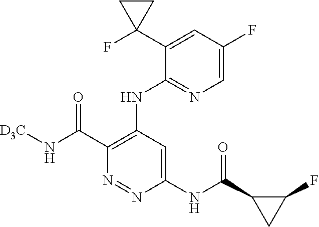 [SMILES: [2H]C([2H])([2H])NC(=O)c1nnc(NC(=O)[C@@H]2C[C@@H]2F)cc1Nc1ncc(F)cc1C1(F)CC1] |
| 22 | 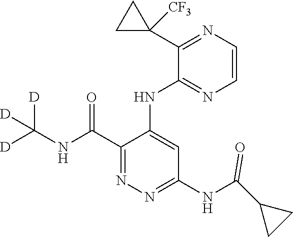 [SMILES: [2H]C([2H])([2H])NC(=O)c1nnc(NC(=O)C2CC2)cc1Nc1nccnc1C1(C(F)(F)F)CC1] |
| 23 | 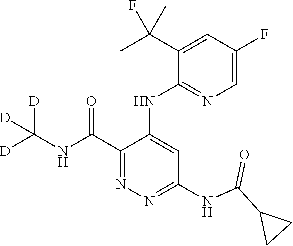 [SMILES: [2H]C([2H])([2H])NC(=O)c1nnc(NC(=O)C2CC2)cc1Nc1ncc(F)cc1C(C)(C)F] |
| 26 | 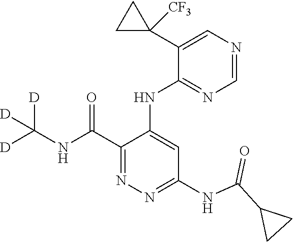 [SMILES: [2H]C([2H])([2H])NC(=O)c1nnc(NC(=O)C2CC2)cc1Nc1ncncc1C1(C(F)(F)F)CC1] |
| 27 | 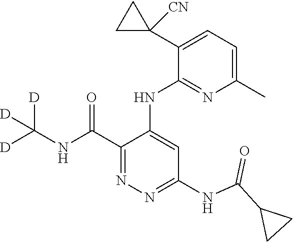 [SMILES: [2H]C([2H])([2H])NC(=O)c1nnc(NC(=O)C2CC2)cc1Nc1nc(C)ccc1C1(C#N)CC1] |
| 28 | 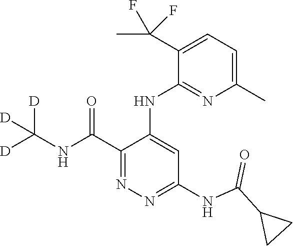 [SMILES: [2H]C([2H])([2H])NC(=O)c1nnc(NC(=O)C2CC2)cc1Nc1nc(C)ccc1C(C)(F)F] |
| 29 | 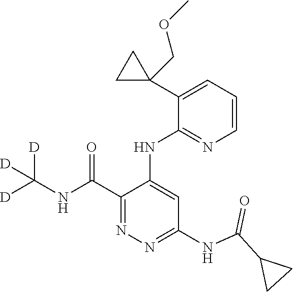 [SMILES: [2H]C([2H])([2H])NC(=O)c1nnc(NC(=O)C2CC2)cc1Nc1ncccc1C1(COC)CC1] |
| 30 | 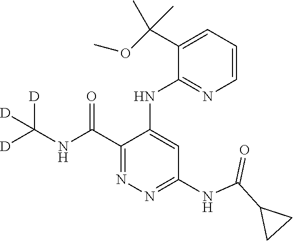 [SMILES: [2H]C([2H])([2H])NC(=O)c1nnc(NC(=O)C2CC2)cc1Nc1ncccc1C(C)(C)OC] |
| 31 | 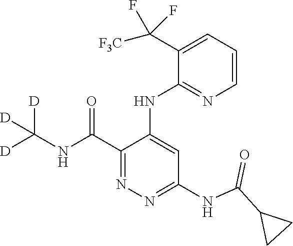 [SMILES: [2H]C([2H])([2H])NC(=O)c1nnc(NC(=O)C2CC2)cc1Nc1ncccc1C(F)(F)C(F)(F)F] |
| 32 | 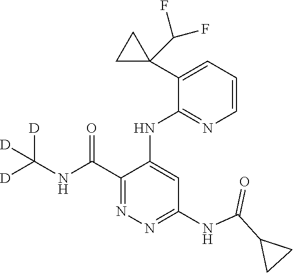 [SMILES: [2H]C([2H])([2H])NC(=O)c1nnc(NC(=O)C2CC2)cc1Nc1ncccc1C1(C(F)F)CC1] |
| 33 | 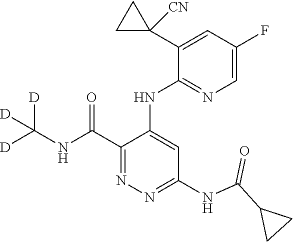 [SMILES: [2H]C([2H])([2H])NC(=O)c1nnc(NC(=O)C2CC2)cc1Nc1ncc(F)cc1C1(C#N)CC1] |
| 34 | 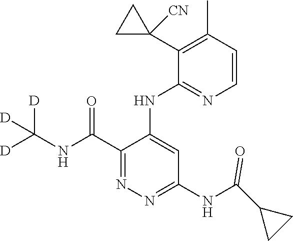 [SMILES: [2H]C([2H])([2H])NC(=O)c1nnc(NC(=O)C2CC2)cc1Nc1nccc(C)c1C1(C#N)CC1] |
| 35 | 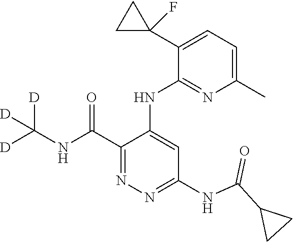 [SMILES: [2H]C([2H])([2H])NC(=O)c1nnc(NC(=O)C2CC2)cc1Nc1nc(C)ccc1C1(F)CC1] |
| 36 | 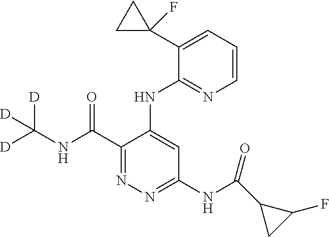 [SMILES: [2H]C([2H])([2H])NC(=O)c1nnc(NC(=O)C2CC2F)cc1Nc1ncccc1C1(F)CC1] |
| 36A | 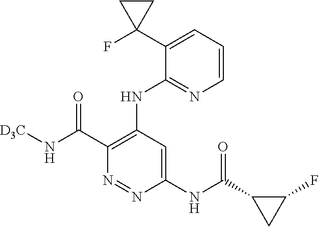 [SMILES: [2H]C([2H])([2H])NC(=O)c1nnc(NC(=O)[C@H]2C[C@H]2F)cc1Nc1ncccc1C1(F)CC1] |
| 37 | 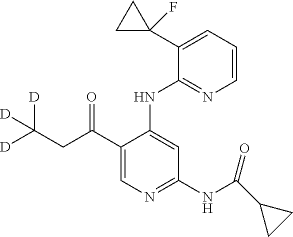 [SMILES: [2H]C([2H])([2H])CC(=O)c1cnc(NC(=O)C2CC2)cc1Nc1ncccc1C1(F)CC1] |
| 38 | 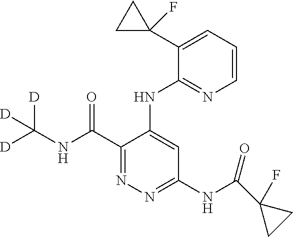 [SMILES: [2H]C([2H])([2H])NC(=O)c1nnc(NC(=O)C2(F)CC2)cc1Nc1ncccc1C1(F)CC1] |
| 39 | 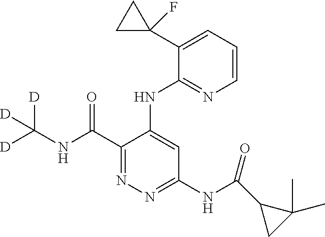 [SMILES: [2H]C([2H])([2H])NC(=O)c1nnc(NC(=O)C2CC2(C)C)cc1Nc1ncccc1C1(F)CC1] |
| 40 | 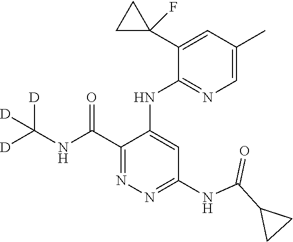 [SMILES: [2H]C([2H])([2H])NC(=O)c1nnc(NC(=O)C2CC2)cc1Nc1ncc(C)cc1C1(F)CC1] |
| 41 | 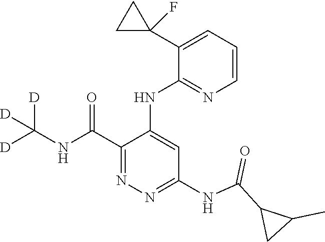 [SMILES: [2H]C([2H])([2H])NC(=O)c1nnc(NC(=O)C2CC2C)cc1Nc1ncccc1C1(F)CC1] |
| 41A | 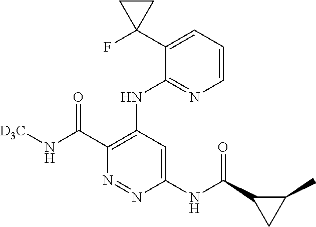 |
| 42 | 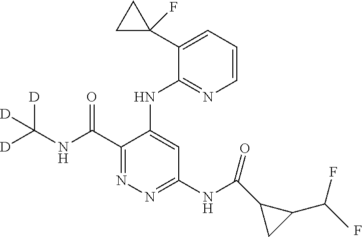 |
| 42A | 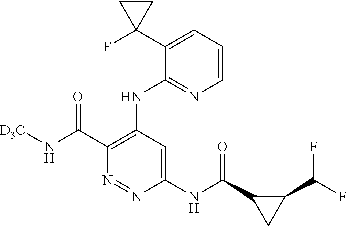 |
| 43 | 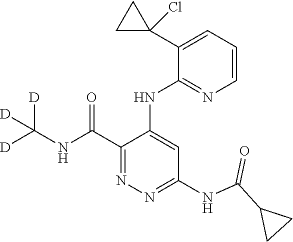 [SMILES: [2H]C([2H])([2H])NC(=O)c1nnc(NC(=O)C2CC2)cc1Nc1ncccc1C1(Cl)CC1] |
| 44 | 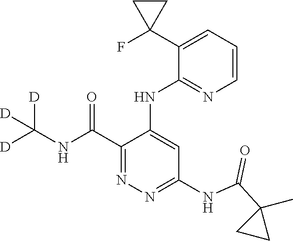 [SMILES: [2H]C([2H])([2H])NC(=O)c1nnc(NC(=O)C2(C)CC2)cc1Nc1ncccc1C1(F)CC1] |
| 45 | 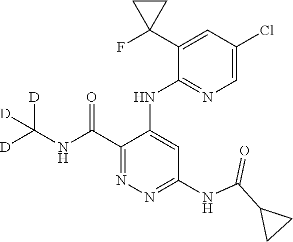 [SMILES: [2H]C([2H])([2H])NC(=O)c1nnc(NC(=O)C2CC2)cc1Nc1ncc(Cl)cc1C1(F)CC1] |
| 46 | 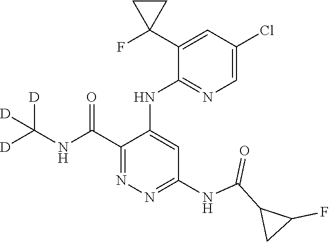 [SMILES: [2H]C([2H])([2H])NC(=O)c1nnc(NC(=O)C2CC2F)cc1Nc1ncc(Cl)cc1C1(F)CC1] |
| 46A | 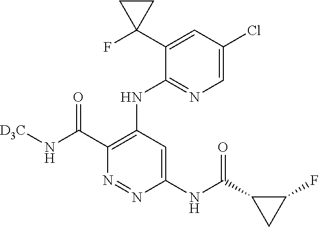 [SMILES: [2H]C([2H])([2H])NC(=O)c1nnc(NC(=O)[C@H]2C[C@H]2F)cc1Nc1ncc(Cl)cc1C1(F)CC1] |
| 47 | 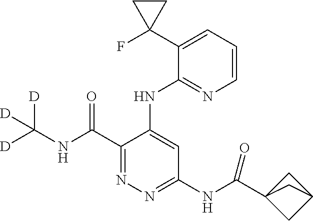 [SMILES: [2H]C([2H])([2H])NC(=O)c1nnc(NC(=O)C23CC(C2)C3)cc1Nc1ncccc1C1(F)CC1] |
| 48 | 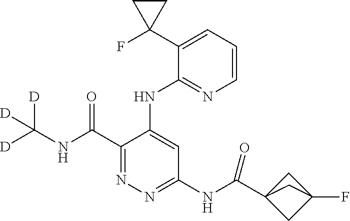 [SMILES: [2H]C([2H])([2H])NC(=O)c1nnc(NC(=O)C23CC(F)(C2)C3)cc1Nc1ncccc1C1(F)CC1] |
| 49 | 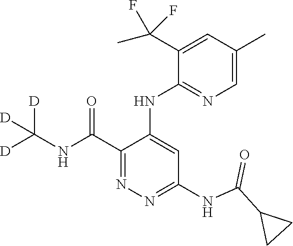 [SMILES: [2H]C([2H])([2H])NC(=O)c1nnc(NC(=O)C2CC2)cc1Nc1ncc(C)cc1C(C)(F)F] |
| 50 | 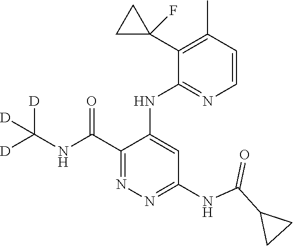 [SMILES: [2H]C([2H])([2H])NC(=O)c1nnc(NC(=O)C2CC2)cc1Nc1nccc(C)c1C1(F)CC1] |
| 51 | 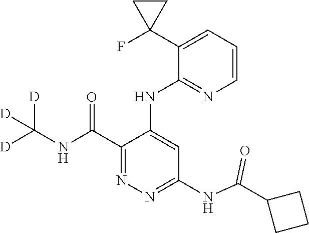 [SMILES: [2H]C([2H])([2H])NC(=O)c1nnc(NC(=O)C2CCC2)cc1Nc1ncccc1C1(F)CC1] |
| 52 | 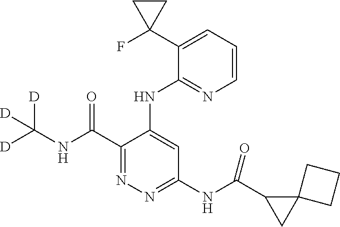 [SMILES: [2H]C([2H])([2H])NC(=O)c1nnc(NC(=O)C2CC23CCC3)cc1Nc1ncccc1C1(F)CC1] |
| 53 | 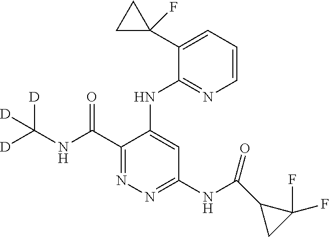 [SMILES: [2H]C([2H])([2H])NC(=O)c1nnc(NC(=O)C2CC2(F)F)cc1Nc1ncccc1C1(F)CC1] |
| 54 | 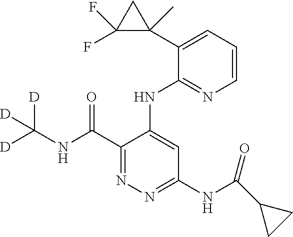 [SMILES: [2H]C([2H])([2H])NC(=O)c1nnc(NC(=O)C2CC2)cc1Nc1ncccc1C1(C)CC1(F)F] |
| 55 | 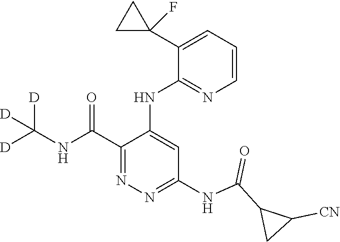 [SMILES: [2H]C([2H])([2H])NC(=O)c1nnc(NC(=O)C2CC2C#N)cc1Nc1ncccc1C1(F)CC1] |
| 55A | 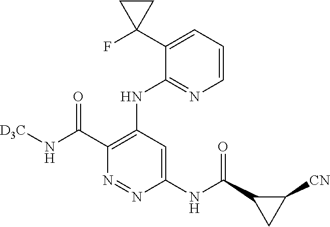 |
| 56 | 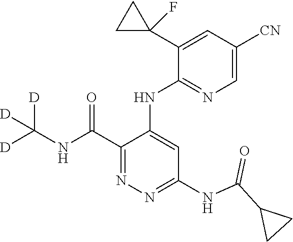 [SMILES: [2H]C([2H])([2H])NC(=O)c1nnc(NC(=O)C2CC2)cc1Nc1ncc(C#N)cc1C1(F)CC1] |
| 57 | 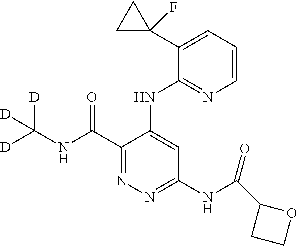 [SMILES: [2H]C([2H])([2H])NC(=O)c1nnc(NC(=O)C2CCO2)cc1Nc1ncccc1C1(F)CC1] |
| 58 | 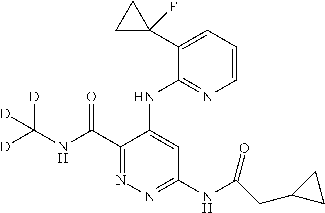 [SMILES: [2H]C([2H])([2H])NC(=O)c1nnc(NC(=O)CC2CC2)cc1Nc1ncccc1C1(F)CC1] |
| 59 | 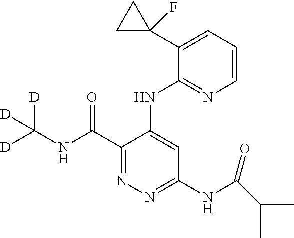 [SMILES: [2H]C([2H])([2H])NC(=O)c1nnc(NC(=O)C(C)C)cc1Nc1ncccc1C1(F)CC1] |
| 60 | 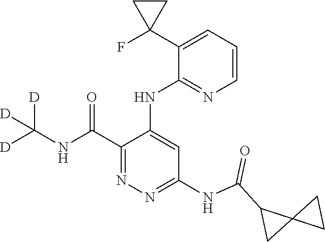 [SMILES: [2H]C([2H])([2H])NC(=O)c1nnc(NC(=O)C2CC23CC3)cc1Nc1ncccc1C1(F)CC1] |
| 60A | 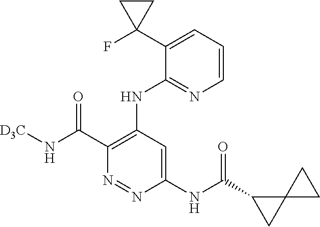 [SMILES: [2H]C([2H])([2H])NC(=O)c1nnc(NC(=O)[C@H]2CC23CC3)cc1Nc1ncccc1C1(F)CC1] |
| 60B | 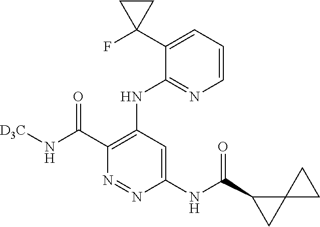 [SMILES: [2H]C([2H])([2H])NC(=O)c1nnc(NC(=O)[C@@H]2CC23CC3)cc1Nc1ncccc1C1(F)CC1] |
| 61 | 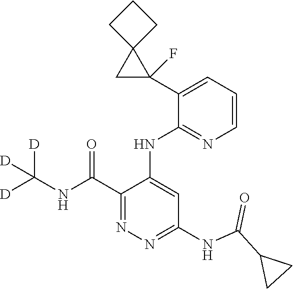 [SMILES: [2H]C([2H])([2H])NC(=O)c1nnc(NC(=O)C2CC2)cc1Nc1ncccc1C1(F)CC12CCC2] |
| 62 | 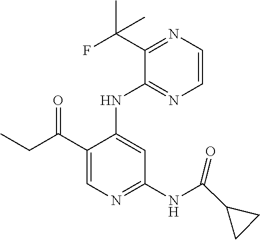 [SMILES: CCC(=O)c1cnc(NC(=O)C2CC2)cc1Nc1nccnc1C(C)(C)F] |
| 63 | 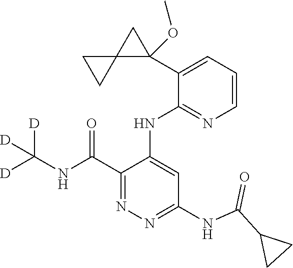 [SMILES: [2H]C([2H])([2H])NC(=O)c1nnc(NC(=O)C2CC2)cc1Nc1ncccc1C1(OC)CC12CC2] |
| 64 | 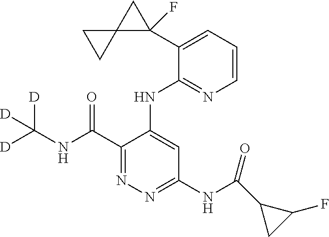 [SMILES: [2H]C([2H])([2H])NC(=O)c1nnc(NC(=O)C2CC2F)cc1Nc1ncccc1C1(F)CC12CC2] |
| 64A | 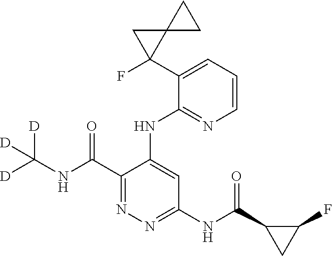 [SMILES: [2H]C([2H])([2H])NC(=O)c1nnc(NC(=O)[C@@H]2C[C@@H]2F)cc1Nc1ncccc1C1(F)CC12CC2] |
| 65 | 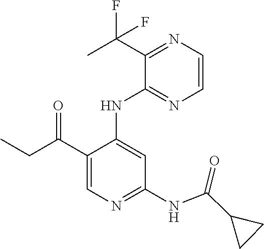 [SMILES: CCC(=O)c1cnc(NC(=O)C2CC2)cc1Nc1nccnc1C(C)(F)F] |
| 66 | 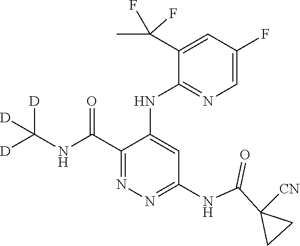 [SMILES: [2H]C([2H])([2H])NC(=O)c1nnc(NC(=O)C2(C#N)CC2)cc1Nc1ncc(F)cc1C(C)(F)F] |
| 67 | 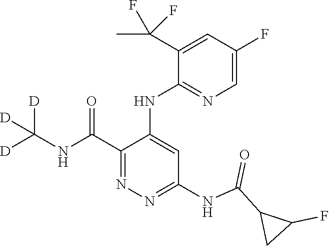 [SMILES: [2H]C([2H])([2H])NC(=O)c1nnc(NC(=O)C2CC2F)cc1Nc1ncc(F)cc1C(C)(F)F] |
| 67A | 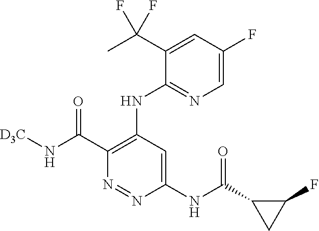 [SMILES: [2H]C([2H])([2H])NC(=O)c1nnc(NC(=O)[C@H]2C[C@@H]2F)cc1Nc1ncc(F)cc1C(C)(F)F] |
| 68 | 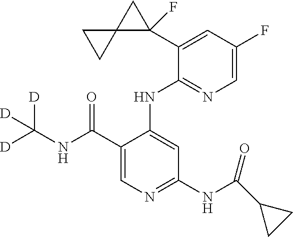 [SMILES: [2H]C([2H])([2H])NC(=O)c1cnc(NC(=O)C2CC2)cc1Nc1ncc(F)cc1C1(F)CC12CC2] |
| 69 | 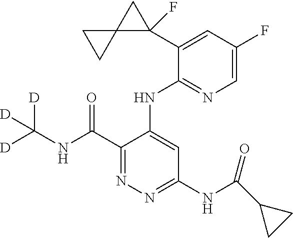 [SMILES: [2H]C([2H])([2H])NC(=O)c1nnc(NC(=O)C2CC2)cc1Nc1ncc(F)cc1C1(F)CC12CC2] |
| 70 | 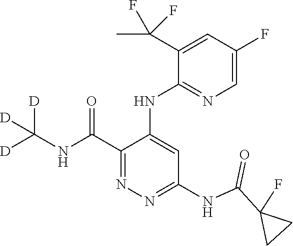 [SMILES: [2H]C([2H])([2H])NC(=O)c1nnc(NC(=O)C2(F)CC2)cc1Nc1ncc(F)cc1C(C)(F)F] |
| 71 | 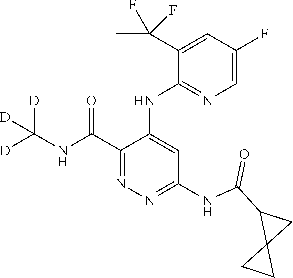 [SMILES: [2H]C([2H])([2H])NC(=O)c1nnc(NC(=O)C2CC23CC3)cc1Nc1ncc(F)cc1C(C)(F)F] |
| 71A | 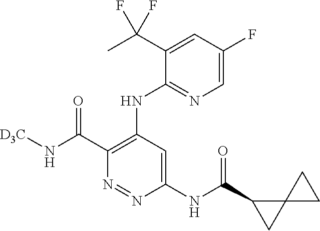 [SMILES: [2H]C([2H])([2H])NC(=O)c1nnc(NC(=O)[C@@H]2CC23CC3)cc1Nc1ncc(F)cc1C(C)(F)F] |
| 72 | 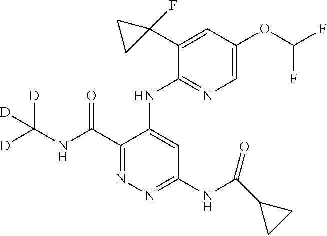 [SMILES: [2H]C([2H])([2H])NC(=O)c1nnc(NC(=O)C2CC2)cc1Nc1ncc(OC(F)F)cc1C1(F)CC1] |
| 73 | 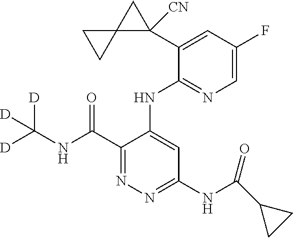 [SMILES: [2H]C([2H])([2H])NC(=O)c1nnc(NC(=O)C2CC2)cc1Nc1ncc(F)cc1C1(C#N)CC12CC2] |
| 74 | 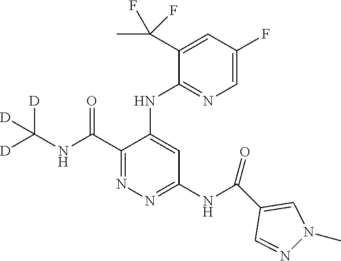 [SMILES: [2H]C([2H])([2H])NC(=O)c1nnc(NC(=O)c2cnn(C)c2)cc1Nc1ncc(F)cc1C(C)(F)F] |
| 75 | 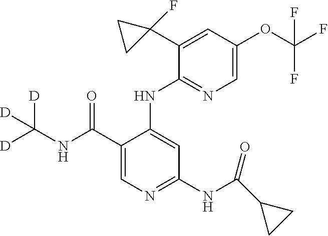 [SMILES: [2H]C([2H])([2H])NC(=O)c1cnc(NC(=O)C2CC2)cc1Nc1ncc(OC(F)(F)F)cc1C1(F)CC1] |
| 76 | 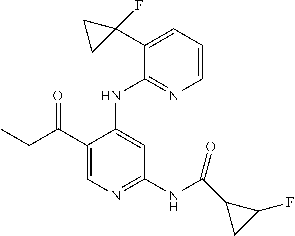 [SMILES: CCC(=O)c1cnc(NC(=O)C2CC2F)cc1Nc1ncccc1C1(F)CC1] |
| 76A | 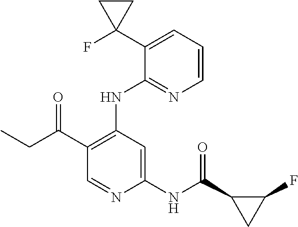 [SMILES: CCC(=O)c1cnc(NC(=O)[C@@H]2C[C@@H]2F)cc1Nc1ncccc1C1(F)CC1] |
| 77 | 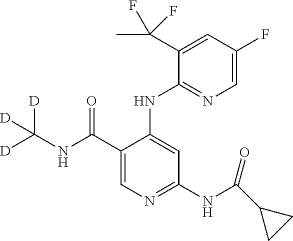 [SMILES: [2H]C([2H])([2H])NC(=O)c1cnc(NC(=O)C2CC2)cc1Nc1ncc(F)cc1C(C)(F)F] |
| 78 | 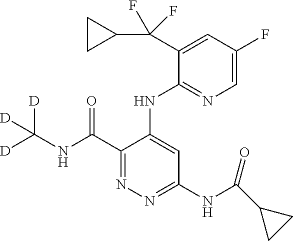 [SMILES: [2H]C([2H])([2H])NC(=O)c1nnc(NC(=O)C2CC2)cc1Nc1ncc(F)cc1C(F)(F)C1CC1] |
| 79 | 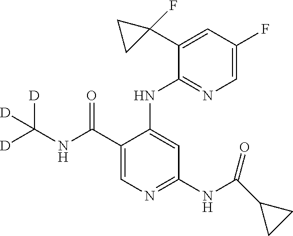 [SMILES: [2H]C([2H])([2H])NC(=O)c1cnc(NC(=O)C2CC2)cc1Nc1ncc(F)cc1C1(F)CC1] |
| 80 | 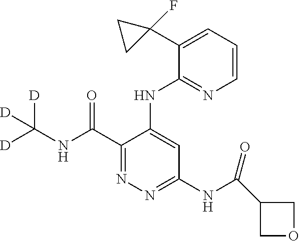 [SMILES: [2H]C([2H])([2H])NC(=O)c1nnc(NC(=O)C2COC2)cc1Nc1ncccc1C1(F)CC1] |
| 81 | 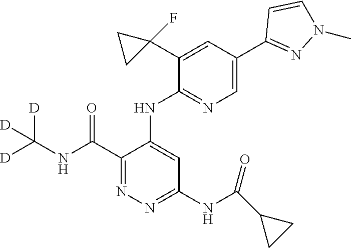 [SMILES: [2H]C([2H])([2H])NC(=O)c1nnc(NC(=O)C2CC2)cc1Nc1ncc(-c2ccn(C)n2)cc1C1(F)CC1] |
| 82 | 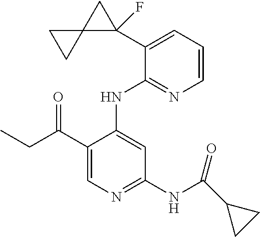 [SMILES: CCC(=O)c1cnc(NC(=O)C2CC2)cc1Nc1ncccc1C1(F)CC12CC2] |
| 83 | 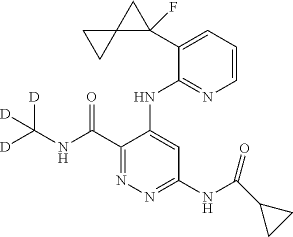 [SMILES: [2H]C([2H])([2H])NC(=O)c1nnc(NC(=O)C2CC2)cc1Nc1ncccc1C1(F)CC12CC2] |
| 84 | 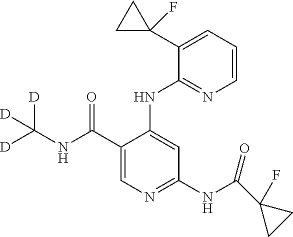 [SMILES: [2H]C([2H])([2H])NC(=O)c1cnc(NC(=O)C2(F)CC2)cc1Nc1ncccc1C1(F)CC1] |
| 85 | 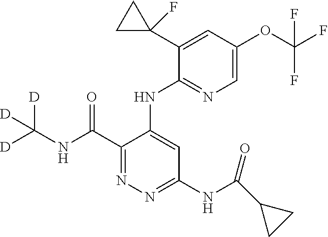 [SMILES: [2H]C([2H])([2H])NC(=O)c1nnc(NC(=O)C2CC2)cc1Nc1ncc(OC(F)(F)F)cc1C1(F)CC1] |
| 86 | 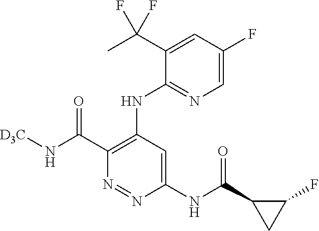 [SMILES: [2H]C([2H])([2H])NC(=O)c1nnc(NC(=O)[C@@H]2C[C@H]2F)cc1Nc1ncc(F)cc1C(C)(F)F] |
| 87 | 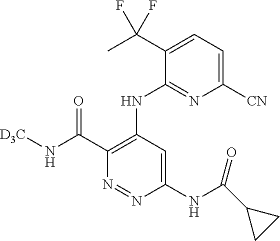 [SMILES: [2H]C([2H])([2H])NC(=O)c1nnc(NC(=O)C2CC2)cc1Nc1nc(C#N)ccc1C(C)(F)F] |
| 88 | 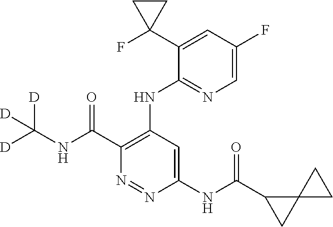 [SMILES: [2H]C([2H])([2H])NC(=O)c1nnc(NC(=O)C2CC23CC3)cc1Nc1ncc(F)cc1C1(F)CC1] |
| 89 | 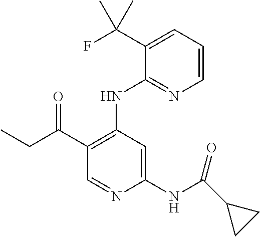 [SMILES: CCC(=O)c1cnc(NC(=O)C2CC2)cc1Nc1ncccc1C(C)(C)F] |
| 90 | 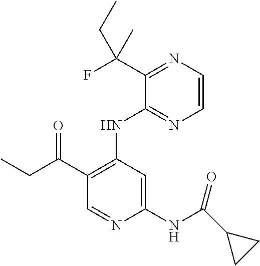 [SMILES: CCC(=O)c1cnc(NC(=O)C2CC2)cc1Nc1nccnc1C(C)(F)CC] |
| 91 | 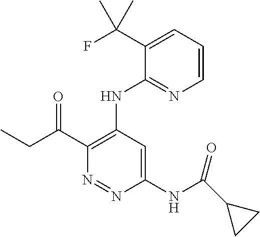 [SMILES: CCC(=O)c1nnc(NC(=O)C2CC2)cc1Nc1ncccc1C(C)(C)F] |
| 92 | 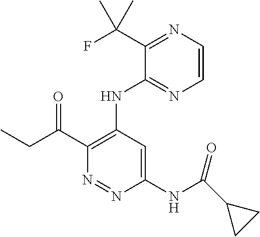 [SMILES: CCC(=O)c1nnc(NC(=O)C2CC2)cc1Nc1nccnc1C(C)(C)F] |
| 93 | 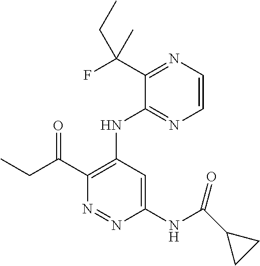 [SMILES: CCC(=O)c1nnc(NC(=O)C2CC2)cc1Nc1nccnc1C(C)(F)CC] |
| 94 | 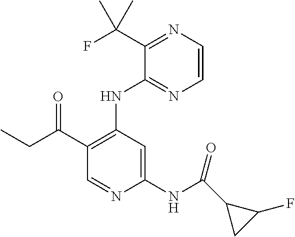 [SMILES: CCC(=O)c1cnc(NC(=O)C2CC2F)cc1Nc1nccnc1C(C)(C)F] |
| 94A | 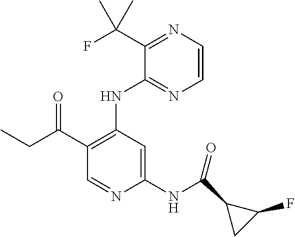 [SMILES: CCC(=O)c1cnc(NC(=O)[C@@H]2C[C@@H]2F)cc1Nc1nccnc1C(C)(C)F] |
| 95 | 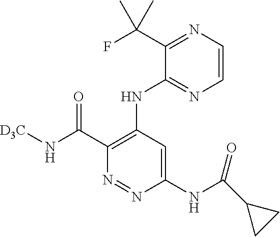 [SMILES: [2H]C([2H])([2H])NC(=O)c1nnc(NC(=O)C2CC2)cc1Nc1nccnc1C(C)(C)F] |
| 96 | 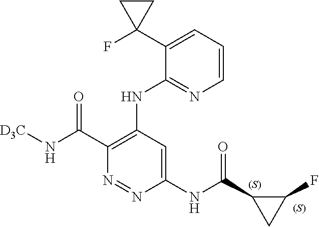 |
| 97A | 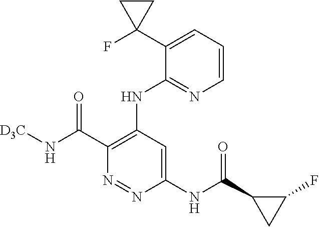 [SMILES: [2H]C([2H])([2H])NC(=O)c1nnc(NC(=O)[C@@H]2C[C@H]2F)cc1Nc1ncccc1C1(F)CC1] |
| 97B | 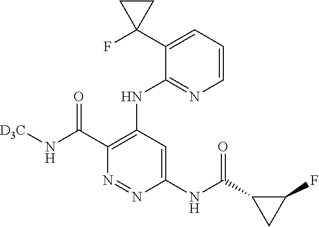 [SMILES: [2H]C([2H])([2H])NC(=O)c1nnc(NC(=O)[C@H]2C[C@@H]2F)cc1Nc1ncccc1C1(F)CC1] |
| 98 | 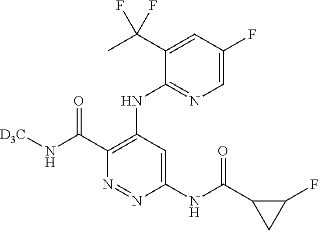 [SMILES: [2H]C([2H])([2H])NC(=O)c1nnc(NC(=O)C2CC2F)cc1Nc1ncc(F)cc1C(C)(F)F] |
| 98A | 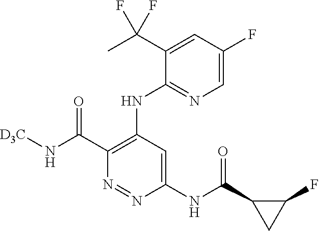 [SMILES: [2H]C([2H])([2H])NC(=O)c1nnc(NC(=O)[C@@H]2C[C@@H]2F)cc1Nc1ncc(F)cc1C(C)(F)F] |
| 99 | 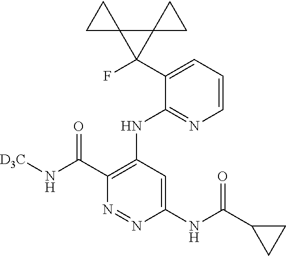 [SMILES: [2H]C([2H])([2H])NC(=O)c1nnc(NC(=O)C2CC2)cc1Nc1ncccc1C1(F)C2(CC2)C12CC2] |
| 100 | 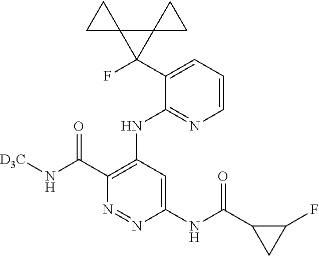 [SMILES: [2H]C([2H])([2H])NC(=O)c1nnc(NC(=O)C2CC2F)cc1Nc1ncccc1C1(F)C2(CC2)C12CC2] |
| 100A | 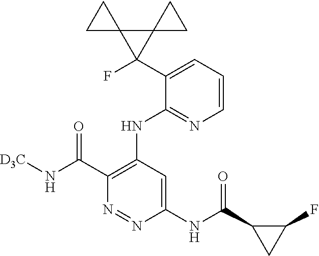 [SMILES: [2H]C([2H])([2H])NC(=O)c1nnc(NC(=O)[C@@H]2C[C@@H]2F)cc1Nc1ncccc1C1(F)C2(CC2)C12CC2] |
| 101 | 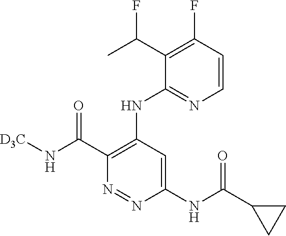 [SMILES: [2H]C([2H])([2H])NC(=O)c1nnc(NC(=O)C2CC2)cc1Nc1nccc(F)c1C(C)F] |
| 102 | 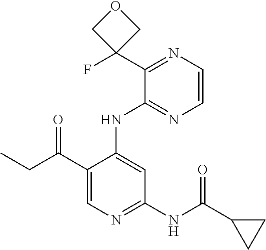 [SMILES: CCC(=O)c1cnc(NC(=O)C2CC2)cc1Nc1nccnc1C1(F)COC1] |
| 103 | 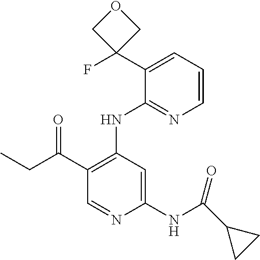 [SMILES: CCC(=O)c1cnc(NC(=O)C2CC2)cc1Nc1ncccc1C1(F)COC1] |
| 104 | 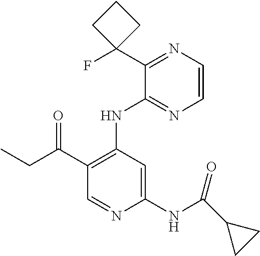 [SMILES: CCC(=O)c1cnc(NC(=O)C2CC2)cc1Nc1nccnc1C1(F)CCC1] |
| 105 | 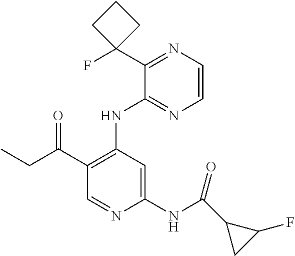 [SMILES: CCC(=O)c1cnc(NC(=O)C2CC2F)cc1Nc1nccnc1C1(F)CCC1] |
| 105A | 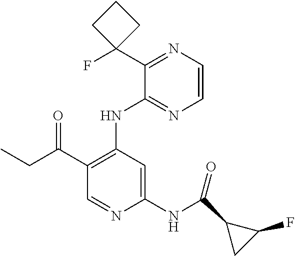 [SMILES: CCC(=O)c1cnc(NC(=O)[C@@H]2C[C@@H]2F)cc1Nc1nccnc1C1(F)CCC1] |
| 106 | 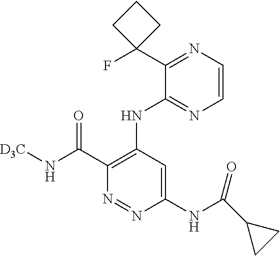 [SMILES: [2H]C([2H])([2H])NC(=O)c1nnc(NC(=O)C2CC2)cc1Nc1nccnc1C1(F)CCC1] |
| 107 | 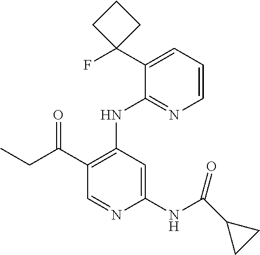 [SMILES: CCC(=O)c1cnc(NC(=O)C2CC2)cc1Nc1ncccc1C1(F)CCC1] |
| 108 | 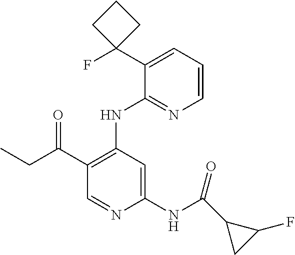 [SMILES: CCC(=O)c1cnc(NC(=O)C2CC2F)cc1Nc1ncccc1C1(F)CCC1] |
| 108A | 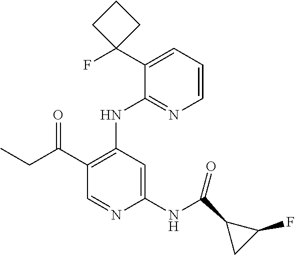 [SMILES: CCC(=O)c1cnc(NC(=O)[C@@H]2C[C@@H]2F)cc1Nc1ncccc1C1(F)CCC1] |
| 109 | 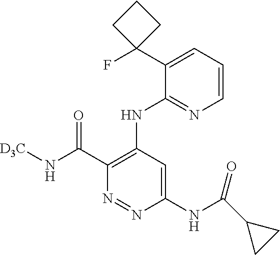 [SMILES: [2H]C([2H])([2H])NC(=O)c1nnc(NC(=O)C2CC2)cc1Nc1ncccc1C1(F)CCC1] |
| 110 | 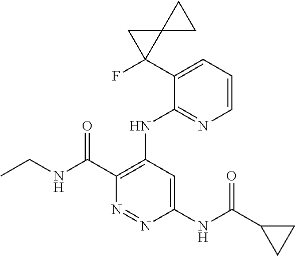 [SMILES: CCNC(=O)c1nnc(NC(=O)C2CC2)cc1Nc1ncccc1C1(F)CC12CC2] |
| 111 | 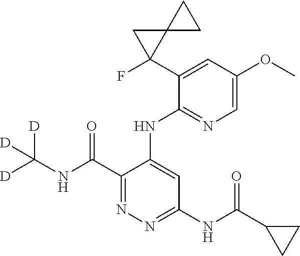 [SMILES: [2H]C([2H])([2H])NC(=O)c1nnc(NC(=O)C2CC2)cc1Nc1ncc(OC)cc1C1(F)CC12CC2] |
| 112 | 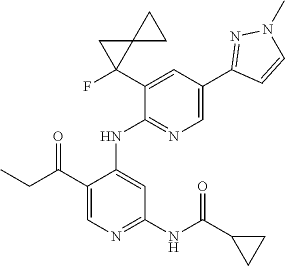 [SMILES: CCC(=O)c1cnc(NC(=O)C2CC2)cc1Nc1ncc(-c2ccn(C)n2)cc1C1(F)CC12CC2] |
| 113 | 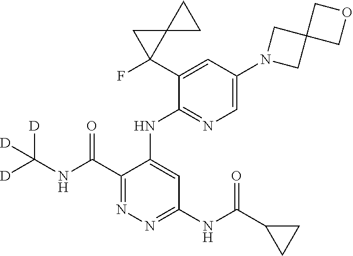 [SMILES: [2H]C([2H])([2H])NC(=O)c1nnc(NC(=O)C2CC2)cc1Nc1ncc(N2CC3(COC3)C2)cc1C1(F)CC12CC2] |
| 114 | 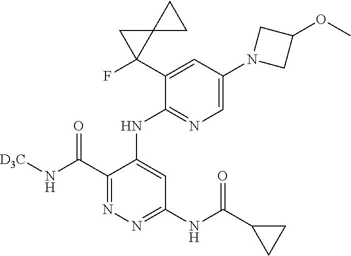 [SMILES: [2H]C([2H])([2H])NC(=O)c1nnc(NC(=O)C2CC2)cc1Nc1ncc(N2CC(OC)C2)cc1C1(F)CC12CC2] |
| 115 | 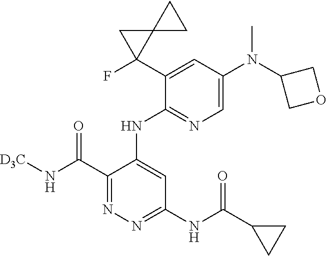 [SMILES: [2H]C([2H])([2H])NC(=O)c1nnc(NC(=O)C2CC2)cc1Nc1ncc(N(C)C2COC2)cc1C1(F)CC12CC2] |

In embodiments, compounds of the present disclosure (e.g., a compound of Formula (I), (IA), (IB), (IIA), (IIB), or Table 1) or a pharmaceutically acceptable salt, deuterated form, or stereoisomer thereof modulate TYK2. In embodiments, the modulating is binding TYK2. In embodiments, the modulating is binding to the pseudokinase domain (JH2) of TYK2. In embodiments, the modulating is inhibiting TYK2. In embodiments, TYK2 is selectively inhibited e.g., over other JAK family members, such as JAK1. In embodiments, compounds of the present disclosure exhibit at least about a 2-fold selectivity for inhibiting TYK2 over JAK1 in an assay described herein e.g., in Example 19. In embodiments, compounds of the present disclosure exhibit at least about a 5-fold selectivity for inhibiting TYK2 over JAK1 in an assay described herein e.g., in Example 19. In embodiments, compounds of the present disclosure exhibit at least about a 10-fold selectivity for inhibiting TYK2 over JAK1 in an assay described herein e.g., in Example 19. In embodiments, compounds of the present disclosure exhibit at least about a 20-fold selectivity for inhibiting TYK2 over JAK1 in an assay described herein e.g., in Example 19. In embodiments, compounds of the present disclosure exhibit at least about a 50-fold selectivity for inhibiting TYK2 over JAK1 in an assay described herein e.g., in Example 19. In embodiments, compounds of the present disclosure exhibit at least about a 100-fold selectivity for inhibiting TYK2 over JAK1 in an assay described herein e.g., in Example 19.

In another embodiment, compounds of the present disclosure have an IC₅₀<1000 nM in at least one of the assays described below. In another embodiment, compounds of the present disclosure have an IC₅₀<500 nM in at least one of the assays described below. In another embodiment, compounds of the present disclosure have an IC₅₀<250 nM in at least one of the assays described below. In another embodiment, compounds of the present disclosure have an IC₅₀<100 nM in at least one of the assays described below. In another embodiment, compounds of the present disclosure have an IC₅₀<50 nM in at least one of the assays described below. In another embodiment, compounds of the present disclosure have an IC₅₀<25 nM in at least one of the assays described below. In another embodiment, compounds of the present disclosure have an IC₅₀<10 nM in at least one of the assays described below.

### Compositions

The present disclosure provides pharmaceutical compositions for modulating Tyrosine kinase 2 (TYK2) in a subject. In embodiments, a pharmaceutical composition comprises one or more compounds of the present disclosure (e.g., a compound of Formula (I), (IA), (IB), (IIA), (IIB), or Table 1) or a pharmaceutically acceptable salt, deuterated form, or stereoisomer thereof. In embodiments, a pharmaceutical composition comprises one or more compounds of the present disclosure (e.g., a compound of Formula (I), (IA), (IB), (IIA), (IIB), or Table 1) or a pharmaceutically acceptable salt, or deuterated form thereof. In embodiments, a pharmaceutical composition comprises one or more compounds of the present disclosure (e.g., a compound of Formula (I), (IA), (IB), (IIA), (IIB), or Table 1) or a pharmaceutically acceptable salt thereof. In embodiments, a pharmaceutical composition comprises one or more compounds of the present disclosure (e.g., a compound of Formula (I), (IA), (IB), (IIA), (IIB), or Table 1)

In embodiments of the present disclosure, a pharmaceutical composition comprises a therapeutically effective amount of one or more compounds of the present disclosure (e.g., a compound of Formula (I), (IA), (IB), (IIA), (IIB), or Table 1) or a pharmaceutically acceptable salt, deuterated form, or stereoisomer thereof. In embodiments of the present disclosure, a pharmaceutical composition comprises a therapeutically effective amount of one or more compounds of the present disclosure (e.g., a compound of Formula (I), (IA), (IB), (IIA), (IIB), or Table 1) or a pharmaceutically acceptable salt or deuterated form thereof. In embodiments of the present disclosure, a pharmaceutical composition comprises a therapeutically effective amounts of one or more compounds of the present disclosure (e.g., a compound of Formula (I), (IA), (IB), (IIA), (IIB), or Table 1) or a pharmaceutically acceptable salt thereof. In embodiments of the present disclosure, a pharmaceutical composition comprises a therapeutically effective amounts of one or more compounds of the present disclosure (e.g., a compound of Formula (I), (IA), (IB), (IIA), (IIB), or Table 1).

In embodiments, a pharmaceutical composition, as described herein, comprises one or more compounds selected from Table 1, or a pharmaceutically acceptable salt, deuterated form, or stereoisomer thereof. In embodiments, a pharmaceutical composition, as described herein, comprises one or more compounds selected from Table 1, or a pharmaceutically acceptable salt, or deuterated form thereof. In embodiments, a pharmaceutical composition, as described herein, comprises one or more compounds selected from Table 1, or a pharmaceutically acceptable salt thereof. In embodiments, a pharmaceutical composition, as described herein, comprises one or more compounds selected from Table 1.

In embodiments of the present disclosure, a pharmaceutical composition comprising one or more compounds of the present disclosure (e.g., a compound of Formula (I), (IA), (IB), (IIA), (IIB), or Table 1) or a pharmaceutically acceptable salt, deuterated form, or stereoisomer thereof, and a pharmaceutically acceptable excipient or adjuvant is provided. In embodiments of the present disclosure, a pharmaceutical composition comprising one or more compounds of the present disclosure (e.g., a compound of Formula (I), (IA), (IB), (IIA), (IIB), or Table 1) or a pharmaceutically acceptable salt or deuterated form thereof, and a pharmaceutically acceptable excipient or adjuvant is provided. In embodiments of the present disclosure, a pharmaceutical composition comprising one or more compounds of the present disclosure (e.g., a compound of Formula (I), (IA), (IB), (IIA), (IIB), or Table 1) or a pharmaceutically acceptable salt thereof, and a pharmaceutically acceptable excipient or adjuvant is provided. In embodiments of the present disclosure, a pharmaceutical composition comprising one or more compounds of the present disclosure (e.g., a compound of Formula (I), (IA), (IB), (IIA), (IIB), or Table 1), and a pharmaceutically acceptable excipient or adjuvant is provided.

The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound -administered, the age, weight, and response of the individual patient, the severity of the patient's symptoms, and the like.

### EMBODIMENTS

### Embodiments A

Embodiment 1A. A compound of Formula (I):

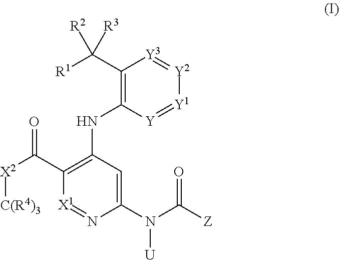

or a pharmaceutically acceptable salt, deuterated form, or stereoisomer thereof;
wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or -CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z} or —C₃₋₆ cycloalkyl, wherein the —C₃₋₆ cycloalkyl is optionally substituted with 1 or more substituents each independently selected from the group consisting of -halo, —C₁₋₆ alkyl, —OC₁₋₆ alkyl, or —OC₃₋₆ cycloalkyl, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.
   Embodiment 2A. The compound of embodiment 1, wherein Z is —OR^{Z} or —C₃₋₆ cycloalkyl, wherein the —C₃₋₆ cycloalkyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents are taken together to form a carbocyclyl.
   Embodiment 3A. The compound of embodiment 1 or 2, wherein Z is —OR^{Z} or cyclopropyl, wherein the cyclopropyl is optionally substituted with 1, 2, or 3 substituents each independently selected from the group consisting of -halo, —C₁₋₆ alkyl, —OC₁₋₆ alkyl, —OC₃₋₆ cycloalkyl, or two substituents are taken together to form a carbocyclyl.
   Embodiment 4A. The compound of embodiment 3, wherein Z is —OR^{Z} or cyclopropyl, wherein the cyclopropyl is optionally substituted with 1, 2, or 3 substituents each independently selected from the group consisting of -halo, —C₁₋₆ alkyl, —OC₁₋₆ alkyl, or —OC₃₋₆ cycloalkyl.
   Embodiment 5A. The compound of any one of embodiments 1-4, wherein U is H.
   Embodiment 6A. The compound of embodiment 1, wherein U and Z are taken together to form a 5-8 membered heterocyclyl, wherein the 5-8 membered heterocyclyl is optionally substituted with 1, 2, or 3 substituents independently selected from the group consisting of -halo, —C₁₋₆ alkyl, or —OC₁₋₆ alkyl.
   Embodiment 7A. The compound of embodiment 6, wherein U and Z are taken together to form a 5-membered saturated heterocyclyl, wherein the 5-membered saturated heterocyclyl is optionally substituted with —C₁₋₆ alkyl.
   Embodiment 8A. The compound of embodiment 1, having a structure of Formula (IA):

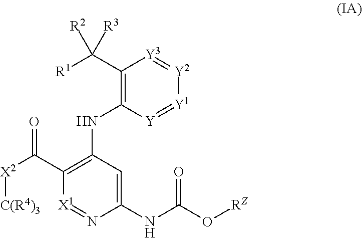

or a pharmaceutically acceptable salt, deuterated form, or stereoisomer thereof.
Embodiment 9A. The compound of any one of embodiments 1-3, having a structure of Formula (IB):

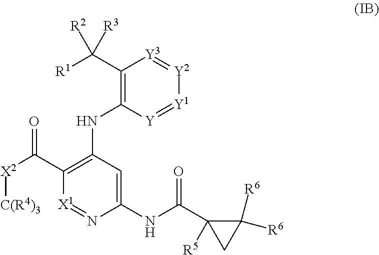

or a pharmaceutically acceptable salt, deuterated form, or stereoisomer thereof;
wherein R⁵, R⁶ and R⁷ are each independently selected from the group consisting of —H, -halo, —C₁₋₆ alkyl, —OC₁₋₆ alkyl, or —OC₃₋₆ cycloalkyl, or R⁶ and R⁷ are taken together to form a carbocyclyl.
Embodiment 10A. The compound of any one of embodiments 1-9, wherein Y, Y¹, Y², and Y³ are not all CH.
Embodiment 11A. The compound of any one of embodiments 1-10, wherein at least one of Y, Y¹, Y², and Y³ is N.
Embodiment 12A. The compound of any one of embodiments 1-11, wherein Y is N.
Embodiment 13A. The compound of any one of embodiments 1-12, wherein one or two of Y¹, Y², and Y³ is N.
Embodiment 14A. The compound of any one of embodiments 1-5 or 8-12, having a structure of Formula (IIA) or (IIB):

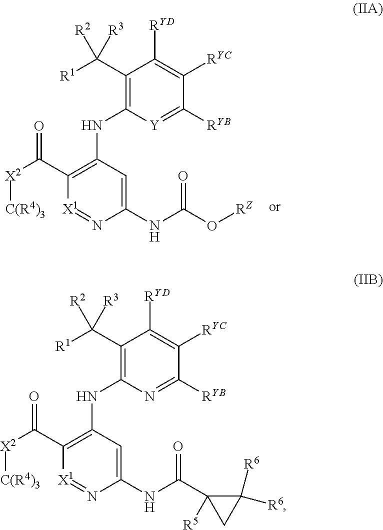

or a pharmaceutically acceptable salt, deuterated form, or stereoisomer thereof.
Embodiment 15A. The compound of any one of embodiments 9-14, wherein R⁵ is —H or halo.
Embodiment 16A. The compound of any one of embodiments 9-15, wherein R⁶ and R⁷ are each independently selected from the group consisting of —H, -halo, —C₁₋₆ alkyl, or —OC₁₋₆ alkyl, or R⁶ and R⁷ are taken together to form a C₃₋₆cycloalkyl.
Embodiment 17A. The compound of embodiment 16, wherein R⁶ and R⁷ are each independently selected from the group consisting of —H, -halo, —C₁₋₆ alkyl, or —OC₁₋₆ alkyl, or R⁶ and R⁷ are taken together to form a cyclopropyl or cyclobutyl.
Embodiment 18A. The compound of embodiment 17, wherein R⁶ and R⁷ are each independently selected from the group consisting of —H, -halo, —C₁₋₆ alkyl, or —OC₁₋₆ alkyl.
Embodiment 19A. The compound of any one of embodiments 9-18, wherein R⁵, R⁶ and R⁷ are H.
Embodiment 20A. The compound of any one of embodiments 1-19, wherein R^{YA}, R^{YB}, and R^{YC} are each independently —H, —CN, -halo, —C₁₋₆ alkyl, —C₁₋₆ alkenyl, —C₁₋₆ alkynyl, —C₃₋₆ cycloalkyl, saturated heterocyclyl, aryl, or heteroaryl and R^{YD} is —H, —CN, -halo, —C₁₋₆ alkyl, —C₁₋₆ alkenyl, —C₁₋₆ alkynyl, —C₃₋₆ cycloalkyl, a saturated heterocyclyl, or aryl.
Embodiment 21A. The compound of any one of embodiments 1-20, wherein R^{YB}, R^{YC}, and R^{YD} are each independently —H, —C₁₋₆ alkyl, —CN, or halo.
Embodiment 22A. The compound of embodiment 21, wherein R^{YB} and R^{YD} are —H and R^{YC} is —H, —C₁₋₆ alkyl, —CN, or —F.
Embodiment 23A. The compound of embodiment 21, wherein two or three of R^{YB}, R^{YC}, and R^{YD} are —H.
Embodiment 24A. The compound of any one of embodiments 1-23, wherein:
R¹ is —H, -halo, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —C₁₋₆ alkyl, —C₁₋₆ alkenyl, —C₁₋₆ alkynyl, —C₃₋₆ cycloalkyl, or a saturated heterocyclyl; wherein the —C₁₋₆ alkyl, —C₁₋₆ alkenyl, —C₁₋₆ alkynyl, —C₃₋₆ cycloalkyl, or saturated heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated heterocyclyl, wherein the carbocyclyl or saturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl.
Embodiment 25A. The compound of any one of embodiments 1-23, wherein:
R¹ is —H, -halo, —OH, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, or —O-saturated heterocyclyl;
R² is —H, -halo, —C₁₋₆ alkyl, —C₁₋₆ alkenyl, —C₁₋₆ alkynyl, —C₃₋₆ cycloalkyl, or saturated heterocyclyl; or
R¹ and R² are taken together to form a carbocyclyl or saturated heterocyclyl.
Embodiment 26A. The compound of any one of embodiments 1-25, wherein:
R¹ is —C₁₋₆ alkyl;
R² is —H, —C₁₋₆ alkyl, or -halo; or
R¹ and R² are taken together to form a carbocyclyl or saturated heterocyclyl.
Embodiment 27A. The compound of any one of embodiments 1-26, wherein R¹ is C₁₋₆ alkyl.
Embodiment 28A. The compound of embodiment 27, wherein R¹ is —CH₃.
Embodiment 29A. The compound of any one of embodiments 1-26, wherein R¹ and R² are taken together to form a carbocyclyl.
Embodiment 30A. The compound of any one of embodiments 1-26, wherein R¹ and R² are taken together to form a —C₃₋₆ cycloalkyl or saturated heterocyclyl.
Embodiment 31A. The compound of embodiment 29 or 30, wherein R¹ and R² are taken together to form a —C₃₋₆ cycloalkyl.
Embodiment 32A. The compound of embodiment 25 or 26, wherein R¹ and R² are taken together to form a cyclopropyl or oxetanyl.
Embodiment 33A. The compound of any one of embodiments 29-32, wherein R¹ and R² are taken together to form a cyclopropyl.
Embodiment 34A. The compound of any one of embodiments 1-28, wherein R² is —H or -halo.
Embodiment 35A. The compound of embodiment 34, wherein R² is —H.
Embodiment 36A. The compound of embodiment 34, wherein R² is halo.
Embodiment 37A. The compound of embodiment 36, wherein R² is F.
Embodiment 38A. The compound of any one of embodiments 1-37, wherein R³ is —H, -halo, —C₁₋₆ alkyl, —C₁₋₆ haloalkyl, —O—C₁₋₆ alkyl, or CN.
Embodiment 39A. The compound of any one of embodiments 1-38, wherein R³ is -halo, —C₁₋₆ haloalkyl, —O—C₁₋₆ alkyl, or CN.
Embodiment 40A. The compound of any one of embodiments 1-39, wherein R³ is C₁₋₆ haloalkyl, —OC₁₋₆ alkyl, or CN.
Embodiment 41A. The compound of any one of embodiments 1-40, wherein R³ is C₁₋₃ haloalkyl, —OC₁₋₃ alkyl, or CN.
Embodiment 42A. The compound of embodiment 38, wherein R³ is —H, —F, —CH₃, —CF₃, —OCH₃, —OCH₂CH₃, or CN.
Embodiment 43A. The compound of embodiment 38, wherein R³ is —F, —CH₃, —CF₃, —OCH₃, —OCH₂CH₃, or CN.
Embodiment 44A. The compound of embodiment 43, wherein R³ is —CH₃, —CF₃, —OCH₃, —OCH₂CH₃, or CN.
Embodiment 45A. The compound of embodiment 38, wherein R³ is H or halo.
Embodiment 46A. The compound of any one of embodiments 1-45, wherein X² is CHR⁸.
Embodiment 47A. The compound of any one of embodiments 1-46, wherein X² is CH₂.
Embodiment 48A. The compound of any one of embodiments 1-45, wherein X² is NH.
Embodiment 49A. The compound of any one of embodiments 1-48, wherein each R⁴ is —H.
Embodiment 50A. The compound of any one of embodiments 1-48, wherein each R⁴ is -D.
Embodiment 51A. The compound of any one of embodiments 1-50, wherein X¹ is CH.
Embodiment 52A. The compound of any one of embodiments 1-50, wherein X¹ is N.
Embodiment 53A. The compound of embodiment 1, wherein the compound is selected from the group consisting of:

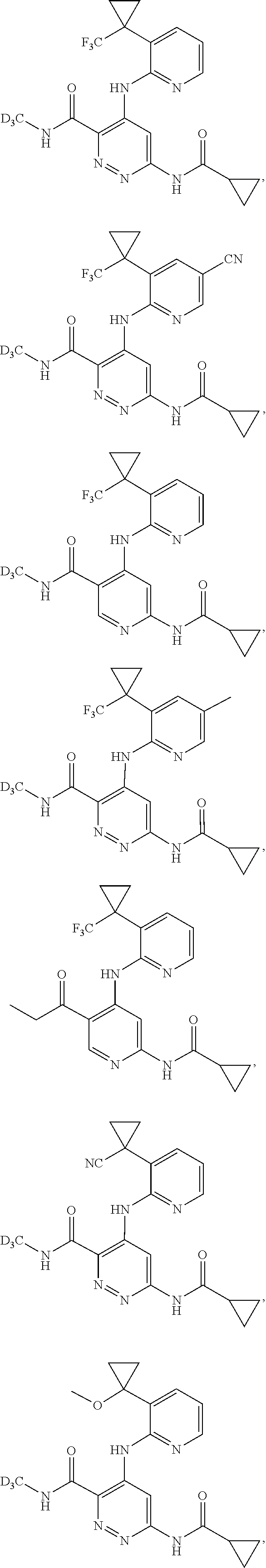

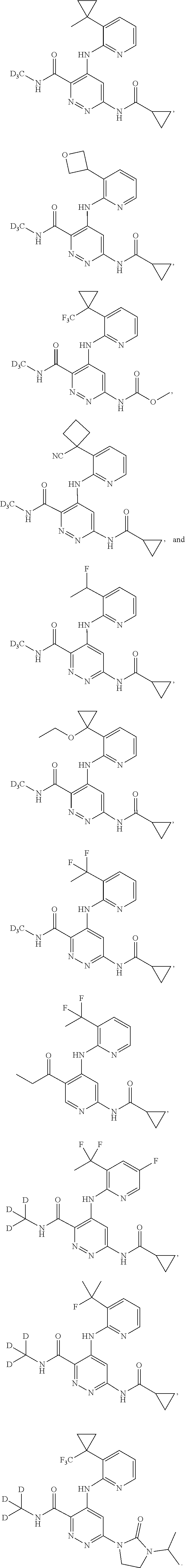

or a pharmaceutically acceptable salt, deuterated form, or stereoisomer thereof.
Embodiment 54A. A pharmaceutical composition comprising a compound of any one of embodiments 1-53, or a pharmaceutically acceptable salt, deuterated form, or stereoisomer thereof, and a pharmaceutically acceptable carrier.

### EMBODIMENTS B

Embodiment 1B. A compound of Formula (I):

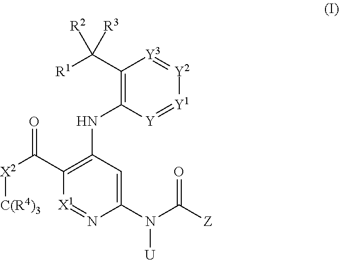

or a pharmaceutically acceptable salt, deuterated form, or stereoisomer thereof;
wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, —C₁₋₆ alkylene-O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl,
C₁₋₆ alkenyl, —C₁₋₆ alkynyl, —C₃₋₆ cycloalkyl, heterocyclyl, or aryl;
U is H;
Z is —OR^{Z}, —(CH₂)₀₋₃—(C₃₋₆ cycloalkyl), or —(CH₂)₀₋₃—(C₃-C₄ heterocyclyl), and wherein the —C₃₋₆ cycloalkyl is optionally substituted with 1 or more substituents each independently selected from the group consisting of -halo, —C₁₋₆ alkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.
   Embodiment 2B. The compound of embodiment 1, wherein Z is —OR^{Z} or —C₃₋₆ cycloalkyl, or —C₃-C₄ heterocyclyl wherein the —C₃₋₆ cycloalkyl or —C₃-C₄ heterocyclyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN or two substituents are taken together to form a carbocyclyl.
   Embodiment 3B. The compound of embodiment 1 or 2, wherein Z is —OR^{Z}, cyclopropyl, cyclobutyl, bicyclopentyl, spirohexyl, or oxetanyl, wherein the cyclopropyl, cyclobutyl, bicyclopentyl, spirohexyl, or oxetanyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN, or two substituents are taken together to form a carbocyclyl.
   Embodiment 4B. The compound of embodiment 3, wherein Z is —OR^{Z}, cyclopropyl, cyclobutyl, bicyclopentyl, spirohexyl, or oxetanyl, wherein the cyclopropyl, cyclobutyl, bicyclopentyl, spirohexyl, or oxetanyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN.
   Embodiment 5B. The compound of any of embodiments 1-4, wherein Z is —(CH₂)₀₋₃—(C₃-C₆ cycloalkyl).
   Embodiment 6B. The compound of any of embodiments 1-4, wherein Z is —(CH₂)—(C₃-C₆ cycloalkyl).
   Embodiment 7B. The compound of any one of embodiments 1-4, wherein U is H.
   Embodiment 8B. The compound of embodiment 1, wherein U and Z are taken together to form a 5-8 membered heterocyclyl, wherein the 5-8 membered heterocyclyl is optionally substituted with 1, 2, or 3 substituents independently selected from the group consisting of -halo, —C₁₋₆ alkyl, or —OC₁₋₆ alkyl.
   Embodiment 9B. The compound of embodiment 8, wherein U and Z are taken together to form a 5-membered saturated heterocyclyl, wherein the 5-membered saturated heterocyclyl is optionally substituted with —C₁₋₆ alkyl.
   Embodiment 10B. The compound of embodiment 9, wherein the 5-membered saturated heterocyclyl is substituted with CH₃, CH₂CH₃, CH₂CH₂CH₃, or CH(CH₃)₂.
   Embodiment 11B. The compound of embodiment 1, having a structure of Formula (IA):

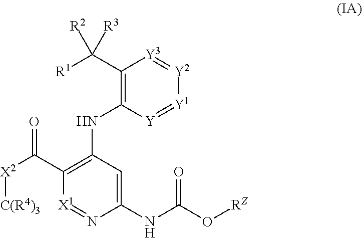

or a pharmaceutically acceptable salt, deuterated form, or stereoisomer thereof.
Embodiment 12B. The compound of any one of embodiments 1-3, having a structure of Formula (IB):

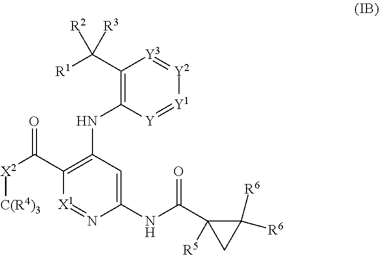

or a pharmaceutically acceptable salt, deuterated form, or stereoisomer thereof;
wherein R⁵, R⁶ and R⁷ are each independently selected from the group consisting of —H, -halo, —C₁₋₆ alkyl, —OC₁₋₆ alkyl, or —OC₃₋₆ cycloalkyl, or R⁶ and R⁷ are taken together to form a carbocyclyl.
Embodiment 13B. The compound of any one of embodiments 1-12, wherein Y, Y¹, Y², and Y³ are not all CH.
Embodiment 14B. The compound of any one of embodiments 1-13, wherein at least one of Y, Y¹, Y², and Y³ is N.
Embodiment 15B. The compound of any one of embodiments 1-14, wherein Y is N.
Embodiment 16B. The compound of any one of embodiments 1-15, wherein one or two of Y¹, Y², and Y³ is N.
Embodiment 17B. The compound of any one of embodiments 1-13, wherein Y is N and Y² is N.
Embodiment 18B. The compound of any one of embodiments 1-13, wherein Y is N and Y³ is N.
Embodiment 19B. The compound of any one of embodiments 1-12, wherein:
Y is N and Y² is CR^{YC} wherein R^{YC} is CH₃, —CN, F, or Cl; or wherein
Y is N and Y³ is CR^{YD} wherein R^{YD} is CH₃, —CN, F, or Cl.
Embodiment 20B. The compound of any one of embodiments 1-7 or 12-15, having a structure of Formula (IIA) or (IIB):

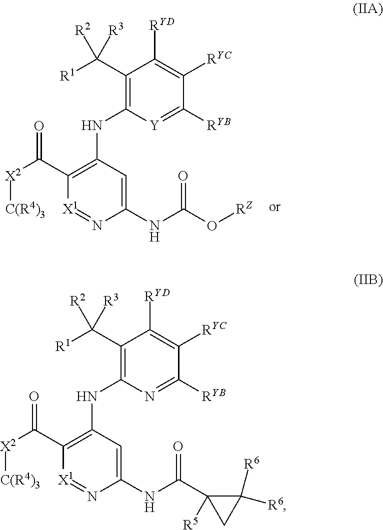

or a pharmaceutically acceptable salt, deuterated form, or stereoisomer thereof.
Embodiment 21B. The compound of any one of embodiments 13-20, wherein R⁵ is —H or halo.
Embodiment 22B. The compound of any one of embodiments 13-20, wherein R⁶ and R⁷ are each independently selected from the group consisting of —H, -halo, —C₁₋₆ alkyl, or —OC₁₋₆ alkyl, or R⁶ and R⁷ are taken together to form a C₃₋₆cycloalkyl.
Embodiment 23B. The compound of embodiment 22, wherein R⁶ and R⁷ are each independently selected from the group consisting of —H, -halo, —C₁₋₆ alkyl, or —OC₁₋₆ alkyl, or R⁶ and R⁷ are taken together to form a cyclopropyl or cyclobutyl.
Embodiment 24B. The compound of embodiment 23, wherein R⁶ and R⁷ are each independently selected from the group consisting of —H, -halo, —C₁₋₆ alkyl, or —OC₁₋₆ alkyl.
Embodiment 25B. The compound of any one of embodiments 13-21, wherein R⁵, R⁶ and R⁷ are H.
Embodiment 26B. The compound of any one of embodiments 1-25, wherein R^{YA}, R^{YB}, and R^{YC} are each independently —H, —CN, -halo, —C₁₋₆ alkyl, —C₁₋₆ alkenyl, —C₁₋₆ alkynyl, —C₃₋₆ cycloalkyl, saturated heterocyclyl, aryl, or heteroaryl and R^{YD} is —H, —CN, -halo, —C₁₋₆ alkyl, —C₁₋₆ alkenyl, —C₁₋₆ alkynyl, —C₃₋₆ cycloalkyl, a saturated heterocyclyl, or aryl.
Embodiment 27B. The compound of any one of embodiments 1-26, wherein R^{YB}, R^{YC}, and R^{YD} are each independently —H, —C₁₋₆ alkyl, —CN, or halo.
Embodiment 28B. The compound of embodiment 27, wherein R^{YB} and R^{YD} are —H and R^{YC} is —H, —C₁₋₆ alkyl, —CN, or —F.
Embodiment 29B. The compound of embodiment 27, wherein two or three of R^{YB}, R^{YC}, and R^{YD} are —H.
Embodiment 30B. The compound of any one of embodiments 1-29, wherein:
R¹ is —H, -halo, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH(CI-6 alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl;
R² is —H, -halo, —C₁₋₆ alkyl, —C₁₋₆ alkenyl, —C₁₋₆ alkynyl, —C₃₋₆ cycloalkyl, or a saturated heterocyclyl; wherein the —C₁₋₆ alkyl, —C₁₋₆ alkenyl, —C₁₋₆ alkynyl, —C₃₋₆ cycloalkyl, or saturated heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated heterocyclyl, wherein the carbocyclyl or saturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl.
Embodiment 31B. The compound of any one of embodiments 1-29, wherein:
R¹ is —H, -halo, —OH, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, or —O-saturated heterocyclyl;
R² is —H, -halo, —C₁₋₆ alkyl, —C₁₋₆ alkenyl, —C₁₋₆ alkynyl, —C₃₋₆ cycloalkyl, or saturated heterocyclyl; or
R¹ and R² are taken together to form a carbocyclyl or saturated heterocyclyl.
Embodiment 32B. The compound of any one of embodiments 1-31, wherein:
R¹ is —C₁₋₆ alkyl;
R² is —H, —C₁₋₆ alkyl, or -halo; or
R¹ and R² are taken together to form a carbocyclyl or saturated heterocyclyl.
Embodiment 33B. The compound of any one of embodiments 1-32, wherein R¹ is C₁₋₆ alkyl.
Embodiment 34B. The compound of embodiment 33, wherein R¹ is —CH₃.
Embodiment 35B. The compound of any one of embodiments 1-34, wherein R¹ and R² are taken together to form a carbocyclyl.
Embodiment 36B. The compound of any one of embodiments 1-32, wherein R¹ and R² are taken together to form a —C₃₋₆ cycloalkyl or saturated heterocyclyl.
Embodiment 37B. The compound of embodiment 32 or 33, wherein R¹ and R² are taken together to form a —C₃₋₆ cycloalkyl.
Embodiment 38B. The compound of embodiment 28 or 29, wherein R¹ and R² are taken together to form a cyclopropyl or oxetanyl.
Embodiment 39B. The compound of any one of embodiments 32-35, wherein R¹ and R² are taken together to form a cyclopropyl.
Embodiment 40B. The compound of any of embodiments 35-39, wherein the carbocycle or heterocycle is substituted with —CH₂OCH₃.
Embodiment 41B. The compound of any of embodiments 35-40, wherein R¹ and R² are taken together to form a cyclopropyl, wherein the carbocycle is substituted by two substituents which come together to form a cyclopropyl or cyclobutyl ring.
Embodiment 42B. The compound of any one of embodiments 1-41, wherein R² is —H or -halo.
Embodiment 43B. The compound of embodiment 42, wherein R² is —H.
Embodiment 44B. The compound of embodiment 42, wherein R² is halo.
Embodiment 45B. The compound of embodiment 42, wherein R² is F.
Embodiment 46B. The compound of any one of embodiments 1-45, wherein R³ is —H, -halo, —C₁₋₆ alkyl, —C₁₋₆ haloalkyl, —O—C₁₋₆ alkyl, or CN.
Embodiment 47B. The compound of any one of embodiments 1-46, wherein R³ is -halo, —C₁₋₆ haloalkyl, —O—C₁₋₆ alkyl, or CN.
Embodiment 48B. The compound of any one of embodiments 1-47, wherein R³ is C₁₋₆ haloalkyl, —OC₁₋₆ alkyl, or CN.
Embodiment 49B. The compound of any one of embodiments 1-48, wherein R³ is C₁₋₃ haloalkyl, —OC₁₋₃ alkyl, or CN.
Embodiment 50B. The compound of embodiment 46, wherein R³ is —H, —F, —CH₃, —CH₂F, CHF₂, —CF₃, —OCH₃, —OCH₂CH₃, or CN.
Embodiment 51B. The compound of embodiment 46, wherein R³ is —F, —CH₃, —CF₃, —OCH₃, —OCH₂CH₃, or CN.
Embodiment 52B. The compound of embodiment 51, wherein R³ is —CH₃, —CF₃, —OCH₃, —OCH₂CH₃, or CN.
Embodiment 53B. The compound of embodiment 46, wherein R³ is H, F, or Cl.
Embodiment 54B. The compound of embodiment 53, wherein R³ is F.
Embodiment 55B. The compound of any one of embodiments 1-54, wherein X² is CHR⁸.
Embodiment 56B. The compound of any one of embodiments 1-55, wherein X² is CH₂.
Embodiment 57B. The compound of any one of embodiments 1-54, wherein X² is NH.
Embodiment 58B. The compound of any one of embodiments 1-57, wherein each R⁴ is —H.
Embodiment 59B. The compound of any one of embodiments 1-57, wherein each R⁴ is -D.
Embodiment 60B. The compound of any one of embodiments 1-59, wherein X¹ is CH.
Embodiment 61B. The compound of any one of embodiments 1-59, wherein X¹ is N.
Embodiment 62B. The compound of embodiment 1, wherein the compound is selected from the group consisting of:

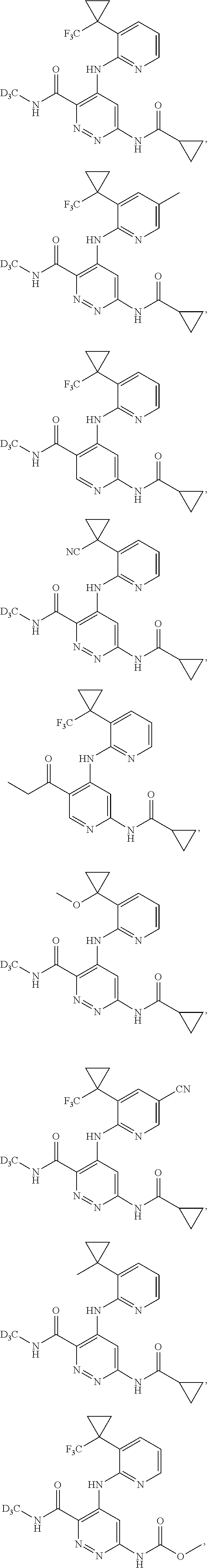

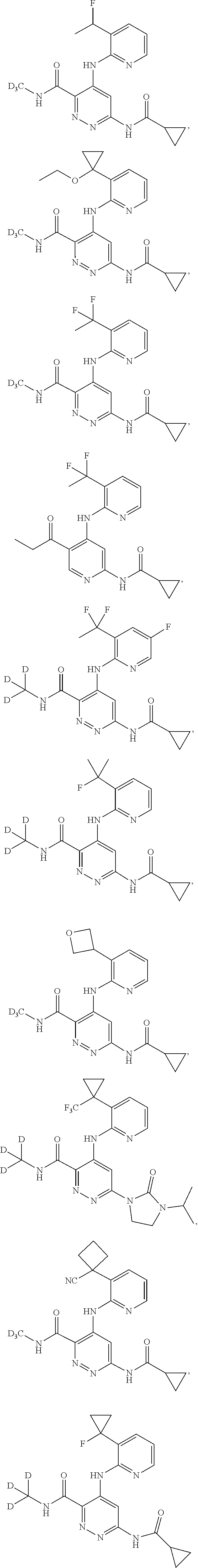

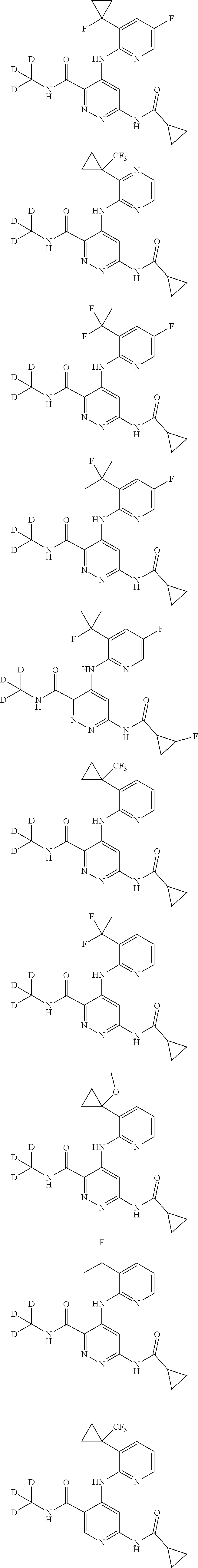

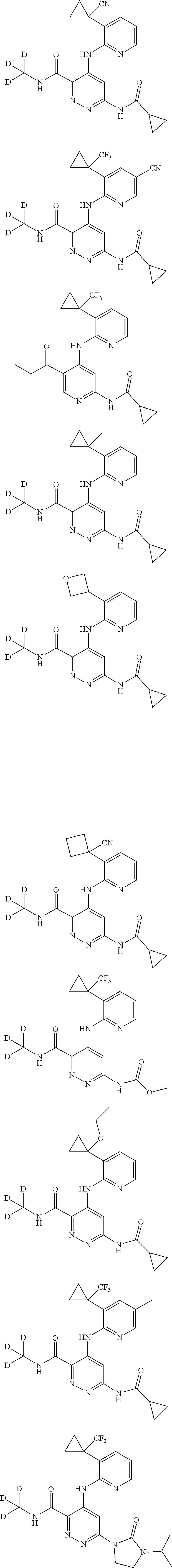

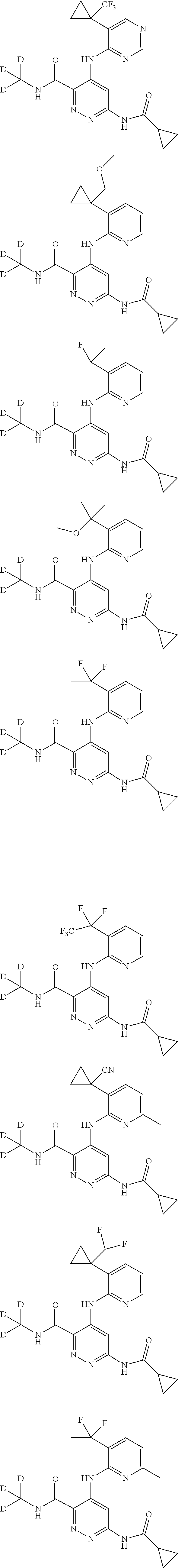

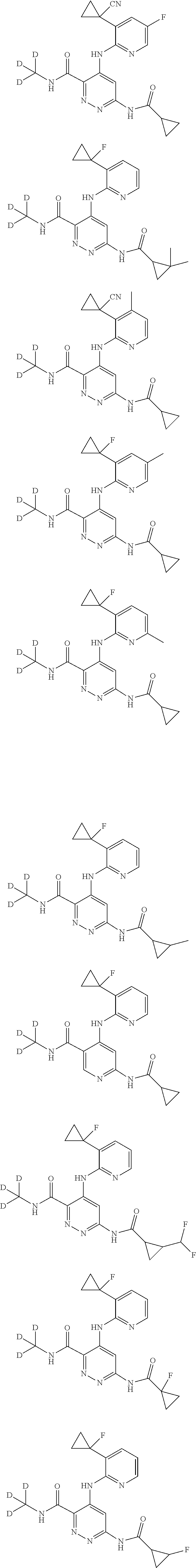

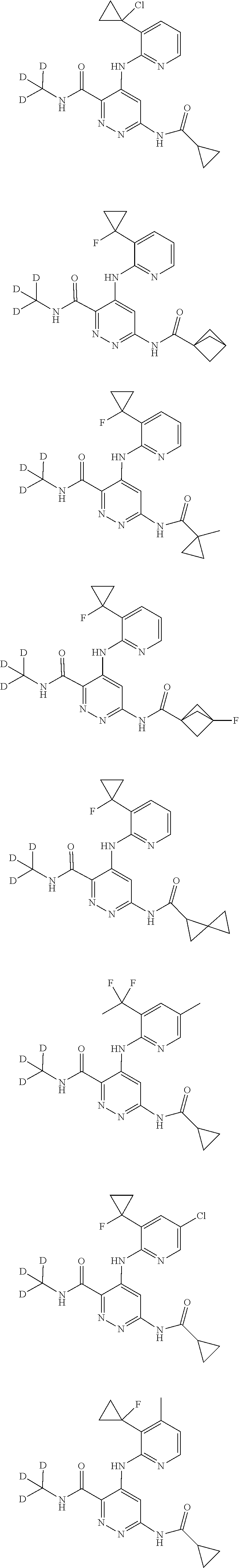

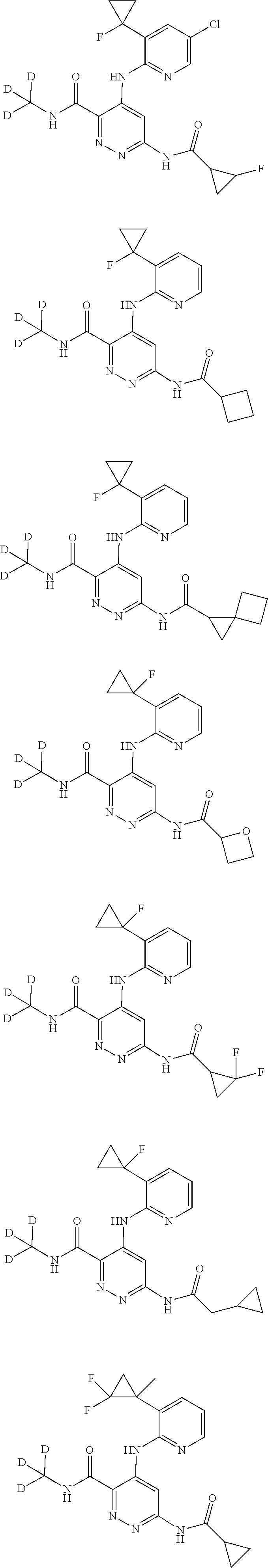

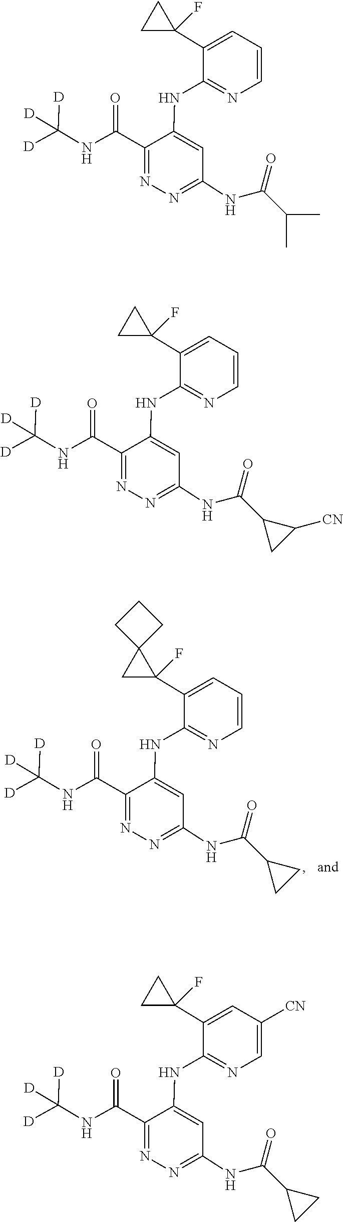 [SMILES: [2H]C([2H])([2H])NC(=O)c1nnc(NC(=O)C(C)C)cc1Nc1ncccc1C1(F)CC1 ; [2H]C([2H])([2H])NC(=O)c1nnc(NC(=O)C2CC2C#N)cc1Nc1ncccc1C1(F)CC1 ; [2H]C([2H])([2H])NC(=O)c1nnc(NC(=O)C2CC2)cc1Nc1ncccc1C1(F)CC12CCC2 ; [2H]C([2H])([2H])NC(=O)c1nnc(NC(=O)C2CC2)cc1Nc1ncc(C#N)cc1C1(F)CC1]

or a pharmaceutically acceptable salt, deuterated form, or stereoisomer thereof.
63. The compound of embodiment 1, wherein the compound is selected from the group consisting of:

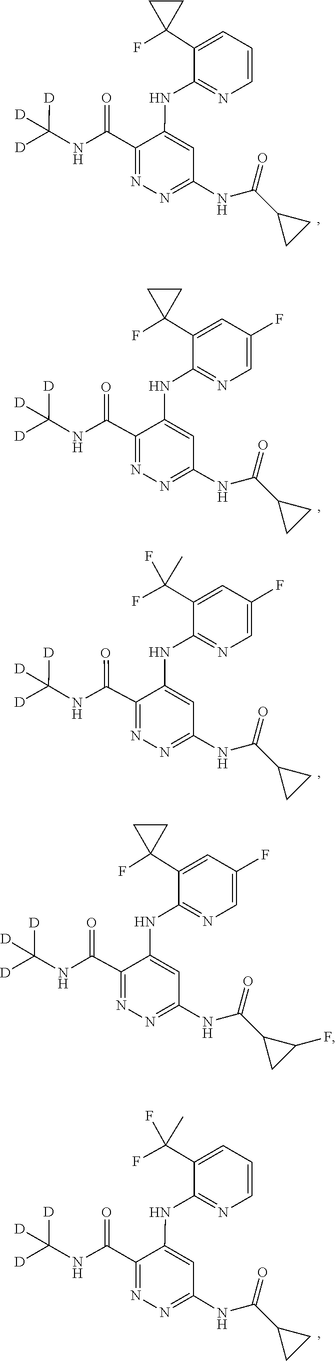 [SMILES: [2H]C([2H])([2H])NC(=O)c1nnc(NC(=O)C2CC2)cc1Nc1ncccc1C1(F)CC1 ; [2H]C([2H])([2H])NC(=O)c1nnc(NC(=O)C2CC2)cc1Nc1ncc(F)cc1C1(F)CC1 ; [2H]C([2H])([2H])NC(=O)c1nnc(NC(=O)C2CC2)cc1Nc1ncc(F)cc1C(C)(F)F ; [2H]C([2H])([2H])NC(=O)c1nnc(NC(=O)C2CC2F)cc1Nc1ncc(F)cc1C1(F)CC1 ; [2H]C([2H])([2H])NC(=O)c1nnc(NC(=O)C2CC2)cc1Nc1ncccc1C(C)(F)F]

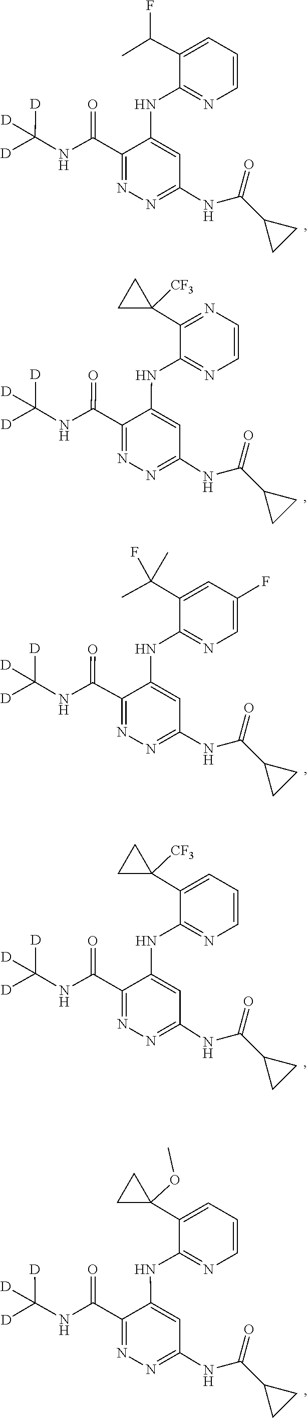 [SMILES: [2H]C([2H])([2H])NC(=O)c1nnc(NC(=O)C2CC2)cc1Nc1ncccc1C(C)F ; [2H]C([2H])([2H])NC(=O)c1nnc(NC(=O)C2CC2)cc1Nc1nccnc1C1(C(F)(F)F)CC1 ; [2H]C([2H])([2H])NC(=O)c1nnc(NC(=O)C2CC2)cc1Nc1ncc(F)cc1C(C)(C)F ; [2H]C([2H])([2H])NC(=O)c1nnc(NC(=O)C2CC2)cc1Nc1ncccc1C1(C(F)(F)F)CC1 ; [2H]C([2H])([2H])NC(=O)c1nnc(NC(=O)C2CC2)cc1Nc1ncccc1C1(OC)CC1]

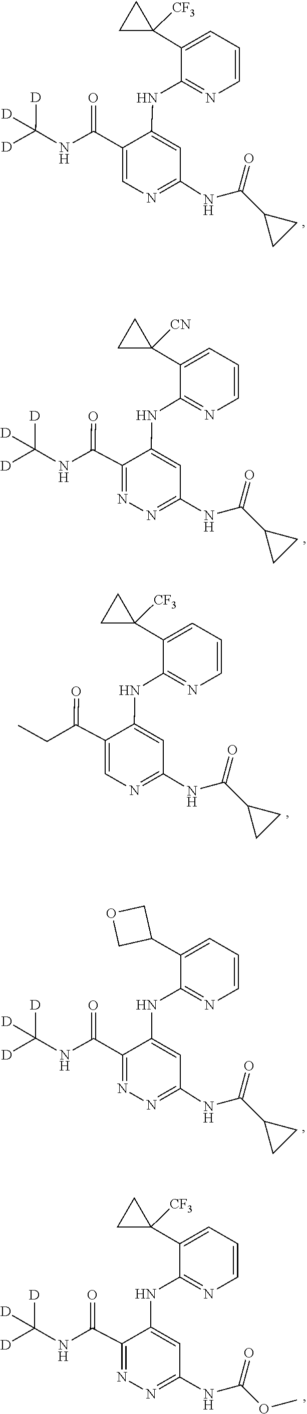 [SMILES: [2H]C([2H])([2H])NC(=O)c1cnc(NC(=O)C2CC2)cc1Nc1ncccc1C1(C(F)(F)F)CC1 ; [2H]C([2H])([2H])NC(=O)c1nnc(NC(=O)C2CC2)cc1Nc1ncccc1C1(C#N)CC1 ; CCC(=O)c1cnc(NC(=O)C2CC2)cc1Nc1ncccc1C1(C(F)(F)F)CC1 ; [2H]C([2H])([2H])NC(=O)c1nnc(NC(=O)C2CC2)cc1Nc1ncccc1C1COC1 ; [2H]C([2H])([2H])NC(=O)c1nnc(NC(=O)OC)cc1Nc1ncccc1C1(C(F)(F)F)CC1]

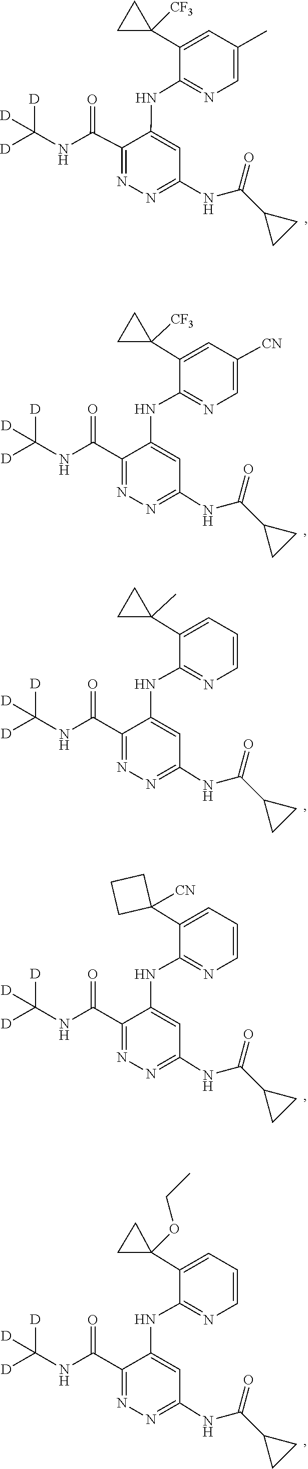 [SMILES: [2H]C([2H])([2H])NC(=O)c1nnc(NC(=O)C2CC2)cc1Nc1ncc(C)cc1C1(C(F)(F)F)CC1 ; [2H]C([2H])([2H])NC(=O)c1nnc(NC(=O)C2CC2)cc1Nc1ncc(C#N)cc1C1(C(F)(F)F)CC1 ; [2H]C([2H])([2H])NC(=O)c1nnc(NC(=O)C2CC2)cc1Nc1ncccc1C1(C)CC1 ; [2H]C([2H])([2H])NC(=O)c1nnc(NC(=O)C2CC2)cc1Nc1ncccc1C1(C#N)CCC1 ; [2H]C([2H])([2H])NC(=O)c1nnc(NC(=O)C2CC2)cc1Nc1ncccc1C1(OCC)CC1]

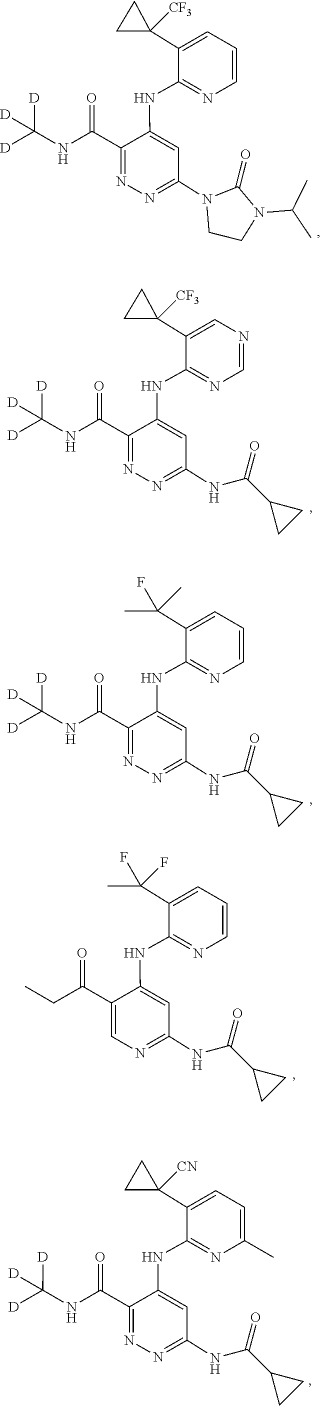

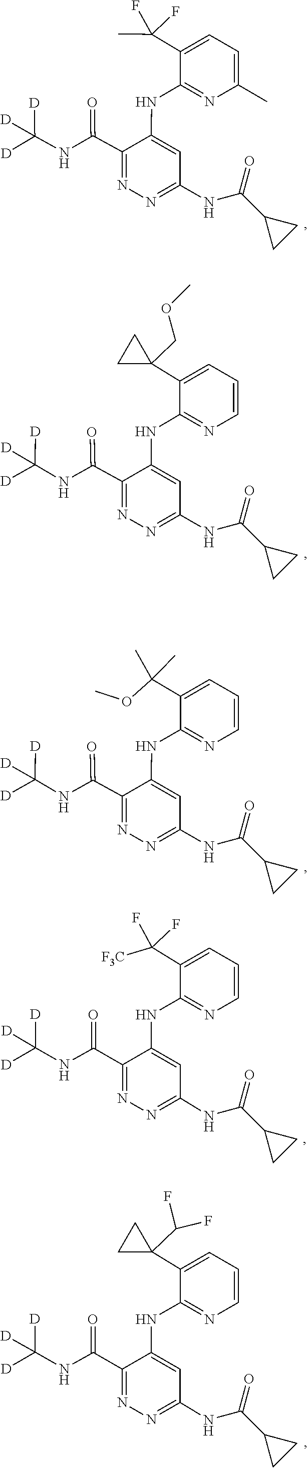 [SMILES: [2H]C([2H])([2H])NC(=O)c1nnc(NC(=O)C2CC2)cc1Nc1nc(C)ccc1C(C)(F)F ; [2H]C([2H])([2H])NC(=O)c1nnc(NC(=O)C2CC2)cc1Nc1ncccc1C1(COC)CC1 ; [2H]C([2H])([2H])NC(=O)c1nnc(NC(=O)C2CC2)cc1Nc1ncccc1C(C)(C)OC ; [2H]C([2H])([2H])NC(=O)c1nnc(NC(=O)C2CC2)cc1Nc1ncccc1C(F)(F)C(F)(F)F ; [2H]C([2H])([2H])NC(=O)c1nnc(NC(=O)C2CC2)cc1Nc1ncccc1C1(C(F)F)CC1]

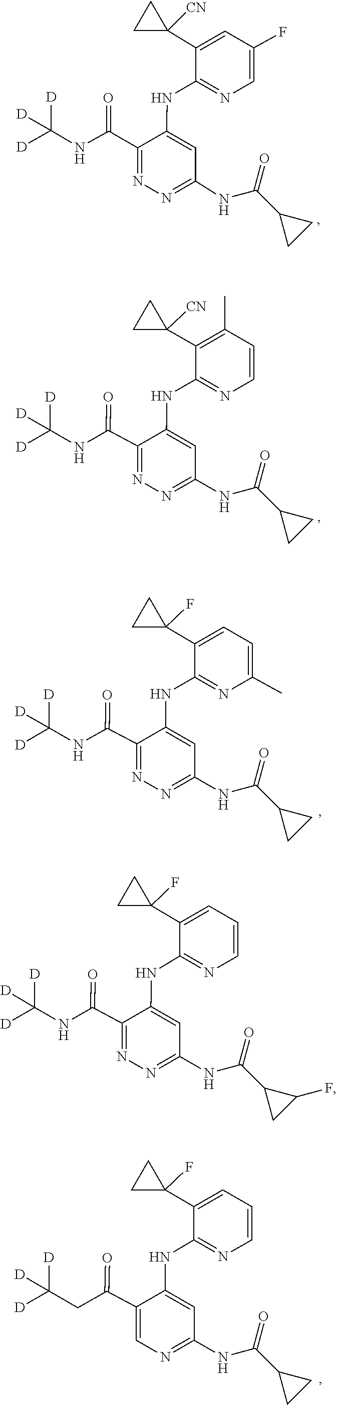 [SMILES: [2H]C([2H])([2H])NC(=O)c1nnc(NC(=O)C2CC2)cc1Nc1ncc(F)cc1C1(C#N)CC1 ; [2H]C([2H])([2H])NC(=O)c1nnc(NC(=O)C2CC2)cc1Nc1nccc(C)c1C1(C#N)CC1 ; [2H]C([2H])([2H])NC(=O)c1nnc(NC(=O)C2CC2)cc1Nc1nc(C)ccc1C1(F)CC1 ; [2H]C([2H])([2H])NC(=O)c1nnc(NC(=O)C2CC2F)cc1Nc1ncccc1C1(F)CC1 ; [2H]C([2H])([2H])CC(=O)c1cnc(NC(=O)C2CC2)cc1Nc1ncccc1C1(F)CC1]

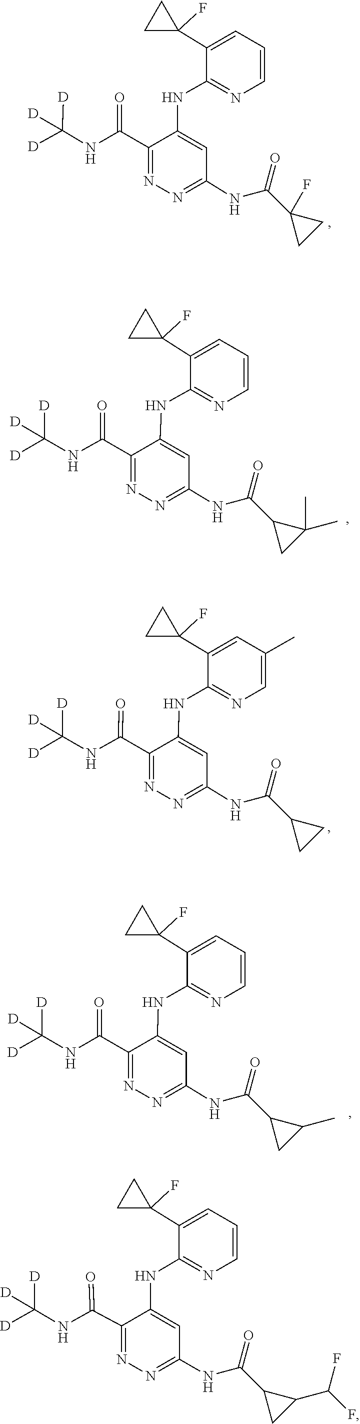 [SMILES: [2H]C([2H])([2H])NC(=O)c1nnc(NC(=O)C2(F)CC2)cc1Nc1ncccc1C1(F)CC1 ; [2H]C([2H])([2H])NC(=O)c1nnc(NC(=O)C2CC2(C)C)cc1Nc1ncccc1C1(F)CC1 ; [2H]C([2H])([2H])NC(=O)c1nnc(NC(=O)C2CC2)cc1Nc1ncc(C)cc1C1(F)CC1 ; [2H]C([2H])([2H])NC(=O)c1nnc(NC(=O)C2CC2C)cc1Nc1ncccc1C1(F)CC1 ; [2H]C([2H])([2H])NC(=O)c1nnc(NC(=O)C2CC2C(F)F)cc1Nc1ncccc1C1(F)CC1]

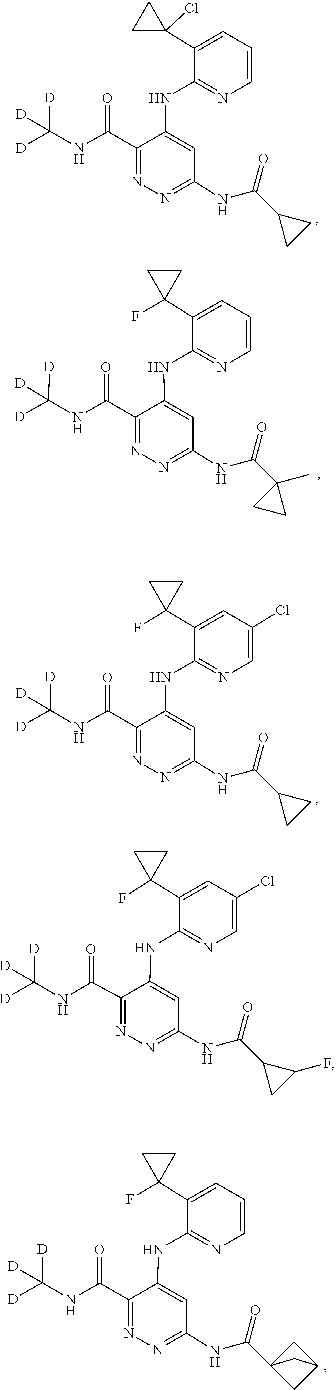 [SMILES: [2H]C([2H])([2H])NC(=O)c1nnc(NC(=O)C2CC2)cc1Nc1ncccc1C1(Cl)CC1 ; [2H]C([2H])([2H])NC(=O)c1nnc(NC(=O)C2(C)CC2)cc1Nc1ncccc1C1(F)CC1 ; [2H]C([2H])([2H])NC(=O)c1nnc(NC(=O)C2CC2)cc1Nc1ncc(Cl)cc1C1(F)CC1 ; [2H]C([2H])([2H])NC(=O)c1nnc(NC(=O)C2CC2F)cc1Nc1ncc(Cl)cc1C1(F)CC1 ; [2H]C([2H])([2H])NC(=O)c1nnc(NC(=O)C23CC(C2)C3)cc1Nc1ncccc1C1(F)CC1]

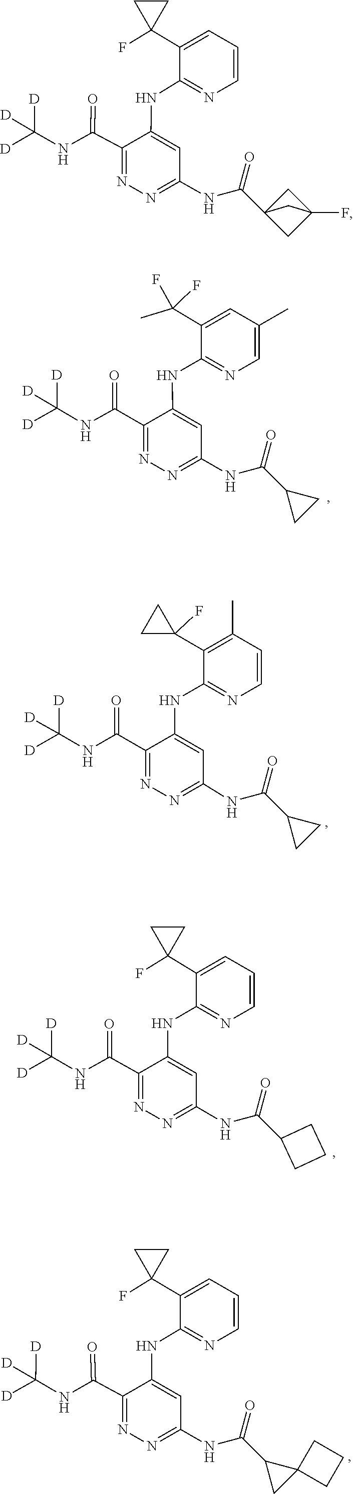 [SMILES: [2H]C([2H])([2H])NC(=O)c1nnc(NC(=O)C23CC(F)(C2)C3)cc1Nc1ncccc1C1(F)CC1 ; [2H]C([2H])([2H])NC(=O)c1nnc(NC(=O)C2CC2)cc1Nc1ncc(C)cc1C(C)(F)F ; [2H]C([2H])([2H])NC(=O)c1nnc(NC(=O)C2CC2)cc1Nc1nccc(C)c1C1(F)CC1 ; [2H]C([2H])([2H])NC(=O)c1nnc(NC(=O)C2CCC2)cc1Nc1ncccc1C1(F)CC1 ; [2H]C([2H])([2H])NC(=O)c1nnc(NC(=O)C2CC23CCC3)cc1Nc1ncccc1C1(F)CC1]

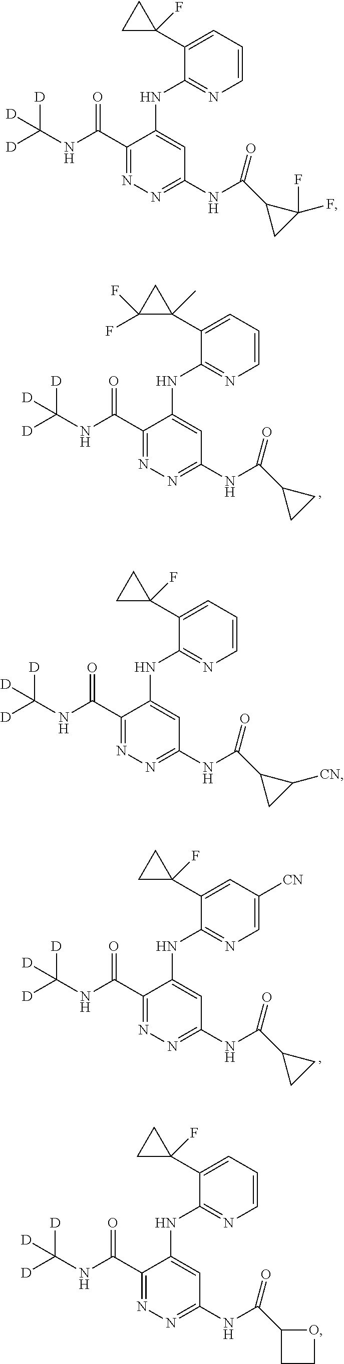 [SMILES: [2H]C([2H])([2H])NC(=O)c1nnc(NC(=O)C2CC2(F)F)cc1Nc1ncccc1C1(F)CC1 ; [2H]C([2H])([2H])NC(=O)c1nnc(NC(=O)C2CC2)cc1Nc1ncccc1C1(C)CC1(F)F ; [2H]C([2H])([2H])NC(=O)c1nnc(NC(=O)C2CC2C#N)cc1Nc1ncccc1C1(F)CC1 ; [2H]C([2H])([2H])NC(=O)c1nnc(NC(=O)C2CC2)cc1Nc1ncc(C#N)cc1C1(F)CC1 ; [2H]C([2H])([2H])NC(=O)c1nnc(NC(=O)C2CCO2)cc1Nc1ncccc1C1(F)CC1]

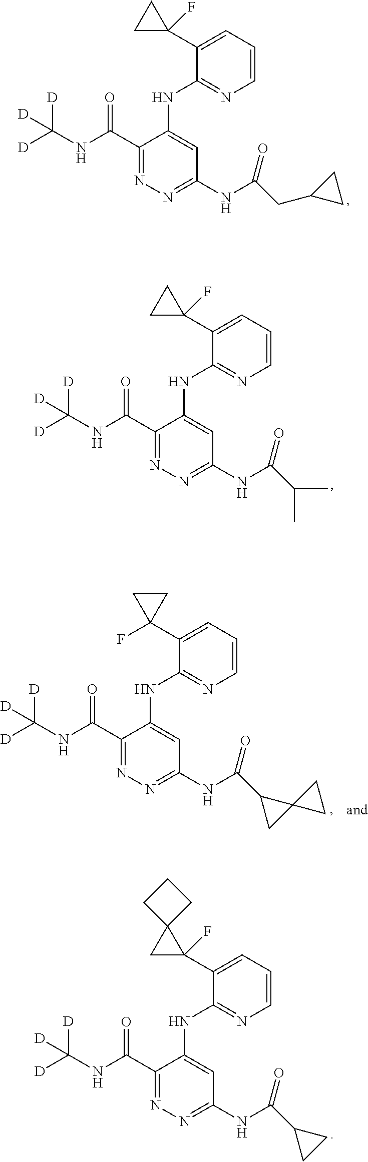 [SMILES: [2H]C([2H])([2H])NC(=O)c1nnc(NC(=O)CC2CC2)cc1Nc1ncccc1C1(F)CC1 ; [2H]C([2H])([2H])NC(=O)c1nnc(NC(=O)C(C)C)cc1Nc1ncccc1C1(F)CC1 ; [2H]C([2H])([2H])NC(=O)c1nnc(NC(=O)C2CC23CC3)cc1Nc1ncccc1C1(F)CC1 ; [2H]C([2H])([2H])NC(=O)c1nnc(NC(=O)C2CC2)cc1Nc1ncccc1C1(F)CC12CCC2]

Embodiment 64B. A pharmaceutical composition comprising a compound of any one of embodiments 1-62, or a pharmaceutically acceptable salt, deuterated form, or stereoisomer thereof, and a pharmaceutically acceptable carrier.

### EMBODIMENTS C

Embodiment 1C. A compound of Formula (I):

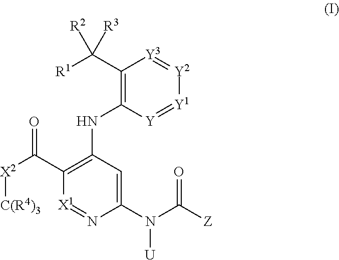

or a pharmaceutically acceptable salt, deuterated form, or stereoisomer thereof; wherein:
R¹ is —H, -halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, -halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, -halo, —C₁₋₆ alkyl, —O—C₁₋₆ alkyl, —C₁₋₆ alkylene-O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
Y is N or CR^{YA};
Y¹ is N or CR^{YB};
Y² is N or CR^{YC}; and
Y³ is N or CR^{YD};
R^{YA}, R^{YB}, R^{YC}, and R^{YD} are each independently —H, —CN, -halo, —C₁₋₆ alkyl, —OC₁₋₆ alkyl, —C₁₋₆ alkenyl, —C₁₋₆ alkynyl, —C₃₋₆ cycloalkyl, heterocyclyl, or aryl;
U is H;
Z is —OR^{Z}, —(CH₂)₀₋₃—(C₃₋₆ cycloalkyl), or —(CH₂)₀₋₃—(C₃-C₄ heterocyclyl), and wherein the —C₃₋₆ cycloalkyl is optionally substituted with 1 or more substituents each independently selected from the group consisting of -halo, —C₁₋₆ alkyl, —OC₁₋₆ alkyl, —OC₃₋₆ cycloalkyl, or —CN, or two substituents are taken together to form a carbocyclyl; or
U and Z are taken together to form a heterocyclyl optionally substituted with 1 or more substituents independently selected from the group consisting of -halo, —C₁₋₆ alkyl, or —OC₁₋₆ alkyl; and
R^{Z} is —C₁₋₆ alkyl, or —C₃₋₆ cycloalkyl;
and provided the compound is not:
3-pyridinecarboxamide, 6-[(cyclopropylcarbonyl)amino]-4-[[2-(1-hydroxy-1-methylethyl)phenyl]amino]-N-methyl-; or
6-(cyclopropanecarboxamido)-4-((2-(1-hydroxyethyl)-3-(1-methyl-1H-1,2,4-triazol-3-yl)phenyl)amino)-N-(methyl-d3)pyridazine-3-carboxamide.
   Embodiment 2C. The compound of embodiment 1, wherein Z is —OR^{Z} or —C₃₋₆ cycloalkyl, or —C₃-C₄ heterocyclyl wherein the —C₃₋₆ cycloalkyl or —C₃-C₄ heterocyclyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN or two substituents are taken together to form a carbocyclyl.
   Embodiment 3C. The compound of embodiment 1 or 2, wherein Z is —OR^{Z}, cyclopropyl, cyclobutyl, bicyclopentyl, spirohexyl, or oxetanyl, wherein the cyclopropyl, cyclobutyl, bicyclopentyl, spirohexyl, or oxetanyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN, or two substituents are taken together to form a carbocyclyl.
   Embodiment 4C. The compound of embodiment 3, wherein Z is —OR^{Z}, cyclopropyl, cyclobutyl, bicyclopentyl, spirohexyl, or oxetanyl, wherein the cyclopropyl, cyclobutyl, bicyclopentyl, spirohexyl, or oxetanyl is optionally substituted with 1, 2, or 3 substituents each independently selected from the group consisting of -halo, —C₁₋₆ alkyl, —OC₁₋₆ alkyl, —OC₃₋₆ cycloalkyl, or —CN.
   Embodiment 5C. The compound of any of embodiments 1-4, wherein Z is —(CH₂)₀₋₃—(C₃-C₆ cycloalkyl).
   Embodiment 6C. The compound of any of embodiments 1-4, wherein Z is —(CH₂)—(C₃-C₆ cycloalkyl).
   Embodiment 7C. The compound of any one of embodiments 1-4, wherein U is H.
   Embodiment 8C. The compound of embodiment 1, wherein U and Z are taken together to form a 5-8 membered heterocyclyl, wherein the 5-8 membered heterocyclyl is optionally substituted with 1, 2, or 3 substituents independently selected from the group consisting of -halo, —C₁₋₆ alkyl, or —OC₁₋₆ alkyl.
   Embodiment 9C. The compound of embodiment 8, wherein U and Z are taken together to form a 5-membered saturated heterocyclyl, wherein the 5-membered saturated heterocyclyl is optionally substituted with —C₁₋₆ alkyl.
   Embodiment 10C. The compound of embodiment 9, wherein the 5-membered saturated heterocyclyl is substituted with CH₃, CH₂CH₃, CH₂CH₂CH₃, or CH(CH₃)₂.
   Embodiment 11C. The compound of embodiment 1, having a structure of Formula (IA):

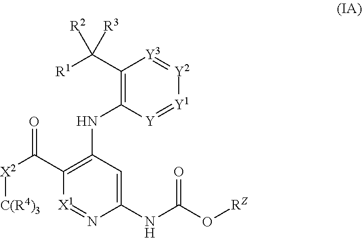

or a pharmaceutically acceptable salt, deuterated form, or stereoisomer thereof.
Embodiment 12C. The compound of any one of embodiments 1-3, having a structure of Formula (IB):

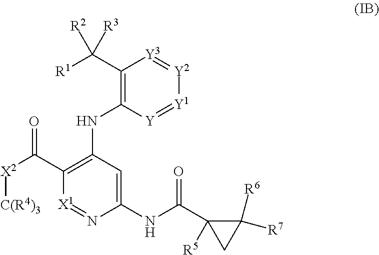

or a pharmaceutically acceptable salt, deuterated form, or stereoisomer thereof;
wherein R⁵, R⁶ and R⁷ are each independently selected from the group consisting of —H, -halo, —C₁₋₆ alkyl, —OC₁₋₆ alkyl, or —OC₃₋₆ cycloalkyl, or R⁶ and R⁷ are taken together to form a carbocyclyl.
Embodiment 13C. The compound of any one of embodiments 1-12, wherein Y, Y¹, Y², and Y³ are not all CH.
Embodiment 14C. The compound of any one of embodiments 1-13, wherein at least one of Y, Y¹, Y², and Y³ is N.
Embodiment 15C. The compound of any one of embodiments 1-14, wherein Y is N.
Embodiment 16C. The compound of any one of embodiments 1-15, wherein one or two of Y¹, Y², and Y³ is N.
Embodiment 17C. The compound of any one of embodiments 1-13, wherein Y is N and Y² is N.
Embodiment 18C. The compound of any one of embodiments 1-13, wherein Y is N and Y³ is N.
Embodiment 19C. The compound of any one of embodiments 1-12, wherein:
   Y is N and Y² is CR^{YC} wherein R^{YC} is CH₃, —CN, F, or Cl; or wherein
   Y is N and Y³ is CR^{YD} wherein R^{YD} is CH₃, —CN, F, or Cl.
Embodiment 20C. The compound of any one of embodiments 1-7 or 12-15, having a structure of Formula (IIA) or (IIB):

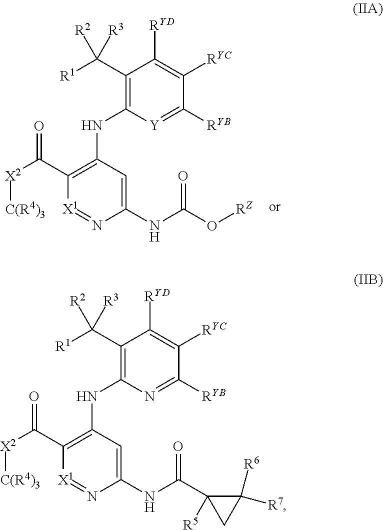

or a pharmaceutically acceptable salt, deuterated form, or stereoisomer thereof.
Embodiment 21C. The compound of any one of embodiments 13-20, wherein R⁵ is —H or halo.
Embodiment 22C. The compound of any one of embodiments 13-20, wherein R⁶ and R⁷ are each independently selected from the group consisting of —H, -halo, —C₁₋₆ alkyl, or —OC₁₋₆ alkyl, or R⁶ and R⁷ are taken together to form a C₃₋₆cycloalkyl.
Embodiment 23C. The compound of embodiment 22, wherein R⁶ and R⁷ are each independently selected from the group consisting of —H, -halo, —C₁₋₆ alkyl, or —OC₁₋₆ alkyl, or R⁶ and R⁷ are taken together to form a cyclopropyl or cyclobutyl.
Embodiment 24C. The compound of embodiment 23, wherein R⁶ and R⁷ are each independently selected from the group consisting of —H, -halo, —C₁₋₆ alkyl, or —OC₁₋₆ alkyl.
Embodiment 25C. The compound of any one of embodiments 13-21, wherein R⁵, R⁶ and R⁷ are H.
Embodiment 26C. The compound of any one of embodiments 1-25, wherein R^{YA}, R^{YB}, and R^{YC} are each independently —H, —CN, -halo, —C₁₋₆ alkyl, —C₁₋₆ alkenyl, —C₁₋₆ alkynyl, —OC₁₋₆ alkyl, —C₃₋₆ cycloalkyl, saturated heterocyclyl, aryl, or heteroaryl and R^{YD} is —H, —CN, -halo, —C₁₋₆ alkyl, —C₁₋₆ alkenyl, —C₁₋₆ alkynyl, —C₃₋₆ cycloalkyl, a saturated heterocyclyl, or aryl.
Embodiment 27C. The compound of any one of embodiments 1-26, wherein R^{YB}, R^{YC}, and R^{YD} are each independently —H, —C₁₋₆ alkyl, —OC₁₋₆ alkyl, —CN, or halo.
Embodiment 28C. The compound of embodiment 27, wherein R^{YB} and R^{YD} are —H and R^{YC} is —H, —C₁₋₆ alkyl, —CN, or —F.
Embodiment 29C. The compound of embodiment 27, wherein two or three of R^{YB}, R^{YC}, and R^{YD} are —H.
Embodiment 30C. The compound of any one of embodiments 1-29, wherein:
   R¹ is —H, -halo, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —O-saturated heterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or —CO₂alkyl;
   R² is —H, -halo, —C₁₋₆ alkyl, —C₁₋₆ alkenyl, —C₁₋₆ alkynyl, —C₃₋₆ cycloalkyl, or a saturated heterocyclyl; wherein the —C₁₋₆ alkyl, —C₁₋₆ alkenyl, —C₁₋₆ alkynyl, —C₃₋₆ cycloalkyl, or saturated heterocyclyl are each independently optionally substituted with 1 or more halo, —CN, —OC₁₋₆ alkyl, —OC₁₋₆ haloalkyl, or -CO₂alkyl;
   or R¹ and R² are taken together to form a carbocyclyl or saturated heterocyclyl, wherein the carbocyclyl or saturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl.
Embodiment 31C. The compound of any one of embodiments 1-29, wherein:
   R¹ is —H, -halo, —OH, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, or —O-saturated heterocyclyl;
   R² is —H, -halo, —C₁₋₆ alkyl, —C₁₋₆ alkenyl, —C₁₋₆ alkynyl, —C₃₋₆ cycloalkyl, or saturated heterocyclyl; or
   R¹ and R² are taken together to form a carbocyclyl or saturated heterocyclyl.
Embodiment 32C. The compound of any one of embodiments 1-31, wherein:
   R¹ is —C₁₋₆ alkyl;
   R² is —H, —C₁₋₆ alkyl, or -halo; or
   R¹ and R² are taken together to form a carbocyclyl or saturated heterocyclyl.
Embodiment 33C. The compound of any one of embodiments 1-32, wherein R¹ is C₁₋₆ alkyl.
Embodiment 34C. The compound of embodiment 33, wherein R¹ is —CH₃.
Embodiment 35C. The compound of any one of embodiments 1-34, wherein R¹ and R² are taken together to form a carbocyclyl.
Embodiment 36C. The compound of any one of embodiments 1-32, wherein R¹ and R² are taken together to form a —C₃₋₆ cycloalkyl or saturated heterocyclyl.
Embodiment 37C. The compound of embodiment 32 or 33, wherein R¹ and R² are taken together to form a —C₃₋₆ cycloalkyl.
Embodiment 38C. The compound of embodiment 37, wherein R¹ and R² are taken together to form a cyclopropyl, spiropentyl, or oxetanyl.
Embodiment 39C. The compound of any one of embodiments 32-35, wherein R¹ and R² are taken together to form a cyclopropyl or spiropentyl.
Embodiment 40C. The compound of any of embodiments 35-39, wherein the carbocycle or heterocycle is substituted with —CH₂OCH₃.
Embodiment 41C. The compound of embodiment 35, wherein R¹ and R² are taken together to form a C₆₋₈ bicyclic cycloalkyl.
Embodiment 42C. The compound of any one of embodiments 1-36, wherein R² is —H or -halo.
Embodiment 43C. The compound of embodiment 42, wherein R² is —H.
Embodiment 44C. The compound of embodiment 42, wherein R² is halo.
Embodiment 45C. The compound of embodiment 42, wherein R² is F.
Embodiment 46C. The compound of any one of embodiments 1-45, wherein R³ is —H, -halo, —C₁₋₆ alkyl, —C₁₋₆ haloalkyl, —O—C₁₋₆ alkyl, or CN.
Embodiment 47C. The compound of any one of embodiments 1-46, wherein R³ is -halo, —C₁₋₆ haloalkyl, —O—C₁₋₆ alkyl, or CN.
Embodiment 48C. The compound of any one of embodiments 1-47, wherein R³ is C₁₋₆ haloalkyl, —OC₁₋₆ alkyl, or CN.
Embodiment 49C. The compound of any one of embodiments 1-48, wherein R³ is C₁₋₃ haloalkyl, —OC₁₋₃ alkyl, or CN.
Embodiment 50C. The compound of embodiment 46, wherein R³ is —H, —F, —CH₃, —CH₂F, CHF₂, —CF₃, —OCH₃, —OCH₂CH₃, or CN.
Embodiment 51C. The compound of embodiment 46, wherein R³ is —F, —CH₃, —CF₃, —OCH₃, —OCH₂CH₃, or CN.
Embodiment 52C. The compound of embodiment 51, wherein R³ is —CH₃, —CF₃, —OCH₃, —OCH₂CH₃, or CN.
Embodiment 53C. The compound of embodiment 46, wherein R³ is H, F, or Cl.
Embodiment 54C. The compound of embodiment 53, wherein R³ is F.
Embodiment 55C. The compound of any one of embodiments 1-54, wherein X² is CHR⁸.
Embodiment 56C. The compound of any one of embodiments 1-55, wherein X² is CH₂.
Embodiment 57C. The compound of any one of embodiments 1-54, wherein X² is NH.
Embodiment 58C. The compound of any one of embodiments 1-57, wherein each R⁴ is —H.
Embodiment 59C. The compound of any one of embodiments 1-57, wherein each R⁴ is -D.
Embodiment 60C. The compound of any one of embodiments 1-59, wherein X¹ is CH.
Embodiment 61C. The compound of any one of embodiments 1-59, wherein X¹ is N.
Embodiment 62C. The compound of embodiment 1, wherein the compound is selected from the group consisting of:

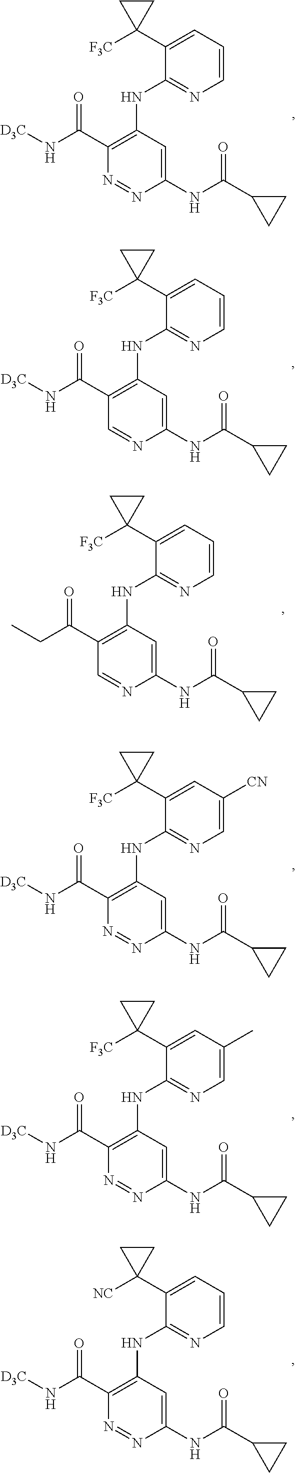 [SMILES: [2H]C([2H])([2H])NC(=O)c1nnc(NC(=O)C2CC2)cc1Nc1ncccc1C1(C(F)(F)F)CC1 ; [2H]C([2H])([2H])NC(=O)c1cnc(NC(=O)C2CC2)cc1Nc1ncccc1C1(C(F)(F)F)CC1 ; CCC(=O)c1cnc(NC(=O)C2CC2)cc1Nc1ncccc1C1(C(F)(F)F)CC1 ; [2H]C([2H])([2H])NC(=O)c1nnc(NC(=O)C2CC2)cc1Nc1ncc(C#N)cc1C1(C(F)(F)F)CC1 ; [2H]C([2H])([2H])NC(=O)c1nnc(NC(=O)C2CC2)cc1Nc1ncc(C)cc1C1(C(F)(F)F)CC1 ; [2H]C([2H])([2H])NC(=O)c1nnc(NC(=O)C2CC2)cc1Nc1ncccc1C1(C#N)CC1]

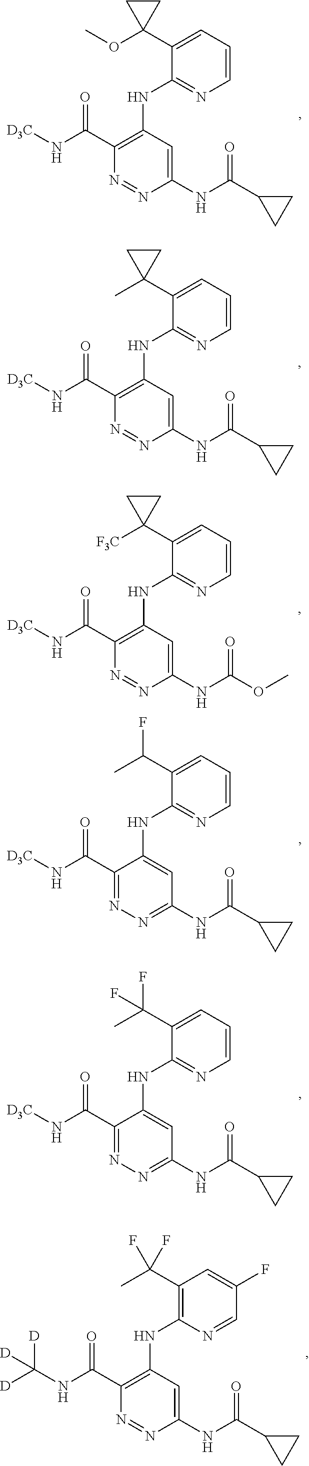

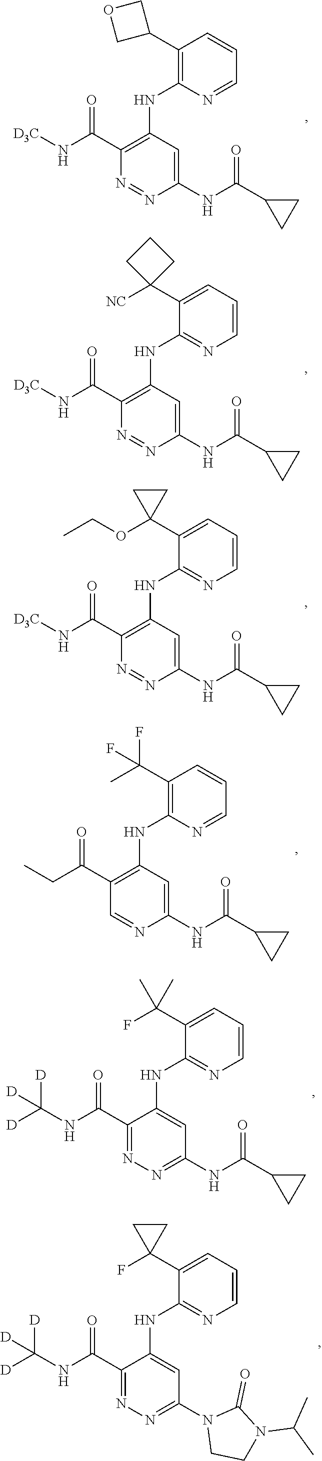

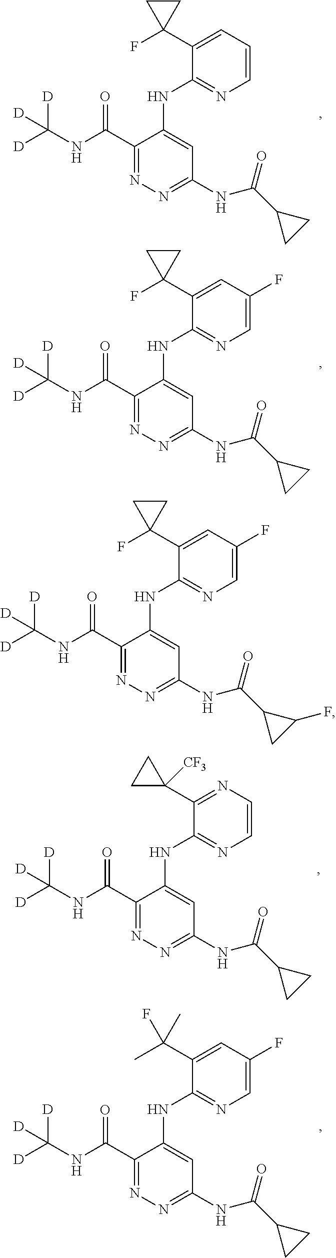

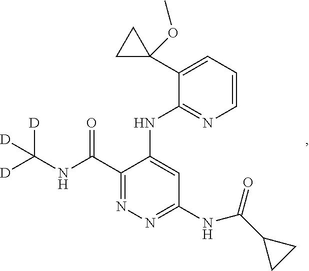 [SMILES: [2H]C([2H])([2H])NC(=O)c1nnc(NC(=O)C2CC2)cc1Nc1ncccc1C1(OC)CC1]

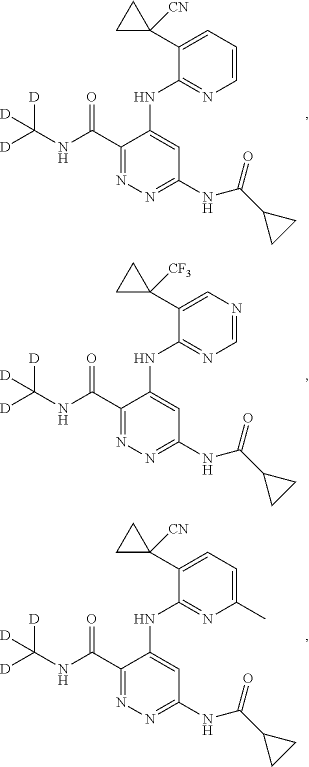 [SMILES: [2H]C([2H])([2H])NC(=O)c1nnc(NC(=O)C2CC2)cc1Nc1ncccc1C1(C#N)CC1 ; [2H]C([2H])([2H])NC(=O)c1nnc(NC(=O)C2CC2)cc1Nc1ncncc1C1(C(F)(F)F)CC1 ; [2H]C([2H])([2H])NC(=O)c1nnc(NC(=O)C2CC2)cc1Nc1nc(C)ccc1C1(C#N)CC1]

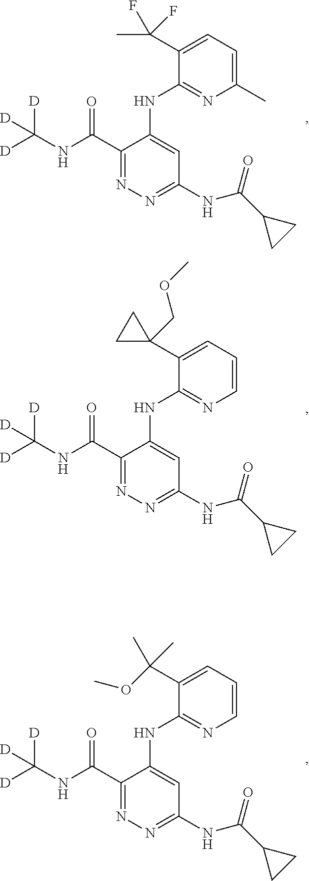 [SMILES: [2H]C([2H])([2H])NC(=O)c1nnc(NC(=O)C2CC2)cc1Nc1nc(C)ccc1C(C)(F)F ; [2H]C([2H])([2H])NC(=O)c1nnc(NC(=O)C2CC2)cc1Nc1ncccc1C1(COC)CC1 ; [2H]C([2H])([2H])NC(=O)c1nnc(NC(=O)C2CC2)cc1Nc1ncccc1C(C)(C)OC]

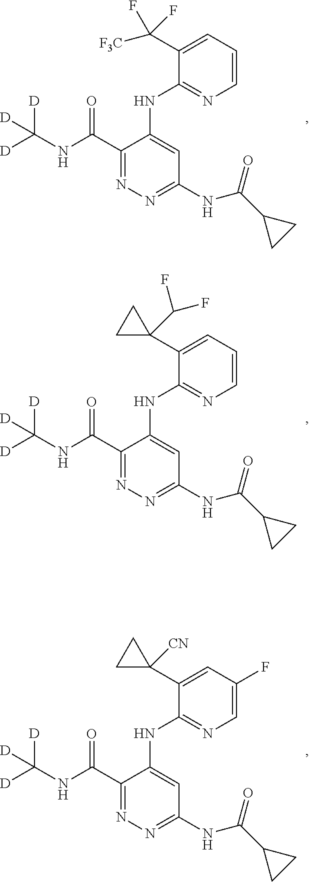 [SMILES: [2H]C([2H])([2H])NC(=O)c1nnc(NC(=O)C2CC2)cc1Nc1ncccc1C(F)(F)C(F)(F)F ; [2H]C([2H])([2H])NC(=O)c1nnc(NC(=O)C2CC2)cc1Nc1ncccc1C1(C(F)F)CC1 ; [2H]C([2H])([2H])NC(=O)c1nnc(NC(=O)C2CC2)cc1Nc1ncc(F)cc1C1(C#N)CC1]

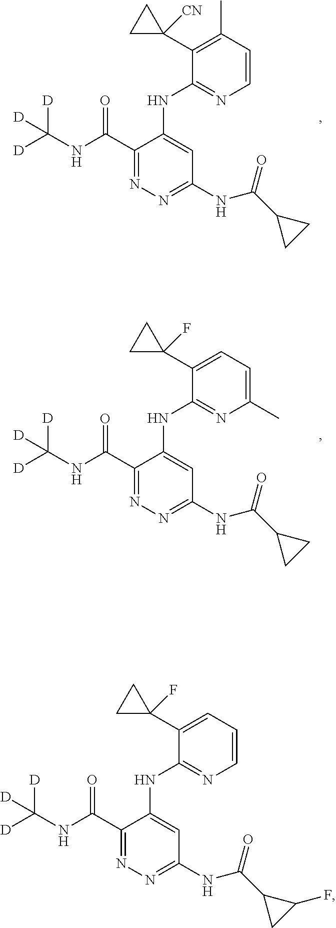 [SMILES: [2H]C([2H])([2H])NC(=O)c1nnc(NC(=O)C2CC2)cc1Nc1nccc(C)c1C1(C#N)CC1 ; [2H]C([2H])([2H])NC(=O)c1nnc(NC(=O)C2CC2)cc1Nc1nc(C)ccc1C1(F)CC1 ; [2H]C([2H])([2H])NC(=O)c1nnc(NC(=O)C2CC2F)cc1Nc1ncccc1C1(F)CC1]

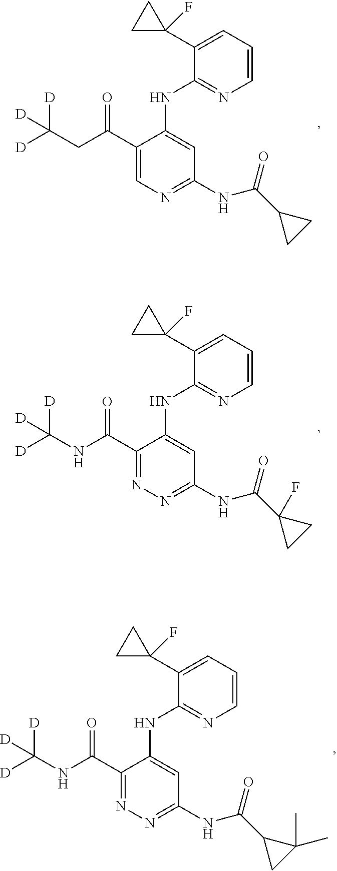 [SMILES: [2H]C([2H])([2H])CC(=O)c1cnc(NC(=O)C2CC2)cc1Nc1ncccc1C1(F)CC1 ; [2H]C([2H])([2H])NC(=O)c1nnc(NC(=O)C2(F)CC2)cc1Nc1ncccc1C1(F)CC1 ; [2H]C([2H])([2H])NC(=O)c1nnc(NC(=O)C2CC2(C)C)cc1Nc1ncccc1C1(F)CC1]

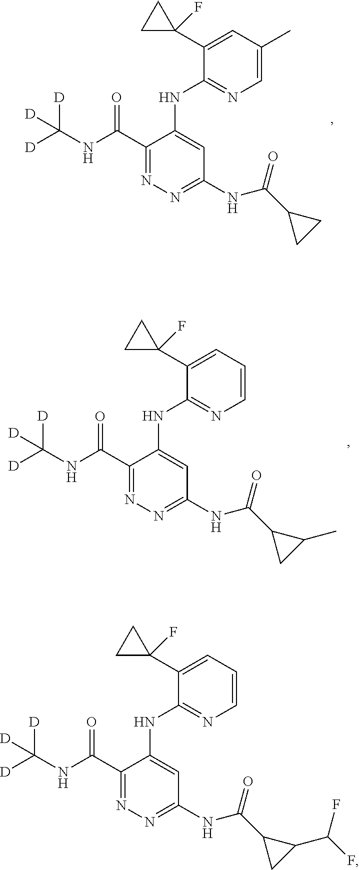 [SMILES: [2H]C([2H])([2H])NC(=O)c1nnc(NC(=O)C2CC2)cc1Nc1ncc(C)cc1C1(F)CC1 ; [2H]C([2H])([2H])NC(=O)c1nnc(NC(=O)C2CC2C)cc1Nc1ncccc1C1(F)CC1 ; [2H]C([2H])([2H])NC(=O)c1nnc(NC(=O)C2CC2C(F)F)cc1Nc1ncccc1C1(F)CC1]

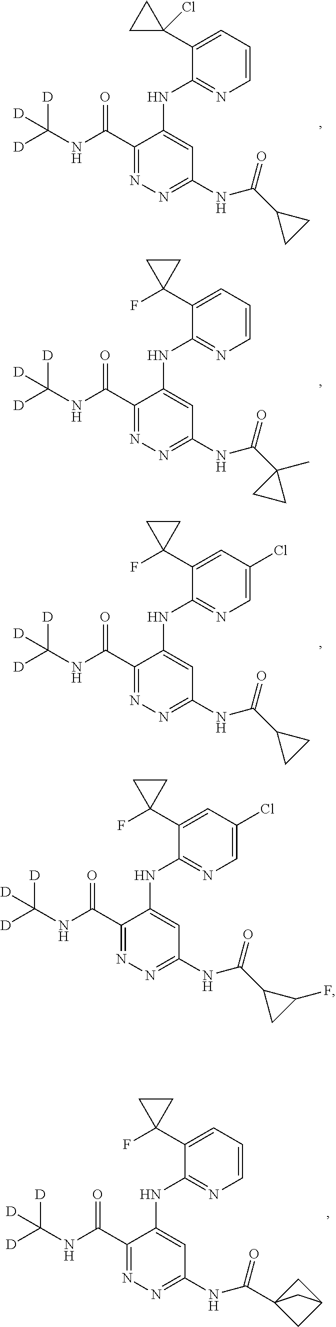 [SMILES: [2H]C([2H])([2H])NC(=O)c1nnc(NC(=O)C2CC2)cc1Nc1ncccc1C1(Cl)CC1 ; [2H]C([2H])([2H])NC(=O)c1nnc(NC(=O)C2(C)CC2)cc1Nc1ncccc1C1(F)CC1 ; [2H]C([2H])([2H])NC(=O)c1nnc(NC(=O)C2CC2)cc1Nc1ncc(Cl)cc1C1(F)CC1 ; [2H]C([2H])([2H])NC(=O)c1nnc(NC(=O)C2CC2F)cc1Nc1ncc(Cl)cc1C1(F)CC1 ; [2H]C([2H])([2H])NC(=O)c1nnc(NC(=O)C23CC(C2)C3)cc1Nc1ncccc1C1(F)CC1]

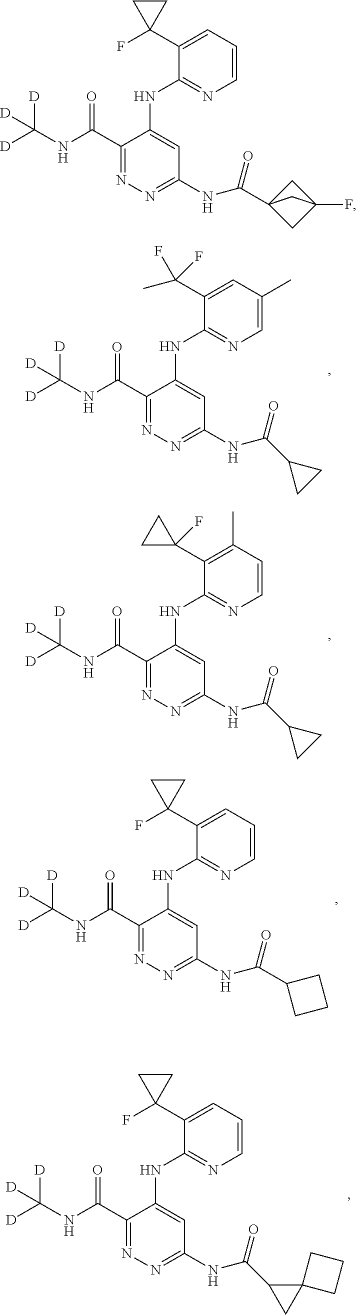 [SMILES: [2H]C([2H])([2H])NC(=O)c1nnc(NC(=O)C23CC(F)(C2)C3)cc1Nc1ncccc1C1(F)CC1 ; [2H]C([2H])([2H])NC(=O)c1nnc(NC(=O)C2CC2)cc1Nc1ncc(C)cc1C(C)(F)F ; [2H]C([2H])([2H])NC(=O)c1nnc(NC(=O)C2CC2)cc1Nc1nccc(C)c1C1(F)CC1 ; [2H]C([2H])([2H])NC(=O)c1nnc(NC(=O)C2CCC2)cc1Nc1ncccc1C1(F)CC1 ; [2H]C([2H])([2H])NC(=O)c1nnc(NC(=O)C2CC23CCC3)cc1Nc1ncccc1C1(F)CC1]

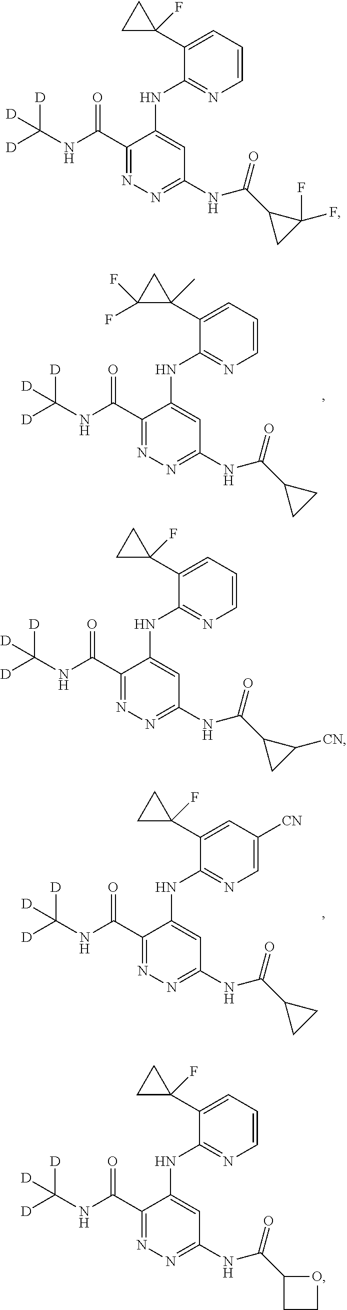 [SMILES: [2H]C([2H])([2H])NC(=O)c1nnc(NC(=O)C2CC2(F)F)cc1Nc1ncccc1C1(F)CC1 ; [2H]C([2H])([2H])NC(=O)c1nnc(NC(=O)C2CC2)cc1Nc1ncccc1C1(C)CC1(F)F ; [2H]C([2H])([2H])NC(=O)c1nnc(NC(=O)C2CC2C#N)cc1Nc1ncccc1C1(F)CC1 ; [2H]C([2H])([2H])NC(=O)c1nnc(NC(=O)C2CC2)cc1Nc1ncc(C#N)cc1C1(F)CC1 ; [2H]C([2H])([2H])NC(=O)c1nnc(NC(=O)C2CCO2)cc1Nc1ncccc1C1(F)CC1]

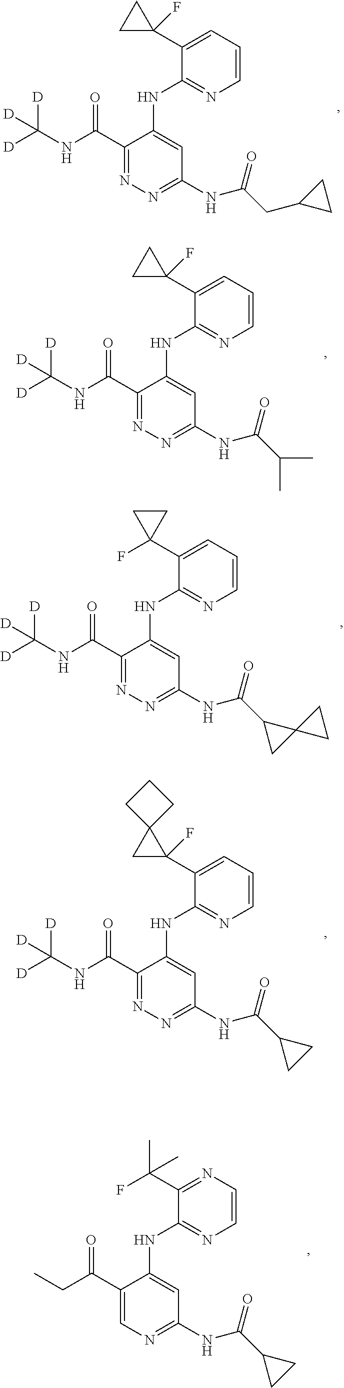

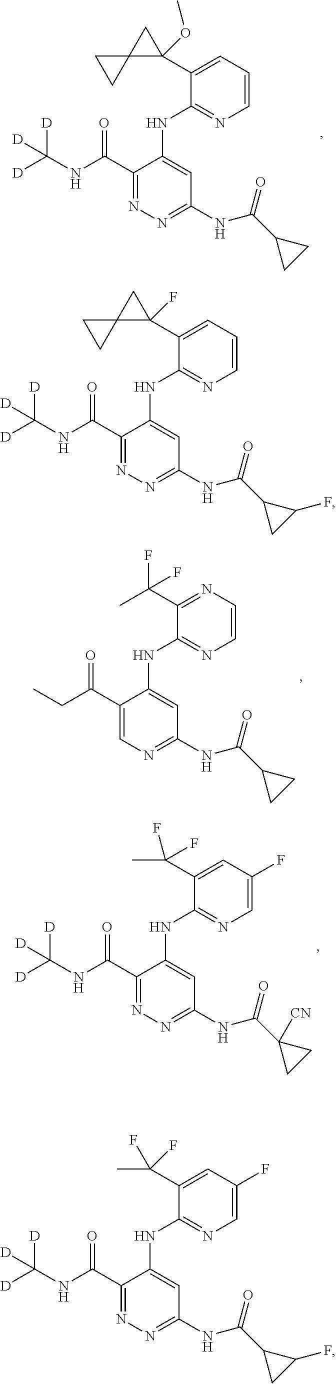 [SMILES: [2H]C([2H])([2H])NC(=O)c1nnc(NC(=O)C2CC2)cc1Nc1ncccc1C1(OC)CC12CC2 ; [2H]C([2H])([2H])NC(=O)c1nnc(NC(=O)C2CC2F)cc1Nc1ncccc1C1(F)CC12CC2 ; CCC(=O)c1cnc(NC(=O)C2CC2)cc1Nc1nccnc1C(C)(F)F ; [2H]C([2H])([2H])NC(=O)c1nnc(NC(=O)C2(C#N)CC2)cc1Nc1ncc(F)cc1C(C)(F)F ; [2H]C([2H])([2H])NC(=O)c1nnc(NC(=O)C2CC2F)cc1Nc1ncc(F)cc1C(C)(F)F]

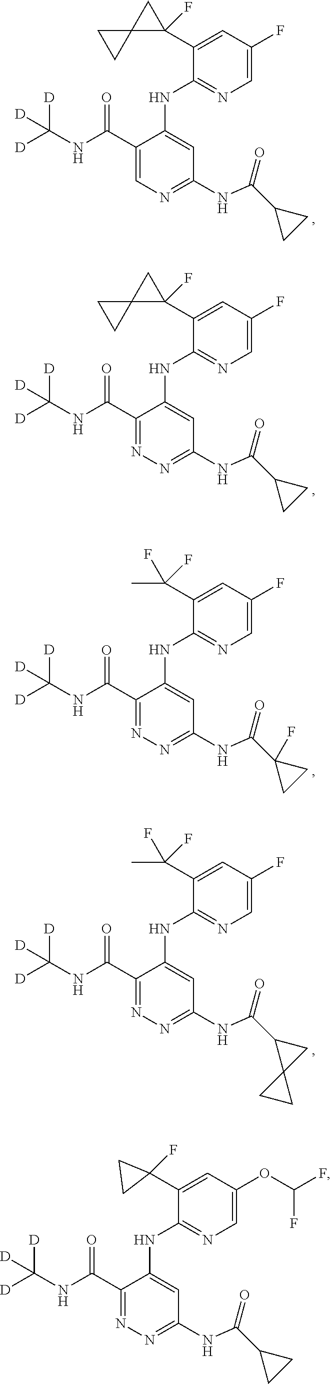 [SMILES: [2H]C([2H])([2H])NC(=O)c1cnc(NC(=O)C2CC2)cc1Nc1ncc(F)cc1C1(F)CC12CC2 ; [2H]C([2H])([2H])NC(=O)c1nnc(NC(=O)C2CC2)cc1Nc1ncc(F)cc1C1(F)CC12CC2 ; [2H]C([2H])([2H])NC(=O)c1nnc(NC(=O)C2(F)CC2)cc1Nc1ncc(F)cc1C(C)(F)F ; [2H]C([2H])([2H])NC(=O)c1nnc(NC(=O)C2CC23CC3)cc1Nc1ncc(F)cc1C(C)(F)F ; [2H]C([2H])([2H])NC(=O)c1nnc(NC(=O)C2CC2)cc1Nc1ncc(OC(F)F)cc1C1(F)CC1]

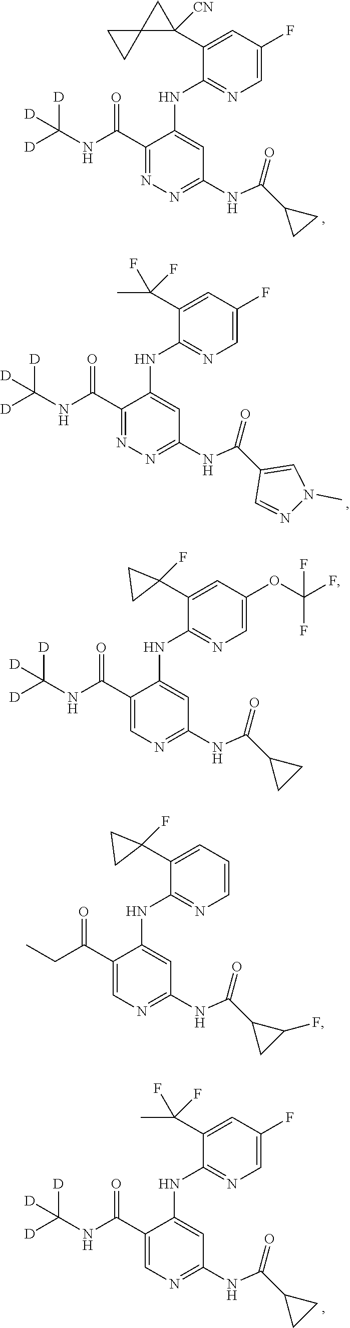

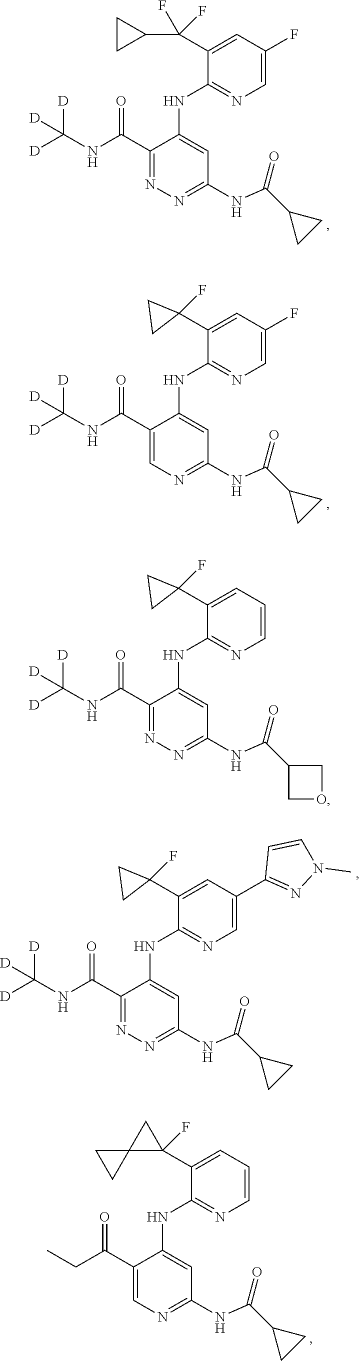

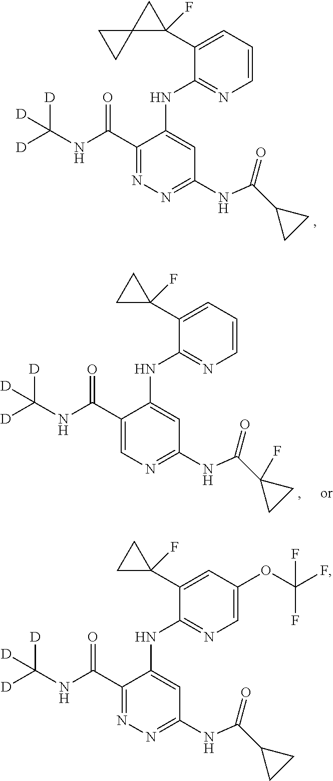 [SMILES: [2H]C([2H])([2H])NC(=O)c1nnc(NC(=O)C2CC2)cc1Nc1ncccc1C1(F)CC12CC2 ; [2H]C([2H])([2H])NC(=O)c1cnc(NC(=O)C2(F)CC2)cc1Nc1ncccc1C1(F)CC1 ; [2H]C([2H])([2H])NC(=O)c1nnc(NC(=O)C2CC2)cc1Nc1ncc(OC(F)(F)F)cc1C1(F)CC1]

or a pharmaceutically acceptable salt, deuterated form, or stereoisomer thereof.
Embodiment 63C. A pharmaceutical composition comprising a compound of any one of embodiments 1-62, or a pharmaceutically acceptable salt, deuterated form, or stereoisomer thereof, and a pharmaceutically acceptable carrier.

### EXAMPLES

The disclosure now being generally described, it will be more readily understood by reference to the following examples which are included merely for purposes of illustration of certain aspects and embodiments of the present invention, and are not intended to limit the invention.

### LC-MS Method A:

### Instrumentation

LC: Waters Acquity UPLC
UV Detection: Waters Acquity PDA (198-360 nm), 20 pts/sec
MS Detection: Waters Acquity QDa, ESI (ES+/ES−, 120-1200 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 0.9 mL/min
Run Time: 2.7 minutes

### LC-MS Method AA:

### Instrumentation

LC: Waters Acquity UPLC
UV Detection: Waters Acquity PDA (198-360 nm), 20 pts/sec
MS Detection: Waters Acquity QDa, ESI (ES+/ES−, 120-1200 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method B:

### Instrumentation

LC: Waters Acquity UPLC
UV Detection: Waters Acquity PDA (198-360 nm), 20 pts/sec
MS Detection: Waters Acquity QDa ESI (ES+/ES−, 120-1200 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 0.9 mL/min
Run Time: 2.7 minutes

### LC-MS Method BB:

### Instrumentation

LC: Waters Acquity UPLC
UV Detection: Waters Acquity PDA (198-360 nm), 20 pts/sec
MS Detection: Waters Acquity QDa ESI (ES+/ES−, 120-1200 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method C:

### Instrumentation

LC: Waters Acquity UPLC
UV Detection: Waters Acquity PDA (198-360 nm), 20 pts/sec
MS Detection: Waters Acquity QDa, ESI (ES+/ES−, 120-1200 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method D:

### Instrumentation

LC: Waters Acquity UPLC
UV Detection: Waters Acquity PDA (198-360 nm), 20 pts/sec
MS Detection: Waters Acquity QDa, ESI (ES+/ES−, 120-1200 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method E:

### Instrumentation

LC: Waters Acquity UPLC
UV Detection: Waters Acquity PDA (198-360 nm), 20 pts/sec
MS Detection: Waters 3100 MS, ESI (ES+/ES−, 120-1900 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 0.9 mL/min
Run Time: 2.7 minutes

### LC-MS Method EE:

### Instrumentation

LC: Waters Acquity UPLC

UV Detection: Waters Acquity PDA (198-360 nm), 20 pts/sec

MS Detection: Waters 3100 MS, ESI (ES+/ES−, 120-1900 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method F:

### Instrumentation

LC: Waters Acquity UPLC
UV Detection: Waters Acquity PDA (198-360 nm), 20 pts/sec
MS Detection: Waters 3100 MS, ESI (ES+/ES−, 120-1900 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 0.9 mL/min
Run Time: 2.7 minutes

### LC-MS Method FF:

### Instrumentation

LC: Waters Acquity UPLC
UV Detection: Waters Acquity PDA (198-360 nm), 20 pts/sec
MS Detection: Waters 3100 MS, ESI (ES+/ES−, 120-1900 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method G:

### Instrumentation

LC: Waters Acquity UPLC
UV Detection: Waters Acquity PDA (198-360 nm), 20 pts/sec
MS Detection: Waters 3100 MS, ESI (ES+/ES−, 120-1900 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method H:

### Instrumentation

LC: Waters Acquity UPLC
UV Detection: Waters Acquity PDA (198-360 nm), 20 pts/sec
MS Detection: Waters 3100 MS, ESI (ES+/ES−, 120-1900 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method I:

### Instrumentation

LC: Waters Acquity UPLC
UV Detection: Waters Acquity PDA (198-360 nm), 20 pts/sec
MS Detection: Waters SQD, ESI (ES+/ES−, 120-1900amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 0.9 mL/min
Run Time: 2.7 minutes

### LC-MS Method II:

### Instrumentation

LC: Waters Acquity UPLC
UV Detection: Waters Acquity PDA (198-360 nm), 20 pts/sec
MS Detection: Waters SQD, ESI (ES+/ES−, 120-1200 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method J:

### Instrumentation

LC: Waters Acquity UPLC
UV Detection: Waters Acquity PDA (198-360 nm), 20 pts/sec
MS Detection: Waters SQD, ESI (ES+/ES−, 120-1200 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 0.9 mL/min
Run Time: 2.7 minutes

### LC-MS Method JJ:

### Instrumentation

LC: Waters Acquity UPLC
UV Detection: Waters Acquity PDA (198-360 nm), 20 pts/sec
MS Detection: Waters SQD, ESI (ES+/ES−, 120-1200 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method K:

### Instrumentation

LC: Waters Acquity UPLC
UV Detection: Waters Acquity PDA (198-360 nm), 20 pts/sec
MS Detection: Waters SQD, ESI (ES+/ES−, 120-1200 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method L:

### Instrumentation

LC: Waters Acquity UPLC
UV Detection: Waters Acquity PDA (198-360 nm), 20 pts/sec
MS Detection: Waters SQD, ESI (ES+/ES−, 120-1200 amu)

### Mobile Phase

Eluent A1: Milli-Q H₂O+10 mM AmB pH: 10
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method M:

### Instrumentation

LC: Waters Acquity H-Class
UV Detection: Waters Acquity PDA (198-360 nm), 20 pts/sec
MS Detection: Waters ZQ 2000, ESI (ES+, 120-1900 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 1.4 minutes; hold 100% B for 0.4 minute
Flow: 1.1 mL/min
Run Time: 1.8 minutes

### LC-MS Method N:

### Instrumentation

LC: Waters Acquity H-Class
UV Detection: Waters Acquity PDA (198-360 nm), 20 pts/sec
MS Detection: Waters ZQ 2000, ESI (ES+, 120-1900 amu)

### Mobile Phase

Eluent A1: Milli-Q H₂O+10 mM AmB pH: 10
Eluent B1: ACN
Column: Waters Acquity UPLC CSH C18, 1.7 μm, 2.1×30 mm at 40° C.
Gradient: 5% to 100% B in 1.4 minutes; hold 100% B for 0.5 minute
Flow: 1.1 mL/min
Run Time: 1.9 minutes

### LC-MS Method O:

### Instrumentation

LC: Waters Acquity H-Class
UV Detection: Waters Acquity PDA (198-360 nm), 20 pts/sec
MS Detection: Waters ZQ 2000, ESI (ES+, 120-1900 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method P:

### Instrumentation

LC: Waters Acquity H-Class
UV Detection: Waters Acquity PDA (198-360 nm), 20 pts/sec
MS Detection: Waters ZQ 2000, ESI (ES+, 120-1900 amu)

### Mobile Phase

Eluent A1: Milli-Q H₂O+10 mM AmB pH: 10
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method Q:

### Instrumentation

LC: Waters Acquity UPLC
UV Detection: Waters Acquity PDA (198-360 nm), 20 pts/sec
MS Detection: Waters Acquity QDa Performance, ESI (ES+/ES−, 120-1200 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method R:

### Instrumentation

LC: Waters Acquity UPLC
UV Detection: Waters Acquity PDA (198-360 nm), 20 pts/sec
MS Detection: Waters Acquity QDa Performance, ESI (ES+/ES−, 120-1200 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 2.0 minutes; hold 100% B for 0.7 minute
Flow: 1.1 mL/min
Run Time: 1.9 minutes

### LC-MS Method S:

### Instrumentation

LC: Waters Acquity UPLC
UV Detection: Waters Acquity PDA (198-360 nm), 20 pts/sec
MS Detection: Waters Acquity QDa Performance, ESI (ES+/ES−, 120-1200 amu)

### Mobile Phase

Eluent A1: Milli-Q H₂O+10 mM AmF pH: 3.8
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### LC-MS Method T:

### Instrumentation

LC: Waters Acquity UPLC
UV Detection: Waters Acquity PDA (198-360 nm), 20 pts/sec
MS Detection: Waters Acquity QDa Performance, ESI (ES+/ES−, 120-1200 amu)

### Mobile Phase

Eluent A1: Milli-Q H₂O+10 mM AmB pH: 10.0
Eluent B1: ACN
Column: Waters Acquity UPLC CSH C18, 1.7 μm, 2.1×30 mm at 40° C.
Gradient: 5% to 100% B in 5.2 minutes; hold 100% B for 1.8 minute
Flow: 0.9 mL/min
Run Time: 7 minutes

### Abbreviations

| | |
|---|---|
| AmB | ammonium bicarbonate |
| AmF | ammonium formate |
| anh. | anhydrous |
| aq. | aqueous |
| bpy | bipyridine |
| dba | dibenzylideneacetone |
| DCM | dichloromethane |
| dcpf | 1,1′-bis(dicyclohexylphosphino)ferrocene |
| DIPEA | N,N-diisopropylethylamine |
| DMA | N,N-dimethylacetamide |
| DMAP | 4-(dimethylamino)pyridine |
| DMB | 2,4-dimethoxybenzyl |
| DMF | N,N-dimethylformamide |
| DMSO | dimethyl sulfoxide |
| dppf | 1,1′-bis(diphenylphosphino)ferrocene |
| EDC | N-(3-dimethylaminopropyl)-N′-ethylcarbodiimide |
| equiv. | equivalent(s) |
| EtOAc | ethyl acetate |
| g | gram(s) |
| h | hour(s) |
| HFIP | 1,1,1,3,3,3-hexafluoroisopropanol |
| IPA | isopropyl alcohol (2-propanol) |
| K₃PO₄ | potassium phosphate tribasic |
| LC | liquid chromatography |
| LiHMDS | lithium bis(trimethylsilyl)amide |
| MeCN | acetonitrile |
| MeOH | methanol |
| mg | milligram(s) |
| min | minute(s) |
| mL | milliliter(s) |
| mmol | millimoles |
| MsCl | methanesulfonyl chloride |
| NaH | sodium hydride |
| NaHMDS | sodium bis(trimethylsilyl)amide |
| NaOtBu | sodium tert-butoxide |
| NMI | 1-methylimidazole |
| Pd₂(dba)₃ | tris(dibenzylideneacetone)dipalladium(0) |
| satd. or sat. | saturated |
| T3P | Propylphosphonic anhydride |
| tBuDavePhos | 2-di-tert-butylphosphino-2′-(N,N- |
| | dimethylamino)biphenyl |
| TCFH | N,N,N′,N′-tetramethylchloroformamidinium |
| | hexafluorophosphate |
| TMSCl | trimethylsilyl chloride |
| uL | microliter(s) |
| umol | micromoles |
| XantPhos | 4,5-bis(diphenylphosphino)-9,9-dimethylxanthene |
| XPhos | (2-dicyclohexylphosphino-2′,4′,6′-triisopropylbiphenyl) |

### Synthetic Procedures

The compounds of the present disclosure can be prepared in a variety of ways known to one skilled in the art of organic synthesis. The compounds of the present disclosure can be synthesized using the methods as hereinafter described below, together with synthetic methods known in the art of synthetic organic chemistry or variations thereon as appreciated by those skilled in the art.

Additionally, preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

The compounds provided herein may be isolated and purified by known standard procedures. Such procedures include (but are not limited to) silica gel chromatography using various organic solvents such as hexane, dichloromethane, ethyl acetate, methanol, isopropyl alcohol, acetonitrile combinations thereof and the like, reverse phase flash chromatography using various solvents such as acetonitrile, AmB buffer, preparative high pressure liquid chromatography, preparative reverse phase high pressure liquid chromatography.

The following schemes are presented with details as to the preparation of representative compounds that have been listed herein.

### Synthesis of Intermediates

The following known intermediates were made according to reported procedures in WO2023027948A1, WO2024257023 A1, WO2022241174A1, US20130225552, WO2023227946, WO2020086616, and WO2018071794 which are each incorporated by reference herein.

| Name | Structure | reference |
|---|---|---|
| 4,6-dichloro-N-(methyl- d₃)pyridazine-3-carboxamide | 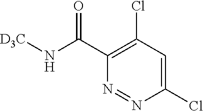 [SMILES: [2H]C([2H])([2H])NC(=O)c1nnc(Cl)cc1Cl] | WO2023027948A1 |
| 4-amino-6- (cyclopropanecarboxamido)- N-(methyl-d₃)pyridazine-3- carboxamide | 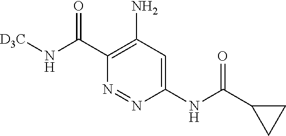 [SMILES: [2H]C([2H])([2H])NC(=O)c1nnc(NC(=O)C2CC2)cc1N] | WO2023027948A1 |
| 4-bromo-6- (cyclopropanecarboxamido)- N-(methyl-d₃)pyridazine-3- carboxamide | 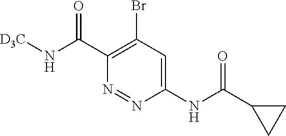 [SMILES: [2H]C([2H])([2H])NC(=O)c1nnc(NC(=O)C2CC2)cc1Br] | WO2023027948A1 |
| 4-chloro-6- (cyclopropanecarboxamido)- N-(methyl-d₃)nicotinamide | 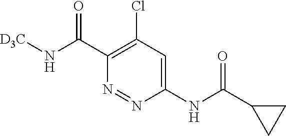 [SMILES: [2H]C([2H])([2H])NC(=O)c1nnc(NC(=O)C2CC2)cc1Cl] | WO2024257023 A1 |
| N-(4-chloro-5- propionylpyridin-2- yl)cyclopropanecarboxamide | 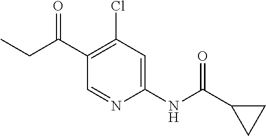 [SMILES: CCC(=O)c1cnc(NC(=O)C2CC2)cc1Cl] | WO2022241174A1 |
| 1-(2-chloro-5-fluoropyridin- 3-yl)cyclopropane-1- carboxylic acid | 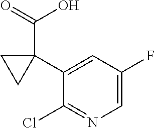 [SMILES: O=C(O)C1(c2cc(F)cnc2Cl)CC1] | US20130225552 A1 |
| 2-chloro-5-fluoro-N- methoxy-N- methylnicotinamide | 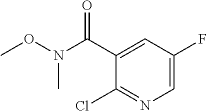 | WO2023227946 A1 |
| N-(4-chloro-5-(propanoyl- 3,3,3-d₃)pyridin-2- yl)cyclopropanecarboxamide | 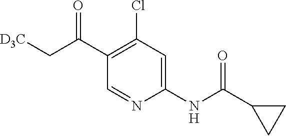 [SMILES: [2H]C([2H])([2H])CC(=O)c1cnc(NC(=O)C2CC2)cc1Cl] | WO2020086616 A1 |
| (1S,2S)-2- fluorocyclopropane-1- carboxamide | 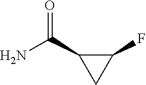 | WO2018071794 A1 |

### Synthesis of 4-amino-6-(cyclopropanecarboxamido)-N-(methyl-d₃)nicotinamide

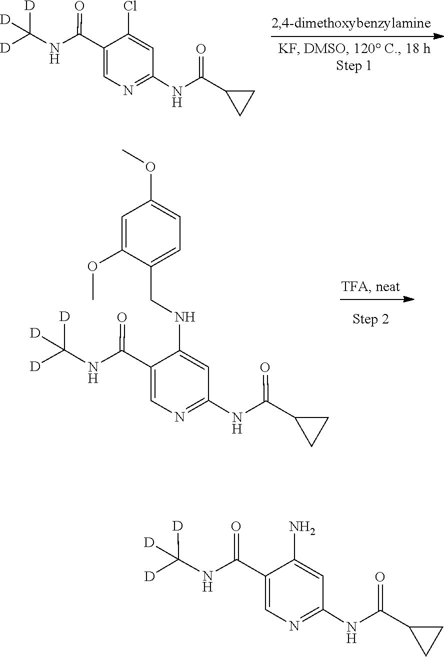 [SMILES: [2H]C([2H])([2H])NC(=O)c1cnc(NC(=O)C2CC2)cc1Cl ; [2H]C([2H])([2H])NC(=O)c1cnc(NC(=O)C2CC2)cc1NCc1ccc(OC)cc1OC ; [2H]C([2H])([2H])NC(=O)c1cnc(NC(=O)C2CC2)cc1N]

### Step 1: Synthesis of 6-(cyclopropanecarboxamido)-4-((2,4-dimethoxybenzyl)amino)-N-(methyl-d₃)nicotinamide

4-chloro-6-(cyclopropanecarboxamido)-N-(methyl-d₃)nicotinamide (1.20 g, 4.67 mmol), potassium fluoride (815 mg, 14.0 mmol), 2,4-dimethoxybenzylamine (3.58 mL, 23.4 mmol) and DMSO (24.0 mL) were stirred at 120° C. overnight (18 h). The reaction was cooled to ambient temperature and ca. 30 mL of water was added. The resulting precipitate was filtered and suction-dried affording the title compound (2.05 g, 113%) as a pale brown solid. LCMS method B (m/z): [M+H]⁺=388.3, retention time=1.01 min. Note: product contaminated with starting material 2,4-dimethoxybenzylamine resulting in higher than 100% yield. Carried forward without purification

### Step 2: Synthesis of 4-amino-6-(cyclopropanecarboxamido)-N-(methyl-d₃)nicotinamide

6-(cyclopropanecarboxamido)-4-((2,4-dimethoxybenzyl)amino)-N-(methyl-d₃)nicotinamide (2.00 g, 5.16 mmol) and TFA (18.0 mL, 235 mmol) were stirred at ambient temperature for 30 mins. The TFA was removed under a stream of compressed air leaving a thick pink suspension which was made basic with 20% NH₄OH resulting in a yellow heterogenous mixture. 10% MeOH in DCM was added and the mixture stirred at room temperature for 15 min. The mixture was filtered and the precipitate was washed with water and suction-dried. The organic portion of the filtrate was separated and extracted with water. The combined aqueous portions were concentrated under a stream of compressed air. The residue was taken up in DMSO and purified by reverse phase flash chromatography (C18 silica with water/ACN: 5%-50%), the fractions containing product were combined and concentrated under vacuum. The resulting white solid was combined with the precipitate and triturated in DCM, filtered and air dried affording the title compound (1.06 g, 87%) as a white solid. LCMS method B (m/z): [M+H]⁺=238.3, retention time=0.47 min.

### Synthesis of (1S,2S)—N-(4-amino-5-propionylpyridin-2-yl)-2-fluorocyclopropane-1-carboxamide

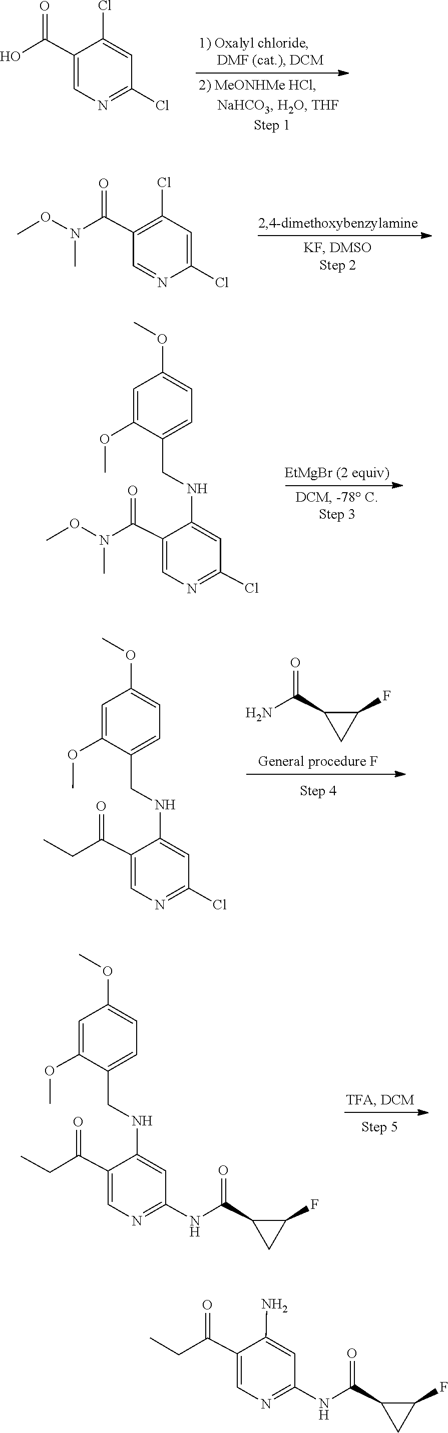

### Step 1: Synthesis of 4,6-dichloro-N-methoxy-N-methylnicotinamide

To a solution of 4,6-dichloronicotinic acid (3.38 g, 17.6 mmol) in DCM (40.0 mL) was added oxalyl chloride (4.00 mL, 46.3 mmol) followed by 2 drops of dimethylformamide (20.0 uL, 255 umol). The reaction was stirred at room temperature for 4 h. The reaction was concentrated and then redissolved in DCM (40.0 mL) to be added dropwise to a mixture of N,O-dimethylhydroxylamine hydrochloride (4.00 g, 40.1 mmol), THF (40.0 mL) and saturated aqueous NaHCO₃ (150 mL, 171 mmol). The mixture was stirred at room temperature for 60 min. The mixture was extracted with DCM (3×100 mL). The combined organic was washed with sat. NaHCO₃, sat. NH₄Cl, H₂O and brine, dried over Na₂SO₄, filtered, concentrated under reduced pressure and to provide the title compound (3.85 g, 93%) as an amber oil which was used without further purification. LCMS Method Q (m/z): [M+H]⁺=234.9, retention time=0.81 min.

### Step 2: Synthesis of 6-chloro-4-((2,4-dimethoxybenzyl)amino)-N-methoxy-N-methylnicotinamide

4,6-dichloro-N-methoxy-N-methylnicotinamide (4.93 g, 21.0 mmol), potassium fluoride (1.64 g, 27.9 mmol), 2,4-dimethoxybenzylamine (5.00 mL, 32.9 mmol) and DMSO (75.0 mL) were stirred at room temperature for 24 h. More 2,4-dimethoxybenzylamine (5.00 mL, 32.9 mmol) was added and the mixture was heated to 40° C. and stirred for 3.5 h and then at 60° C. for 6 h and back at 30° C. overnight (18 h). The mixture was diluted with water and extracted with DCM (3×100 mL). The combined organic was washed with NH₄Cl sat. (3×100 mL), half brine (1×100 mL) and brine (100 mL). The organic was dried over Na₂SO₄, filtered, concentrated under reduced pressure and dried under high vacuum to provide the title compound (9.06 g, 100%) as a viscous yellow oil which was used without purification. LCMS Method Q (m/z): [M+H]⁺=366.2, retention time=1.13 min.

### Step 3: Synthesis of 1-(6-chloro-4-((2,4-dimethoxybenzyl)amino)pyridin-3-yl)propan-1-one

A stirring solution of 6-chloro-4-((2,4-dimethoxybenzyl)amino)-N-methoxy-N-methylnicotinamide (7.67 g, 21.0 mmol) in DCM (200 mL) was cooled at −78° C. in a dry ice/acetone bath for 30 min under nitrogen. A solution of 3M ethylmagnesium bromide (27.0 mL, 80.9 mmol) in Et₂O was added dropwise and the mixture was stirred at −78° C. for 2.5 h. More ethylmagnesium bromide, 3 M in ether (2.00 mL, 6.00 mmol) was added and the mixture was stirred at −78° C. for 2.5 h. The mixture was stored in the freezer (−20° C.) 18 h. The mixture was then cooled back at −78° C., quenched with 50 mL sat. NH₄Cl added dropwise and then allowed to warm to room temperature. Water and DCM were added. The organic phase was separated, washed with NH₄Cl half sat. (3×), water, NaHCO₃ sat. and brine, dried over Na₂SO₄, filtered and concentrated under reduced pressure and dried under high vacuum to provide the title compound (7.56 g, 108%) as a yellow solid which was used without further purification. LCMS Method Q (m/z): [M+H]⁺=335.0, retention time=1.28 min.

### Step 4: Synthesis of (1S,2S)—N-(4-((2,4-dimethoxybenzyl)amino)-5-propionylpyridin-2-yl)-2-fluorocyclopropane-1-carboxamide

The title compound was prepared according to General Procedure F

To a degassed mixture of 1-(6-chloro-4-((2,4-dimethoxybenzyl)amino)pyridin-3-yl)propan-1-one (5.85 g, 17.5 mmol), (1S,2S)-2-fluorocyclopropane-1-carboxamide (1.80 g, 17.5 mmol) and K₃PO₄ (10.3 g, 47.5 mmol) in dioxane (200 mL) was added Pd₂(dba)₃ (803 mg, 859 umol) and dcpf (1.07 g, 1.76 mmol). Nitrogen was immediately bubbled in the mixture for 25 min and the mixture was heated at 100° C. for 10 h. The mixture was cooled, diluted with EtOAc and filtered on Celite®. The Celite® cake was rinsed with 10% MeOH in EtOAc and the combined organic was concentrated under reduced pressure. The residue was purified by silica gel chromatography (0-5% MeOH:DCM) to provide the title compound (5.44 g, 76%) as a yellow solid. LCMS Method Q (m/z): [M+H]⁺=402.2, retention time=1.13 min.

### Step 5: Synthesis of (1S,2S)—N-(4-amino-5-propionylpyridin-2-yl)-2-fluorocyclopropane-1-carboxamide

To a stirring solution of (1S,2S)—N-(4-((2,4-dimethoxybenzyl)amino)-5-propionylpyridin-2-yl)-2-fluorocyclopropane-1-carboxamide (5.44 g, 13.6 mmol) in DCM (100 mL) was added trifluoroacetic acid (5.00 mL, 65.3 mmol) at room temperature. The mixture was stirred at room temperature for 1 h. More trifluoroacetic acid (5.00 mL, 65.3 mmol) was added and the mixture was stirred at room temperature for 4.5 h. The mixture was added to stirring ice/water (300 mL) and was stirred for 2 h. The pH was adjusted to 12 with NaOH (5N and 1N). The mixture was filtered and the solid washed with water and DCM. The solid was dried under high vacuum for 2 days to provide the title compound (5.18 g, 152%) as a off-white solid which was used without further purification. LCMS Method Q (m/z): [M+H]⁺=252.0, retention time=0.68 min.

The following compound was prepared in a similar manner using the corresponding 4,6-dichloropyridazine-3-carboxylic acid

| | | | (m/z) [M + H]⁺ |
|---|---|---|---|
| Name | Structure | MW (g/mol) | (method) |
| N-(5-amino-6-propionylpyridazin-3- yl)cyclopropanecarboxamide | 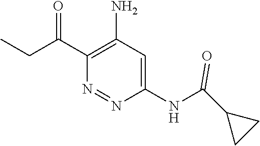 [SMILES: CCC(=O)c1nnc(NC(=O)C2CC2)cc1N] | 234.3 | 235.0 (Q) |

### General Procedure A

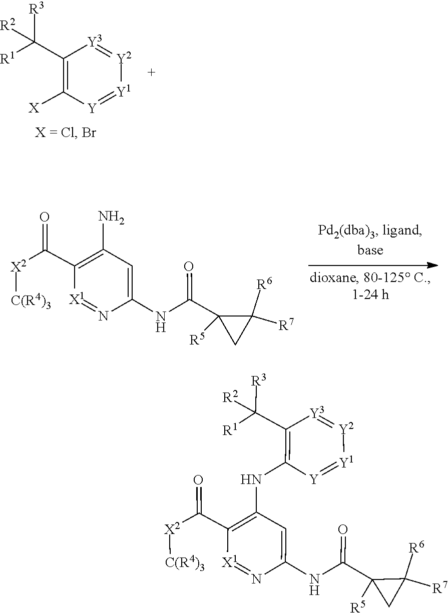

A mixture of an appropriately substituted heteroaryl aniline (1.1 equiv.), 2-halo-heteroarene (1.0 equiv.), Pd2(dba)3, dcpf or XantPhos (0.2-0.4 equiv.), K₃PO₄ or Cs₂CO₃ or K₂CO₃ (2.5-4.0 equiv.), and dioxane (0.1-0.2 M) in a pressure vessel was degassed with nitrogen gas for 5 mins. The vessel was sealed and placed on a heating block at 80-125° C. for 1-24 h. The reaction was cooled to ambient temperature and filtered through ca. 10 g of silica gel topped with Celite® using EtOAc as eluent. The filtrate was adsorbed onto silica gel and purified over normal phase flash chromatography to afford the final product. Oftentimes the final compound required another purification by reverse phase flash chromatography on C18 column to provide the product with acceptable purity.

### General Procedure B

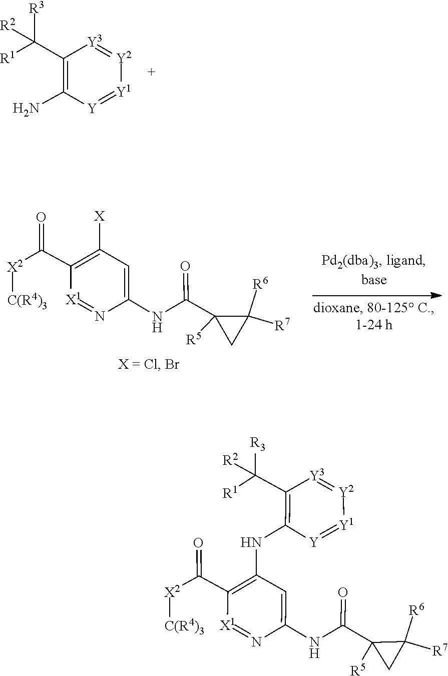

A mixture of an appropriately substituted heteroaryl chloride (1.1 equiv.), 2-aminoheteroarene or pyrazine (1.0 equiv.), Pd2(dba)3 (0.1-0.2 equiv.), dcpf or XantPhos (0.2-0.4 equiv.), K3PO4 or Cs2CO3 (2.5-4.0 equiv.), and dioxane (0.1-0.2 M) in a pressure vessel was degassed with nitrogen gas for 5 mins. The vessel was sealed and placed on a heating block at 80-125° C. for 1-24 h. The reaction was cooled to ambient temperature and filtered through ca. 10 g of silica gel topped with Celite® using EtOAc as eluent. The filtrate was adsorbed onto silica gel and purified over normal phase flash chromatography to afford the final product. Oftentimes the final compound required another purification by reverse phase flash chromatography on C18 column to provide the product with acceptable purity.

### General Procedure C

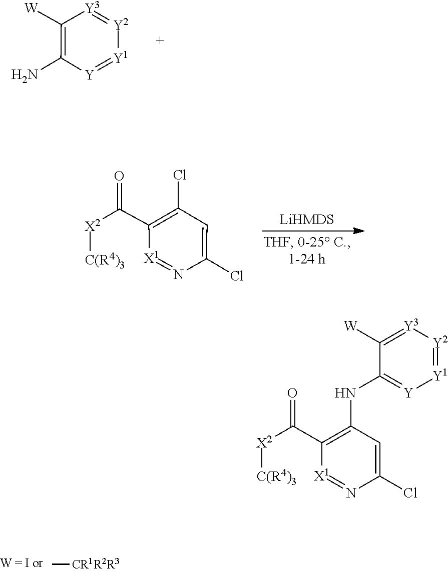

A mixture of an appropriately substituted 2-aminopyridine (1.0 equiv.), 4,6-dichloro-N-(methyl-d3)pyridazine-3-carboxamide (1.0 equiv.), and THF (0.1-0.3 M) were added to a flask and cooled in an ice bath. The vessel was purged with nitrogen and then 1 M LiHMDS solution in THF (2-4 equiv.) was added dropwise. The reaction was allowed to warm to ambient temperature and stirring continued for 1-24 h. The mixture was acidified to pH~3, and the resulting precipitate was collected by suction filtration, washed with water, and dried under vacuum affording the product with adequate purity.

### General Procedure D

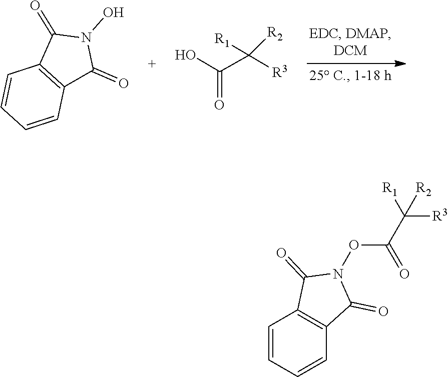

EDC (1.10 equiv.) was added to a stirring solution of carboxylic acid (1.00 equiv.), N-hydroxyphthalimide (1.05 equiv.), and DMAP (0.2 equiv.) in DCM at room temperature. Stirring continued overnight, diluted with DCM, and was washed with 1 M HCl (×1), brine (×1), dried over anh. magnesium sulfate, filtered, and concentrated under reduced pressure to afford the desired product in adequate purity. In some cases, the product required further purification by reverse phase flash chromatography on C18 column using a gradient of ACN in 10 mM AmF buffer.

The following activated esters were synthesized in this fashion from their corresponding commercially available carboxylic acids:

| Name | Structure | 1H NMR |
|---|---|---|
| 1,3-dioxoisoindolin-2-yl 1- methoxycyclopropane-1- carboxylate: | 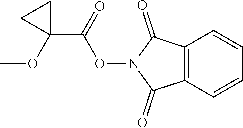 [SMILES: COC1(C(=O)ON2C(=O)c3ccccc3C2=O)CC1] | ¹H NMR (500 MHz, DMSO-d₆) δ 8.01- 7.94 (m, 4H), 3.45 (s, 3H), 1.55- 1.51 (m, 2H), 1.47-1.41 (m, 2H). |
| 1,3-dioxoisoindolin-2-yl 1- methylcyclopropane-1- carboxyate: | 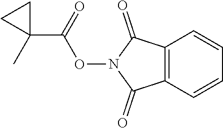 [SMILES: CC1(C(=O)ON2C(=O)c3ccccc3C2=O)CC1] | ¹H NMR (400 MHz, CDCl₃) δ 7.90- 7.85 (m, 2H), 7.80-7.75 (m, 2H), 1.59- 1.55 (m, 2H), 1.48 (s, 3H), 0.99- 0.95 (m, 2H). |
| 1,3-dioxoisoindolin-2-yl 1- ethoxycyclopropane-1- carboxylate | 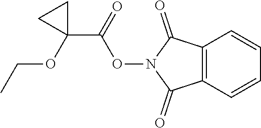 [SMILES: CCOC1(C(=O)ON2C(=O)c3ccccc3C2=O)CC1] | ¹H NMR (500 MHz, DMSO-d₆) δ 8.05- 7.92 (m, 4H), 3.72 (q, J = 7.0 Hz, 2H), 1.57-1.50 (m, 2H), 1.48-1.41 (m, 2H), 1.16 (t, J = 7.0 Hz, 3H). (m, 2H). |
| 1,3-dioxoisoindolin-2-yl 1- (methoxymethyl)cyclopropane- 1-carboxylate | 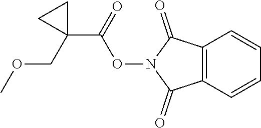 [SMILES: COCC1(C(=O)ON2C(=O)c3ccccc3C2=O)CC1] | ¹H NMR (500 MHz, CDCl₃) δ 7.92- 7.84 (m, 2H), 7.81-7.73 (m, 2H), 3.69 (s, 2H), 3.40 (s, 3H), 1.62 (q, J = 4.4 Hz, 2H), 1.22 (q, J = 4.5 Hz, 2H). |
| 1,3-dioxoisoindolin-2-yl 1- (difluoromethyl)cyclopropane- 1-carboxylate | 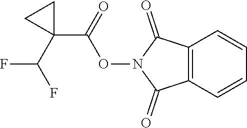 [SMILES: O=C1c2ccccc2C(=O)N1OC(=O)C1(C(F)F)CC1] | ¹H NMR (500 MHz, CDCl₃) δ 7.93- 7.87 (m, 2H), 7.83-7.77 (m, 2H), 6.52 (t, J = 56.8 Hz, 1H), 1.72-1.67 (m, 2H), 1.55 (dd, J = 8.1, 4.8 Hz, 2H). |
| 1,3-dioxoisoindolin-2-yl 2,2- difluoro-1- methylcyclopropane-1- carboxylate | 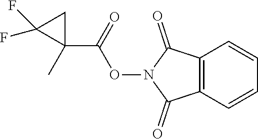 [SMILES: CC1(C(=O)ON2C(=O)c3ccccc3C2=O)CC1(F)F] | ¹H NMR (500 MHz, CDCl₃) δ 7.92- 7.89 (m, 1H), 7.82-7.79 (m, 1H), 2.42 (ddd, J = 12.1, 8.1, 6.8 Hz, 1H), 1.67- 1.65 (m, 2H), 1.60 (ddd, J = 10.7, 8.2, 5.9 Hz, 1H). |
| 1,3-dioxoisoindolin-2-yl 1- fluorospiro[2.3]hexane-1- carboxylate | 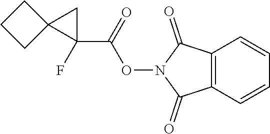 [SMILES: O=C1c2ccccc2C(=O)N1OC(=O)C1(F)CC12CCC2] | ¹H NMR (400 MHz, DMSO-d₆) δ 8.05- 7.93 (m, 4H), 2.48-2.40 (m, 1H), 2.38-2.23 (m, 2H), 2.18-2.03 (m, 3H), 1.90-1.72 (m, 2H). |
| 1,3-dioxoisoindolin-2-yl 1- fluorocyclopropane-1- carboxylate | 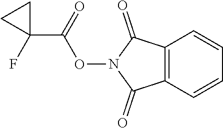 [SMILES: O=C1c2ccccc2C(=O)N1OC(=O)C1(F)CC1] | ¹H NMR (500 MHz, CDCl₃) δ 7.94- 7.88 (m, 2H), 7.85-7.78 (m, 2H), 1.70- 1.64 (m, 4H). ¹⁹F NMR (471 MHz, CDCl₃) δ −200.33. |

### General Procedure E

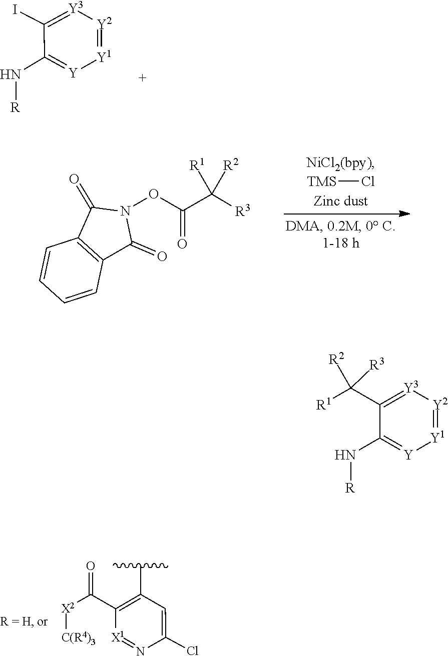

A reaction vessel containing 6-chloro-4-((3-iodopyridin-2-yl)amino)-N-(methyl-d3)pyridazine-3-carboxamide or the corresponding 2-amino pyridazine (1 equiv.), N-hydroxyphthalimide ester (1.2-2 equiv.), NiCl2(bpy) (15 mol %), and zinc dust (8 equiv.) was evacuated and backfilled with nitrogen gas (×3) before DMA (0.2 M) was added, followed by TMS-Cl (3 equiv.). The reaction vessel was quickly placed in an ice bath (0° C.) and stirred vigorously for 0.1-4 h. The reaction mixture was diluted with EtOAc and passed over a plug of silica gel or Celite® (eluting with EtOAc). The volatiles were removed under reduced pressure, and the crude material was purified by flash chromatography to afford the desired product.

### General Procedure F

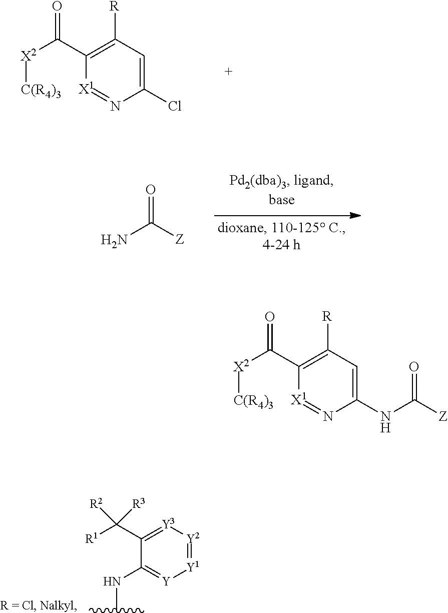

Chloropyridazine or chloropyridine (1.0 equiv.), an appropriate carboxamide (2 equiv.), Pd2(dba)3 (0.15 equiv.), -XantPhos or dcpf (0.3 equiv.), Cs2CO3 or K3PO4 (2.5 equiv.), and dioxane (0.05-0.1 M) were added to a pressure vial. Nitrogen gas was bubbled through the mixture for 5 mins, the vial sealed and heated to 110-125° C. for 4-18 h with stirring on a heating block. The reaction was cooled to room temperature and filtered through ca. 1Og of silica gel topped with Celite® using EtOAc or DCM as eluent. The filtrate was adsorbed onto silica gel and purified by flash chromatography to afford the final product. Oftentimes the final compound required another purification by reverse phase flash chromatography on C18 column to provide the product with acceptable purity.

The following intermediates were prepared according to general procedure F from commercially available starting materials:

| | | | (m/z) |
|---|---|---|---|
| | | MW | [M + H]⁺ |
| Name | Structure | (g/mol) | (method) |
| 4-chloro-6-(1- fluorocyclopropane-1- carboxamido)-N-(methyl- d₃)nicotinamide | 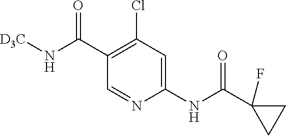 [SMILES: [2H]C([2H])([2H])NC(=O)c1cnc(NC(=O)C2(F)CC2)cc1Cl] | 234.3 | 235.0 (Q) |
| (1S,2S)-N-(4-chloro-5- propionylpyridin-2-yl)-2- fluorocyclopropane-1- carboxamide | 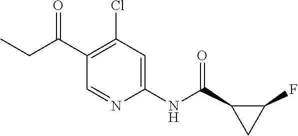 [SMILES: CCC(=O)c1cnc(NC(=O)[C@@H]2C[C@@H]2F)cc1Cl] | 270.7 | 271.2 (H) |

### General Procedure G

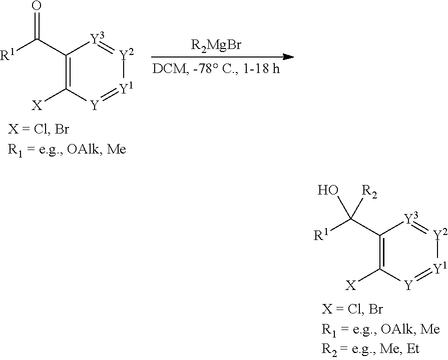

A mixture of an appropriately substituted heteroaryl ketone or ester (1.0 equiv.) in DCM (0.1-0.2 M) was cooled at −78° C. in a dry ice/acetone bath for 30 min under nitrogen. A solution of alkyl (Me or Et) magnesium bromide in Et₂O (1.2-3 equiv.) was added dropwise and the mixture was stirred at −78° C. for 1-6 h. A saturated solution of NH₄Cl was added and the mixture was allowed to warm to room temperature. Water and DCM were added. The organic phase was separated, washed with brine, dried over anh. Na₂SO₄, filtered and concentrated under reduced pressure. Oftentimes the product required a purification by reverse or normal phase flash chromatography. (SiO₂, 0-50% EtOAc in Heptanes).

### General Procedure H

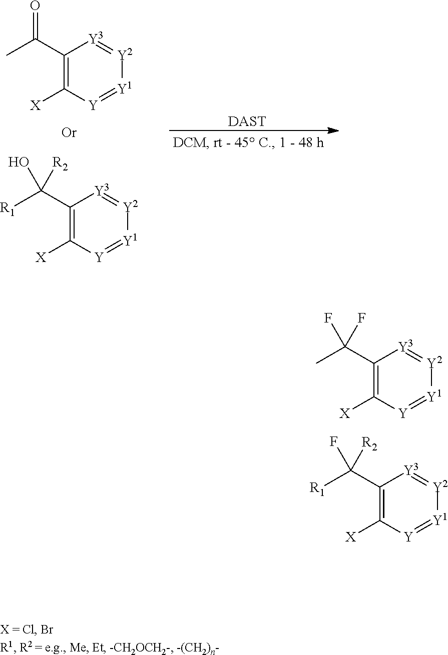

To a solution of an appropriately substituted heteroaryl ketone or carbinol (1.0 equiv.) in DCM or DCE (0.1-0.5 M) was added (diethylamino)sulfur trifluoride (1.5-10 equiv.). The solution was stirred at a temperature ranging from room temperature to 60° C. for 1-48 h. The mixture was added to a stirred solution of NaHCO₃ sat. at room temperature and stirred for 1 h. The mixture was extracted with DCM (2×). The combined organic was washed with aq. NaHCO₃ sat., brine, dried over anh. Na₂SO₄, filtered and concentrated under reduced pressure. The residue was adsorbed onto silica gel and purified over normal phase flash chromatography to afford the final product.

### General Procedure I

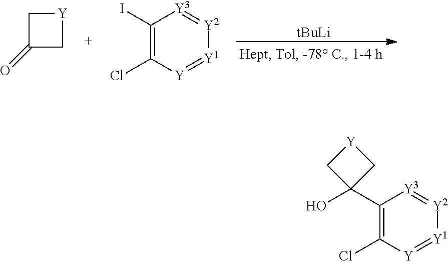

A dried vessel was charged with 2-chloro-3-iodoheteroarene (1.0 equiv.) in a 1:1 mixture of dry heptane and toluene (0.1-0.2 M) and was cooled at −78° C. in a dry ice/acetone bath for 30 min under nitrogen. A solution of 1.7 M tert-butyllithium in pentane (2.5 equiv.) was added dropwise and the mixture was stirred at −78° C. for 5-15 min. Cyclobutanone (or Oxetane-3-one) (3-5 equiv.) was added and the mixture was stirred at −78° C. for 1-4 h. NH₄Cl sat. was added dropwise at −78° C. and the mixture was allowed to warm to room temperature. The mixture was partitioned between EtOAc, water and brine. The aqueous phase was extracted with EtOAc (3×). The combined organic was washed with brine, dried over Na₂SO₄, filtered and concentrated under reduced pressure. The residue was used as-is in the next step.

### General Procedure J

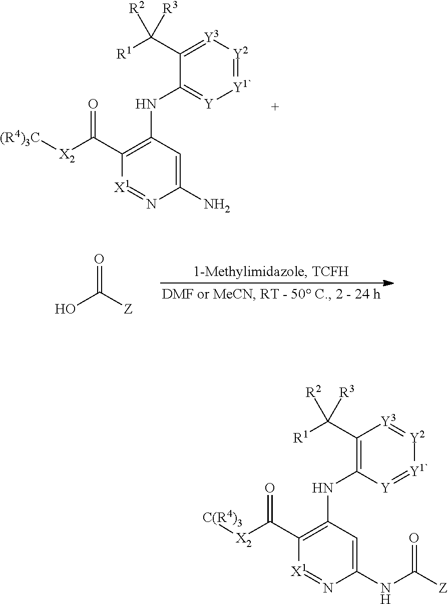

Aminopyridazine or aminopyridine (1.0 equiv.), and a corresponding carboxylic acid (2-5 equiv.), were dissolved in DMF or MeCN (0.05-0.2 M). 1-Methylimidazole (10-40 equiv.) and TCFH (5-20 equiv.) were added and the reactions were allowed to stir at 23-50° C. for 2-24 h. The cooled solutions were diluted with EtOAc, and washed with saturated ammonium chloride and brine, dried over anh. Na₂SO₄ and filtered. The filtrate was adsorbed onto silica gel and purified by flash chromatography to afford the final product. Oftentimes the final compound required another purification by reverse phase flash chromatography on C18 column to provide the product with acceptable purity.

### General Procedure K

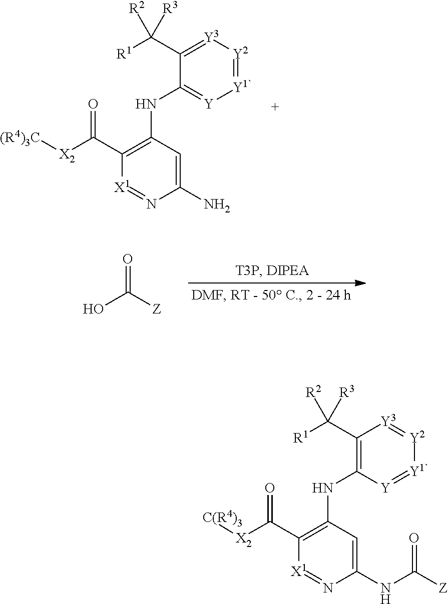

Aminopyridazine or aminopyridine ((1.0 equiv.),), and a corresponding carboxylic acid (2-5 equiv.), were dissolved in DMF (0.05-0.2 M). DIPEA (5-10 equiv.) and 50% T3P solution in DMF (5-10 equiv.) were added and the reactions were allowed to stir at 23-50° C. for 2-24 h. The cooled solutions were diluted with EtOAc, washed with saturate ammonium chloride and brine, dried over anh. Na₂SO₄ and filtered. The filtrate was adsorbed onto silica gel and purified by flash chromatography to afford the final product. Oftentimes the final compound required another purification by reverse phase flash chromatography on C18 column to provide the product with acceptable purity.

### Example 1A: 6-(cyclopropanecarboxamido)-N-(methyl-d3)-4-((3-(1-(trifluoromethyl)cyclopropyl)pyridin-2-yl)amino)pyridazine-3-carboxamide (Compound 1)

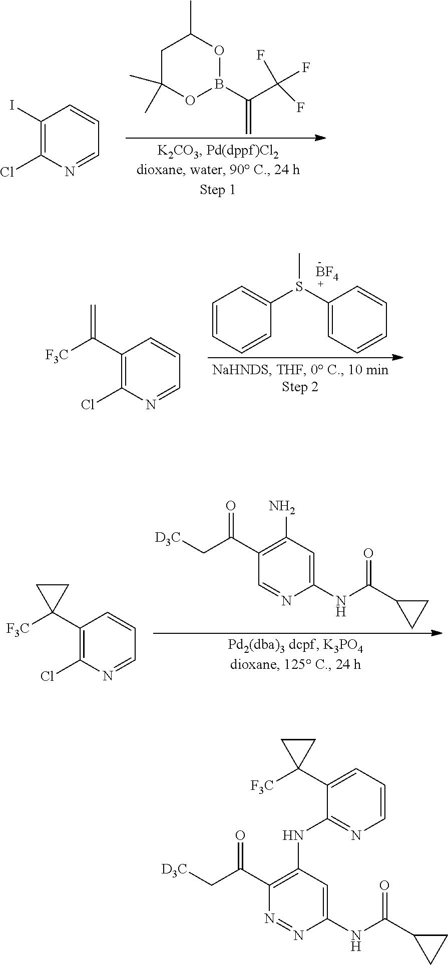

### Step 1: Synthesis of 2-chloro-3-(3,3,3-trifluoroprop-1-en-2-yl)pyridine

2-chloro-3-iodopyridine (1.50 g, 6.26 mmol), 1-(trifluoromethyl)vinylboronic acid hexylene glycol ester (1.71 mL, 8.14 mmol), Pd(dppf)Cl₂ (327 mg, 439 umol), K₂CO₃ (2.60 g, 18.8 mmol), dioxane (16.0 mL), and water (4.00 mL) were stirred in a sealed tube at 75° C. for 24 h. The reaction was cooled to ambient temperature and filtered through a plug of ca. 10 g silica gel topped with Celite® using DCM as eluent. The filtrate was adsorbed onto silica gel and purified by normal phase flash chromatography using DCM in hexanes (40%-100%) as eluent. Fractions containing product were combined and the solvent removed under reduced pressure, affording the title compound (1.05 g, 81%) as a pale brown oil. LCMS Method B (m/z): no ionization, retention time=1.14 min.

### Step 2: Synthesis of 2-chloro-3-(1-(trifluoromethyl)cyclopropyl)pyridine

2-Chloro-3-(3,3,3-trifluoroprop-1-en-2-yl)pyridine (1.05 g, 5.06 mmol) and diphenyl(methyl)sulfonium tetrafluoroborate (3.00 g, 10.1 m mol) were suspended in dry THF (12.0 mL) and the flask was purged with nitrogen gas under so nication for 2 mins. The mixture was cooled in an ice bath with stirring under nitrogen atmosphere, and NaHMDS (10.1 mL, 10.1 mmol) 1 M in THF was added dropwise. The reaction was allo wed to warm to ambient temperature, stirring continued for another hour, and then quenched with sat. aq. NH₄Cl. The mixture was extracted with EtOAc, the combined organics were washed with brine, dried over anh. Na₂SO₄ filtered and adsorbed onto silica gel. Purified over normal phase flash chromatography using DCM in heptanes (5%-100%) as eluent. The fractions containing product were combined and the solvent removed under reduced pressure affording the title comp ound (550 mg, 49%) as a pale brown oil. LCMS Method B (m/z): no ionization, retention time=1.17 min.

### Step 3: Synthesis of 6-(cyclopropanecarboxamido)-N-(methyl-d₃)-4-((3-(1-(trifluoromethyl)cyclopropyl)pyridin-2-yl)amino)pyridazine-3-carboxamide

The title compound was prepared according to General Procedure A using 4-amino-6-(cyclopropanecarboxamido)-N-(methyl-d₃)pyridazine-3-carboxamide (461 mg, 1.94 mmol) and 2-chloro-3-(1-(trifluoromethyl)cyclopropyl)pyridine (390 mg, 1.76 mmol). The product was purified by normal phase flash chromatography (dry load) using IPA in DCM (0.1%-10%) as eluent. Fractions containing product were combined and the solvent removed under reduced pressure. The reside was further purified by reverse phase flash chromatography on C18 column using MeCN in 10 mM AmB buffer (5%-30%) as eluent. Concentration of the pure fractions afforded the title compound (180 mg, 31%). LCMS Method G (m/z): [M+H]⁺=424.3, retention time=2.44 min. ¹H NMR (400 MHz, DMSO-d₆) δ 12.10 (s, 1H), 11.29 (s, 1H), 9.74 (s, 1H), 9.18 (s, 1H), 8.35 (dd, J=4.8, 1.8 Hz, 1H), 7.87 (dd, J=7.6, 1.8 Hz, 1H), 7.10 (dd, J=7.5, 4.9 Hz, 1H), 2.14-2.07 (m, 1H), 1.70-1.51 (m, 2H), 1.31-1.17 (m, 2H), 0.92-0.79 (m, 4H).

### Examples 1B-1C

The following compounds were prepared in a similar manner to Compound 1 described in Example 1A.

TABLE 2

| | | | | (m/z) | |
|---|---|---|---|---|---|
| Ex. | Compd | | MW | [M + H]⁺ | |
| No. | No. | Structure | (g/mol) | (method) | ¹H NMR |
| 1B | 26 | 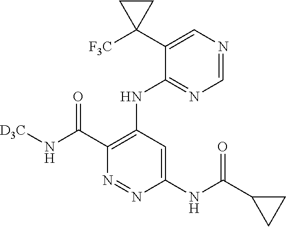 [SMILES: [2H]C([2H])([2H])NC(=O)c1nnc(NC(=O)C2CC2)cc1Nc1ncncc1C1(C(F)(F)F)CC1] | 424.4 | 425.2 (G) | ¹H NMR (500 MHz, DMSO-d₆) δ 12.59 (s, 1H), 11.47 (s, 1H), 9.94 (s, 1H), 9.32 (s, 1H), 8.86 (s, 1H), 8.65 (s, 1H), 2.20-2.08 (m, 1H), 1.62 (s, 2H), 1.34 (s, 2H), 0.96-0.81 (m, 4H). |
| 1C | 22 | 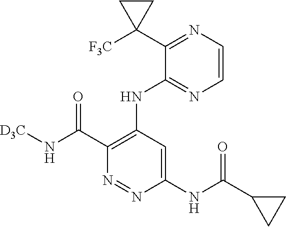 [SMILES: [2H]C([2H])([2H])NC(=O)c1nnc(NC(=O)C2CC2)cc1Nc1nccnc1C1(C(F)(F)F)CC1] | 424.4 | 425.2 (G) | ¹H NMR (500 MHz, DMSO-d₆) δ 12.42 (s, 1H), 11.40 (s, 1H), 9.72 (s, 1H), 9.26 (s, 1H), 8.42 (d, J = 2.5 Hz, 1H), 8.30 (d, J = 2.5 Hz, 1H), 2.19-2.08 (m, 1H), 1.68-1.59 (m, 2H), 1.42- 1.33 (m, 2H), 0.94-0.80 (m, 4H). |

### Example 2: 6-(cyclopropanecarboxamido)-N-(methyl-d3)-4-((3-(1-(trifluoromethyl)cyclopropyl)pyridin-2-yl)amino)nicotinamide (Compound 2)

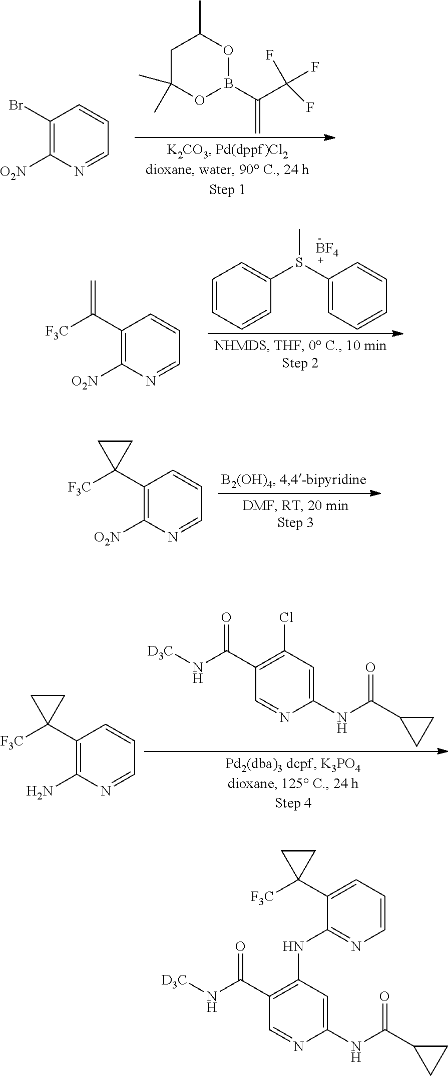

### Step 1: Synthesis of 2-nitro-3-(3,3,3-trifluoroprop-1-en-2-yl)pyridine

3-Bromo-2-nitropyridine (350 mg, 1.72 mmol), 1-(trifluoromethyl)vinylboronic acid hexylene glycol ester (470 uL, 2.24 mmol), Pd(dppf)Cl2 (90.1 mg, 121 umol), K₂CO₃ (715 mg, 5.17 mmol), dioxane (4.40 mL), water (1.10 mL) were stirred in a sealed tube at 90° C. for 24 h. The reaction was cooled to ambient temperature and filtered through a plug of ca. 10 g silica gel topped with Celite® using EtOAc as eluent. The filtrate was adsorbed onto silica gel and purified over normal phase flash chromatography using DCM in hexanes (40%-100%) as eluent. Fractions containing product were combined and the solvent removed under reduced pressure affording the title compound (295 mg, 78%) as a pale brown oil. LCMS Method F (m/z): no ionization, retention time=1.06 min.

### Step 2: Synthesis of 2-nitro-3-(1-(trifluoromethyl)cyclopropyl)pyridine

2-Nitro-3-(3,3,3-trifluoroprop-1-en-2-yl)pyridine (290 mg, 1.33 mmol) and diphenyl(methyl)sulfonium tetrafluoroborate (790 mg, 2.66 mmol) were suspended in dry THF (3.15 mL) and flask was purged with nitrogen gas under sonication for 2 mins. The mixture was cooled in an ice bath with stirring under nitrogen atmosphere, NaHMDS (2.66 mL, 2.66 mmol) 1 M in THF was added dropwise, stirring continued for 10 min, and then was quenched with satd. aq. NH4Cl. The mixture was extracted with EtOAc, the combined organics were washed with brine, dried over anh. Na2SO4, filtered and concentrated under reduced pressure affording the title compound (305 mg, 99%) as an orange oil which was used as is in the next step. Assumed quantitative yield. LCMS Method F (m/z): no ionization, retention time=1.12 min.

### Step 3: Synthesis of 3-(1-(trifluoromethyl)cyclopropyl)pyridin-2-amine

2-Nitro-3-(1-(trifluoromethyl)cyclopropyl)pyridine (300 mg, 1.29 mmol) was stirred in DMF (5.95 mL) and tetrahydroxydiboron (466 mg, 5.17 mmol) was added at room temperature. The mixture was cooled in an ice bath and 4,4′-dipyridyl (204 uL, 258 umol) was added (exotherm, color changed to deep purple, and then back to light brown). The reaction was stirred at room temperature for 4 h, then volatiles were removed under reduced pressure. The residue was taken up in EtOAc and the product was extracted into 2 M HCl (aq.). The organic layer was discarded, and the aqueous layer pH was adjusted to ~12 by adding KOH pellets under stirring. The product was extracted into EtOAc and the aqueous layer was discarded. The organic portion was washed with brine, dried over anh. Na2SO4, filtered, and concentrated under reduced pressure affording the title compound (205 mg, 78%) as a brown wax which was used in the next step without further purification. LCMS Method E (m/z): [M+H]+=203.2, retention time=0.54 min.

### Step 4: Synthesis of 6-(cyclopropanecarboxamido)-N-(methyl-d3)-4-((3-(1-(trifluoromethyl)cyclopropyl)pyridin-2-yl)amino)nicotinamide

The title compound was prepared according to General Procedure B using 4-chloro-6-(cyclopropanecarboxamido)-N-(methyl-d3)nicotinamide (120 mg, 467 umol) and 3-(1-(trifluoromethyl)cyclopropyl)pyridin-2-amine (85.9 mg, 425 umol). The product was purified by normal phase flash chromatography (dry load) using IPA in DCM (0.1%-10%) as an eluent. Fractions containing product were combined and the solvent removed under reduced pressure. The residue was further purified over reverse phase flash chromatography on C18 column, using MeCN in 10 mM AmB buffer (5%-30%). Concentration of the pure fractions afforded the title compound (19.5 mg, 9.9% yield). LCMS Method G (m/z): [M+H]+=423.3, retention time=2.11 min. 1H NMR (400 MHz, acetone-d6) δ 11.68 (s, 1H), 9.68 (d, J=3.6 Hz, 1H), 9.52 (s, 1H), 8.51 (s, 1H), 8.35 (dd, J=4.8, 1.9 Hz, 1H), 7.85 (d, J=1.8 Hz, 1H), 7.83 (d, J=1.8 Hz, 1H), 7.02 (dd, J=7.5, 4.8 Hz, 1H), 2.77 (s, 3H), 2.03-1.99 (m, 1H), 1.73-1.66 (m, 2H), 1.26-1.22 (m, 2H), 0.95-0.91 (m, 2H), 0.85-0.80 (m, 2H). Note: Cyclopropyl amide peak is obscured by solvent signal. Peak @2.77 ppm is the result of deuterium-proton exchange on the methyl amide.

### Example 3: N-(5-propionyl-4-((3-(1-(trifluoromethyl)cyclopropyl)pyridin-2-yl)amino)pyridin-2-yl)cyclopropanecarboxamide (Compound 3)

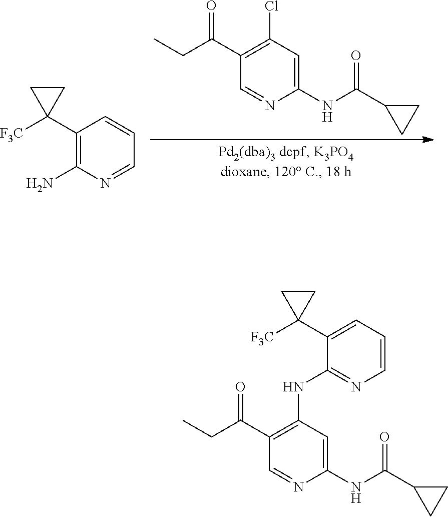 [SMILES: Nc1ncccc1C1(C(F)(F)F)CC1 ; CCC(=O)c1cnc(NC(=O)C2CC2)cc1Cl ; CCC(=O)c1cnc(NC(=O)C2CC2)cc1Nc1ncccc1C1(C(F)(F)F)CC1]

The title compound was prepared according to General Procedure B using N-(4-chloro-5-propionylpyridin-2-yl)cyclopropanecarboxamide (45 mg, 178 umol) and 3-(1-(trifluoromethyl)cyclopropyl)pyridin-2-amine (39.6 mg, 196 umol). The product was purified by normal phase flash chromatography (dry load) using IPA in DCM (0.1%-10%) as an eluent. Fractions containing product were combined and the solvent removed under reduced pressure. The reside was further purified by reverse phase flash chromatography on C18 column, using MeCN in 10 mM AmB buffer (5%-30%) as eluent. Concentration of the pure fractions afforded the title compound (5.0 mg, 6.7%). LCMS Method C (m/z): [M+H]⁺=419.3, retention time=2.81 min. ¹H NMR (500 MHz, DMSO-d₆) δ 11.90 (s, 1H), 10.80 (s, 1H), 9.41 (s, 1H), 8.86 (s, 1H), 8.31 (dd, J=4.8, 1.8 Hz, 1H), 7.82 (dd, J=7.5, 1.8 Hz, 1H), 7.06 (dd, J=7.5, 4.8 Hz, 1H), 3.07 (q, J=7.2 Hz, 2H), 2.04-1.93 (m, 1H), 1.62-1.49 (m, 2H), 1.23-1.13 (m, 2H), 1.06 (t, J=7.2 Hz, 3H), 0.83-0.69 (m, 4H).

### Example 4: 4-((5-cyano-3-(1-(trifluoromethyl)cyclopropyl)pyridin-2-yl)amino)-6-(cyclopropanecarboxamido)-N-(methyl-d3)pyridazine-3-carboxamide (Compound 4)

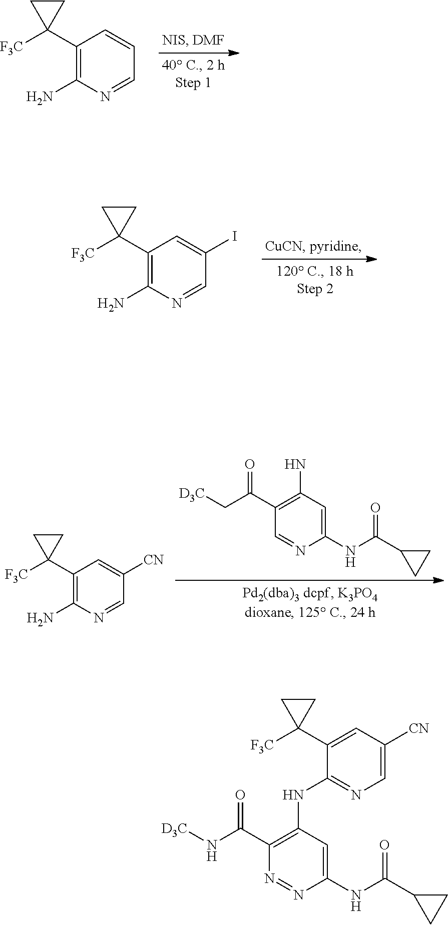 [SMILES: Nc1ncccc1C1(C(F)(F)F)CC1 ; Nc1ncc(I)cc1C1(C(F)(F)F)CC1 ; N#Cc1cnc(N)c(C2(C(F)(F)F)CC2)c1 ; [2H]C([2H])([2H])CC(=O)c1cnc(NC(=O)C2CC2)cc1N ; [2H]C([2H])([2H])NC(=O)c1nnc(NC(=O)C2CC2)cc1Nc1ncc(C#N)cc1C1(C(F)(F)F)CC1]

### Step 1: Synthesis of 5-iodo-3-(1-(trifluoromethyl)cyclopropyl)pyridin-2-amine

3-(1-(trifluoromethyl)cyclopropyl)pyridin-2-amine (500 mg, 2.47 mmol) and DMF (8.00 mL) were stirred at room temperature and N-iodosuccinimide (869 mg, 3.71 mmol) was added. The reaction was heated to 40° C. for 2 h. The reaction was concentrated to near dryness under reduced pressure and the residue was taken up in EtOAc. The organic portion was washed with satd. aq. NaHCO3 and brine, the organic portion was dried over anh. Na2SO4, filtered and adsorbed onto silica gel. Purified by normal phase flash chromatography using IPA in DCM (0%-10%) as an eluent. Fractions containing product were combined and the solvent removed under reduced pressure affording the title compound (485 mg, 60%) as a brown solid. LCMS Method B (m/z): [M+H]⁺=329.1, retention time=1.21 min.

### Step 2: Synthesis of 6-amino-5-(1-(trifluoromethyl)cyclopropyl)nicotinonitrile

5-iodo-3-(1-(trifluoromethyl)cyclopropyl)pyridin-2-amine (100 mg, 305 umol), copper(I) cyanide (41.4 mg, 457 umol), and pyridine (1.84 mL) were stirred in a sealed tube at 120° C. for 18 h on a heating block. The solvent was removed under reduced pressure and the residue was taken up in EtOAc, adsorbed onto silica gel, and purified over normal phase flash chromatography using IPA in DCM (0%-10%). Fractions containing product were combined and the solvent removed under reduced pressure affording the title compound (40 mg, 58%) as a pale brown solid. LCMS Method B (m/z): [M−H]−=226.1, retention time=0.93 min. Step 3: Synthesis of 4-((5-cyano-3-(1-(trifluoromethyl)cyclopropyl)pyridin-2-yl)amino)-6-(cyclopropanecarboxamido)-N-(methyl-d₃)pyridazine-3-carboxamide:

The title compound was prepared according to General Procedure B using 4-bromo-6-(cyclopropanecarboxamido)-N-(methyl-d₃)pyridazine-3-carboxamide (53.2 mg, 176 umol) and 6-amino-5-(1-(trifluoromethyl)cyclopropyl)nicotinonitrile (40.0 mg, 176 umol). The product was purified by reverse phase flash chromatography on C18 column using ACN in 10 mM AmB buffer (20%-60%). Concentration of the pure fractions afforded the title compound (19.5 mg, 9.9%). LCMS Method G (m/z): [M+H]+=449.3, retention time=2.51 min. ¹H NMR (500 MHz, DMSO-d₆) δ 12.17 (s, 1H), 11.00 (s, 1H), 9.37 (s, 1H), 8.85 (s, 1H), 8.35 (d, J=1.7 Hz, 1H), 7.91 (d, J=1.6 Hz, 1H), 1.74-1.63 (m, 1H), 1.18 (br s, 2H), 0.96-0.85 (m, 2H), 0.48-0.36 (m, 4H).

### Example 5: 6-(cyclopropanecarboxamido)-N-(methyl-d3)-4-((5-methyl-3-(1-(trifluoromethyl)cyclopropyl)pyridin-2-yl)amino)pyridazine-3-carboxamide (Compound 5)

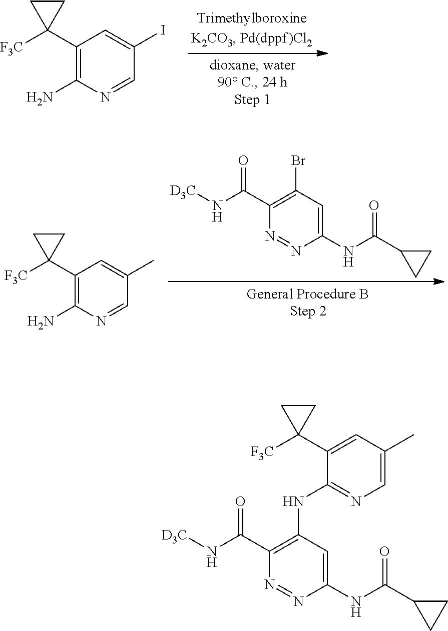 [SMILES: Nc1ncc(I)cc1C1(C(F)(F)F)CC1 ; Cc1cnc(N)c(C2(C(F)(F)F)CC2)c1 ; [2H]C([2H])([2H])NC(=O)c1nnc(NC(=O)C2CC2)cc1Br ; [2H]C([2H])([2H])NC(=O)c1nnc(NC(=O)C2CC2)cc1Nc1ncc(C)cc1C1(C(F)(F)F)CC1]

### Step 1: Synthesis of 5-methyl-3-(1-(trifluoromethyl)cyclopropyl)pyridin-2-amine

5-iodo-3-(1-(trifluoromethyl)cyclopropyl)pyridin-2-amine (85.0 mg, 259 umol), trimethylboroxine (724 uL, 2.59 mmol), K₂CO₃ (107 mg, 777 umol), Pd(dppf)Cl₂ (19.3 mg, 25.9 umol), dioxane (1.31 mL) and water (261 uL) were added to a vial fitted with a stir bar. The vial was sealed and heated to 90° C. for 24 h with stirring. The mixture was filtered through a plug of ca. 5 g silica gel with EtOAc as eluent, filtrate was adsorbed onto silica gel and purified over normal phase flash chromatography using IPA in DCM (0%-10%) as a mobile phase. Concentration of the pure fractions afforded the title compound (35.0 mg, 62%). LCMS Method A (m/z): [M+H]+=217.3, retention time=0.67 min.

### Step 2: Synthesis of 6-(cyclopropanecarboxamido)-N-(methyl-d₃)-4-((5-methyl-3-(1-(trifluoromethyl)cyclopropyl)pyridin-2-yl)amino)pyridazine-3-carboxamide

The title compound was prepared according to General Procedure B using 4-bromo-6-(cyclopropanecarboxamido)-N-(methyl-d₃)pyridazine-3-carboxamide (48.9 mg, 162 umol) and 5-methyl-3-(1-(trifluoromethyl)cyclopropyl)pyridin-2-amine (35.0 mg, 162 umol). The product was purified by reverse phase flash chromatography on C18 column using ACN in 10 mM AmB buffer (20%-60%) as an eluent. Concentration of the pure fractions afforded the title compound (4.5 mg, 6.4%). LCMS Method G (m/z): [M+H]+=438.3, retention time=2.11 min. ¹H NMR (500 MHz, DMSO-d₆) δ 11.98 (s, 1H), 11.27 (s, 1H), 9.65 (s, 1H), 9.16 (s, 1H), 8.22 (d, J=1.6 Hz, 1H), 7.73 (d, J=1.9 Hz, 1H), 2.28 (s, 3H), 2.17-2.05 (m, 1H), 1.66-1.51 (m, 2H), 1.27-1.18 (m, 2H), 0.91-0.79 (m, 4H).

### Example 6: 4-((3-(1-cyanocyclopropyl)pyridin-2-yl)amino)-6-(cyclopropanecarboxamido)-N-(methyl-d3)pyridazine-3-carboxamide (Compound 6)

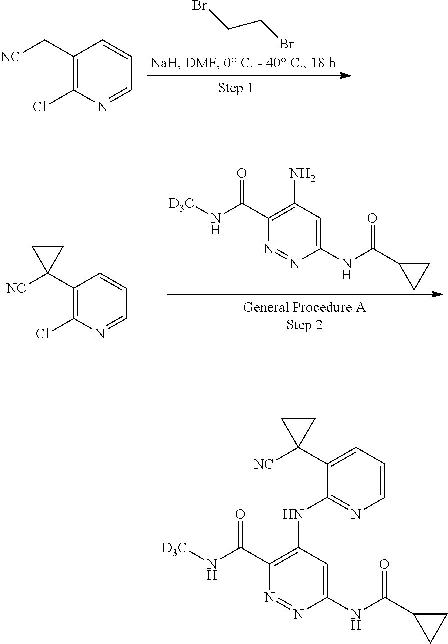

### Step 1: Synthesis of 1-(2-chloropyridin-3-yl)cyclopropane-1-carbonitrile

(2-Chloropyridin-3-yl)acetonitrile (1.00 g, 6.36 mmol) and DMF (15.0 mL) were cooled in an ice bath and 60% NaH in mineral oil (1.27 g, 31.8 mmol) was added with stirring. The reaction was stirred at room temperature for 15 mins then 1,2-dibromoethane (603 uL, 6.99 mmol) was added dropwise. The reaction was heated to 40° C. on a heating block for 18 h, then cooled to room temperature. The excess NaH was quenched with slow addition of MeOH until gas evolution ceased, then the mixture was concentrated to near-dryness under reduced pressure. The residue was taken up in EtOAc and washed with brine. The organic portion was dried over anh. Na₂SO₄, filtered and concentrated under vacuum. The residue was taken up in DMSO and purified by reverse phase flash chromatography on C18 column using ACN in 10 mM AmB buffe r (5%-30%) as an eluent. Fractions containing product were combined, extracted with EtOAc, organics were washed with water and brine, dried over anh. Na₂SO₄ and vacuum filtered. The filtrate was concentrated under reduced pressure affording the title compound (810 mg, 71%) as a yellow solid. LCMS Method B (m/z): no ionization, retention time=0.76 min.

### Step 2: 4-((3-(1-cyanocyclopropyl)pyridin-2-yl)amino)-6-(cyclopropanecarboxamido)-N-(methyl-d3)pyridazine-3-carboxamide

The title compound was prepared according to General Procedure A 4-amino-6-(cyclopropanecarboxamido)-N-(methyl-d3)pyridazine-3-carboxamide (88.0 mg, 370 umol) and 1-(2-chloropyridin-3-yl)cyclopropane-1-carbonitrile (60 mg, 336 umol). The product was purified over reverse phase flash chromatography on C18 column using MeCN in 10 mM AmB buffer (25%-60%) as an eluent. Concentration of the pure fractions afforded the title compound (22.4 mg, 22%). LCMS Method G (m/z): [M+H]+=381.2, retention time=1.84 min. 1H NMR (500 MHz, DMSO-d6) δ 12.36 (s, 1H), 11.36 (s, 1H), 9.77 (s, 1H), 9.28 (s, 1H), 8.35 (dd, J=4.9, 1.7 Hz, 1H), 7.86 (dd, J=7.6, 1.8 Hz, 1H), 7.09 (dd, J=7.6, 4.9 Hz, 1H), 2.17-2.09 (m, 1H), 1.92-1.84 (m, 2H), 1.56-1.49 (m, 2H), 0.92-0.82 (m, 4H).

### Example 7: 6-(cyclopropanecarboxamido)-4-((3-(1-methoxycyclopropyl)pyridin-2-yl)amino)-N-(methyl-d3)pyridazine-3-carboxamide (Compound 7)

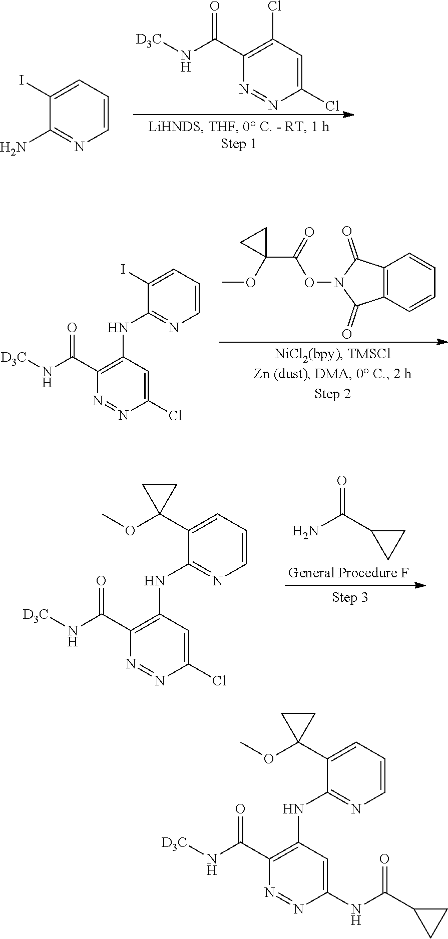

### Step 1: Synthesis of 6-chloro-4-((3-iodopyridin-2-yl)amino)-N-(methyl-d3)pyridazine-3-carboxamide

The synthesis was carried out according to General Procedure C using 2-amino-3-iodopyridine (447 mg, 1.99 mmol) affording the title compound (710 mg, 91%) as a beige solid. LCMS Method C (m/z): [M+H]+=393.1, retention time=1.27 min.

### Step 2: Synthesis of 6-chloro-4-((3-(1-methoxycyclopropyl)pyridin-2-yl)amino)-N-(methyl-d3)pyridazine-3-carboxamide

The synthesis was carried out according to General Procedure E using 1,3-dioxoisoindolin-2-yl 1-methoxycyclopropane-1-carboxylate (106 mg, 408 umol) and 6-chloro-4-((3-iodopyridin-2-yl)amino)-N-(methyl-d3)pyridazine-3-carboxamide (100 mg, 255 umol) affording the title compound (45 mg, 52%) as a pale yellow solid. LCMS Method A (m/z): [M+H]+=337.3 retention time=1.25 min.

### Step 3: Synthesis of 6-(cyclopropanecarboxamido)-4-((3-(1-methoxycyclopropyl)pyridin-2-yl)amino)-N-(methyl-d3)pyridazine-3-carboxamide

The synthesis was carried out according to General Procedure F using 6-chloro-4-((3-(1-methoxycyclopropyl)pyridin-2-yl)amino)-N-(methyl-d3)pyridazine-3-carboxamide (54.4 mg, 161 umol). The product was purified over normal phase flash chromatography (dry load) using IPA in DCM (0.1%-10%) as an eluent. Fractions containing product were combined and the solvent removed under reduced pressure. The residue was further purified over reverse phase flash chromatography on C18 column using MeCN in 10 mM AmB buffer (5%-30%). Concentration of the pure fractions afforded the title compound (6.4 mg, 10%). LCMS Method C (m/z): [M+H]+=386.3, retention time=2.08 min. 1H NMR (500 MHz, acetone-d6) δ 12.08 (s, 1H), 10.04 (s, 1H), 9.93 (s, 1H), 8.46 (s, 1H), 8.23 (dd, J=4.9, 1.9 Hz, 1H), 7.64 (dd, J=7.4, 1.9 Hz, 1H), 6.91 (dd, J=7.4, 4.9 Hz, 1H), 3.00 (s, 3H), 2.66 (s, 3H), 2.05-1.98 (m, 1H), 1.26-1.21 (m, 2H), 0.90-0.82 (m, 4H), 0.81-0.74 (m, 2H). Note: Peak @2.66 ppm is the result of deuterium-proton exchange on the methyl amide.

### Example 8A: 6-(cyclopropanecarboxamido)-N-(methyl-d3)-4-((3-(1-methylcyclopropyl)pyridin-2-yl)amino)pyridazine-3-carboxamide (Compound 8)

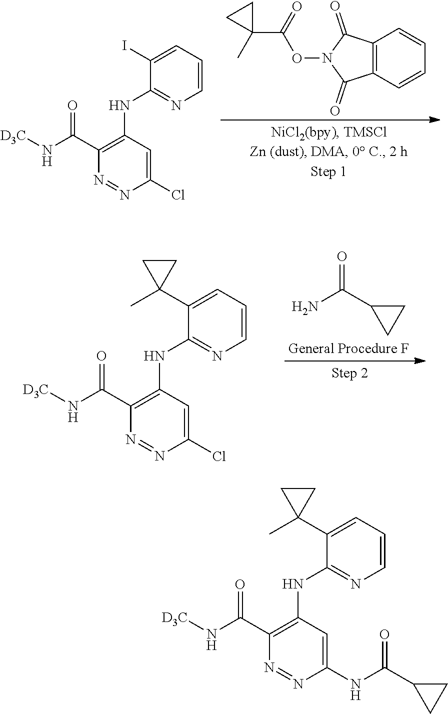

### Step 1: Synthesis of 6-chloro-N-(methyl-d3)-4-((3-(1-methylcyclopropyl)pyridin-2-yl)amino)pyridazine-3-carboxamide

The synthesis was carried out according to General Procedure E using 1,3-dioxoisoindolin-2-yl 1-methylcyclopropane-1-carboxylate and 6-chloro-4-((3-iodopyridin-2-yl)amino)-N-(methyl-d₃)pyridazine-3-carboxamide (100 mg, 255 umol) affording the title compound (70.0 mg, 57%) as a solid.

### Step 2: Synthesis of 6-(cyclopropanecarboxamido)-N-(methyl-d₃)-4-((3-(1-methylcyclopropyl)pyridin-2-yl)amino)pyridazine-3-carboxamide

The synthesis was carried out according to General Procedure F using 6-chloro-N-(methyl-d₃)-4-((3-(1-methylcyclopropyl)pyridin-2-yl)amino)pyridazine-3-carboxamide (54.4 mg, 161 umol). The product was purified by normal phase flash chromatography using IPA in DCM (0.1%-10%) as an eluent. Fractions containing product were combined and the solvent removed under reduced pressure. The reside was further purified over reverse phase flash chromatography on C18 column using ACN in 10 mM AmB buffer (5%-30%). Concentration of the pure fractions afforded the title compound (6.4 mg, 10%). LCMS Method C (m/z): [M+H]⁺=370.4, retention time=2.60 min. ¹H NMR (500 MHz, DMSO-d₆) δ 11.98 (s, 1H), 11.27 (s, 1H), 9.80 (s, 1H), 9.17 (s, 1H), 8.20 (dd, J=4.9, 1.9 Hz, 1H), 7.69 (dd, J=7.4, 1.9 Hz, 1H), 7.00 (dd, J=7.4, 4.9 Hz, 1H), 2.17-2.08 (m, 1H), 1.34 (s, 3H), 0.98-0.92 (m, 2H), 0.91-0.80 (m, 4H), 0.82-0.75 (in, 2H).

### Examples 8B-8P

The following compounds were prepared in a similar fashion to that described for Compound 7 in Example 7 and Compound 8 in Example 8A

TABLE 3

| | | | | (m/z) | |
|---|---|---|---|---|---|
| Ex. | Compd | | MW | [M + H]⁺ | |
| No | No. | Structure | (g/mol) | (method) | ¹H NMR |
| 8B | 15 | 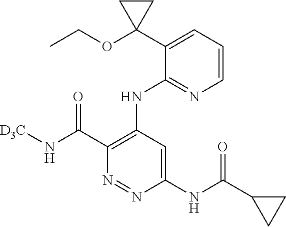 [SMILES: [2H]C([2H])([2H])NC(=O)c1nnc(NC(=O)C2CC2)cc1Nc1ncccc1C1(OCC)CC1] | 399.5 | 400.3 (G) | ¹H NMR (500 MHz, DMSO-d₆) δ 11.91 (s, 1H), 11.27 (s, 1H), 9.72 (s, 1H), 9.07 (s, 1H), 8.28 (dd, J = 4.9, 1.8 Hz, 1H), 7.73 (dd, J = 7.4, 1.8 Hz, 1H), 7.04 (dd, J = 7.4, 4.9 Hz, 1H), 3.28-3.25 (m, 2H), 2.16- 2.08 (m, 1H), 1.29-1.21 (m, 2H), 0.96 (t, J = 7.0 Hz, 3H), 0.93- 0.89 (m, 2H), 0.89-0.81 (m, 4H). |
| 8C | 19 | 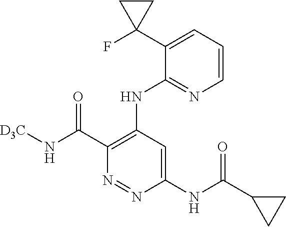 [SMILES: [2H]C([2H])([2H])NC(=O)c1nnc(NC(=O)C2CC2)cc1Nc1ncccc1C1(F)CC1] | 373.4 | 374.0 (L) | ¹H NMR (500 MHz, DMSO-d₆) δ 12.31 (s, 1H), 11.32 (s, 1H), 9.82 (s, 1H), 9.19 (s, 1H), 8.38 (d, J = 4.0 Hz, 1H), 7.89 (d, J = 7.3 Hz, 1H), 7.12-7.00 (m, 1H), 2.16- 2.08 (m, 1H), 1.62-1.51 (m, 2H), 1.19-1.09 (m, 2H), 0.93-0.78 (m, 4H). |
| 8D | 29 | 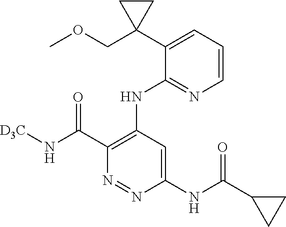 [SMILES: [2H]C([2H])([2H])NC(=O)c1nnc(NC(=O)C2CC2)cc1Nc1ncccc1C1(COC)CC1] | 399.5 | 400.3 (D) | ¹H NMR (500 MHz, DMSO-d₆) δ 11.29 (s, 1H), 11.07 (s, 1H), 9.15 (s, 1H), 8.01 (s, 1H), 7.51-7.45 (m, 3H), 7.27 (ddd, J = 7.8, 5.3, 3.5 Hz, 1H), 2.10-2.03 (m, 1H), 1.71-1.67 (m, 2H), 1.47-1.43 (m, 2H), 0.84-0.76 (m, 4H). |
| 8E | 32 | 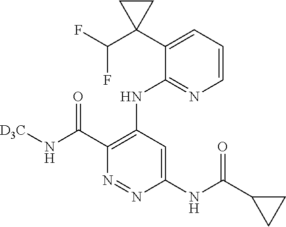 [SMILES: [2H]C([2H])([2H])NC(=O)c1nnc(NC(=O)C2CC2)cc1Nc1ncccc1C1(C(F)F)CC1] | 405.4 | 406.3 (D) | ¹H NMR (500 MHz, DMSO-d₆) δ 12.00 (s, 1H), 11.29 (s, 1H), 9.73 (s, 1H), 9.16 (s, 1H), 8.32 (dd, J = 4.8, 1.8 Hz, 1H), 7.76 (dd, J = 7.5, 1.9 Hz, 1H), 7.08 (dd, J = 7.5, 4.9 Hz, 1H), 5.93 (t, J = 56.0 Hz, 1H), 2.11 (tt, J = 7.4, 5.2 Hz, 1H), 1.42- 1.36 (m, 2H), 1.09-1.02 (m, 2H), 0.88-0.83 (m, 4H). |
| 8F | 35 | 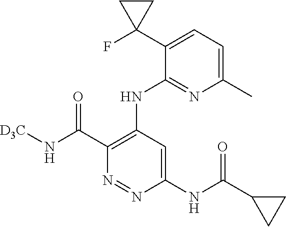 [SMILES: [2H]C([2H])([2H])NC(=O)c1nnc(NC(=O)C2CC2)cc1Nc1nc(C)ccc1C1(F)CC1] | 387.4 | 388.4 (L) | ¹H NMR (500 MHz, DMSO-d₆) δ 12.27 (s, 1H), 11.29 (s, 1H), 10.13 (s, 1H), 9.17 (s, 1H), 7.75 (dd, J = 7.5, 1.3 Hz, 1H), 6.93 (d, J = 7.6 Hz, 1H), 2.51-2.50 (m, 3H), 2.13 (tt, J = 7.1, 5.5 Hz, 1H), 1.62- 1.48 (m, 2H), 1.14-1.06 (m, 2H), 0.90-0.83 (m, 4H). |
| 8G | 38 | 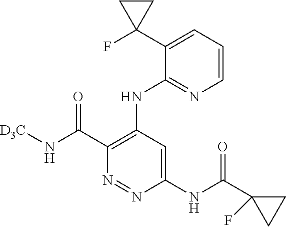 [SMILES: [2H]C([2H])([2H])NC(=O)c1nnc(NC(=O)C2(F)CC2)cc1Nc1ncccc1C1(F)CC1] | 391.4 | 392.0 (L) | ¹H NMR (500 MHz, DMSO-d₆) δ 12.37 (s, 1H), 10.77 (s, 1H), 9.80 (s, 1H), 9.27 (s, 1H), 8.39 (d, J = 4.9 Hz, 1H), 7.91 (dt, J = 7.5, 1.8 Hz, 1H), 7.11 (dd, J = 7.3, 5.1 Hz, 1H), 1.63-1.53 (m, 2H), 1.53- 1.44 (m, 2H), 1.44-1.37 (m, 2H), 1.21-1.09 (m, 2H). |
| 8H | 39 | 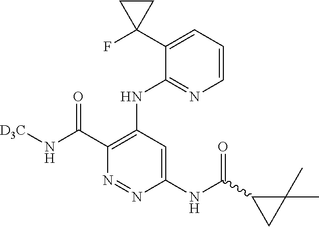 [SMILES: [2H]C([2H])([2H])NC(=O)c1nnc(NC(=O)C2CC2(C)C)cc1Nc1ncccc1C1(F)CC1] | 401.5 | 402.1 (L) | ¹H NMR (500 MHz, DMSO-d₆) δ 12.30 (s, 1H), 11.15 (s, 1H), 9.81 (s, 1H), 9.20 (s, 1H), 8.39 (dt, J = 4.9, 1.6 Hz, 1H), 7.89 (dt, J = 7.5, 1.9 Hz, 1H), 7.09 (dd, J = 7.2, 5.2 Hz, 1H), 2.01 (dd, J = 7.8, 5.5 Hz, 1H), 1.57 (dt, J = 18.1, 6.9 Hz, 2H), 1.19-1.12 (m, 8H), 1.05 (dd, J = 5.3, 4.1 Hz, 1H), 0.86 (dd, J = 7.8, 3.9 Hz, 1H). |
| 8I | 40 | 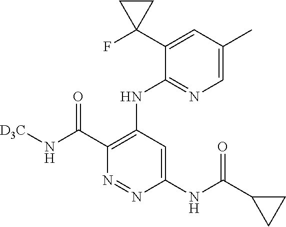 [SMILES: [2H]C([2H])([2H])NC(=O)c1nnc(NC(=O)C2CC2)cc1Nc1ncc(C)cc1C1(F)CC1] | 387.4 | 388.3 (D) | ¹H NMR (500 MHz, DMSO-d₆) δ = 12.17 (s, 1H), 11.29 (s, 1H), 9.74 (s, 1H), 9.16 (s, 1H), 8.23 (s, 1H), 7.74 (s, 1H), 2.28 (s, 3H), 2.16-2.08 (m, 1H), 1.59-1.50 (m, 2H), 1.18-1.10 (m, 2H), 0.90- 0.83 (m, 4H). |
| 8J | 44 | 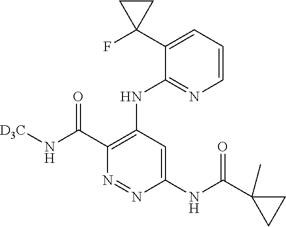 [SMILES: [2H]C([2H])([2H])NC(=O)c1nnc(NC(=O)C2(C)CC2)cc1Nc1ncccc1C1(F)CC1] | 387.4 | 388.3 (L) | ¹H NMR (500 MHz, DMSO-d₆) δ 12.31 (s, 1H), 10.08 (s, 1H), 9.74 (s, 1H), 9.17 (s, 1H), 8.40 (d, J = 4.9 Hz, 1H), 7.93-7.86 (m, 1H), 7.10 (dd, J = 7.2, 5.1 Hz, 1H), 1.63- 1.50 (m, 2H), 1.47 (s, 3H), 1.23- 1.10 (m, 4H), 0.74-0.68 (m, 2H). |
| 8K | 88 | 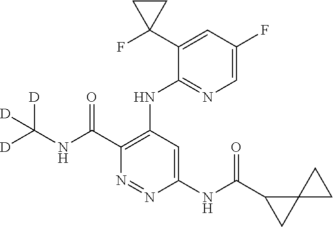 [SMILES: [2H]C([2H])([2H])NC(=O)c1nnc(NC(=O)C2CC23CC3)cc1Nc1ncc(F)cc1C1(F)CC1] | 399.4 | 400.1 (L) | ¹H NMR (500 MHz, DMSO-d₆) δ 12.31 (s, 1H), 11.12 (s, 1H), 9.82 (s, 1H), 9.19 (s, 1H), 8.41 (dt, J = 2.7, 1.6 Hz, 1H), 7.90 (dt, J = 7.4, 1.7 Hz, 1H), 7.10 (dd, J = 7.2, 5.0 Hz, 1H), 2.48-2.46 (m, 1H), 1.66- 1.49 (m, 2H), 1.45 (t, J = 3.8 Hz, 1H), 1.38 (dd, J = 7.4, 3.4 Hz, 1H), 1.18-1.12 (m, 2H), 0.95- 0.83 (m, 3H), 0.82-0.75 (m, 1H). |
| 8L | 54 | 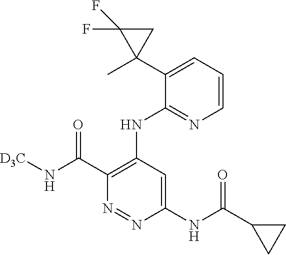 [SMILES: [2H]C([2H])([2H])NC(=O)c1nnc(NC(=O)C2CC2)cc1Nc1ncccc1C1(C)CC1(F)F] | 405.4 | 406.0 (L) | ¹H NMR (500 MHz, DMSO-d₆) δ 11.93 (s, 1H), 11.30 (s, 1H), 9.69 (s, 1H), 9.22 (s, 1H), 8.30 (dd, J = 4.8, 1.7 Hz, 1H), 7.74 (d, J = 7.6 Hz, 1H), 7.08 (dd, J = 7.5, 4.9 Hz, 1H), 2.15-2.08 (m, 1H), 1.86- 1.79 (m, 2H), 1.53 (s, 3H), 0.90- 0.84 (m, 4H). |
| 8M | 61 | 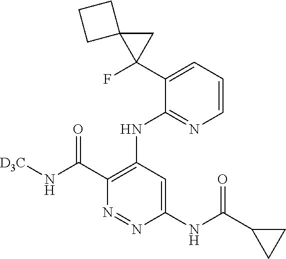 [SMILES: [2H]C([2H])([2H])NC(=O)c1nnc(NC(=O)C2CC2)cc1Nc1ncccc1C1(F)CC12CCC2] | 413.5 | 414.0 (G) | ¹H NMR (400 MHz, DMSO-d₆) δ 11.93 (s, 1H), 11.32 (s, 1H), 9.50 (s, 1H), 9.16 (s, 1H), 8.45-8.28 (m, 1H), 7.83 (dt, J = 7.5, 1.9 Hz, 1H), 7.20-7.03 (m, 1H), 2.85- 2.73 (m, 1H), 2.26-2.16 (m, 1H), 2.14-2.02 (m, 1H), 1.95-1.81 (m, 1H), 1.79-1.63 (m, 2H), 1.48- 1.32 (m, 2H), 1.24 (dd, J = 19.6, 7.8 Hz, 1H), 0.91-0.72 (m, 4H). |
| 8N | 85 | 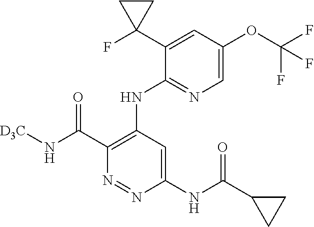 [SMILES: [2H]C([2H])([2H])NC(=O)c1nnc(NC(=O)C2CC2)cc1Nc1ncc(OC(F)(F)F)cc1C1(F)CC1] | 457.4 | 458.3 (G) | ¹H NMR (400 MHz, DMSO-d₆) δ = 12.52 (s, 1H), 11.40 (s, 1H), 9.78 (s, 1H), 9.25 (s, 1H), 8.47 (s, 1H), 8.02 (s, 1H), 2.19-2.08 (m, 1H), 1.66-1.54 (m, 2H), 1.30- 1.20 (m, 2H), 0.92-0.83 (m, 4H). |
| 8O | 75 | 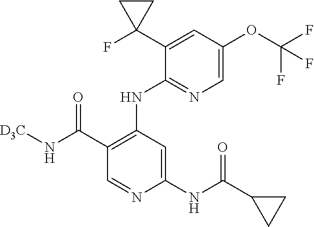 [SMILES: [2H]C([2H])([2H])NC(=O)c1cnc(NC(=O)C2CC2)cc1Nc1ncc(OC(F)(F)F)cc1C1(F)CC1] | 456.4 | 457.3 (C) | ¹H NMR (400 MHz, DMSO-d₆) δ 11.99 (s, 1H), 10.80 (s, 1H), 9.41 (s, 1H), 8.67 (s, 1H), 8.60 (s, 1H), 8.41 (s, 1H), 7.96 (s, 1H), 2.07- 1.96 (m, 1H), 1.64-1.49 (m, 2H), 1.27-1.14 (m, 2H), 0.87-0.75 (m, 4H). |
| 8P | 72 | 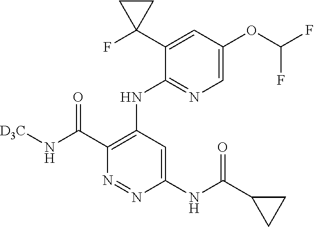 [SMILES: [2H]C([2H])([2H])NC(=O)c1nnc(NC(=O)C2CC2)cc1Nc1ncc(OC(F)F)cc1C1(F)CC1] | 439.4 | 440.3 | ¹H NMR (400 MHz, DMSO-d₆) δ 12.35 (s, 1H), 11.32 (s, 1H), 9.71 (s, 1H), 9.18 (s, 1H), 8.26 (s, 1H), 7.79 (s, 1H), 7.26 (t, J = 73.7 Hz, 1H), 2.14-2.05 (m, 1H), 1.62- 1.48 (m, 2H), 1.23-1.14 (m, 2H), 0.88-0.79 (m, 4H). |

### Example 9A: methyl (6-((methyl-d3)carbamoyl)-5-((3-(1-(trifluoromethyl)cyclopropyl)pyridin-2-yl)amino)pyridazin-3-yl)carbamate (Compound 9)

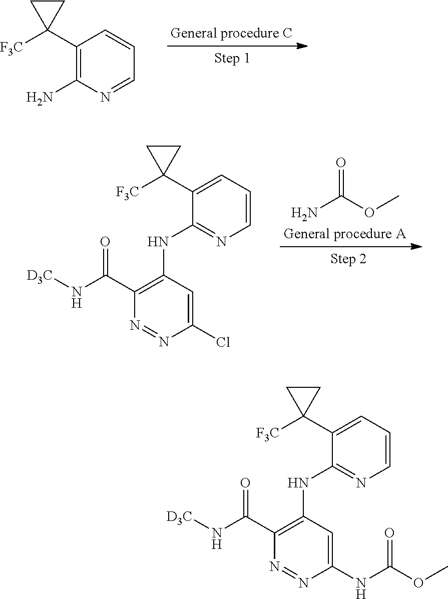

### Step 1: 6-chloro-N-(methyl-d3)-4-((3-(1-(trifluoromethyl)cyclopropyl)pyridin-2-yl)amino)pyridazine-3-carboxamide

The synthesis was carried out according to General Procedure C using 3-(1-(trifluoromethyl)cyclopropyl)pyridin-2-amine (450 mg, 2.3 mmol) and 4,6-dichloro-N-(methyl-d₃)pyridazine-3-carboxamide (558 mg, 2.78 mmol) to afford 6-chloro-N-(methyl-d₃)-4-((3-(1-(trifluoromethyl)cyclopropyl)pyridin-2-yl)amino)pyridazine-3-carboxamide (75 mg, 9%) as a white powder. LCMS Method F (m/z): [M+H]+=375.2, retention time=1.41 min.

### Step 2: Synthesis of methyl (6-((methyl-d₃)carbamoyl)-5-((3-(1-(trifluoromethyl)cyclopropyl)pyridin-2-yl)amino)pyridazin-3-yl)carbamate

The synthesis was carried out according to General Procedure F using 6-chloro-N-(methyl-d₃)-4-((3-(1-(trifluoromethyl)cyclopropyl)pyridin-2-yl)amino)pyridazine-3-carboxamide (32 mg, 104 umol) and methyl carbamate (32 mg, 104 umol) to afford the titled product (11.0 mg, 13%). LCMS Method H (m/z): [M+H]+=414.2, retention time=2.31 min. ¹H NMR (500 MHz, DMSO-d₆)=12.14 (s, 1H), 10.79 (s, 1H), 9.62 (s, 1H), 9.16 (s, 1H), 8.40 (dd, J=4.8, 1.8, 1H), 7.89 (dd, J=7.5, 1.8, 1H), 7.13 (dd, J=7.5, 4.8, 1H), 3.72 (s, 3H), 1.67-1.57 (m, 2H), 1.30-1.22 (m, 2H).

### Example 9B: 6-(3-isopropyl-2-oxoimidazolidin-1-yl)-N-(methyl-d3)-4-((3-(1-(trifluoromethyl)cyclopropyl)pyridin-2-yl)amino)pyridazine-3-carboxamide (Compound 18)

The following compounds were prepared in a manner similar to that of Compound 9 described in Example 9A using the corresponding amide

TABLE 4

| | | | (m/z) | |
|---|---|---|---|---|
| Compound | | MW | [M + H]⁺ | |
| No. | Structure | (g/mol) | (method) | ¹H NMR |
| 18 | 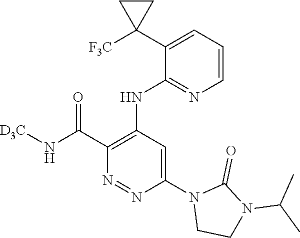 [SMILES: [2H]C([2H])([2H])NC(=O)c1nnc(N2CCN(C(C)C)C2=O)cc1Nc1ncccc1C1(C(F)(F)F)CC1] | 466.5 | 467.6 (L) | 1H NMR (500 MHz, DMSO-d₆) δ = 12.02 (s, 1H), 9.87 (s, 1H), 9.22 (s, 1H), 8.37 (dd, J = 4.8, 1.8, 1H), 7.88 (dd, J = 7.5, 1.8, 1H), 7.11 (dd, J = 7.5, 4.8, 1H), 4.14-4.08 (m, 3H), 3.52- 3.46 (m, 2H), 1.66-1.58 (m, 2H), 1.26- 1.24 (m, 2H), 1.15 (d, J = 6.8, 6H). |

### Example 10A: 6-(cyclopropanecarboxamido)-4-((3-(1-fluoroethyl)pyridin-2-yl)amino)-N-(methyl-d3)pyridazine-3-carboxamide (Compound 10)

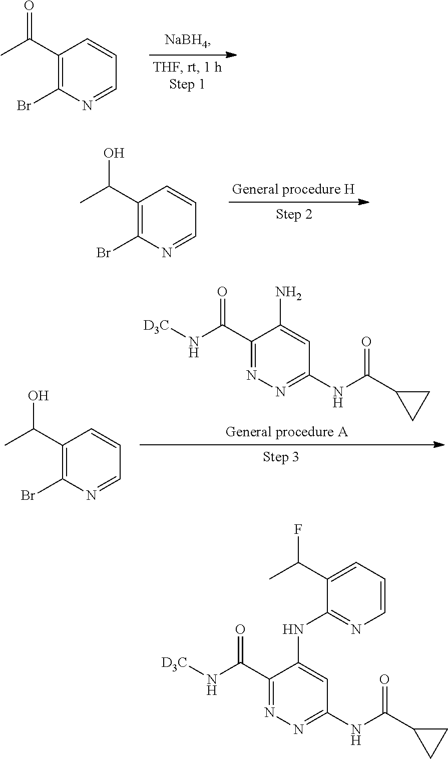

### Step 1: Synthesis of 1-(2-bromopyridin-3-yl)ethan-1-ol

To a stirred solution of 3-acetyl-2-bromopyridine (335 mg, 1.64 mmol) in THF (8.00 mL) was added NaBH4 (110 mg, 2.79 mmol) at room temperature. Stirring continued for 90 minutes, and a sample was injected on LC (incomplete conversion). Additional quantity of NaBH4 (112 mg, 2.84 mmol) was added and stirring continued for another 20 h. Water (10 mL) was slowly added followed by satd. aq. NH4Cl (10 mL) and DCM (10 mL). The mixture was stirred for 1 h. The mixture was extracted with DCM (2×50 mL). The combined organics were washed with brine, dried over anh. Na2SO4, filtered and concentrated under reduced pressure to provide 1-(2-bromopyridin-3-yl)ethan-1-ol (293 mg, 88%) as a yellow oil. LCMS Method K (m/z): 204.0, retention time=1.01 min. 1H NMR (400 MHz, DMSO-d6) δ 8.26 (d, J=4.6 Hz, 1H), 7.98-7.90 (m, 1H), 7.47 (dd, J=7.6, 4.6 Hz, 1H), 5.57 (d, J=4.2 Hz, 1H), 4.92-4.83 (m, 1H), 1.32 (d, J=6.4 Hz, 3H).

### Step 2: Synthesis of 2-bromo-3-(1-fluoroethyl)pyridine

The title compound was synthesized according to General Procedure H. To a solution of 1-(2-bromopyridin-3-yl)ethan-1-ol (290 mg, 1.44 mmol) in DCM (20.0 mL) was added (Diethylamino)sulfur trifluoride (701 uL, 5.38 mmol). The solution was stirred at rt for 40 min. The mixture was added dropwise to a stirred aq. NaHCO₃ sat. at 0° C. and stirred for 1 h. The mixture was extracted with DCM (2×50 mL). The combined organic solution was washed with NaHCO₃ sat. (2×), NH₄Cl sat., brine, dried over anh. Na₂SO₄, filtered and concentrated under reduced pressure to provide the title compound (179 mg, 61%) as a brown oil. LCMS Method C (m/z): [M+H]⁺=206.1, retention time=1.68 min. 1H NMR (400 MHz, DMSO-d6) δ 8.39 (d, J=3.9 Hz, 1H), 7.95 (d, J=7.7 Hz, 1H), 7.55 (dd, J=7.7, 4.7 Hz, 1H), 5.85 (dq, J=46.4, 6.3 Hz, 1H), 1.62 (dd, J=24.3, 6.4 Hz, 3H).

### Step 3: 6-(cyclopropanecarboxamido)-4-((3-(1-fluoroethyl)pyridin-2-yl)amino)-N-(methyl-d3)pyridazine-3-carboxamide

The title compound was synthesized according to General Procedure A. To a degassed mixture of 2-bromo-3-(1-fluoroethyl)pyridine (222 mg, 999 umol), XantPhos (51.0 mg, 86.4 umol), 4-amino-6-(cyclopropanecarboxamido)-N-(methyl-d3)pyridazine-3-carboxamide (98.0 mg, 411 umol) and K2CO3 (157 mg, 1.11 mmol) in dioxane (1.50 mL) was added Pd2(dba)3 (41.4 mg, 45.2 umol). Nitrogen was immediately bubbled to the mixture for 2 min and was stirred at 65° C. for 1 h, then at 75° C. for 16 h. The mixture was removed from the heating source, more Pd2(dba)3 (103 mg, 109 umol) and XantPhos (59.5 mg, 101 umol) were added and nitrogen was immediately bubbled to the mixture for 2 min. The mixture was heated again to 80° C. for 8 h. The mixture was cooled, diluted with EtOAc and filtered on Celite®. The Celite® cake was rinsed with DCM and EtOAc and the combined organic was concentrated under reduced pressure. The residue was purified by silica gel chromatography (0-10% IPA:DCM) to provide a solid. The residue was sonicated with ACN and concentrated to provide the title compound (36.9 mg, 23%). LCMS Method C (m/z): [M+H]+=362.4, retention time=2.05 min. 1H NMR (500 MHz, DMSO-d6) δ 11.94 (s, 1H), 11.32 (s, 1H), 9.56 (s, 1H), 9.24 (s, 1H), 8.36 (d, J=4.8 Hz, 1H), 7.87 (d, J=7.6 Hz, 1H), 7.15 (dd, J=7.5, 5.0 Hz, 1H), 5.93 (dq, J=46.8, 6.4 Hz, 1H), 2.15-2.08 (m, 1H), 1.71 (dd, J=24.0, 6.4 Hz, 3H), 0.91-0.81 (m, 4H).

### Example 10B: Synthesis of 1-(2-chloro-4-fluoropyridin-3-yl)ethan-1-one (Compound 101)

Pyridyl ketones that were not commercially available were synthesized in a two step, on pot procedure as follows:

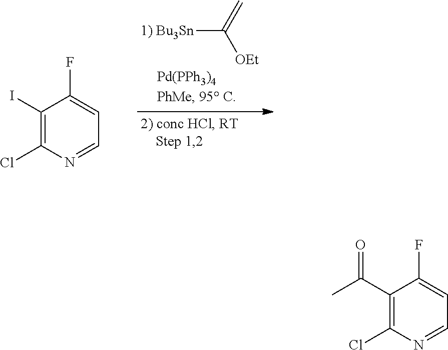

### Steps 1, 2: Synthesis of 1-(2-chloro-4-fluoropyridin-3-yl)ethan-1-one

2-Chloro-4-fluoro-3-iodopyridine (1.10 g, 4.19 mmol) and Tributyl(1-ethoxyvinyl)tin (1.75 g, 4.61 mmol) were dissolved in toluene (22.0 mL) and Tetrakis(triphenylphosphine)palladium(0) (370 mg, 314 umol) was added. Nitrogen was bubbled through the mixture for 5 mins, the vial was sealed and heated to 80° C. for 36 h with stirring. Reaction incomplete so more Tetrakis(triphenylphosphine)palladium(0) (370 mg, 314 umol) was added and the heat increased to 95° C. and the mixture was left to stir additional 24 h at which point full conversion was observed by LCMS. The reaction was cooled to room temperature and conc HCl (523 uL, 6.28 mmol) was added and the mixture stirred for 1 h. The reaction was poured over ca. 15 mL of saturated sodium bicarbonate, and the aqueous layer was extracted with EtOAc. The combined organic portions were washed with saturated sodium bicarbonate and brine, dried over anh. Na2SO4, filtered and adsorbed onto silica gel. Purified by normal phase flash chromatography (Dry Pack) (SiO2 with Heptanes/EtOAc: 0%-100%), the fractions containing product were combined and the solvent removed in vacuo affording the title compound (475 mg, 65%) a pale yellow oil. LCMS Method Q (m/z): [M+H]+=174.4, retention time=0.76 min.

The following example was prepared in a manner similar to Compound 10 described in Example 10A from their corresponding acetylpyridines.

| | | | (m/z) | |
|---|---|---|---|---|
| Cmpd | | MW | [M + H]⁺ | |
| No. | Structure | (g/mol) | (method) | ¹H NMR |
| 101 | 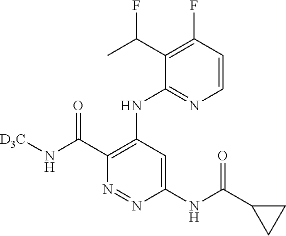 [SMILES: [2H]C([2H])([2H])NC(=O)c1nnc(NC(=O)C2CC2)cc1Nc1nccc(F)c1C(C)F] | 379.4 | 380.4 (S) | ¹HNMR (400 MHz, DMSO-d₆) δ 12.04 (d, J = 3.1 Hz, 1H), 11.36 (s, 8.36 (dd, J = 8.1, 5.8 Hz, 1H), 7.07 (dd, J = 9.4, 5.6 Hz, 1H), 6.13 (dq, J = 45.5, 6.6 Hz, 1H), 2.16-2.08 (m, 1H), 1.76 (dd, J = 23.1, 6.6 Hz, 1H), 9.53 (s, 1H), 9.24 (s, 1H), 3H), 0.95-0.77 (m, 4H). |

### Example 11A: 6-(cyclopropanecarboxamido)-4-((3-(1,1-difluoroethyl)pyridin-2-yl)amino)-N-(methyl-d3)pyridazine-3-carboxamide (Compound 11)

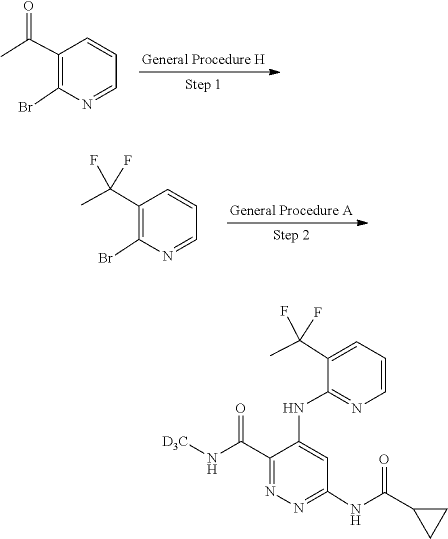

### Step 1: Synthesis of 2-bromo-3-(1,1-difluoroethyl)pyridine

The title compound was synthesized according to General Procedure H. To a solution of 3-acetyl-2-bromopyridine (1.03 mL, 7.76 mmol) in DCM (4.00 mL), was added (diethylamino)sulfur trifluoride (3.59 mL, 27.5 mmol). The solution was stirred at 50° C. for 24 h. The mixture was added dropwise to a stirring solution of ice-cold satd. aq. NaHCO₃, stirring continued for an hour, and was extracted with DCM (50 mL). The organic layer was washed with satd. aq. NH4Cl, followed by brine, dried over Na2SO4, filtered and concentrated under reduced pressure to provide the title compound (1.43 g, 83%) as a yellow oil. LCMS Method K (m/z): [M+H]+=no-ionization, retention time=1.97 min. 1H NMR (400 MHz, DMSO-d6) δ 8.51 (d, J=4.4 Hz, 1H), 8.05 (d, J=7.8 Hz, 1H), 7.59 (dd, J=7.7, 4.7 Hz, 1H), 2.09 (t, J=19.1 Hz, 3H).

### Step 2: Synthesis of 6-(cyclopropanecarboxamido)-4-((3-(1,1-difluoroethyl)pyridin-2-yl)amino)-N-(methyl-d3)pyridazine-3-carboxamide

The title compound was prepared according to General Procedure A. To a degassed mixture of 2-bromo-3-(1,1-difluoroethyl)pyridine (222 mg, 999 umol), XantPhos (51.0 mg, 86.4 umol), 4-amino-6-(cyclopropanecarboxamido)-N-(methyl-d3)pyridazine-3-carboxamide (98.0 mg, 411 umol) and K2CO3 (157 mg, 1.11 mmol) in dioxane (1.50 mL), was added Pd2(dba)3 (41.4 mg, 45.2 umol). Nitrogen was immediately bubbled through mixture for 2 min and was stirred at 65° C. for 1 h, then at 75° C. for 16 h. The mixture was removed from the heating source, more Pd2(dba)3 (103 mg, 109 umol) and XantPhos (59.5 mg, 101 umol) were added and nitrogen was immediately bubbled through mixture for 2 min. The mixture was heated again at 80° C. for 8 h. The mixture was cooled, diluted with EtOAc and filtered on Celite® and the Celite® cake was rinsed with DCM and EtOAc and the combined organics were concentrated under reduced pressure. The residue was purified over normal phase flash chromatography using IPA in DCM (0-10%) as an eluent. Fractions containing product were concentrated under reduced pressure to afford a solid, which was sonicated in ACN/water mixture and lyophilized to provide the title compound (36.9 mg, 23%) as an off-white solid. LCMS Method C (m/z): [M+H]+=380.3, retention time=2.05 min. 1H NMR (500 MHz, DMSO-d6) δ 11.99 (s, 1H), 11.34 (s, 1H), 9.52 (s, 1H), 9.20 (s, 1H), 8.49-8.43 (m, 1H), 7.97 (dd, J=7.8, 1.7 Hz, 1H), 7.20 (dd, J=7.7, 4.8 Hz, 1H), 2.15-2.08 (m, 1H), 2.07 (t, J=19.2 Hz, 3H), 0.88-0.81 (m, 4H).

### Examples 11B-12H

The following examples were prepared in a manner similar to Example 11A from their corresponding commercially available 2-halo, 3-acetylheteroarenes.

TABLE 5

| | | | | (m/z) | |
|---|---|---|---|---|---|
| Ex. | Compd | | | [M + H]⁺ | |
| No. | No. | Structure | MW | (method) | 1H NMR |
| 11B | 12 | 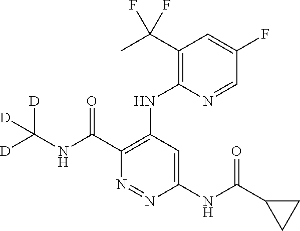 [SMILES: [2H]C([2H])([2H])NC(=O)c1nnc(NC(=O)C2CC2)cc1Nc1ncc(F)cc1C(C)(F)F] | 397.38 | 398.30 (D) | ¹H NMR (500 MHz, DMSO-D₆) δ 11.99 (s, 1H), 11.35 (s, 1H), 9.33 (s, 1H), 9.21 (s, 1H), 8.50 (d, J = 2.9 Hz, 1H), 7.96 (dd, J = 8.9, 2.9 Hz, 1H), 2.14-2.03 (m, 4H), 0.87- 0.83 (m, 4H). |
| 11C | 16 | 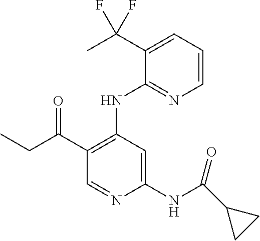 [SMILES: CCC(=O)c1cnc(NC(=O)C2CC2)cc1Nc1ncccc1C(C)(F)F] | 374.39 | 375.3 (K) | ¹H NMR (500 MHz, DMSO-d₆) δ = 11.29 (s, 1H), 10.86 (s, 1H), 9.11 (s, 1H), 7.91 (s, 1H), 7.66 (dd, J=7.9, 1.2, 1H), 7.60- 7.53 (m, 1H), 7.41- 7.34 (m, 1H), 2.11-2.01 (m, 1H), 1.92 (t, JH-F = 19.1, 3H), 0.91-0.74 (m, 4H). |
| 11D | 28 | 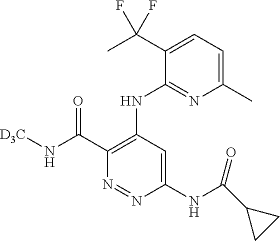 [SMILES: [2H]C([2H])([2H])NC(=O)c1nnc(NC(=O)C2CC2)cc1Nc1nc(C)ccc1C(C)(F)F] | 393.4 | 394.3 (H) | ¹H NMR (500 MHz, DMSO-d₆) δ 12.00 (s, 1H), 11.31 (s, 1H), 9.88 (s, 1H), 9.18 (s, 1H), 7.82 (d, J = 7.9 Hz, 1H), 7.03 (d, J = 7.8 Hz, 1H), 2.50 (submerged s, 3H), 2.17- 2.11 (m, 1H), 2.05 (t, J = 19.1 Hz, 3H), 0.88-0.83 (m, 4H). |
| 11E | 49 | 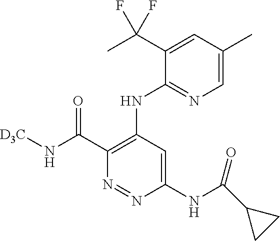 [SMILES: [2H]C([2H])([2H])NC(=O)c1nnc(NC(=O)C2CC2)cc1Nc1ncc(C)cc1C(C)(F)F] | 393.41 | 394.3 (H) | ¹H NMR (500 MHz, DMSO-d₆) δ 11.85 (s, 1H), 11.31 (s, 1H), 9.40 (s, 1H), 9.17 (s, 1H), 8.30 (d, J = 1.3 Hz, 1H), 7.81 (d, J = 1.7 Hz, 1H), 2.32 (s, 3H), 2.11 (m, 1H), 2.05 (t, J = 19.2 Hz, 3H), 0.84 (m, 4H). |
| 11F | 77 | 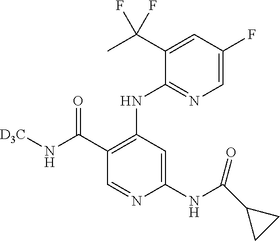 [SMILES: [2H]C([2H])([2H])NC(=O)c1cnc(NC(=O)C2CC2)cc1Nc1ncc(F)cc1C(C)(F)F] | 396.38 | 397.3 (D) | ¹H NMR (400 MHz, DMSO-d₆) δ 11.46 (s, 1H), 10.76 (s, 1H), 8.85 (s, 1H), 8.64 (s, 1H), 8.57 (s, 1H), 8.45 (d, J = 2.8 Hz, 1H), 7.91 (dd, J = 8.8, 2.9 Hz, 1H), 2.12-1.96 (m, 4H), 0.82-0.74 (m, 4H). |
| 11G | 65 | 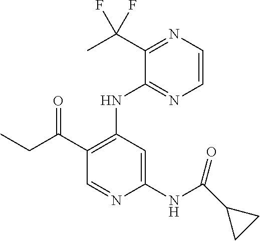 [SMILES: CCC(=O)c1cnc(NC(=O)C2CC2)cc1Nc1nccnc1C(C)(F)F] | 375.38 | 376.1 (G) | ¹H NMR (400 MHz, DMSO-d₆) δ 12.20 (s, 1H), 11.00 (s, 1H), 9.31 (s, 1H), 8.98 (s, 1H), 8.55 (s, 1H), 8.32 (d, J = 2.0 Hz, 1H), 3.15 (q, J = 7.1 Hz, 2H), 2.13 (t, J = 19.7 Hz, 3H), 2.08-2.03 (m, 1H), 1.11 (t, J = 7.1 Hz, 3H), 0.86-0.81 (m, 4H). |
| 11H | 87 | 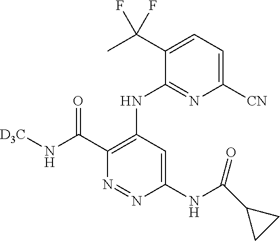 [SMILES: [2H]C([2H])([2H])NC(=O)c1nnc(NC(=O)C2CC2)cc1Nc1nc(C#N)ccc1C(C)(F)F] | 404.4 | 405.1 (T) | ¹H NMR (400 MHz, DMSO-d₆) δ 12.35 (s, 1H), 11.44 (s, 1H), 9.56 (s, 1H), 9.29 (s, 1H), 8.14 (d, J = 7.8 Hz, 1H), 7.79 (d, J = 7.8 Hz, 1H), 2.19- 1.98 (m, 4H), 0.92-0.79 (m, 4H). |

### Example 12: 6-(cyclopropanecarboxamido)-N-(methyl-d3)-4-((3-(oxetan-3-yl)pyridin-2-yl)amino)pyridazine-3-carboxamide (Compound 13)

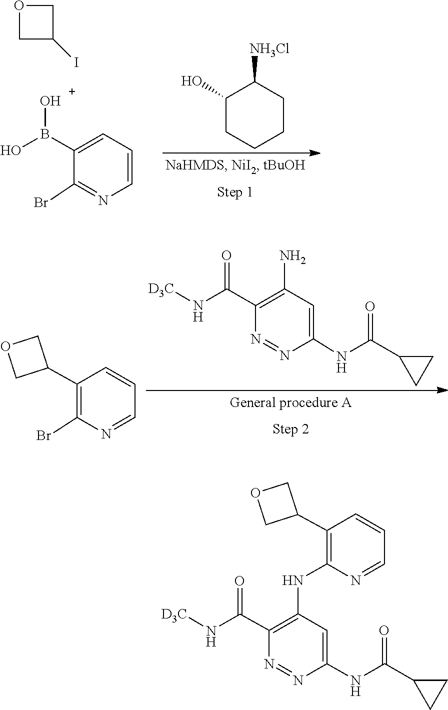

### Step 1: Synthesis of 2-bromo-3-(oxetan-3-yl)pyridine

To a degassed solution of (2-bromopyridin-3-yl)boronic acid (1.80 g, 8.85 mmol), nickel (II) iodide (174 mg, 557 umol) and trans-2-aminocyclohexanol hydrochloride (87.8 mg, 579 umol), in t-BuOH (6.21 mL), was added NaHMDS in THF (10.5 mL, 10.5 mmol) followed by 3-iodooxetane (0.500 mL, 5.57 mmol). Nitrogen was immediately bubbled through the mixture for 2 min, and then heated at 70° C. for 6.5 h. The mixture was diluted with DCM, filtered through Celite®, washed with DCM, and the filtrate was concentrated under reduced pressure and the residue was triturated with 0.5 mL of dioxane to afford 2-bromo-3-(oxetan-3-yl)pyridine (40.0 mg, 3.4%), which was used as-is in the next step. LCMS Method C (m/z): [M+H]+=no-ionization, retention time=0.98 min. ¹H NMR (400 MHz, DMSO-D₆) δ 8.29 (d, J=4.2 Hz, 1H), 7.96 (d, J=7.5 Hz, 1H), 7.51 (dd, J=7.2, 4.8 Hz, 1H), 4.98-4.91 (m, 2H), 4.69 (t, J=6.6 Hz, 2H), 4.50-4.38 (m, 1H).

### Step 2: Synthesis of 6-(cyclopropanecarboxamido)-N-(methyl-d₃)-4-((3-(oxetan-3-yl)pyridin-2-yl)amino)pyridazine-3-carboxamide

Synthesized according to General Procedure A. To a degassed solution of 2-bromo-3-(oxetan-3-yl)pyridine (40.0 mg, 187 umol), 4-amino-6-(cyclopropanecarboxamido)-N-(methyl-d₃)pyridazine-3-carboxamide (44.5 mg, 187 umol), 4,5-bis(diphenylphosphino)-9,9-dimethylxanthene (22.1 mg, 37.4 umol), and Cs₂CO₃ (154 mg, 467 umol) in dioxane (934 uL), was added tris(dibenzylideneacetone)dipalladium(0) (17.1 mg, 18.7 umol). Nitrogen was immediately bubbled through the mixture for 2 min, and then heated at 100° C. for 1.25 h. The mixture was cooled, diluted with EtOAc, and filtered through Celite®, rinsed with a mixture of MeOH and EtOAc (25% MeOH, 4×50 mL), and the filtrate was concentrated under reduced pressure. The residue was purified by normal phase flash chromatography using IPA in DCM (0-20%) as an eluent to provide 24 mg of a mixture of 39% purity. The residue was further purified over Prep-HPLC to provide 6-(cyclopropanecarboxamido)-N-(methyl-d₃)-4-((3-(oxetan-3-yl)pyridin-2-yl)amino)pyridazine-3-carboxamide (2.50 mg, 8.1%). LCMS Method K (m/z): [M+H]+=372.2, retention time=1.90 min. ¹H NMR (500 MHz, DMSO-D₆) δ 11.63 (s, 1H), 11.30 (s, 1H), 9.64 (s, 1H), 9.29 (s, 1H), 8.26 (dd, J=4.9, 1.5 Hz, 1H), 7.90 (d, J=7.7 Hz, 1H), 7.13 (dd, J=7.5, 4.9 Hz, 1H), 5.06 (dd, J=8.2, 6.1 Hz, 2H), 4.75-4.70 (m, 2H), 4.52-4.43 (m, 1H), 2.12 (ddd, J=12.4, 7.4, 5.1 Hz, 1H), 0.91-0.80 (m, 4H).

### Example 13: 6-(cyclopropanecarboxamido)-4-((3-(2-methoxypropan-2-yl)pyridin-2-yl)amino)-N-(methyl-d3)pyridazine-3-carboxamide (Compound 30)

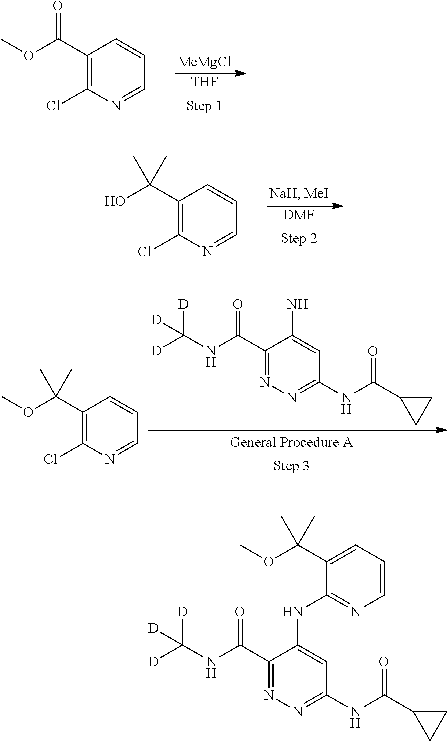

### Step 1: Synthesis of 2-(2-chloropyridin-3-yl)propan-2-ol

Synthesized according to general procedure G. To a solution of methyl-2-chloropyridine-3-carboxylate (2.28 mL, 17.5 mmol) in THF (69.9 mL) was added 3.0 M methylmagnesium chloride in THF (17.5 mL, 52.5 mmol) at 0° C. The reaction mixture was allowed to stir at room temperature for 1 h. The reaction mixture was quenched with sat. aq. NH₄Cl then extracted with ethyl acetate. The combined organic layers were dried over anh. Na₂SO₄, filtered and concentrated under reduced pressure. The residue was dry-loaded onto silica and purified by normal phase chromatography (0-50% EtOAc/Heptanes) to afford 2-(2-chloropyridin-3-yl)propan-2-ol (2.71 g, 90%) as a colorless oil. LCMS Method B (m/z): [M+H]⁺=172.2, retention time=0.87 min.

### Step 2: Synthesis of 2-chloro-3-(2-methoxypropan-2-yl)pyridine

2-(2-chloropyridin-3-yl)propan-2-ol (240 mg, 1.40 mmol) and DMF (2.80 mL) were cooled in an ice bath and sodium hydride 60% in dispersion in mineral oil (83.9 mg, 2.10 mmol) was added and stirred for 15 minutes in an ice bath. To this mixture was added iodomethane (132 uL, 2.10 mmol) at 0° C. The reaction mixture was then stirred at room temperature for 2 h. To this mixture was added sat. aq. NH₄Cl, extracted with DCM and dried over Na₂SO₄, filtered, and concentrated under reduced pressure. The residue was dry-loaded onto silica and purified by normal phase chromatography (0-20% EtOAc/Heptanes) to afford 2-chloro-3-(2-methoxypropan-2-yl)pyridine (203 mg, 78%) as a colorless oil. LCMS Method B (m/z): [M+H]⁺=186.2, retention time=0.93 min.

### Step 3: Synthesis of 6-(cyclopropanecarboxamido)-4-((3-(2-methoxypropan-2-yl)pyridin-2-yl)amino)-N-(methyl-d₃)pyridazine-3-carboxamide

The title compound was synthesized according to general procedure A

LCMS Method D (m/z): [M+H]⁺=388.3, retention time=2.14 min. ¹H NMR (500 MHz, DMSO-d₆) δ=11.59 (s, 1H), 11.23 (s, 1H), 9.30 (s, 1H), 8.96 (s, 1H), 8.24 (dd, J=4.8, 1.7, 1H), 7.73 (dd, J=7.8, 1.8, 1H), 7.09 (dd, J=7.7, 4.8, 1H), 3.11 (s, 3H), 2.13-2.06 (m, 1H), 1.54 (s, 6H), 0.90-0.79 (m, 4H).

### Example 14: 6-(cyclopropanecarboxamido)-N-(methyl-d3)-4-((3-(perfluoroethyl)pyridin-2-yl)amino)nicotinamide (Compound 31)

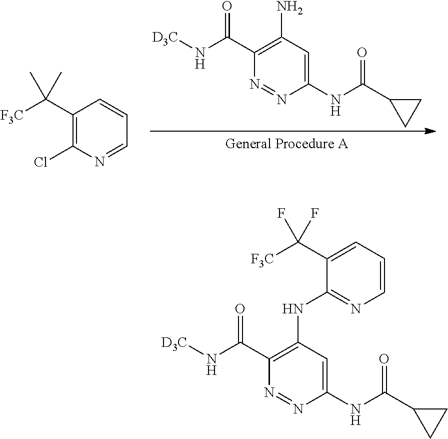 [SMILES: CC(C)(c1cccnc1Cl)C(F)(F)F ; [2H]C([2H])([2H])NC(=O)c1nnc(NC(=O)C2CC2)cc1N ; [2H]C([2H])([2H])NC(=O)c1nnc(NC(=O)C2CC2)cc1Nc1ncccc1C(F)(F)C(F)(F)F]

### Synthesis of 6-(cyclopropanecarboxamido)-N-(methyl-d₃)-4-((3-(perfluoroethyl)pyridin-2-yl)amino)nicotinamide

Prepared from commercially available pyridine according to General Procedure A

A flame-dried 2-5 mL microwave vial equipped with a Teflon®-coated stir bar and rubber septum was charged with 4-amino-6-(cyclopropanecarboxamido)-N-(methyl-d₃)pyridazine-3-carboxamide (100 mg, 420 umol), followed by XantPhos (99.1 mg, 168 umol), K₃PO₄ (364 mg, 1.68 mmol), 2-chloro-3-(pentafluoroethyl)pyridine (256 mg, 1.05 mmol), and dioxane (1.40 mL). The heterogeneous reaction mixture was degassed by bubbling with nitrogen gas (balloon) for five minutes, and then Pd₂(dba)₃ (76.9 mg, 83.9 umol) was added. The vial was sealed with a crimp cap with PTFE septum in it, placed in a pre-heated oil bath (105° C.), stirring continued for 4 h, and a sample was injected on LCMS (complete conversion). The reaction mixture was diluted in DCM (100 mL), adsorbed on silica gel (~25 g) via concentration under reduced pressure, purified over normal phase flash chromatography (SiO₂, 0-100% EtOAc in heptanes mobile phase) to afford the title compound (25.0 mg, 14%). LCMS Method D (m/z): [M+H]⁺=434.3, retention time=2.44 min. ¹H NMR (400 MHz, DMSO-d₆) δ 12.23 (s, 1H), 11.41 (s, 1H), 9.47 (s, 1H), 9.25 (s, 1H), 8.64 (d, J=3.6 Hz, 1H), 8.15 (d, J=6.7 Hz, 1H), 7.33 (dd, J=7.8, 4.8 Hz, 1H), 2.16-2.08 (m, 1H), 0.88-0.836 (m, 4H).

### Example 15: 4-((3-(1-cyanocyclobutyl)pyridin-2-yl)amino)-6-(cyclopropanecarboxamido)-N-(methyl-d3)pyridazine-3-carboxamide (Compound 14)

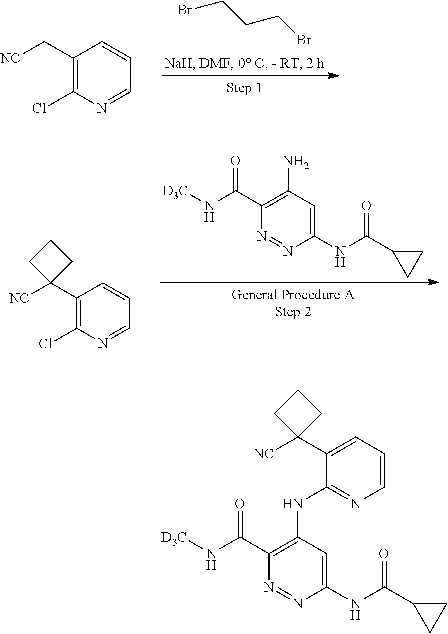 [SMILES: BrCCCBr ; N#CCc1cccnc1Cl ; N#CC1(c2cccnc2Cl)CCC1 ; [2H]C([2H])([2H])NC(=O)c1nnc(NC(=O)C2CC2)cc1N ; [2H]C([2H])([2H])NC(=O)c1nnc(NC(=O)C2CC2)cc1Nc1ncccc1C1(C#N)CCC1]

### Step 1: 1-(2-chloropyridin-3-yl)cyclobutane-1-carbonitrile

(2-Chloropyridin-3-yl)acetonitrile (0.500 g, 3.18 mmol) and DMF (7.50 mL) were cooled in an ice bath and NaH (636 mg, 15.9 mmol) was added with stirring. The reaction was stirred at room temperature for 15 mins then 1,3-Dibromopropane (358 uL, 3.50 mmol) was added dropwise. The reaction was stirred at room temperature for 2 h. The excess NaH was quenched with slow addition of methanol until gas evolution ceased then the mixture was concentrated to near-dryness in vacuo The residue was taken up in EtOAc and washed with brine. The organic portion was dried over anh. Na₂SO₄, filtered and adsorbed onto silica gel. Purified by normal phase flash chromatography (Dry Pack) (SiO₂ with Heptanes/EtOAc: 5%-100%), the fractions containing product were combined and the solvent removed in vacuo affording the title compound (280 mg, 46%) as a pale brown oil. LCMS Method A (m/z): no ionization, retention time=1.00 min.

### Step 2: 4-((3-(1-cyanocyclobutyl)pyridin-2-yl)amino)-6-(cyclopropanecarboxamido)-N-(methyl-d₃)pyridazine-3-carboxamide

The title compound was prepared according to General Procedure A using 4-amino-6-(cyclopropanecarboxamido)-N-(methyl-d₃)pyridazine-3-carboxamide (88.4 mg, 371 umol) and 1-(2-chloropyridin-3-yl)cyclobutane-1-carbonitrile (65.0 mg, 337 umol). The product was purified over reverse phase flash chromatography on C18 column using MeCN in 10 mM AmB buffer (25%-60%) as an eluent to afford the title compound (10.0 mg, 9.5%). LCMS Method F (m/z): [M+H]⁺=395.3, retention time=1.84 min. ¹H NMR (500 MHz, DMSO-d₆) δ 11.38 (s, 1H), 11.34 (s, 1H), 9.32 (s, 1H), 9.25 (s, 1H), 8.38 (dd, J=4.8, 1.7 Hz, 1H), 7.85 (dd, J=7.8, 1.7 Hz, 1H), 7.22 (dd, J=7.7, 4.8 Hz, 1H), 3.05-2.97 (m, 2H), 2.74-2.65 (m, 2H), 2.40-2.29 (m, 1H), 2.14-2.08 (m, 1H), 1.98-1.90 (m, 1H), 0.87-0.82 (m, 4H).

### Example 16A: 4-((3-(1-cyanocyclopropyl)-4-methylpyridin-2-yl)amino)-6-(cyclopropanecarboxamido)-N-(methyl-d3)nicotinamide (Compound 34)

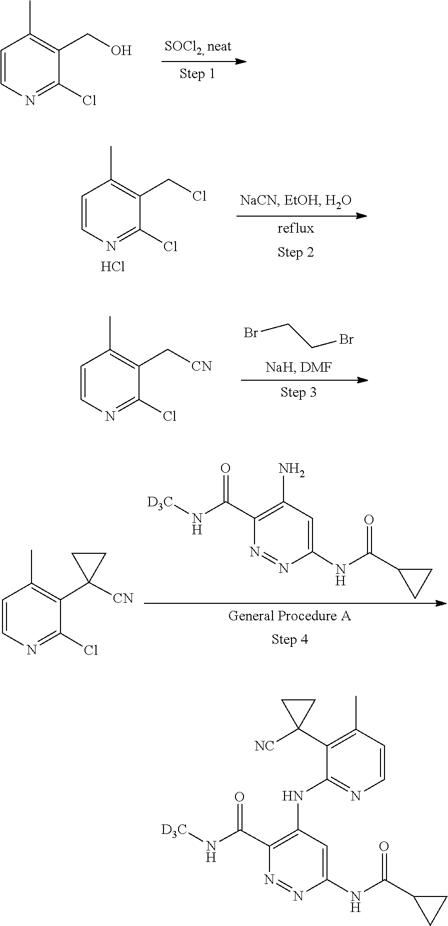

### Step 1: Synthesis of 2-chloro-3-(chloromethyl)-4-methylpyridine hydrochloride

(2-Chloro-4-methylpyridin-3-yl)methanol (361 mg, 2.22 mmol) was stirred in neat thionyl chloride (9.77 mL, 133 mmol) at 40° C. for 20 mins. Full conversion observed—the thionyl chloride was removed under a stream of compressed air affording the title compound (320 mg, 68%) as a white solid which was carried forward without purification. LCMS Method B (m/z): [M+H]⁺=176.2, retention time=1.05 min.

### Step 2: Synthesis of 2-(2-chloro-4-methylpyridin-3-yl)acetonitrile

2-chloro-3-(chloromethyl)-4-methylpyridine hydrochloride (400 mg, 1.88 mmol), sodium cyanide (266 mg, 5.27 mmol), ethanol (8.00 mL) and water (4.00 mL) were stirred at 90° C. for 18 h. The reaction was cooled to ambient temperature and the ethanol was removed under vacuum. The aqueous layer was diluted with water extracted with EtOAc. The combined organics were washed with brine, dried over anh. Na₂SO₄, filtered and concentrated under vacuum. The residue was taken up in DMSO and purified by reverse phase flash chromatography (C18 silica with 10 mM Ammonium Bicarbonate buffer/ACN: 5%-30%), the fractions containing product were combined and lyophilized affording the title compound (90.0 mg, 29%) as a colorless oil. LCMS Method B (m/z): [M+H]⁺=165.0, retention time=0.67 min.

### Step 3: Synthesis of 1-(2-chloro-4-methylpyridin-3-yl)cyclopropane-1-carbonitrile

2-(2-chloro-4-methylpyridin-3-yl)acetonitrile (285 mg, 1.71 mmol) and DMF (4.04 mL) were cooled in an ice bath and NaH (342 mg, 8.55 mmol) was added with stirring. The reaction was stirred at room temperature for 15 mins then 1,2-dibromoethane (164 uL, 1.88 mmol) was added dropwise. The reaction was stirred for 1 h at room temperature and the excess NaH was quenched with slow addition of methanol until gas evolution ceased. The mixture was concentrated to near dryness in vacuo. The residue was taken up in EtOAc and washed with brine. The organic portion was dried over anh. Na₂SO₄, filtered and concentrated under vacuum. The residue was taken up in DMSO and purified by reverse phase flash chromatography (C18 silica with 10 mM Ammonium Bicarbonate buffer/MeCN: 5%-30%), the fractions containing product were combined and extracted with EtOAc. The organic portion was separated and washed with water and brine and isolated affording the title compound (190 mg, 58%) as a pale yellow semi-solid. LCMS Method B (m/z): [M+H]⁺=193.0.0, retention time=0.84 min.

### Step 4: 4-((3-(1-cyanocyclobutyl)pyridin-2-yl)amino)-6-(cyclopropanecarboxamido)-N-(methyl-d₃)pyridazine-3-carboxamide

The title compound was prepared according to General Procedure A using 4-amino-6-(cyclopropanecarboxamido)-N-(methyl-d₃)pyridazine-3-carboxamide (117 mg, 493 umol) and 1-(2-chloropyridin-3-yl)cyclobutane-1-carbonitrile (95 mg, 493 umol). The product was purified over reverse phase flash chromatography on C18 column using MeCN in 10 mM AmB buffer (25%-60%) as an eluent to afford the title compound (10.0 mg, 9.5%). LCMS Method G (m/z): [M+H]⁺=395.2, retention time=2.03 min. ¹H NMR (500 MHz, DMSO-d6) δ 12.20 (s, 1H), 11.32 (s, 1H), 9.75 (s, 1H), 9.26 (s, 1H), 8.22 (d, J=5.0 Hz, 1H), 7.00 (d, J=5.0 Hz, 1H), 2.49 (s, 3H), 2.19-2.08 (m, 1H), 2.06-1.94 (m, 2H), 1.50-1.39 (m, 2H), 0.94-0.80 (m, 4H).

### Examples 16B-16C

The following compounds were prepared in a similar manner to Compound 34 described in example 16A.

TABLE 6

| | | | | (m/z) | |
|---|---|---|---|---|---|
| | Cmpd | | MW | [M + H]⁺ | |
| Ex. No. | No. | Structure | (g/mol) | (method) | ¹H NMR |
| 16B | 33 | 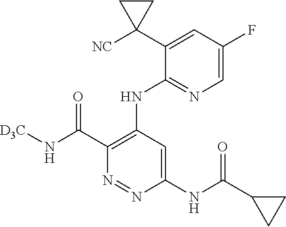 [SMILES: [2H]C([2H])([2H])NC(=O)c1nnc(NC(=O)C2CC2)cc1Nc1ncc(F)cc1C1(C#N)CC1] | 398.4 | 399.3 (G) | ¹H NMR (500 MHz, DMSO-d₆) δ 12.59 (s, 1H), 11.47 (s, 1H), 9.94 (s, 1H), 9.32 (s, 1H), 8.86 (s, 1H), 8.65 (s, 1H), 2.20-2.08 (m, 1H), 1.62 (s, 2H), 1.34 (s, 2H), 0.96- 0.81 (m, 4H). |
| 16C | 27 | 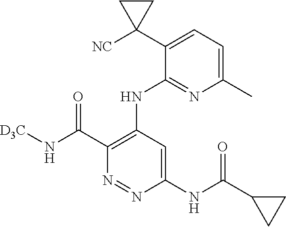 [SMILES: [2H]C([2H])([2H])NC(=O)c1nnc(NC(=O)C2CC2)cc1Nc1nc(C)ccc1C1(C#N)CC1] | 394.5 | 395.3 (G) | ¹H NMR (500 MHz, DMSO-d₆) δ 12.32 (s, 1H), 11.32 (s, 1H), 10.07 (s, 1H), 9.26 (s, 1H), 7.72 (d, J = 7.7 Hz, 1H), 6.93 (d, J = 7.7 Hz, 1H), 2.48 (s, 3H), 2.19-2.08 (m, 1H), 1.92-1.78 (m, 2H), 1.53- 1.41 (m, 2H), 0.92-0.81 (m, 4H). |

### Example 17A: 6-(cyclopropanecarboxamido)-4-((3-(1-fluorocyclopropyl)pyridin-2-yl)amino)-N-(methyl-d₃)pyridazine-3-carboxamide (Compound 17)

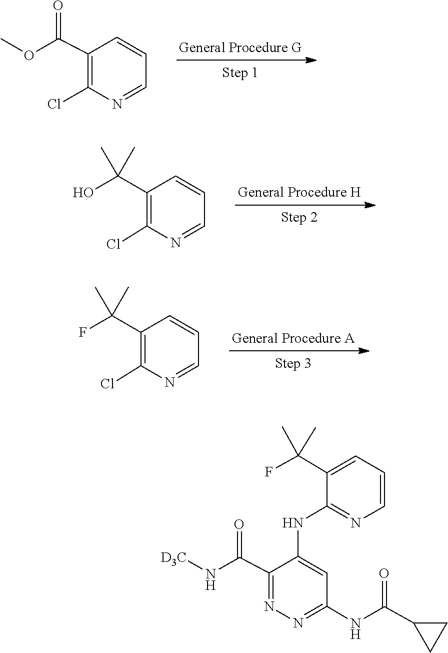

### Step 1: Synthesis of 2-(2-chloropyridin-3-yl)propan-2-ol

The title compound was prepared according to General Procedure G. To a solution of methyl-2-chloropyridine-3-carboxylate (761 uL, 5.83 mmol) in THF (23.3 mL) was added 3.0 M methylmagnesium chloride in THF (5.83 mL, 17.5 mmol) at 0° C. The solution was stirred at room temperature for 1 h. The reaction mixture was quenched with saturated aq. NH₄Cl and then extracted with DCM. The combined organic layers were dried over anh. Na₂SO₄, filtered, and concentrated under reduced pressure. This residue was purified using flash chromatography (dry load silica, Biotage, 40 g, silica gel column, 0 to 100% EtOAc/Heptanes) (DP came out at 50% EtOAc). The appropriate fractions that contained product were concentrated under vacuum to obtain the title compound (514 mg, 51%) as a brown liquid. LCMS Method B (m/z): [M+H]⁺=172.2, retention time=0.77 min.

### Step 2: Synthesis of 2-chloro-3-(2-fluoropropan-2-yl)pyridine

The title compound was prepared according to General Procedure H. To a solution of 2-(2-chloropyridin-3-yl)propan-2-ol (360 mg, 2.10 mmol) in DCM (4.20 mL) was added (Diethylamino)sulfur trifluoride (328 uL, 2.52 mmol) at −78° C. The solution was stirred at room temperature for 30 min. The mixture was added dropwise to a stirred sat. aq. NaHCO₃ solution and stirred for 10 min. The mixture was extracted with DCM. The combined organic layers were dried over Na₂SO₄, filtered, and concentrated under reduced pressure. This residue was purified using flash chromatography (dry load silica, Biotage, 40 g, silica gel column, 0 to 70% EtOAc/Heptanes). The appropriate fractions that contained product were concentrated to obtain the title compound (214 mg, 59%) as a brown liquid. LCMS Method B (m/z): [M+H]⁺=174.3, retention time=1.15 min.

### Step 3: Synthesis of 6-(cyclopropanecarboxamido)-4-((3-(1-fluorocyclopropyl)pyridin-2-yl)amino)-N-(methyl-d₃)pyridazine-3-carboxamide

The title compound was prepared according to General Procedure A. To a mixture of 4-amino-6-(cyclopropanecarboxamido)-N-(methyl-d₃)pyridazine-3-carboxamide (125 mg, 525 umol), 2-chloro-3-(2-fluoropropan-2-yl)pyridine (137 mg, 787 umol), Pd₂(dba)₃ (96.1 mg, 105 umol), XantPhos (124 mg, 210 umol) and K₂CO₃ (148 mg, 1.05 mmol) was added dioxane (1.91 mL). The reaction mixture was stirred at 100° C. for 5 h under N₂ atmosphere. The reaction was cooled to room temperature, diluted with water and extracted with EtOAc. The combined organic layers were dried over Na₂SO₄, filtered, and concentrated under reduced pressure. The residue was purified by normal phase flash chromatography using MeOH in DCM (0-100%) as an eluent. The residue was further purified over Prep-HPLC to provide the title compound (10.0 mg, 5.1%). LCMS Method H (m/z): [M+H]⁺=376.3, retention time=1.95 min. ¹H NMR (500 MHz, DMSO-d₆) δ=11.68 (d, J=7.3, 1H), 11.28 (s, 1H), 9.36 (s, 1H), 9.12 (s, 1H), 8.33-8.29 (m, 1H), 7.85-7.81 (m, 1H), 7.16-7.12 (m, 1H), 2.14-2.08 (m, 1H), 1.79 (d, JH-F=22.1, 6H), 0.87-0.82 (m, 4H).

### Examples 17B-17J

The following examples were prepared in a manner similar to Compound 17 disclosed in Example 17A from their corresponding substituted 2-haloheteroarenes and 4-aminoheteroarenes:

TABLE 7

| | | | | (m/z) | |
|---|---|---|---|---|---|
| Ex. | Cmpd | | MW | [M + H]⁺ | |
| No. | No. | Structure | (g/mol) | (method) | ¹H NMR |
| 17B | 23 | 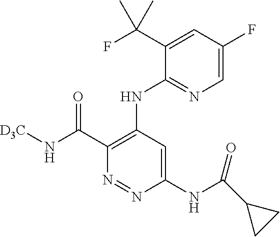 [SMILES: [2H]C([2H])([2H])NC(=O)c1nnc(NC(=O)C2CC2)cc1Nc1ncc(F)cc1C(C)(C)F] | 393.4 | 394.4 (L) | ¹H NMR (400 MHz, DMSO-d₆) δ = 11.67 (d, J = 6.8, 1H), 11.31 (s, 1H), 9.17 (s, 1H), 9.14 (s, 1H), 8.35 (d, J = 2.7, 1H), 7.84-7.76 (m, 1H), 2.15-2.06 (m, 1H), 1.79 (d, J = 22.2, 6H), 0.90-0.80 (m, 4H). |
| 17C | 62 | 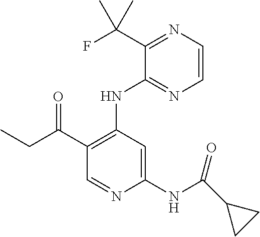 [SMILES: CCC(=O)c1cnc(NC(=O)C2CC2)cc1Nc1nccnc1C(C)(C)F] | 371.42 | 372.1 (S) | ¹H NMR (400 MHz, DMSO-d₆) δ 11.90 (d, J = 9.1 Hz, 1H), 10.95 (s, 1H), 9.15 (s, 1H), 8.95 (s, 1H), 8.38 (d, J = 2.0 Hz, 1H), 8.27 (dd, J = 2.5, 0.9 Hz, 1H), 3.13 (q, J = 7.2 Hz, 2H), 2.09-2.02 (m, 1H), 1.79 (d, J = 22.1 Hz, 6H), 1.10 (t, J = 7.2 Hz, 3H), 0.82 (d, J = 6.2 Hz, 4H). |
| 17D | 89 | 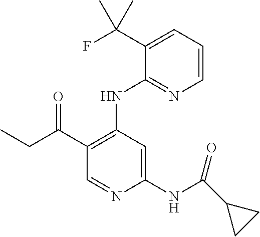 [SMILES: CCC(=O)c1cnc(NC(=O)C2CC2)cc1Nc1ncccc1C(C)(C)F] | 370.43 | 371.6 (O) | ¹H NMR (400 MHz, DMSO-d₆) δ 11.60 (d, J = 8.0 Hz, 1H), 10.84 (s, 1H), 8.90 (s, 1H), 8.90 (s, 1H), 8.33 (d, J = 4.6 Hz, 1H), 7.83 (d, J = 7.8 Hz, 1H), 7.16 (ddd, J = 7.7, 4.8, 1.3 Hz, 1H), 3.11 (q, J = 7.2 Hz, 2H), 2.08-1.98 (m, 1H), 1.77 (d, J = 22.0 Hz, 6H), 1.09 (t, J = 7.2 Hz, 3H), 0.88-0.71 (m, 4H). |
| 17E | 90 | 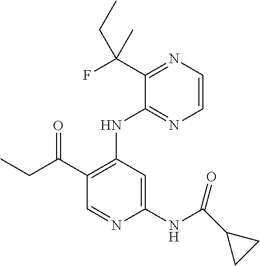 [SMILES: CCC(=O)c1cnc(NC(=O)C2CC2)cc1Nc1nccnc1C(C)(F)CC] | 385.44 | 386.6 (O) | ¹H NMR (400 MHz, DMSO-d₆) δ 11.89 (d, J = 10.1 Hz, 1H), 10.94 (s, 1H), 9.15 (s, 1H), 8.94 (s, 1H), 8.37 (d, J = 2.4 Hz, 1H), 8.32- 8.26 (m, 1H), 3.13 (q, J = 7.2 Hz, 4H), 2.30-2.06 (m, 2H), 2.06- 1.98 (m, 1H), 1.75 (d, J = 22.9 Hz, 3H), 0.98 (dt, J = 90.7, 7.3 Hz, 3H), 0.82 (d, J = 6.1 Hz, 4H). |
| 17F | 91 | 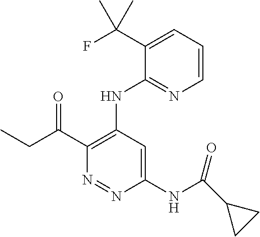 [SMILES: CCC(=O)c1nnc(NC(=O)C2CC2)cc1Nc1ncccc1C(C)(C)F] | 371.42 | 372.3 (S) | ¹H NMR (400 MHz, DMSO-d₆) δ 11.51 (s, 1H), 11.36 (d, J = 9.9 Hz, 1H), 9.37 (s, 1H), 8.34 (d, J = 4.6 Hz, 1H), 7.85 (d, J = 7.8 Hz, 1H), 7.24-7.13 (m, 1H), 3.34 (q, J = 7.3 Hz, 3H), 2.19-2.09 (m, 1H), 1.79 (d, J = 22.1 Hz, 6H), 1.12 (t, J = 7.3 Hz, 3H), 0.85 (d, J = 7.4 Hz, 4H). |
| 17G | 92 | 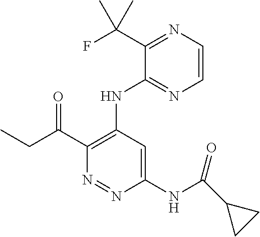 [SMILES: CCC(=O)c1nnc(NC(=O)C2CC2)cc1Nc1nccnc1C(C)(C)F] | 372.4 | 373.3 (S) | ¹H NMR (400 MHz, DMSO-d₆) δ 11.63 (d, J = 10.6 Hz, 1H), 11.58 (s, 1H), 9.54 (s, 1H), 8.41 (d, J = 2.4 Hz, 1H), 8.31 (d, J = 1.6 Hz, 1H), 3.36 (q, 2H), 2.16-2.08 (m, 1H), 1.81 (d, J = 22.4 Hz, 6H), 1.13 (t, J = 7.3 Hz, 3H), 0.93-0.85 (m, 4H). |
| 17H | 93 | 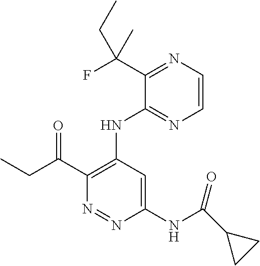 [SMILES: CCC(=O)c1nnc(NC(=O)C2CC2)cc1Nc1nccnc1C(C)(F)CC] | 386.43 | 387.5 (G) | ¹H NMR (400 MHz, DMSO-d₆) δ 11.63 (d, J = 11.8 Hz, 1H), 11.57 (s, 1H), 9.54 (s, 1H), 8.40 (d, J = 2.4 Hz, 1H), 8.33 (d, J = 1.4 Hz, 1H), 3.38-3.34 (m, 2H)*, 2.32- 2.00 (m, 2H), 2.18-2.07 (m, 1H), 1.76 (d, J = 23.1 Hz, 3H), 1.13 (t, J = 7.3 Hz, 3H), 0.90-0.85 (m, 7H). *under water peak (expecting q, J = 7.3 Hz, 2H) |
| 17I | 94A | 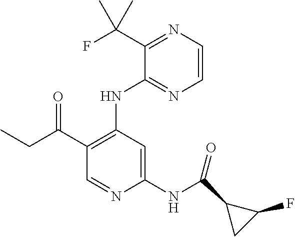 [SMILES: CCC(=O)c1cnc(NC(=O)[C@@H]2C[C@@H]2F)cc1Nc1nccnc1C(C)(C)F] | 389.41 | 390.2 (S) | ¹H NMR (400 MHz, DMSO-d₆) δ 11.91 (d, J = 9.1 Hz, 1H), 10.99 (s, 1H), 9.15 (s, 1H), 8.96 (s, 1H), 8.40 (d, J = 2.0 Hz, 1H), 8.28 (dd, J = 2.5, 0.9 Hz, 1H), 4.93 (dtd, J = 66.1, 6.2, 3.8 Hz, 1H), 3.14 (q, J = 7.2 Hz, 2H), 2.24 (dtd, J = 8.9, 7.0,45.0 Hz, 1H), 1.80 (dd, J = 22.1, 2.7 Hz, 6H), 1.63 (dtd, J = 23.4, 6.8, 3.8 Hz, 1H), 1.17 (ddd, J = 12.4, 6.2, 2.8 Hz, 1H), 1.10 (t, J = 7.2 Hz, 3H). |
| 17J | 95 | 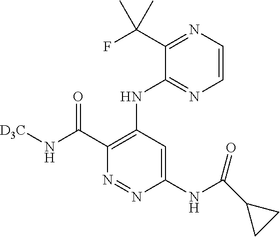 [SMILES: [2H]C([2H])([2H])NC(=O)c1nnc(NC(=O)C2CC2)cc1Nc1nccnc1C(C)(C)F] | 376.41 | 377.1 (S) | ¹H NMR (400 MHz, DMSO-d₆) δ 12.09 (d, J = 8.3 Hz, 1H), 11.39 (s, 1H), 9.54 (s, 1H), 9.18 (s, 1H), 8.37 (d, J = 2.3 Hz, 1H), 8.26 (d, J = 1.8 Hz, 1H), 2.16-2.06 (m, 1H), 1.80 (d, J = 22.0 Hz, 6H), 0.89-0.84 (m, 4H). |

### Example 18A: 6-(cyclopropanecarboxamido)-4-((5-fluoro-3-(1-fluorocyclopropyl)pyridin-2-yl)amino)-N-(methyl-d3)pyridazine-3-carboxamide (Compound 20)

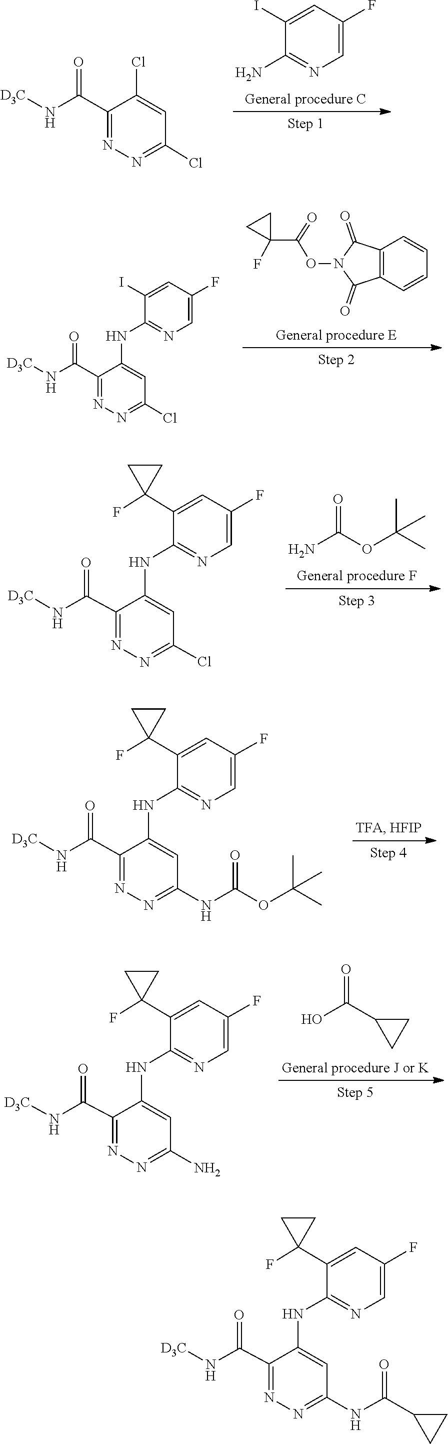

### Step 1: 6-chloro-4-((5-fluoro-3-iodopyridin-2-yl)amino)-N-(methyl-d₃)pyridazine-3-carboxamide

The synthesis was carried out according to General Procedure C using 5-fluoro-3-iodo-pyridin-2-ylamine (1 g, 403 umol) and 4,6-dichloro-N-(methyl-d₃)pyridazine-3-carboxamide (1.01 g, 484 umol) affording the title compound (1.49 g, 90%) as a tan powder. LCMS Method B (m/z): [M+H]⁺=411.1, retention time=1.35 min.

### Step 2: 6-chloro-4-((5-fluoro-3-(1-fluorocyclopropyl)pyridin-2-yl)amino)-N-(methyl-d₃)pyridazine-3-carboxamide

The synthesis was carried out according to General Procedure E using 6-chloro-4-((5-fluoro-3-iodopyridin-2-yl)amino)-N-(methyl-d₃)pyridazine-3-carboxamide (4.02 g, 9.79 mmol) and 1,3-dioxoisoindolin-2-yl 1-fluorocyclopropane-1-carboxylate (7.32 g, 29.4 mmol). The reaction mixture was flushed through a silica plug with EtOAc (200 mL), dried and redissolved in DCM, washed with 1M NaOH (aq.) (200 mL) (discarding the aqueous layer), purified by flash column chromatography (0-10% iPrOH in DCM) and finally by recrystallization from hot iPrOH to afford the title compound (1.8 g, 35%) as an off-white solid. LCMS Method B (m/z): [M+H]+=325.3, retention time=1.33 min. ¹H NMR (400 MHz, DMSO-d₆) δ 12.55 (s, 1H), 9.46 (s, 1H), 9.09 (s, 1H), 8.53 (s, 1H), 8.00 (d, J=7.8 Hz, 1H), 1.64-1.53 (m, 2H), 1.28-1.19 (m, 2H).

### Step 3: tert-butyl (5-((5-fluoro-3-(1-fluorocyclopropyl)pyridin-2-yl)amino)-6-((methyl-d₃)carbamoyl)pyridazin-3-yl)carbamate

The synthesis was carried out according to General Procedure F using 6-chloro-4-((5-fluoro-3-(1-fluorocyclopropyl)pyridin-2-yl)amino)-N-(methyl-d₃)pyridazine-3-carboxamide (686 mg, 2.00 mmol), tert-Butyl carbamate (598 mg, 5.00 mmol), Pd₂(dba)₃ (289 mg, 300 umol), dcpf (364 mg, 600 umol), K₃PO₄ (1.73 g, 8.01 mmol) in dry dioxane (15.5 mL). After heating the reaction mixture at 110° C. for 3.5 h, LCMS indicated the reaction had completed. The reaction mixture was flushed through a silica plug with EtOAc (200 mL) and evaporated to dryness under reduced pressure to give a brown residue, which was used in the next step without further purification. LCMS Method J (m/z): [M+H]⁺=424.1, retention time=1.70 min.

### Step 4: 6-amino-4-((5-fluoro-3-(1-fluorocyclopropyl)pyridin-2-yl)amino)-N-(methyl-d₃)pyridazine-3-carboxamide

To this brown residue obtained from the previous step was added HFIP (25.2 mL) followed by TFA (1.53 mL, 20.0 mmol), and the mixture was stirred for 16 h at 25° C., after which LCMS indicated the second step had completed. The reaction mixture was evaporated to dryness under reduced pressure in a room temperature water bath. DCM was added and evaporated several times to remove residual TFA. The residue was partitioned between DCM (130 mL) and saturated NaHCO₃ solution (150 mL). The aqueous layer was extracted further with DCM (2×50 mL). The combined organic layers were dried over Na₂SO₄ and concentrated under vacuum. The residue was purified by column chromatography (Normal phase: 0-5% MeOH in DCM, followed by Reversed phase: 20-50% MeCN in 10 mM AmB) to yield the title compound (393 mg, 61% over two steps) as a white powder. LCMS Method J (m/z): [M+H]⁺=324.1, retention time=1.24 min. ¹H NMR (400 MHz, DMSO-d₆) δ 12.21 (s, 1H), 8.93 (s, 1H), 8.35 (dd, J=2.5, 1.7 Hz, 1H), 8.09 (s, 1H), 7.90 (dt, J=8.6, 2.2 Hz, 1H), 6.72 (s, 2H), 1.64-1.51 (m, 2H), 1.23-1.14 (m, 2H).

### Step 5: Synthesis of 6-(cyclopropanecarboxamido)-4-((5-fluoro-3-(1-fluorocyclopropyl)pyridin-2-yl)amino)-N-(methyl-d₃)pyridazine-3-carboxamide

The synthesis was carried out according to General Procedure J. In a first vial, to a mixture of Cyclopropanecarboxylic acid (31.1 uL, 371 umol), NMI (74.7 uL, 928 umol) and DMF (160 uL) was added TCFH (268 mg, 928 umol). The mixture was stirred at room temperature for 10 minutes.

In a second vial, 6-amino-4-((5-fluoro-3-(1-fluorocyclopropyl)pyridin-2-yl)amino)-N-(methyl-d3)pyridazine-3-carboxamide (30.0 mg, 92.8 umol) was dissolved in DMF (140 uL) and NMI (187 uL, 2.32 mmol) was added. Then the activated acid mixture (first vial) was added to the second vial. The mixture was stirred at room temperature for 90 minutes, then heated to 40° C. for 90 minutes. Product was precipitated with addition of water and washed with 5×2 mL of 1:1 MeCN:H₂O to give the title compound (22.0 mg, 61%). LCMS Method L (m/z): [M+H]⁺=392.1, retention time=2.58 min. ¹H NMR (500 MHz, DMSO-d₆) δ 12.32 (s, 1H), 11.33 (s, 1H), 9.67 (s, 1H), 9.20 (s, 1H), 8.40 (dd, J=2.7, 1.5 Hz, 1H), 7.93 (ddd, J=8.6, 2.6, 1.8 Hz, 1H), 2.17-2.07 (m, 1H), 1.62-1.51 (m, 2H), 1.26-1.18 (m, 2H), 0.91-0.82 (m, 4H).

### Example 18B: 4-((5-fluoro-3-(1-fluorocyclopropyl)pyridin-2-yl)amino)-6-((1S,2S)-2-fluorocyclopropane-1-carboxamido)-N-(methyl-d3)pyridazine-3-carboxamide (Compound 21)

The following examples were prepared in a similar manner to Compound 20 described in Example 18A, using the corresponding chloropyridazines or chloropyridines and commercially available carboxylic acids

TABLE 8
Synthesis of compound 21A

| | | | | (m/z) | |
|---|---|---|---|---|---|
| Cmpd | | General | MW | [M + H]⁺ | |
| No. | Structure | Procedures | (g/mol) | (method) | ¹H NMR |
| 21A | 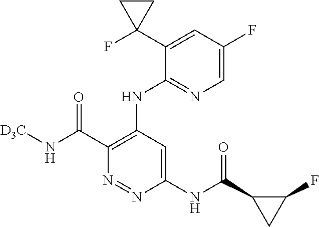 [SMILES: [2H]C([2H])([2H])NC(=O)c1nnc(NC(=O)[C@@H]2C[C@@H]2F)cc1Nc1ncc(F)cc1C1(F)CC1] | E, K | 409.4 | 410.3 (L) | ¹H NMR (400 MHZ, DMSO-d₆) δ 12.34 (s, 1H), 11.39 (s, 1H), 9.66 (s, 1H), 9.22 (s, 1H), 8.42 (s, 1H), 7.94 (d, J = 8.4 Hz, 1H), 4.96 (d, J = 66.2 Hz, 1H), 2.37-2.25 (m, 1H), 1.73-1.47 (m, 3H), 1.27-1.15 (m, 3H). |

### Example 19: 6-(1-fluorocyclopropane-1-carboxamido)-4-((3-(1-fluorocyclopropyl)pyridin-2-yl)amino)-N-(methyl-d3)nicotinamide (Compound 84)

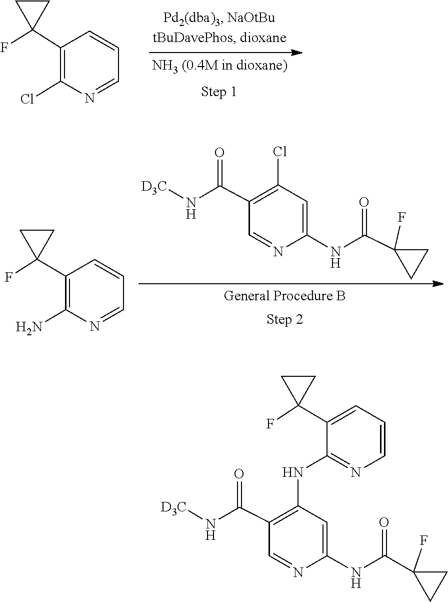 [SMILES: FC1(c2cccnc2Cl)CC1 ; Nc1ncccc1C1(F)CC1 ; [2H]C([2H])([2H])NC(=O)c1cnc(NC(=O)C2(F)CC2)cc1Cl ; [2H]C([2H])([2H])NC(=O)c1cnc(NC(=O)C2(F)CC2)cc1Nc1ncccc1C1(F)CC1]

### Step 1: Synthesis of 3-(1-fluorocyclopropyl)pyridin-2-amine

In a pressure tube, to a mixture of Pd₂(dba)₃ (27.7 mg, 30.3 umol), tBuDavePhos (21.1 mg, 60.6 umol) and sodium tert-butoxide (36.0 mg, 364 umol) was added dioxane (2.55 mL). This reaction mixture was bubbled with nitrogen for 5 min. To this mixture was then added 2-chloro-3-(1-fluorocyclopropyl)pyridine (100 mg, 303 umol) and Ammonia (3.79 mL, 1.52 mmol) 0.4 M solution in dioxane, the vial was sealed and stirred at 80° C. for 3 h. This residue was directly purified using flash chromatography (dry load silica, Biotage, 40 g, silica gel column, 0 to 10% DCM/MeOH). The appropriate fractions that were concentrated to obtain the title compound (35.0 mg, 39%) as a pale brown oil. LCMS Method B (m/z): [M+H]⁺=153.3,

### Step 2: Synthesis of 6-(1-fluorocyclopropane-1-carboxamido)-4-((3-(1-fluorocyclopropyl)pyridin-2-yl)amino)-N-(methyl-d₃)nicotinamide

The title compound was prepared according to General Procedure B using [1,1′-Bis(di-cyclohexylphosphino)ferrocene]dichloropalladium(II) (22.5 mg, 29.1 umol) and cesium carbonate (119 mg, 364 umol). LCMS Method D (m/z): [M+H]⁺=391.3, retention time=1.97 min. ¹H NMR (500 MHz, DMSO-d₆) δ=11.82 (s, 1H), 10.05 (s, 1H), 9.46 (s, 1H), 8.70 (s, 1H), 8.61 (s, 1H), 8.38-8.32 (m, 1H), 7.88-7.83 (m, 1H), 7.07-7.00 (m, 1H), 1.60-1.50 (m, 2H), 1.49-1.40 (m, 2H), 1.39-1.32 (m, 2H), 1.16-1.08 (m, 2H).

### Example 20: (1S,2S)-2-fluoro-N-(4-((3-(1-fluorocyclopropyl)pyridin-2-yl)amino)-5-propionylpyridin-2-yl)cyclopropane-1-carboxamide (Compound 76A)

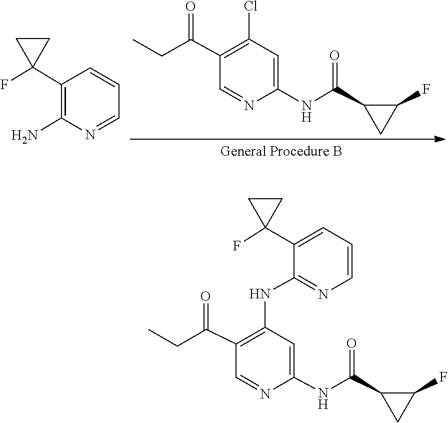 [SMILES: Nc1ncccc1C1(F)CC1 ; CCC(=O)c1cnc(NC(=O)[C@@H]2C[C@@H]2F)cc1Cl ; CCC(=O)c1cnc(NC(=O)[C@@H]2C[C@@H]2F)cc1Nc1ncccc1C1(F)CC1]

The title compound was prepared according to General Procedure B using [1,1′-Bis(di-cyclohexylphosphino)ferrocene]dichloropalladium(II) (22.9 mg, 29.6 umol), K₃PO₄ (128 mg, 591 umol). LCMS Method C (m/z): [M+H]⁺=387.4, retention time=2.24 min. ¹H NMR (500 MHz, DMSO-d₆) δ 12.18 (s, 1H), 10.94 (s, 1H), 9.56 (s, 1H), 8.95 (s, 1H), 8.48-8.35 (m, 1H), 7.89 (dt, J=7.5, 2.0 Hz, 1H), 7.10 (dd, J=6.7, 5.0 Hz, 1H), 4.93 (dtd, J=66.1, 6.2, 3.8 Hz, 1H), 3.15 (q, J=7.2 Hz, 2H), 2.32-2.19 (m, 1H), 1.73-1.52 (m, 3H), 1.24-1.08 (m, 6H).

### Example 21A: 6-(cyclobutanecarboxamido)-4-((3-(1-fluorocyclopropyl)pyridin-2-yl)amino)-N-(methyl-d3)pyridazine-3-carboxamide (Compound 51)

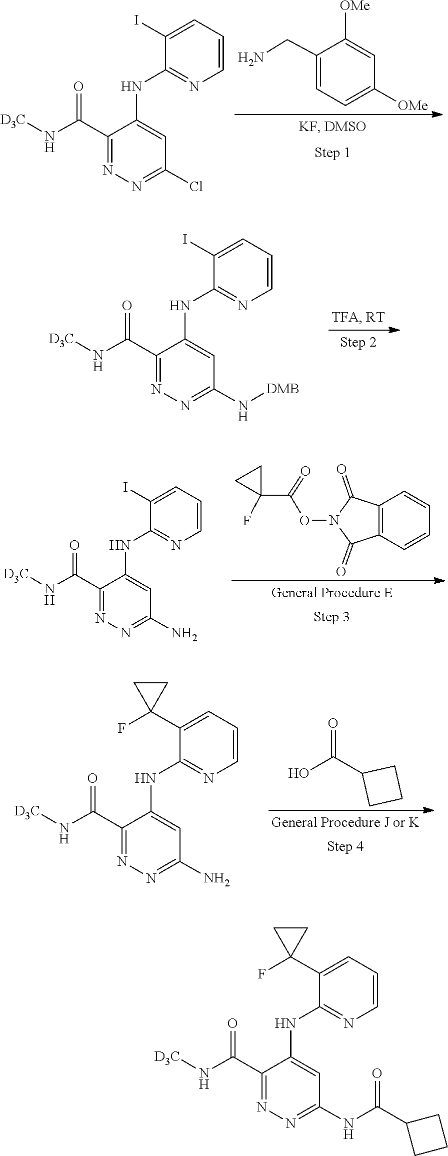

### Step 1: Synthesis of 6-((2,4-dimethoxybenzyl)amino)-4-((3-iodopyridin-2-yl)amino)-N-(methyl-d₃)pyridazine-3-carboxamide

6-Chloro-4-((3-iodopyridin-2-yl)amino)-N-(methyl-d₃)pyridazine-3-carboxamide (1.20 g, 3.06 mmol), 2,4-dimethoxybenzylamine (2.32 mL, 15.3 mmol), potassium fluoride (533 mg, 9.17 mmol) and DMSO (28.8 mL) were stirred at 120° C. for 6 h. The reaction was cooled to ambient temperature and poured over ca. 100 mL of water. The resulting precipitate was isolated by vacuum filtration and suction-dried affording the title compound (1.25 g, 78%) as a white solid. LCMS Method B (m/z): [M+H]⁺=524.2, retention time=1.39 min.

### Step 2: Synthesis of 6-amino-4-((3-iodopyridin-2-yl)amino)-N-(methyl-d₃)pyridazine-3-carboxamide

6-((2,4-dimethoxybenzyl)amino)-4-((3-iodopyridin-2-yl)amino)-N-(methyl-d₃)pyridazine-3-carboxamide (1.25 g, 2.39 mmol) and TFA (185 uL, 2.39 mmol) were stirred at room temperature for 45 mins and the TFA was removed under a stream of compressed air. The resulting red solid was stirred between EtOAc and NaHCO₃ (sat.) for 30 mins sonicating intermittently to break up the solid. The organic portion was washed with NaHCO₃ (sat.) and brine and the aqueous portion was back extracted with EtOAc. The combined organics were dried over anh. Na₂SO₄, filtered and adsorbed onto silica gel. Purified by normal phase flash chromatography (Dry Pack) (SiO₂ with DCM/IPA in DCM): 0%-10%), the fractions containing product were combined and the solvent removed in vacuo affording the title compound (620 mg, 70%) a yellow solid. LCMS Method B (m/z): [M+H]⁺=374.0, retention time=0.75 min.

### Step 3: Synthesis of 6-amino-4-((3-(1-fluorocyclopropyl)pyridin-2-yl)amino)-N-(methyl-d₃)pyridazine-3-carboxamide

The title compound was prepared according to General Procedure E

6-((2,4-dimethoxybenzyl)amino)-4-((3-iodopyridin-2-yl)amino)-N-(methyl-d₃)pyridazine-3-carboxamide (800 mg, 2.14 mmol) and 1,3-dioxoisoindolin-2-yl 1-fluorocyclopropane-1-carboxylate (1.60 g, 6.43 mmol) were dissolved in DMA (8.00 mL) and the solution was degassed with nitrogen for 10 mins while cooling in a brine/ice bath. In a separated flask fitted with a stir bar. Zinc (1.12 g, 17.2 mmol) and NiCl₂bpy (124 mg, 429 umol) were added and the flask was purged with nitrogen 10 mins while cooling in a brine/ice bath. The DMA solution was cannulated into the flask containing BIPY and zinc, and chlorotrimethylsilane (972 uL, 7.50 mmol) was added concurrently with vigorous stirring. The reaction was stirred for 30 mins then opened to air and filtered through ca. 25 g of silica gel topped with Celite® using EtOAc as eluent. The filtrate was concentrated to near-dryness under vacuum and loaded directly onto the flash column. Purified by reverse phase flash chromatography (C18 silica with 10 mM ammonium formate buffer/ACN: 5%-50%), the fractions containing product were combined and extracted into EtOAc. The organic portion was washed with water and brine and isolated affording the title compound (295 mg, 23%) as a yellow solid which was carried forward without further purification. LCMS Method C (m/z): [M+H]⁺=306.3, retention time=1.52 min.

### Step 4: Synthesis of 6-(cyclobutanecarboxamido)-4-((3-(1-fluorocyclopropyl)pyridin-2-yl)amino)-N-(methyl-d₃)pyridazine-3-carboxamide

The title compound was prepared according to general procedure J

Cyclobutanecarboxylic acid (31.3 uL, 328 umol), 1-methylimidazole (68.0 uL, 819 umol) and TCFH (232 mg, 819 umol) were stirred in MeCN (4.17 mL) at ambient temperature for 15 mins then 6-amino-4-((3-(1-fluorocyclopropyl)pyridin-2-yl)amino)-N-(methyl-d₃)pyridazine-3-carboxamide (25.0 mg, 81.9 umol) in MeCN (4.17 mL) was added and the reaction stirred at 50° C. for 2 h. The reaction was concentrated to ca. 1 mL under vacuum and injected directly into the purification system. Purification using Büchi C-850 Benchtop Prep chromatography (5-100% 10 mM AMB buffer in MeCN) afforded the title compound (7.40 mg, 23%). LCMS Method C (m/z): [M+H]⁺ 388.3, retention time=1.52 min ¹H NMR (400 MHz, DMSO-d₆) δ 12.32 (s, 1H), 10.85 (s, 1H), 9.88 (s, 1H), 9.19 (s, 1H), 8.44 (d, J=4.8 Hz, 1H), 7.90 (d, J=7.5 Hz, 1H), 7.11 (dd, J=7.2, 5.2 Hz, 1H), 3.53-3.41 (m, 1H), 2.31-−2.19 (m, 2H), 2.19-2.08 (m, 2H), 2.02-1.88 (m, 1H), 1.88-1.74 (m, 1H), 1.64-1.50 (m, 2H), 1.21-1.10 (in, 2H).

### Examples 21B-21R

The following examples were prepared in a similar manner to Compound 51 described in Example 21A using the corresponding chloropyridazines or chloropyridines and commercially available carboxylic acids

TABLE 9

| | | | | | (m/z) | |
|---|---|---|---|---|---|---|
| Ex. | Cmpd | Structure | General | MW | [M + H]⁺ | |
| No. | No. | | Proc. | (g/mol) | (method) | ¹H NMR |
| 21B | 53 | 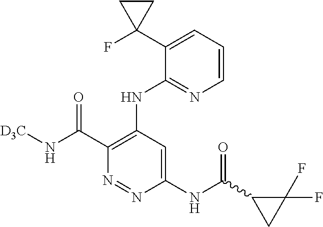 [SMILES: [2H]C([2H])([2H])NC(=O)c1nnc(NC(=O)C2CC2(F)F)cc1Nc1ncccc1C1(F)CC1] | E, K | 409.4 | 410.2 (L) | ¹H NMR (400 MHZ, DMSO-d₆) δ 12.37 (s, 1H), 11.54 (s, 1H), 9.84 (s, 1H), 9.25 (s, 1H), 8.40 (d, J = 4.8 Hz, 1H), 7.90 (d, J = 7.4 Hz, 1H), 7.11 (dd, J = 7.2, 5.2 Hz, 1H), 3.11 (ddd, J = 13.5, 10.7, 8.2 Hz, 1H), 2.15-1.99 (m, 2H), 1.64-1.51 (m, 2H), 1.21-1.09 (m, 2H). |
| 21C | 36A | 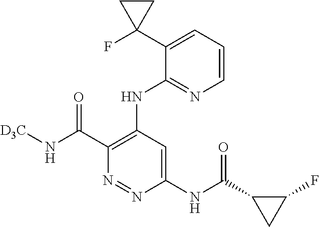 [SMILES: [2H]C([2H])([2H])NC(=O)c1nnc(NC(=O)[C@H]2C[C@H]2F)cc1Nc1ncccc1C1(F)CC1] | E, K | 391.4 | 392.3 (G) | ¹H NMR (400 MHZ, DMSO-d₆) δ 12.34 (s, 1H), 11.38 (s, 1H), 9.84 (s, 1H), 9.22 (s, 1H), 8.41 (d, J = 4.9 Hz, 1H), 7.91 (d, J = 7.5 Hz, 1H), 7.11 (dd, J = 7.4, 5.0 Hz, 1H), 5.18-4.73 (m, 1H), 2.36- 2.29 (m, 1H), 1.76- 1.65 (m, 1H), 1.63-1.53 (m, 2H), 1.29-1.20 (m, 1H), 1.20-1.13 (m, 2H). |
| 21D | 96 | 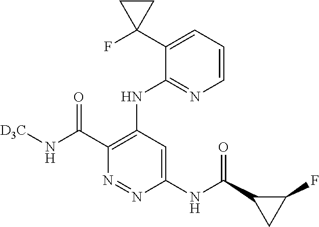 | E, K | 391.4 | 392.2 (G) | ¹H NMR (400 MHZ, DMSO-d₆) δ 12.33 (s, 1H), 11.37 (s, 1H), 9.83 (s, 1H), 9.21 (s, 1H), 8.44- 8.36 (m, 1H), 7.90 (dt, J = 7.6, 1.9 Hz, 1H), 7.10 (dd, J = 7.3, 5.0 Hz, 1H), 5.07-4.86 (m, 1H), 2.35- 2.30 (m, 1H), 1.75- 1.65 (m, 1H), 1.63-1.52 (m, 2H), 1.28-1.19 (m, 1H), 1.19-1.12 (m, 2H). |
| 21E | 41A | 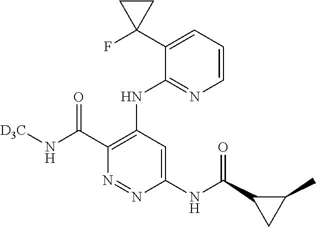 | E, K | 387.4 | 388.3 (G) | ¹H NMR (400 MHZ, DMSO-d₆) δ 12.23 (s, 1H), 11.14 (s, 1H), 9.74 (s, 1H), 9.12 (s, 1H), 8.39- 8.23 (m, 1H), 7.83 (dt, J = 7.5, 1.9 Hz, 1H), 7.02 (dd, J = 7.2, 5.1 Hz, 1H), 2.12-2.02 (m, 1H), 1.57- 1.43 (m, 2H), 1.31- 1.20 (m, 1H), 1.13-1.07 (m, 2H), 1.06 (d, J = 6.2 Hz, 3H), 0.98-0.91 (m, 1H), 0.82-0.74 (m, 1H). |
| 21F | 42A | 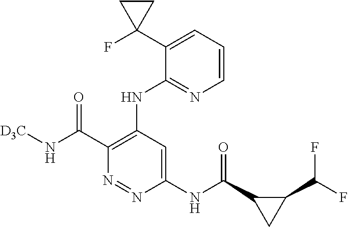 | E, K | 423.4 | 424.3 (G) | ¹H NMR (400 MHZ, DMSO-d₆) δ 12.27 (s, 1H), 11.43 (s, 1H), 9.76 (s, 1H), 9.16 (s, 1H), 8.31 (d, J = 4.9 Hz, 1H), 7.83 (d, J = 7.5 Hz, 1H), 7.03 (dd, J = 7.2, 5.1 Hz, 1H), 5.96 (td, J = 56.7, 4.6 Hz, 1H), 2.41-2.34 (m, 1H), 1.91-1.77 (m, 1H), 1.56- 1.45 (m, 2H), 1.18 1.06 (m, 4H). |
| 21G | 97A | 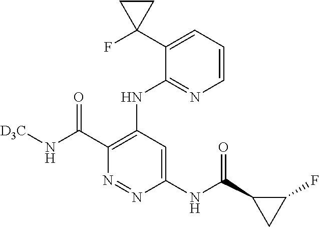 [SMILES: [2H]C([2H])([2H])NC(=O)c1nnc(NC(=O)[C@@H]2C[C@H]2F)cc1Nc1ncccc1C1(F)CC1] | E, K | 391.4 | 392.2 (G) | ¹H NMR (400 MHZ, DMSO-d₆) δ 12.27 (s, 1H), 11.42 (s, 1H), 9.75 (s, 1H), 9.15 (s, 1H), 8.37- 8.21 (m, 1H), 7.82 (dt, J = 7.5, 1.9 Hz, 1H), 7.02 (dd, J = 7.3, 5.1 Hz, 1H), 5.00-4.75 (m, 1H), 2.65- 2.55 (m, 1H), 1.67- 1.41 (m, 3H), 1.29-1.19 (m, 1H), 1.12-1.04 (m, 2H). |
| 21H | 97B | 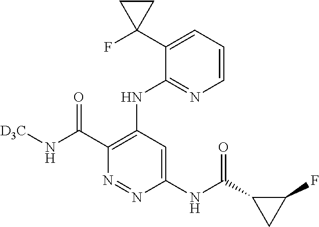 [SMILES: [2H]C([2H])([2H])NC(=O)c1nnc(NC(=O)[C@H]2C[C@@H]2F)cc1Nc1ncccc1C1(F)CC1] | E, K | 391.4 | 392.3 (G) | ¹H NMR (400 MHZ, DMSO-d₆) δ 12.27 (s, 1H), 11.41 (s, 1H), 9.75 (s, 1H), 9.15 (s, 1H), 8.30 (d, J = 4.9 Hz, 1H), 7.82 (d, J = 7.5 Hz, 1H), 7.02 (dd, J = 7.3, 5.0 Hz, 1H), 4.99-4.78 (m, 1H), 2.68- 2.53 (m, 1H), 1.57- 1.43 (m, 3H), 1.30-1.19 (m, 1H), 1.14-1.03 (m, 2H). |
| 21I | 47 | 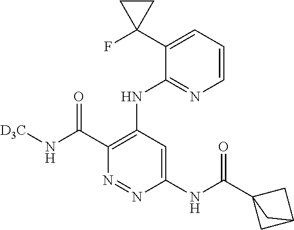 [SMILES: [2H]C([2H])([2H])NC(=O)c1nnc(NC(=O)C23CC(C2)C3)cc1Nc1ncccc1C1(F)CC1] | E, J | 399.4 | 400.4 (G) | ¹H NMR (400 MHZ, DMSO-d₆) δ 12.32 (s, 1H), 10.77 (s, 1H), 9.83 (s, 1H), 9.16 (s, 1H), 8.41 (d, J = 4.9 Hz, 1H), 7.90 (d, J = 7.5 Hz, 1H), 7.10 (dd, J = 7.3, 5.1 Hz, 1H), 2.46 (s, 1H), 2.16 (s, 6H), 1.65-1.50 (m, 2H), 1.22- 1.09 (m, 2H). |
| 21J | 48 | 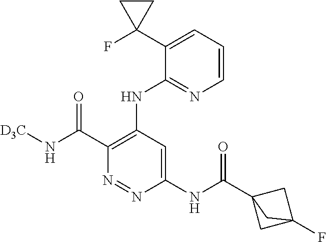 [SMILES: [2H]C([2H])([2H])NC(=O)c1nnc(NC(=O)C23CC(F)(C2)C3)cc1Nc1ncccc1C1(F)CC1] | E, J | 417.4 | 418.3 (C) | ¹H NMR (400 MHZ, DMSO-d₆) δ 12.31 (s, 1H), 11.03 (s, 1H), 9.80 (s, 1H), 9.16 (s, 1H), 8.40 (d, J = 4.9 Hz, 1H), 7.89 (d, J = 7.5 Hz, 1H), 7.09 (dd, J = 7.3, 5.1 Hz, 1H), 2.47 (d, J = 2.3 Hz, 6H), 1.65-1.51 (m, 2H), 1.22- 1.09 (m, 2H). |
| 21K | 52 | 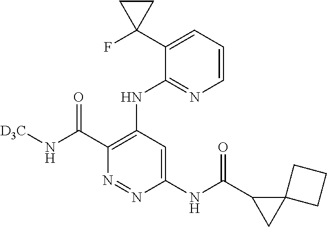 [SMILES: [2H]C([2H])([2H])NC(=O)c1nnc(NC(=O)C2CC23CCC3)cc1Nc1ncccc1C1(F)CC1] | E, J | 413.5 | 414.4 (C) | ¹H NMR (400 MHZ, DMSO-d₆) δ 12.27 (s, 1H), 11.16 (s, 1H), 9.74 (s, 1H), 9.17 (s, 1H), 8.34 (d, J = 4.9 Hz, 1H), 7.86 (d, J = 7.5 Hz, 1H), 7.05 (dd, J = 7.2, 5.1 Hz, 1H), 2.25-2.14 (m, 1H), 2.14- 1.88 (m, 6H), 1.62- 1.44 (m, 2H), 1.19-1.05 (m, 3H), 1.06-0.95 (m, 1H). |
| 21L | 55A | 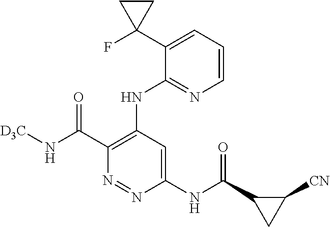 | E, J | 398.4 | 399.0 (K) | ¹H NMR (400 MHZ, DMSO-d₆) δ 12.30 (s, 1H), 11.59 (s, 1H), 9.77 (s, 1H), 9.19 (s, 1H), 8.35 (d, J = 4.8 Hz, 1H), 7.87 (d, J = 7.5 Hz, 1H), 7.07 (dd, J = 7.2, 5.1 Hz, 1H), 2.60 (dd, J = 14.5, 7.6 Hz, 1H), 2.26 (dd, J = 15.4, 8.2 Hz, 1H), 1.62-1.38 (m, 4H), 1.19-1.07 (m, 2H). |
| 21M | 57 | 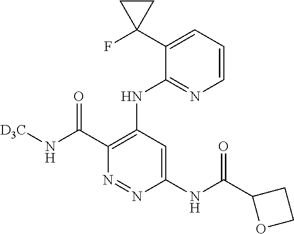 [SMILES: [2H]C([2H])([2H])NC(=O)c1nnc(NC(=O)C2CCO2)cc1Nc1ncccc1C1(F)CC1] | E, J | 389.4 | 390.3 (K) | ¹H NMR (400 MHZ, DMSO-d₆) δ 12.37 (s, 1H), 10.51 (s, 1H), 9.92 (s, 1H), 9.24 (s, 1H), 8.41 (d, J = 4.9 Hz, 1H), 7.89 (d, J = 7.5 Hz, 1H), 7.11 (dd, J = 6.8, 5.0 Hz, 1H), 5.26 (dd, J = 8.9, 6.7 Hz, 1H), 4.64 (t, J = 7.8 Hz, 2H), 3.02-2.88 (m, 1H), 2.76-2.62 (m, 1H), 1.65- 1.46 (m, 2H), 1.18- 1.10 (m, 2H). |
| 21N | 58 | 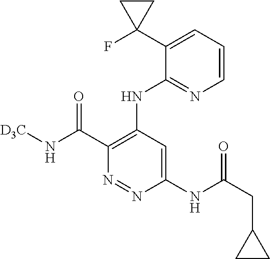 [SMILES: [2H]C([2H])([2H])NC(=O)c1nnc(NC(=O)CC2CC2)cc1Nc1ncccc1C1(F)CC1] | E, K | 387.4 | 388.5 (K) | ¹H NMR (400 MHZ, DMSO-d₆) δ 12.30 (s, 1H), 10.90 (s, 1H), 9.86 (s, 1H), 9.17 (s, 1H), 8.39 (d, J = 4.9 Hz, 1H), 7.88 (dd, J = 5.7, 3.7 Hz, 1H), 7.09 (dd, J = 7.1, 5.2 Hz, 1H), 2.35 (d, J = 7.1 Hz, 2H), 1.61-1.49 (m, 2H), 1.18-1.11 (m, 2H), 1.11- 1.00 (m, 1H), 0.52- 0.42 (m, 2H), 0.24-0.13 (m, 2H). |
| 210 | 59 | 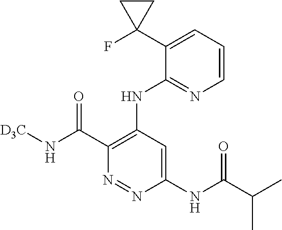 [SMILES: [2H]C([2H])([2H])NC(=O)c1nnc(NC(=O)C(C)C)cc1Nc1ncccc1C1(F)CC1] | E, J | 375.4 | 376.4 (C) | ¹H NMR (400 MHZ, DMSO-d₆) δ 12.32 (s, 1H), 10.97 (s, 1H), 9.87 (s, 1H), 9.19 (s, 1H), 8.42 (dd, J = 3.2, 1.7 Hz, 1H), 7.90 (dt, J = 7.5, 1.9 Hz, 1H), 7.11 (dd, J = 7.0, 5.1 Hz, 1H), 2.92-2.80 (m, 1H), 1.64-1.50 (m, 2H), 1.20-1.06 (m, 8H). |
| 21P | 60B | 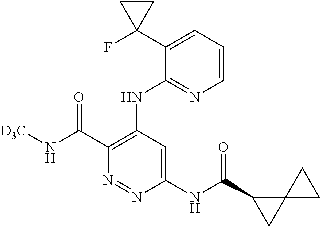 [SMILES: [2H]C([2H])([2H])NC(=O)c1nnc(NC(=O)[C@@H]2CC23CC3)cc1Nc1ncccc1C1(F)CC1] | E, J | 399.4 | 400.4 (G) | ¹H NMR (400 MHZ, DMSO-d₆) δ 12.31 (s, 1H), 11.12 (s, 1H), 9.83 (s, 1H), 9.19 (s, 1H), 8.41 (d, J = 4.9 Hz, 1H), 7.90 (d, J = 7.5 Hz, 1H), 7.10 (dd, J = 7.1, 5.2 Hz, 1H), 2.49-2.44 (m, 1H), 1.57 (dt, J = 18.2, 7.0 Hz, 2H), 1.45 (t, J = 3.7 Hz, 1H), 1.38 (dd, J = 7.4, 3.4 Hz, 1H), 1.15 (q, J = 8.4 Hz, 2H), 0.97-0.72 (m, 4H). |
| 21Q | 60A | 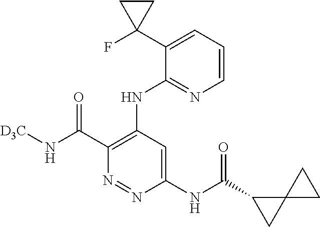 [SMILES: [2H]C([2H])([2H])NC(=O)c1nnc(NC(=O)[C@H]2CC23CC3)cc1Nc1ncccc1C1(F)CC1] | E, k | 399.4 | 400.4 (H) | ¹H NMR (400 MHZ, DMSO-d₆) δ 12.31 (s, 1H), 11.12 (s, 1H), 9.83 (s, 1H), 9.19 (s, 1H), 8.41 (d, J = 4.9 Hz, 1H), 7.90 (d, J = 7.5 Hz, 1H), 7.10 (dd, J = 7.1, 5.2 Hz, 1H), 2.49-2.44 (m, 1H), 1.57 (dt, J = 18.2, 7.0 Hz, 2H), 1.45 (t, J = 3.7 Hz, 1H), 1.38 (dd, J = 7.4, 3.4 Hz, 1H), 1.15 (q, J = 8.4 Hz, 2H), 0.97-0.72 (m, 4H). |
| 21R | 80 | 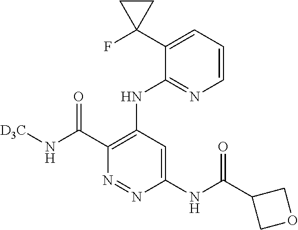 [SMILES: [2H]C([2H])([2H])NC(=O)c1nnc(NC(=O)C2COC2)cc1Nc1ncccc1C1(F)CC1] | E, K | 389.4 | 390.3 (G) | ¹H NMR (400 MHZ, DMSO-d₆) δ 12.36 (s, 1H), 11.06 (s, 1H), 9.94 (s, 1H), 9.22 (s, 1H), 8.45 (d, J = 4.9 Hz, 1H), 7.91 (d, J = 7.5 Hz, 1H), 7.12 (dd, J = 7.1, 5.3 Hz, 1H), 4.76-4.67 (m, 4H), 4.21- 4.11 (m, 1H), 1.64- 1.51 (m, 2H), 1.21-1.12 (m, 2H). |

### Example 22A: 4-((5-chloro-3-(1-fluorocyclopropyl)pyridin-2-yl)amino)-6-((1R,2R)-2-fluorocyclopropane-1-carboxamido)-N-(methyl-d₃)pyridazine-3-carboxamide (Compound 46A)

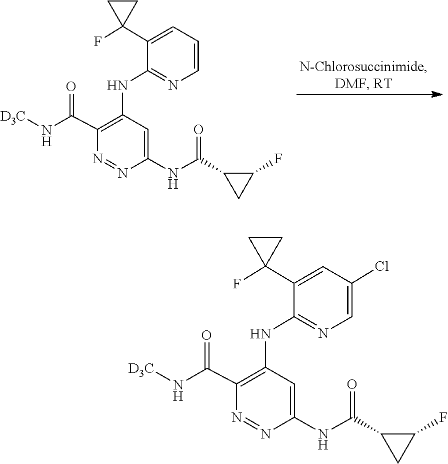

### Synthesis of 4-((5-chloro-3-(1-fluorocyclopropyl)pyridin-2-yl)amnion)-6-((1R,2R)-2-fluorocyclopropane-1-carboxamido)-N-(methyl-d₃)pyridazine-3-carboxamide

6-((1R,2R)-2-fluorocyclopropane-1-carboxamido)-4-((3-(1-fluorocyclopropyl)pyridin-2-yl)amino)-N-(methyl-d₃)pyridazine-3-carboxamide (30.0 mg, 76.6 umol), N-chlorosuccinimide (11.5 mg, 84.3 umol) and DMF (1.00 mL) were stirred at 50° C. 18 h, no reaction observed so more N-Chlorosuccinimide (11.5 mg, 84.3 umol) was added. The reaction was stirred an additional 3 h then cooled to ambient temperature and injected directly into the purification system. Purification using Büchi Pure C850 Benchtop Semi-Prep (C18 silica with 10 mM Ammonium Bicarbonate Buffer pH 10.3/MeCN 5%-95%) afforded the title compound (2.01 mg, 6.2%). LCMS Method D (m/z): [M+H]⁺=426.3, retention time=2.63 min. ¹H NMR (400 MHz, DMSO-d₆) δ 12.43 (s, 1H), 11.42 (s, 1H), 9.71 (s, 1H), 9.24 (s, 1H), 8.43 (dd, J=2.4, 1.6 Hz, 1H), 8.07-8.00 (m, 1H), 5.08-4.87 (m, 1H), 2.35-2.29 (m, 1H), 1.76-1.63 (m, 1H), 1.62-1.50 (m, 2H), 1.29-1.17 (m, 3H).

### Example 22B-22C

The following compounds were prepared in a similar fashion as Compound 46A described in Example 22A using the corresponding N-halosuccinimides:

TABLE 10

| | | | | (m/z) | |
|---|---|---|---|---|---|
| Ex. | Cmpd | | MW | [M + H]⁺ | |
| No. | No. | Structure | (g/mol) | (method) | ¹H NMR |
| 22B | 45 | 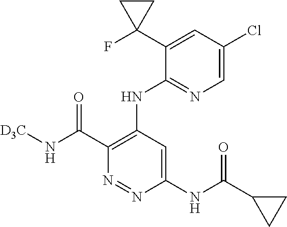 [SMILES: [2H]C([2H])([2H])NC(=O)c1nnc(NC(=O)C2CC2)cc1Nc1ncc(Cl)cc1C1(F)CC1] | 407.9 | 408.2 (D) | ¹H NMR (500 MHZ, DMSO-d₆) δ = 12.41 (s, 1H), 11.37 (s, 1H), 9.70 (s, 1H), 9.22 (s, 1H), 8.43- 8.40 (m, 1H), 8.04-8.01 (m, 1H), 2.15-2.09 (m, 1H), 1.61-1.52 (m, 2H), 1.26-1.20 (m, 2H), 0.91- 0.83 (m, 4H) |
| 22C | INT-X | 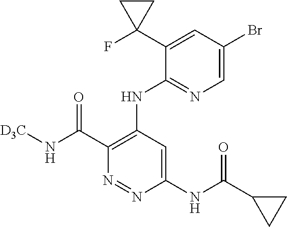 [SMILES: [2H]C([2H])([2H])NC(=O)c1nnc(NC(=O)C2CC2)cc1Nc1ncc(Br)cc1C1(F)CC1] | 452.3 | 452.2, 454.2 (B) | ND |

### Example 23: 4-((5-cyano-3-(1-fluorocyclopropyl)pyridin-2-yl)amino)-6-(cyclopropanecarboxamido)-N-(methyl-d₃)pyridazine-3-carboxamide (Compound 56)

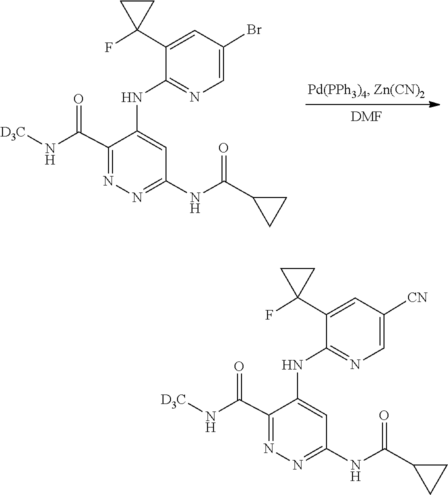

To a mixture of 4-((5-bromo-3-(1-fluorocyclopropyl)pyridin-2-yl)amino)-6-(cyclopropanecarboxamido)-N-(methyl-d₃)pyridazine-3-carboxamide (16.0 mg, 35.4 umol), tetrakis(triphenylphosphine)palladium(0) (4.17 mg, 3.54 umol) and zinc cyanide (21.2 mg, 177 umol) was added DMF (800 uL). The reaction mixture was purged with N₂ for 10 minutes and then allowed to stir at 135° C. for 6 h. The mixture was directly purified by reverse phase flash chromatography (C18 silica with 10 mM Ammonium Bicarbonate buffer/MeCN: 10%-100%) to afford 4-((5-cyano-3-(1-fluorocyclopropyl)pyridin-2-yl)amino)-6-(cyclopropanecarboxamido)-N-(methyl-d₃)pyridazine-3-carboxamide (2.01 mg, 13%). LCMS Method D (m/z): [M+H]⁺=399.3, retention time=2.07 min. 1H NMR (400 MHz, DMSO) δ=12.79 (s, 1H), 11.47 (s, 1H), 9.85 (s, 1H), 9.31 (s, 1H), 8.81 (s, 1H), 8.37-8.33 (m, 1H), 7.66-7.51 (m, 1H), 2.19-2.10 (m, 1H), 1.63-1.54 (m, 2H), 1.29-1.20 (m, 2H, grease overlap), 0.92-0.84 (m, 4H).

### Example 24: 6-(cyclopropanecarboxamido)-4-((3-(1-fluorocyclopropyl)-5-(1-methyl-1H-pyrazol-3-yl)pyridin-2-yl)amino)-N-(methyl-d3)pyridazine-3-carboxamide (Compound 81)

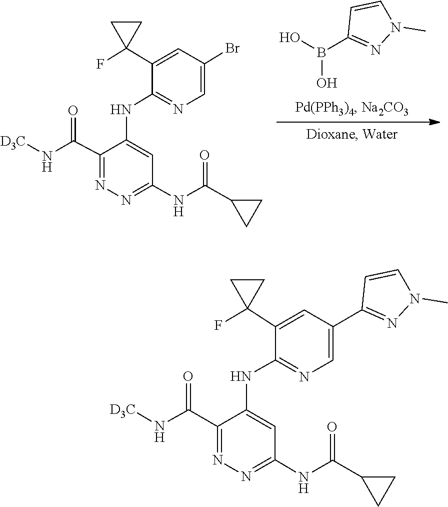

To a mixture of 4-((5-bromo-3-(1-fluorocyclopropyl)pyridin-2-yl)amino)-6-(cyclopropanecarboxamido)-N-(methyl-d₃)pyridazine-3-carboxamide (15.0 mg, 33.2 umol), (1-methyl-1H-pyrazol-3-yl)boronic acid (8.79 mg, 66.3 umol), Na₂CO₃ (10.8 mg, 99.5 umol) and tetrakis(triphenylphosphine)palladium(0) (3.87 mg, 3.32 umol) was added dioxane (750 uL) and water (75.0 uL) in a pressure vial fitted with a stir bar. The reaction mixture was purged with N₂ for 10 minutes and then allowed to stir at 110° C. for 1 h. The mixture was directly purified by reverse phase flash chromatography (C18 silica with 10 mM Ammonium Bicarbonate buffer/ACN: 10%-100%) to afford 6-(cyclopropanecarboxamido)-4-((3-(1-fluorocyclopropyl)-5-(1-methyl-1H-pyrazol-3-yl)pyridin-2-yl)amino)-N-(methyl-d₃)pyridazine-3-carboxamide (2.42 mg, 16%). LCMS Method D (m/z): [M+H]⁺=454.3, retention time=2.09 min. ¹H NMR (400 MHz, DMSO-d₆) δ=12.37 (s, 1H), 11.34 (s, 1H), 9.86 (s, 1H), 9.21 (s, 1H), 8.79 (s, 1H), 8.18 (s, 1H), 7.77 (d, J=2.2, 1H), 6.87 (d, J=2.2, 1H), 3.90 (s, 3H), 2.17-2.10 (m, 1H), 1.65-1.55 (m, 2H), 1.29-1.17 (m, 2H), 0.93-0.82 (m, 4H).

### Example 25A: 4-((3-(1,1-difluoroethyl)-5-fluoropyridin-2-yl)amino)-6-(1-fluorocyclopropane-1-carboxamido)-N-(methyl-d₃)pyridazine-3-carboxamide (Compound 70)

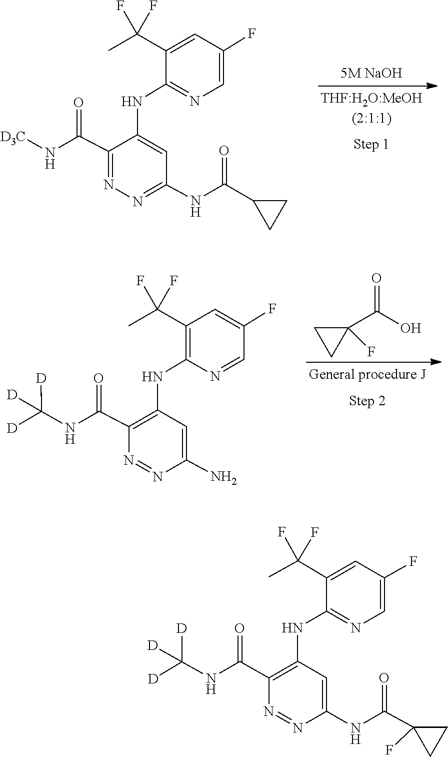 [SMILES: [2H]C([2H])([2H])NC(=O)c1nnc(NC(=O)C2CC2)cc1Nc1ncc(F)cc1C(C)(F)F ; [2H]C([2H])([2H])NC(=O)c1nnc(N)cc1Nc1ncc(F)cc1C(C)(F)F ; O=C(O)C1(F)CC1 ; [2H]C([2H])([2H])NC(=O)c1nnc(NC(=O)C2(F)CC2)cc1Nc1ncc(F)cc1C(C)(F)F]

### Step 1: Synthesis of 2-6-amino-4-((3-(1,1-difluoroethyl)-5-fluoropyridin-2-yl)amino)-N-(methyl-d₃)pyridazine-3-carboxamide

To a solution of 6-(cyclopropanecarboxamido)-4-((3-(1,1-difluoroethyl)-5-fluoropyridin-2-yl)amino)-N-(methyl-d₃)pyridazine-3-carboxamide (100 mg, 0.234 mmol) in THF (851 uL) MeOH (426 uL) and Water (426 uL) was added aqueous 5 M NaOH (468 uL, 2.34 mmol). The mixture was allowed to stir at room temperature for 96 h. The organics were removed under vacuum and the mixture was diluted with 10% MeOH/DCM solution and was transferred into a separatory funnel. The organic layer was washed with water (2×), dried over anh. Na₂SO₄, filtered, and concentrated under vacuum to afford crude 6-amino-4-((3-(1,1-difluoroethyl)-5-fluoropyridin-2-yl)amino)-N-(methyl-d₃)pyridazine-3-carboxamide (70.0 mg, 91%) as an off-white solid. Used as-is without further purification. LCMS Method R (m/z): [M+H]⁺=330.3, retention time=0.92 min.

### Step 2: Synthesis of 4-((3-(1,1-difluoroethyl)-5-fluoropyridin-2-yl)amino)-6-(1-fluorocyclopropane-1-carboxamido)-N-(methyl-d₃)pyridazine-3-carboxamide

The synthesis was carried out according to General Procedure J. In a vial, to a mixture of 1-fluorocyclopropanecarboxylic acid (117 uL, 1.21 mmol), NMI (245 uL, 3.04 mmol) and DMF (525 uL) was added TCFH (878 mg, 3.04 mmol). The mixture was stirred at room temperature for 10 minutes.

In a second vial, 6-amino-4-((3-(1,1-difluoroethyl)-5-fluoropyridin-2-yl)amino)-N-(methyl-d₃)pyridazine-3-carboxamide (100 mg, 304 umol) was dissolved in DMF (457 uL) and NMI (611 uL, 7.59 mmol) was added. Then the activated acid mixture (first vial) was added to the second vial.

After 40 minutes, LCMS analysis indicated the reaction had completed. The product was precipitated by adding water (8 mL) and washed with water (2×8 mL), then purified by C18 reversed phased column chromatography (10-100% MeCN in 10 mM AmB (aq.)) to give the title compound (85.0 mg, 67%). LCMS Method L (m/z): [M+H]⁺=416.2, retention time=2.99 min. ¹H NMR (400 MHz, DMSO-d₆) δ 12.06 (s, 1H), 10.87 (s, 1H), 9.30 (s, 1H), 9.28 (s, 1H), 8.51 (d, J=2.8 Hz, 1H), 7.98 (dd, J=8.9, 2.9 Hz, 1H), 2.09 (t, J=19.3 Hz, 3H), 1.54-1.43 (m, 2H), 1.43-1.33 (m, 2H). ¹⁹F NMR (376 MHz, DMSO-d₆) δ−85.72 (q, J=19.3 Hz, 2F), −134.22 (d, J=8.9 Hz, 1F), −195.32 (s, 1F).

### Example 25B-25F

The following examples were prepared in a similar manner to Compound 70 described in Example 25A using the corresponding commercially available carboxylic acids:

TABLE 11

| | | | | | (m/z) | |
|---|---|---|---|---|---|---|
| Ex. | Cmpd | | General | MW | [M + H]⁺ | |
| No. | No. | Structure | Proc. | (g/mol) | (method) | ¹H NMR |
| 25B | 86 | 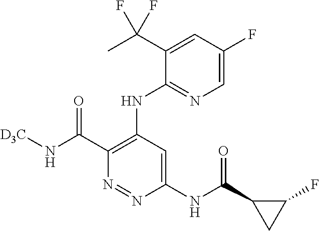 [SMILES: [2H]C([2H])([2H])NC(=O)c1nnc(NC(=O)[C@@H]2C[C@H]2F)cc1Nc1ncc(F)cc1C(C)(F)F] | J | 415.4 | 416.1 (K) | ¹H NMR (400 MHZ, DMSO-d₆) δ 12.03 (s, 1H), 11.52 (s, 1H), 9.31 (s, 1H), 9.24 (s, 1H), 8.48 (d, J = 2.6 Hz, 1H), 7.96 (dd, J = 8.9, 2.8 Hz, 1H), 4.91 (d, J = 65.3 Hz, 1H), 2.76- 2.57 (m, 1H), 2.08 (t, J = 19.3 Hz, 3H), 1.67- 1.49 (m, 1H), 1.27 (dq, J = 13.0, 6.4 Hz, 1H). |
| 25C | 67A | 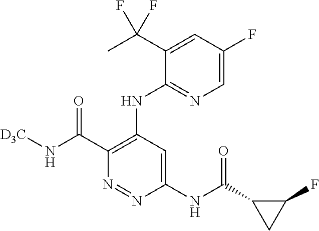 [SMILES: [2H]C([2H])([2H])NC(=O)c1nnc(NC(=O)[C@H]2C[C@@H]2F)cc1Nc1ncc(F)cc1C(C)(F)F] | J | 415.4 | 416.1 (K) | ¹H NMR (400 MHZ, DMSO-d₆) δ 12.02 (s, 1H), 11.52 (s, 1H), 9.31 (s, 1H), 9.23 (s, 1H), 8.48 (d, J = 2.8 Hz, 1H), 7.96 (dd, J = 8.9, 2.8 Hz, 1H), 5.02-4.80 (m, 1H), 2.66 (ddd, J = 17.9, 10.4, 7.2 Hz, 1H), 2.08 (t, J = 19.3 Hz, 3H), 1.57 (dddd, J = 13.7, 10.0, 6.4, 3.2 Hz, 1H), 1.27 (tt, J = 10.8, 5.4 Hz, 1H). |
| 25D | 66 | 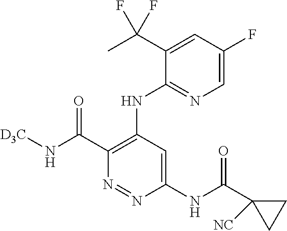 [SMILES: [2H]C([2H])([2H])NC(=O)c1nnc(NC(=O)C2(C#N)CC2)cc1Nc1ncc(F)cc1C(C)(F)F] | J | 422.4 | 423.2 (H) | ¹H NMR (400 MHZ, DMSO-d₆) δ 11.99 (s, 1H), 10.99 (s, 1H), 9.22 (s, 1H), 9.12 (s, 1H), 8.51 (d, J = 2.6 Hz, 1H), 7.96 (dd, J = 8.9, 2.7 Hz, 1H), 2.09 (t, J = 19.3 Hz, 3H), 1.76- 1.59 (m, 4H). |
| 25E | 71A | 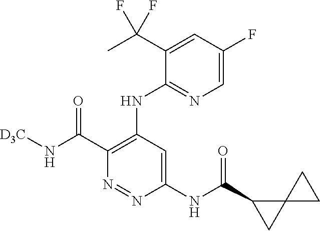 [SMILES: [2H]C([2H])([2H])NC(=O)c1nnc(NC(=O)[C@@H]2CC23CC3)cc1Nc1ncc(F)cc1C(C)(F)F] | J | 423.4 | 424.2 (C) | ¹H NMR (400 MHZ, DMSO-d₆) δ 11.99 (s, 1H), 11.16 (s, 1H), 9.33 (s, 1H), 9.20 (s, 1H), 8.53 (d, J = 2.9 Hz, 1H), 7.97 (dd, J = 8.9, 2.9 Hz, 1H), 2.46 (dd, J = 7.4, 4.3 Hz, 1H), 2.08 (t, J = 19.3 Hz, 3H), 1.44- 1.40 (m, 1H), 1.40- 1.34 (m, 1H), 0.92- 0.77 (m, 4H). |
| 25F | 98A | 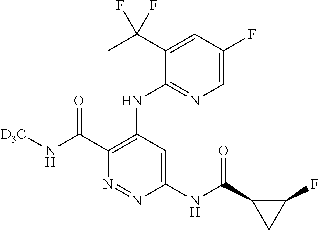 [SMILES: [2H]C([2H])([2H])NC(=O)c1nnc(NC(=O)[C@@H]2C[C@@H]2F)cc1Nc1ncc(F)cc1C(C)(F)F] | J | 415.4 | 416.3 (L) | ¹H NMR (400 MHZ, DMSO-d₆) δ 12.01 (s, 1H), 11.41 (s, 1H), 9.33 (d, J = 2.6 Hz, 1H), 9.23 (s, 1H), 8.53 (s, 1H), 7.97 (dt, J = 4.6, 2.5 Hz, 1H), 5.08-4.86 (m, 1H), 2.36-2.27 (m, 1H), 2.15-2.03 (m, 4H), 1.73-1.58 (m, 1H). |

### Example 26A: 6-(cyclopropanecarboxamido)-4-((3-(1-fluorospiro[2.2]pentan-1-yl)pyridin-2-yl)amino)-N-(methyl-d3)pyridazine-3-carboxamide (Compound 83)

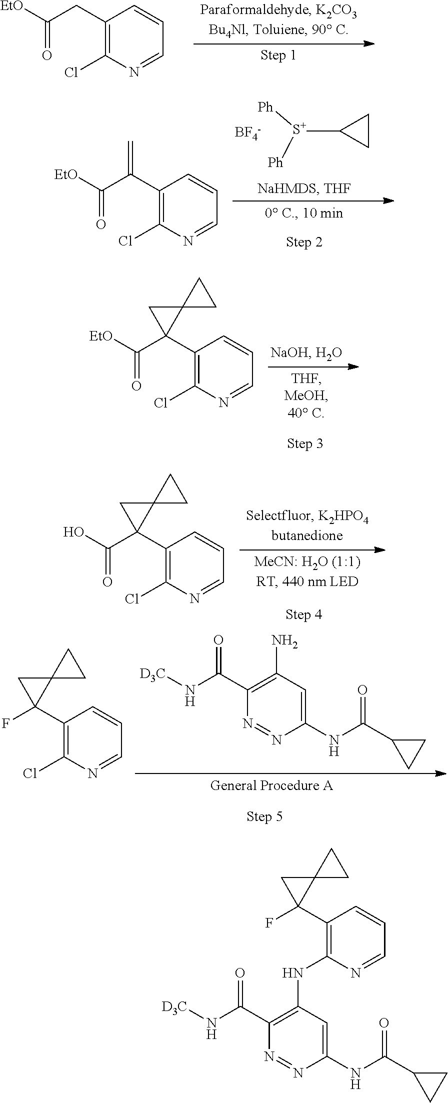

### Step 1: Synthesis of ethyl 2-(2-chloropyridin-3-yl)acrylate

Dry toluene (98.2 mL) was added to a mixture of ethyl 2-(2-chloropyridin-3-yl)acetate (1.00 g, 4.91 mmol), K₂CO₃ (2.22 g, 15.7 mmol), tetrabutylammonium iodide (92.5 mg, 0.245 mmol) and paraformaldehyde (3.49 g, 110 mmol). The mixture was stirred at 90° C. 18 h. The mixture was then cooled to room temperature, filtered, and the filtrate was concentrated under reduced pressure and adsorbed onto silica gel. Purified by column chromatography on silica (0-100% EtOAc) to obtain ethyl 2-(2-chloropyridin-3-yl)acrylate (800 mg, 77%) as a colourless oil. LCMS Method B (m/z): [M+H]⁺=212.2, retention time=0.92 min.

### Step 2: Synthesis of ethyl 1-(2-chloropyridin-3-yl)spiro[2.2]pentane-1-carboxylate

Ethyl 2-(2-chloropyridin-3-yl)acrylate (700 mg, 3.31 mmol) and cyclopropyldiphenylsulfonium tetrafluoroborate (1.59 g, 4.96 mmol) were suspended in dry THF (16.5 mL) and flask was purged with nitrogen and sonicated 2 mins. The mixture was cooled in an ice bath with stirring under nitrogen and NaHMDS (4.96 mL, 4.96 mmol) 1 M in THF was added dropwise. The reaction was complete within 10 mins after base addition and quenched with saturated NH₄Cl. The mixture was extracted with EtOAc, the combined organics were washed with brine, dried over anh. Na₂SO₄ filtered and concentrated under vacuum. The residue was taken up in DCM and filtered through 30 g of silica gel using Heptanes as eluent, this removed most of the diphenyl sulfide byproduct. The column was flushed with 50/50 Heptanes/DCM to remove the desired product. This filtrate was concentrated under vacuum to afford ethyl 1-(2-chloropyridin-3-yl)spiro[2.2]pentane-1-carboxylate (720 mg, 86%) as a pale yellow oil which was used as is in the next step without further purification. LCMS Method B (m/z): [M+H]⁺=252.2, retention time=1.09 min.

### Step 3: Synthesis of 1-(2-chloropyridin-3-yl)spiro[2.2]pentane-1-carboxylic acid

To a solution of ethyl 1-(2-chloropyridin-3-yl)spiro[2.2]pentane-1-carboxylate (720 mg, 2.86 mmol) in THF (9.53 mL), water (4.77 mL), and MeOH (4.77 mL) was added 5 M aq. NaOH (5.72 mL, 28.6 mmol). The reaction mixture was stirred at 40° C. for 16 h. 1 M aq. HCl was added and brought pH~1-3 and the aqueous portion was extracted with DCM:MeOH (4:1). The combined organic layers were dried over Na₂SO₄, filtered and concentrated under reduced pressure to afford 1-(2-chloropyridin-3-yl)spiro[2.2]pentane-1-carboxylic acid (630 mg, 98%) as a white solid. The residue was used as it is in the next step without any further purification. LCMS Method F (m/z): [M+H]⁺=224.1, retention time=0.40 min.

### Step 4: Synthesis of 2-chloro-3-(1-fluorospiro[2.2]pentan-1-yl)pyridine

To a mixture of 1-(2-chloropyridin-3-yl)spiro[2.2]pentane-1-carboxylic acid (630 mg, 2.82 mmol), Selectfluor® (3.19 g, 9.01 mmol), and K₂HPO₄ (1.05 g, 5.92 mmol) was added water (11.3 mL)/MeCN (11.3 mL). The reaction mixture was degassed with nitrogen for 5 min. To this mixture was then added 2,3-butanedione (25.0 mg, 0.282 mmol) and stirred for 1 h under the irradiation of 440 nm LED. The reaction mixture was extracted with DCM and washed with sat. aq. NaHCO₃. The combined organic layers were dried over Na₂SO₄, filtered and concentrated under reduced pressure to afford 2-chloro-3-(1-fluorospiro[2.2]pentan-1-yl)pyridine (400 mg, 72%) as a pale yellow oil. This residue was used as it is in the next step without any further purification. LCMS Method F (m/z): [M+H]⁺=no ionization, retention time=1.13 min.

### Step 5: Synthesis of 6-(cyclopropanecarboxamido)-4-((3-(1-fluorospiro[2.2]pentan-1-yl)pyridin-2-yl)amino)-N-(methyl-d₃)pyridazine-3-carboxamide

The title compound was prepared according to General Procedure A.

To a degassed mixture of 2-chloro-3-(1-fluorospiro[2.2]pentan-1-yl)pyridine (99.5 mg, 0.504 mmol), 4-amino-6-(cyclopropanecarboxamido)-N-(methyl-d₃)pyridazine-3-carboxamide (100 mg, 0.420 mmol), dcpf (102 mg, 0.168 mmol), and K₃PO₄ (364 mg, 1.68 mmol) in dioxane (4.20 mL) was added Pd₂(dba)₃ (76.9 mg, 0.0839 mmol). Nitrogen was immediately bubbled to the mixture for 2 min and the reaction was stirred at 120° C. for 1 h. The mixture was cooled, diluted with 50% EtOAc/MeOH and filtered on Celite®. The Celite® cake was rinsed with EtOAc and the combined organic was concentrated under reduced pressure. The residue was purified by normal phase chromatography (0-20% iPrOH/DCM) to afford racemic 6-(cyclopropanecarboxamido)-4-((3-(1-fluorospiro[2.2]pentan-1-yl)pyridin-2-yl)amino)-N-(methyl-d₃)pyridazine-3-carboxamide (57.5 mg, 34%). LCMS Method D (m/z): [M+H]⁺=400.3, retention time=2.27 min. ¹H NMR (400 MHz, DMSO-d₆): δ 11.79 (s, 1H), 11.33 (s, 1H), 9.63 (s, 1H), 9.19 (s, 1H), 8.42-8.35 (m, 1H), 7.94 (dt, J=7.5, 1.8 Hz, 1H), 7.12 (dd, J=7.1, 5.3 Hz, 1H), 2.15-2.08 (m, 1H), 1.80 (dd, J=13.8, 6.7 Hz, 1H), 1.72-1.67 (m, 1H), 1.55-1.48 (m, 1H), 1.17-1.11 (m, 1H), 0.89-0.84 (m, 4H), 0.84-0.75 (m, 2H).

### Example 26B

The following examples were prepared in a manner similar to Compound 83 described in Example 26A:

TABLE 12

| | | | (m/z) | |
|---|---|---|---|---|
| Cmpd | | MW | [M + H]⁺ | |
| No. | Structure | (g/mol) | (method) | ¹H NMR |
| 82 | 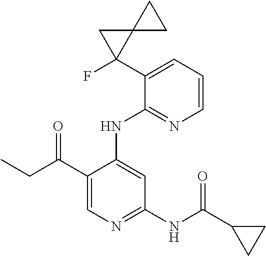 [SMILES: CCC(=O)c1cnc(NC(=O)C2CC2)cc1Nc1ncccc1C1(F)CC12CC2] | 394.44 | 395.3 (H) | ¹H NMR (400 MHZ, DMSO-d₆) δ 11.65 (s, 1H), 10.89 (s, 1H), 9.26- 9.20 (m, 1H), 8.95-8.90 (m, 1H), 8.45-8.34 (m, 1H), 7.99-7.92 (m, 1H), 7.16-7.11 (m, 1H), 3.23- 3.06 (m, 2H), 2.08-2.00 (m, 1H), 1.85-1.76 (m, 1H), 1.73-1.65 (m, 1H), 1.29-1.22 (m, 1H), 1.18- 1.06 (m, 4H), 0.85-0.73 (m, 6H). |

### Example 27: N-(4-((3-(1-fluorocyclopropyl)pyridin-2-yl)amino)-5-(propanoyl-3,3,3-d3)pyridin-2-yl)cyclopropanecarboxamide: (Compound 37)

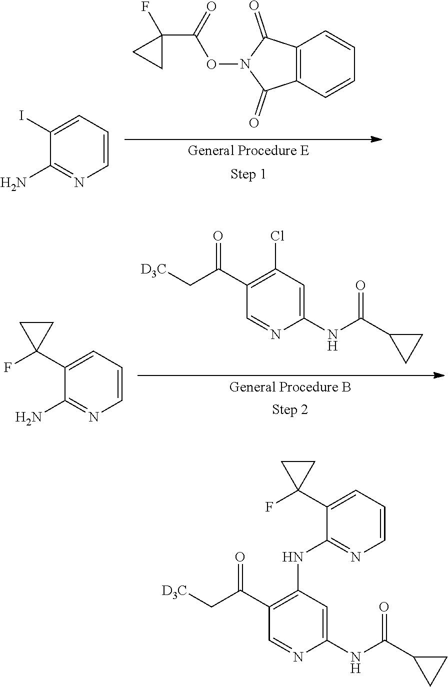 [SMILES: Nc1ncccc1I ; O=C1c2ccccc2C(=O)N1OC(=O)C1(F)CC1 ; Nc1ncccc1C1(F)CC1 ; [2H]C([2H])([2H])CC(=O)c1cnc(NC(=O)C2CC2)cc1Cl ; [2H]C([2H])([2H])CC(=O)c1cnc(NC(=O)C2CC2)cc1Nc1ncccc1C1(F)CC1]

### Step 1: 3-(1-fluorocyclopropyl)pyridin-2-amine

### Synthesized According to General Procedure E

2-Amino-3-iodopyridine (330 mg, 1.47 mmol), 1,3-dioxoisoindolin-2-yl 1-fluorocyclopropane-1-carboxylate (495 mg, 1.99 mmol), zinc (744 mg, 11.4 mmol) and NiCl₂bpy (74.3 mg, 257 umol) were added to a microwave vial and placed under a stream of nitrogen for 5 minutes. Then, dry DMA (11.0 mL) was added, followed immediately by chlorotrimethylsilane (530 uL, 4.09 mmol), at 0° C. The mixture was stirred at 0° C. for 1 h. The mixture was diluted with EtOAc and flushed through a silica plug with 8:2 EtOAc:MeOH (150 mL). The solvents were evaporated then the residue was taken up in EtOAc (100 mL) and washed with Sat. aq. NaHCO₃ solution (20 mL). The aqueous layer was extracted with EtOAc (3×20 mL). The combined organic layers were then washed with water (2×20 mL) then brine (1×20 mL), then dried over Na₂SO₄, filtered and concentrated under reduced pressure. The residue was purified by C18 reversed phase chromatography (10-40% MeCN in 10 mM AmB(aq.). The combined fractions were concentrated under reduced pressure to remove organic solvents, then extracted into EtOAc and concentrated to give the title compound (28.0 mg, 9.3%) as a solution in EtOAc. LCMS Method B (m/z): [M+H]⁺=153.1, retention time=0.83 min. ¹H NMR (500 MHz, CDCl3) δ 8.10-8.00 (m, 1H), 7.47 (dt, J=7.3, 2.0 Hz, 1H), 6.66 (ddd, J=7.2, 5.2, 1.2 Hz, 1H), 5.18 (br s, 2H), 1.49-1.40 (m, 2H), 1.04-0.96 (m, 2H)¹⁹F NMR (471 MHz, CDCl3) δ−174.03.

### Step 2: Synthesis of N-(4-((3-(1-fluorocyclopropyl)pyridin-2-yl)amino)-5-(propanoyl-3,3,3-d₃)pyridin-2-yl)cyclopropanecarboxamide

The synthesis was carried out according to General Procedure B, with modifications. To a degassed mixture of N-(4-chloro-5-(propanoyl-3,3,3-d₃)pyridin-2-yl)cyclopropanecarboxamide (100 mg, 391 umol), 3-(1-fluorocyclopropyl)pyridin-2-amine (95.2 mg, 626 umol) and cesium carbonate (334 mg, 1.02 mmol) in dioxane (1.57 mL) was added [1,1-Bis(di-cyclohexylphosphino)ferrocene]dichloropalladium(II) (66.9 mg, 86.4 umol). The reaction mixture was sparged with N₂ for 5 minutes, and then sealed and heated at 80° C. for 2 h. The mixture was then flushed through a silica plug using EtOAc (100 mL), concentrated and purified by reverse phase flash chromatography (C18 silica with 10 mM Ammonium Bicarbonate buffer/ACN: 10% -55%) to give the title compound (25.0 mg, 17%). LCMS Method L (m/z): [M+H]⁺=372.3, retention time=2.86 min. ¹H NMR (500 MHz, DMSO-d₆) δ 12.17 (s, 1H), 10.89 (s, 1H), 9.55 (s, 1H), 8.94 (s, 1H), 8.38 (dt, J=4.8, 1.6 Hz, 1H), 7.88 (dt, J=7.5, 1.9 Hz, 1H), 7.09 (dd, J=7.2, 5.1 Hz, 1H), 3.12 (s, 2H), 2.13-1.99 (m, 1H), 1.64-1.52 (m, 2H), 1.21-1.10 (m, 1H), 0.96-0.69 (m, 4H). ¹⁹F NMR (471 MHz, DMSO-d₆) δ−170.33.

### Example 28: 6-(cyclopropanecarboxamido)-4-((5-fluoro-3-(1-fluorocyclopropyl)pyridin-2-yl)amino)-N-(methyl-d3)nicotinamide (Compound 79)

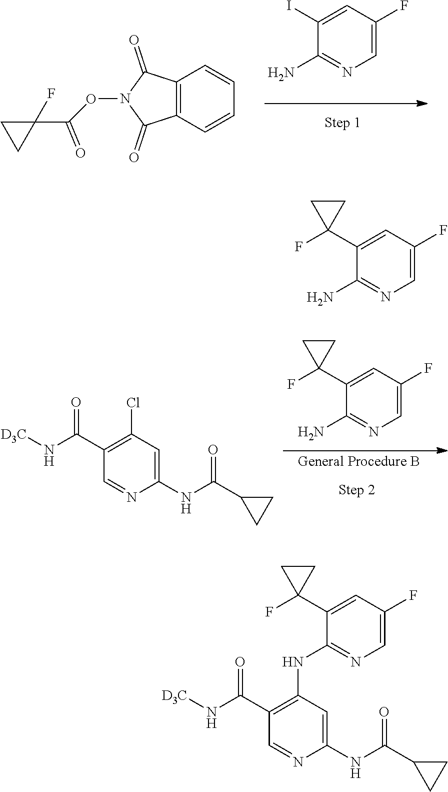

### Step 1: 2-chloro-5-fluoro-3-(1-fluorocyclopropyl)pyridine

To a mixture of 1-(2-chloro-5-fluoropyridin-3-yl)spiro[2.2]pentane-1-carboxylic acid (0.845 g, 3.50 mmol), Selectfluor® (4.71 g, 13.3 mmol) and K₂HPO₄ (1.58 g, 8.92 mmol) was added MeCN (26.2 mL)/Water (8.74 mL). The reaction mixture was degassed with nitrogen for 5 min. To this mixture was then added 2,3-Butanedione (80.2 uL, 0.894 mmol) and stirred for 1 h under the irradiation of 440 nm LED light. The reaction mixture was extracted with EtOAc three times, concentrated under reduced pressure, and the crude was resubmitted to the above conditions. After two workups and resubmissions, 90% conversion to the desired product was observed. The final reaction mixture was extracted with EtOAc three times, washed with sat. NaHCO₃(aq.), brine, and dried over anh. Na₂SO₄. The organics were filtered and concentrated under vacuum to afford 2-chloro-5-fluoro-3-(1-fluorocyclopropyl)pyridine (660 mg, 88%) as a beige oil. LCMS Method R (m/z): [M+H]⁺=no ionization, retention time=1.15 min. ¹H NMR (400 MHz, CDCl3) δ 8.27 (s, 1H), 7.56 (d, J=7.6 Hz, 1H), 1.57-1.46 (m, 2H), 1.14-1.05 (m, 2H). J

### Step 2: Synthesis of 6-(cyclopropanecarboxamido)-4-((5-fluoro-3-(1-fluorocyclopropyl)pyridin-2-yl)amino)-N-(methyl-d₃)nicotinamide

The synthesis was carried out according to General Procedure A. 2-chloro-5-fluoro-3-(1-fluorocyclopropyl)pyridine (19.2 mg, 101 umol), 4-amino-6-(cyclopropanecarboxamido)-N-(methyl-d₃)nicotinamide (30.1 mg, 127 umol), Pd₂(dba)₃ (19.9 mg, 21.3 umol), dcpf (25.8 mg, 42.6 umol), K₃PO₄ (92.2 mg, 426 umol) and dioxane (761 uL) were added to a microwave vial fitted with a stir bar and nitrogen was bubbled through the mixture for 5 mins. The tube was sealed and heated at 120° C. for 5 h in an oil bath. The crude reaction mixture was adsorbed directly onto silica gel and purified by normal phase flash column chromatography (0-20% MeOH in DCM). The fractions containing product were concentrated and then purified by C18 reversed phase column chromatography (10-50% MeCN in 10 mM AmB) to afford the title compound (22.1 mg, 44%). LCMS Method L (m/z): [M+H]⁺=391.2, retention time=2.50 min. ¹H NMR (400 MHz, DMSO-d₆) δ 11.78 (s, 1H), 10.74 (s, 1H), 9.28 (s, 1H), 8.62 (s, 1H), 8.56 (s, 1H), 8.34 (dd, J=2.7, 1.7 Hz, 1H), 7.87 (ddd, J=8.7, 2.6, 2.0 Hz, 1H), 2.01 (tt, J=7.2, 5.4 Hz, 1H), 1.61-1.48 (m, 2H), 1.22-1.13 (m, 2H), 0.86-0.75 (m, 4H).

### Example 29: 6-(cyclopropanecarboxamido)-4-((3-(cyclopropyldifluoromethyl)-5-fluoropyridin-2-yl)amino)-N-(methyl-d3)pyridazine-3-carboxamide (Compound 78)

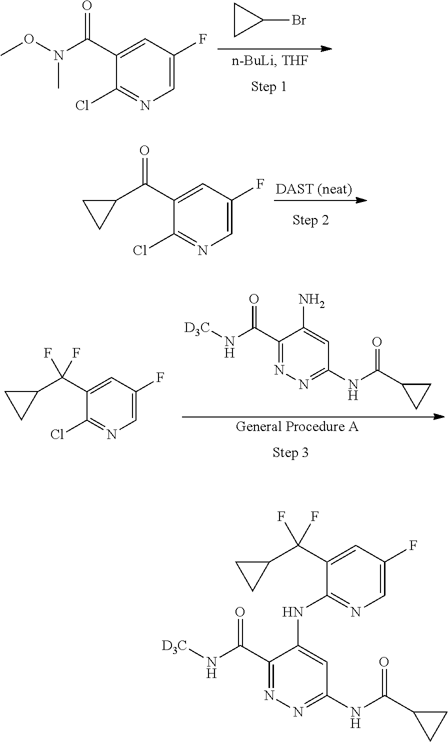

### Step 1: (2-chloro-5-fluoropyridin-3-yl)(cyclopropyl)methanone

To a solution of bromocyclopropane (449 uL, 5.49 mmol) in THF (5.48 mL), under N₂ at −78° C., was added n-Butyllithium (2.05 mL, 5.12 mmol) (1.6 M in Hexanes) dropwise and the reaction was stirred for 1 h at −78° C. Then a solution of 2-chloro-5-fluoro-N-methoxy-N-methylnicotinamide (800 mg, 3.66 mmol) in THF (3.66 mL) (also cooled to −78° C.) was added quickly, and the cooling bath was removed. Stirring was continued for 1 h then the reaction mixture was poured into sat. NH₄Cl (aq.) (60 mL), extracted with ethyl acetate (3×35 mL), and the organic layers were dried over brine, Na₂SO₄ and concentrated in vacuo. Purified by normal phase column chromatography (0-4% EtOAc in DCM) to give the title compound (394 mg, 32%) as a light pink oil LCMS Method II (m/z): [M+H]⁺=no ionization, retention time=1.31 min. ¹H NMR (400 MHz, CDCl3) δ 8.33 (d, J=2.9 Hz, 1H), 7.61 (dd, J=8.1, 3.0 Hz, 1H), 1.94-1.86 (m, 1H), 0.85-0.79 (m, 2H), 0.75-0.68 (m, 2H).

### Step 2: Synthesis of 2-chloro-3-(cyclopropyldifluoromethyl)-5-fluoropyridine

To 2-chloro-3-(cyclopropyldifluoromethyl)-5-fluoropyridine (100 mg, 295 umol), in a dry flask under N₂, was added DAST (614 uL, 4.72 mmol). Two reaction mixtures were made in this manner. The two reaction vessels were sealed and stirred at 75° C. for 16 h, at which time, LCMS analysis indicated 57% conversion.

The two reaction mixtures were combined, diluted with DCM and slowly added into Sat. NaHCO₃(aq.) (~100 mL) and shaken until no gas evolution was observed. The mixture was extracted with DCM (3×30 mL), dried over anh. Na₂SO₄ and evaporated to near-dryness to give a brown liquid, which was purified by C18 reversed phased column chromatography (35-62% MeCN in 10 mM AmB). The fractions containing desired product were added to a mixture of water (40 mL) and DCM (40 mL) and the aqueous layer was further extracted with DCM (2×40 mL). Combined organic layers were dried over Na₂SO₄ and carefully concentrated at 20° C. due to product volatility to give the title compound (43.5 mg, 33%) as a light yellow solution in MeCN/DCM. LCMS Method II (m/z): [M+H]⁺=no ionization, retention time=1.31 min. ¹H NMR (400 MHz, CDCl3) δ 8.33 (d, J=2.9 Hz, 1H), 7.61 (dd, J=8.1, 3.0 Hz, 1H), 1.94-1.86 (m, 1H), 0.85-0.79 (m, 2H), 0.75-0.68 (m, 2H).

### Step 3: Synthesis of 6-(cyclopropanecarboxamido)-4-((3-(cyclopropyldifluoromethyl)-5-fluoropyridin-2-yl)amino)-N-(methyl-d₃)pyridazine-3-carboxamide

The synthesis was carried out according to General Procedure A. 4-amino-6-(cyclopropanecarboxamido)-N-(methyl-d₃)pyridazine-3-carboxamide (49.3 mg, 207 umol), XantPhos (48.8 mg, 82.7 umol), K₂CO₃ (72.9 mg, 517 umol), 2-chloro-3-(cyclopropyldifluoromethyl)-5-fluoropyridine (43.5 mg, 196 umol) and dioxane (1.00 mL) were charged in a dry flask. The mixture was sparged with N₂ for 5 minutes, and then Pd₂(dba)₃ (37.9 mg, 41.3 umol) was added. The reaction mixture was sparged with N₂ for another 30 seconds, and then was sealed and heated to 80° C. for 18 h. The crude reaction mixture was loaded directly onto silica gel and purified repeatedly by column chromatography (Normal phase: 0-20% MeOH in DCM, followed by reversed phase: 10-100% MeCN in 10 mM AmB, followed again by normal phase: 0-100% EtOAc in Heptanes, and finally by reversed phase: 10-100% MeCN in 10 mM AmB) to give 6-(cyclopropanecarboxamido)-4-((3-(cyclopropyldifluoromethyl)-5-fluoropyridin-2-yl)amino)-N-(methyl-d₃)pyridazine-3-carboxamide (5.00 mg, 6.9%). LCMS Method L (m/z): [M+H]⁺=424.2, retention time=2.96 min. ¹H NMR (400 MHz, DMSO-d₆) δ 11.99 (s, 1H), 11.36 (s, 1H), 9.27 (s, 1H), 9.19 (s, 1H), 8.51 (d, J=2.8 Hz, 1H), 8.02 (dd, J=8.9, 2.9 Hz, 1H), 2.16-2.05 (m, 1H), 1.98-1.82 (m, 1H), 0.92-0.80 (m, 4H), 0.80-0.67 (m, 4H).

### Example 30A: 6-(cyclopropanecarboxamido)-4-((3-(7-fluorodispiro[2.0.24.13]heptan-7-yl)pyridin-2-yl)amino)-N-(methyl-d3)pyridazine-3-carboxamide (Compound 99)

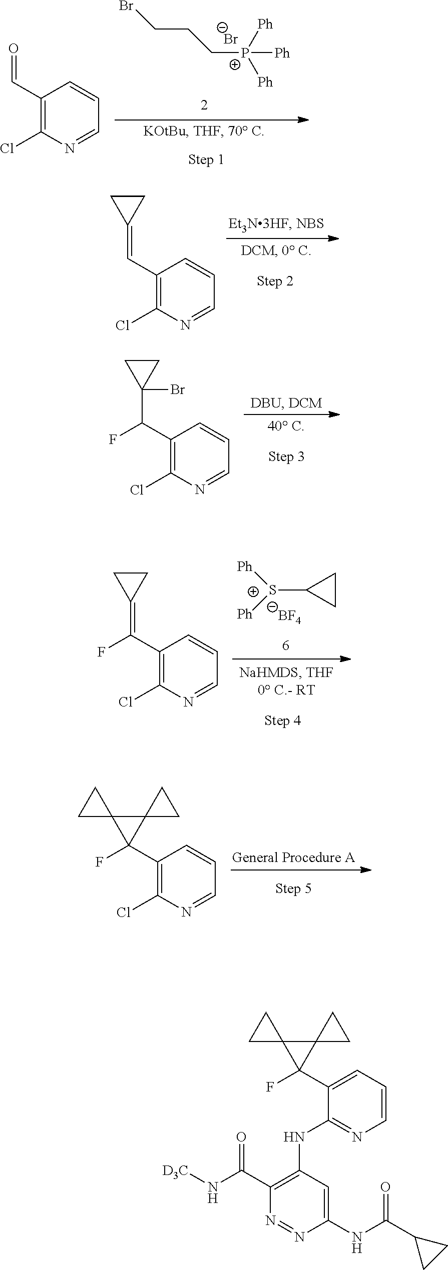

### Step 1: Synthesis of 2-chloro-3-(cyclopropylidenemethyl)pyridine

(3-bromo-propyl)-triphenyl-phosphonium bromide (49.7 g, 104 mmol) and potassium tert-butoxide (23.8 g, 208 mmol) were suspended in THF (300 mL) and nitrogen was bubbled through the mixture for 5 ins. The mixture was stirred at 70° C. for 2 h under nitrogen then was cooled to ambient temperature. 2-chloro-3-pyridinecarboxaldehyde (10.0 g, 69.2 mmol) and more potassium tert-butoxide (7.93 g, 69.2 mmol) were then added and the reaction was stirred at 75° C. for 18 h, cooled to room temperature and filtered through ca. 150 g of Celite® using EtOAc as eluent. The filtrate was adsorbed onto silica gel and purified by normal phase flash chromatography (SiO₂ with Heptanes/EtOAc: 0%-25%), the fractions containing product were combined and the solvent removed in vacuo affording the title compound (6.10 g, 53%) as a pale yellow wax LCMS Method R (m/z): [M+H]⁺=165.9, retention time=1.08 min.

### Step 2: Synthesis of 3-((1-bromocyclopropyl)fluoromethyl)-2-chloropyridine

2-chloro-3-(cyclopropylidenemethyl)pyridine (7.80 g, 47.1 mmol) and DCM (284 mL) were cooled in an ice bath with stirring and triethylamine trihydrofluoride (26.9 mL, 165 mmol) was added followed by NBS (12.7 g, 70.6 mmol). The reaction was then stirred at 0° C. for 2 h, diluted with DCM and quenched with slow addition of 20% aqueous K₂CO₃. The mixture was extracted with DCM and the organic portion was adsorbed onto silica gel and purified by normal phase flash chromatography (Dry Pack) (SiO₂ with Heptanes/EtOAc: 0%-100%), the fractions containing product were combined and the solvent removed in vacuo affording a the title compound (9.00 g, 51%) as yellow oil. LCMS Method R (m/z): [M+H]⁺=263.8, 265.8, retention time=1.11 min. ¹H NMR (400 MHz, DMSO-d₆) δ 8.46 (dd, J=4.7, 1.9 Hz, 1H), 8.03 (dd, J=7.7, 1.9 Hz, 1H), 7.55 (dd, J=7.7, 4.7 Hz, 1H), 5.67 (d, J=44.0 Hz, 1H), 1.50-1.29 (m, 4H).

### Step 3: Synthesis of 2-chloro-3-(cyclopropylidenefluoromethyl)pyridine

3-((1-Bromocyclopropyl)fluoromethyl)-2-chloropyridine (9.00 g, 34.0 mmol) and 1,8-diazabicyclo[5.4.0]undec-7-ene (11.2 mL, 86.8 mmol) in DCM (62.4 mL) were stirred at 50° C. for 30 h in a sealed tube. the reaction was cooled to room temperature, diluted with DCM and washed with saturated aqueous NH₄Cl and brine. The organic portion was dried over anh. Na₂SO₄, filtered and adsorbed onto silica gel. Purified by normal phase flash chromatography (Dry Pack) (SiO₂ with Heptanes/EtOAc: 0%-15%), the fractions containing product were combined and the solvent removed in vacuo affording the title compound (2.80 g, 45%) as a pale yellow oil. LCMS Method R (m/z): [M+H]⁺=183.9, retention time=1.03 min

### Step 4: Synthesis of 2-chloro-3-(7-fluorodispiro[2.0.24.13]heptan-7-yl)pyridine

A flask containing 2-chloro-3-(cyclopropylidenefluoromethyl)pyridine (400 mg, 2.18 mmol) and cyclopropyldiphenylsulfonium tetrafluoroborate (1.15 g, 3.49 mmol) was purged and filled with N₂ three times, and then dry THF (21.8 mL) was added. The mixture was cooled in an ice bath with stirring under nitrogen and NaHMDS 1 M in THF was added dropwise, and the reaction was stirred at 0° C. for 1 h then quenched with saturated NH₄Cl. The aqueous layer was extracted into EtOAc and the organic portion was washed with brine, dried over anh. Na₂SO₄, filtered and adsorbed onto silica gel. Purified by normal phase flash chromatography (dry pack) (SiO₂ with Heptanes/EtOAc: 0%-30%), the fractions containing product were combined and the solvent removed in vacuo affording a yellow oil which was taken up in DMSO and further purified by reverse phase flash chromatography (C18 silica with 10 mM Ammonium Bicarbonate buffer/ACN: 5%-30%), the fractions containing product were combined and the organic portion was removed under vacuum at room temperature due to product volatility. The aqueous portion was extracted into EtOAc and the combined organics were washed with brine, dried over anh. Na₂SO₄, filtered and concentrated under vacuum affording the title compound (210 mg, 52%) as a pale yellow oil. LCMS Method R (m/z): [M+H]⁺=223.9, retention time=1.15 min.

### Step 5: Synthesis of 6-(cyclopropanecarboxamido)-4-((3-(7-fluorodispiro[2.0.24.13]heptan-7-yl)pyridin-2-yl)amino)-N-(methyl-d₃)pyridazine-3-carboxamide

### Synthesized According to General Procedure A

4-amino-6-(cyclopropanecarboxamido)-N-(methyl-d₃)pyridazine-3-carboxamide (58.6 mg, 246 umol), 2-chloro-3-(7-fluorodispiro[2.0.24.13]heptan-7-yl)pyridine (50.0 mg, 224 umol), Pd₂(dba)₃ (32.3 mg, 33.5 umol), dcpf (40.7 mg, 67.1 umol), K₃PO₄ (194 mg, 894 umol) and dioxane (1.60 mL) were added to a pressure tube fitted with a stir bar and nitrogen was bubbled through the mixture for 5 mins. The tube was sealed and placed on a heating block at 110° C. for 48 h. The reaction was cooled to ambient temperature and filtered through ca. 10 g of silica gel topped with Celite® using EtOAc as eluent. The filtrate was adsorbed onto silica gel and purified by normal phase flash chromatography (Dry Pack) (SiO₂ with DCM/IPA in DCM): 0%-10%), the fractions containing product were combined and the solvent removed in vacuo. The residue taken up in DMSO and further purified by reverse phase flash chromatography (C18 silica with 10 mM Ammonium Bicarbonate buffer/ACN: 10%-40%) to afford the title compound (5.60 mg, 5.9%). LCMS Method T (m/z): [M+H]⁺=426.4, retention time=2.83 min. ¹H NMR (400 MHz, DMSO-d₆) δ 11.58 (s, 1H), 11.32 (s, 1H), 9.46 (s, 1H), 9.17 (s, 1H), 8.36 (d, J=4.8 Hz, 1H), 7.92 (dd, J=6.0, 1.6 Hz, 1H), 7.10 (dd, J=7.2, 5.1 Hz, 1H), 2.15-2.03 (m, 1H), 1.41-1.33 (m, 2H), 1.20-1.08 (m, 2H), 0.97-0.87 (m, 2H), 0.87-0.81 (m, 4H), 0.81-0.71 (m, 2H).

### Example 30B

The following example was prepared in a manner similar to Compound 99 in Example 30A, from coupling the appropriate aminopyridazine and pyridyl chloride at the final step:

TABLE 13

| | | | | (m/z) | |
|---|---|---|---|---|---|
| Ex | | | MW | [M + H]⁺ | |
| No. | Cmpd No. | Structure | (g/mol) | (method) | ¹H NMR |
| 31B | 100A | 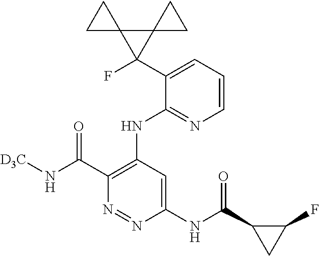 [SMILES: [2H]C([2H])([2H])NC(=O)c1nnc(NC(=O)[C@@H]2C[C@@H]2F)cc1Nc1ncccc1C1(F)C2(CC2)C12CC2] | 443.5 | 444.2 (T) | ¹H NMR (400 MHZ, DMSO-d₆) δ 11.62 (s, 1H), 11.39 (s, 1H), 9.49 (s, 1H), 9.21 (s, 1H), 8.40 (dt, J = 4.8, 1.5 Hz, 1H), 7.95 (dt, J = 7.6, 1.7 Hz, 1H), 7.13 (ddd, J = 7.5, 4.9, 0.9 Hz, 1H), 4.96 (dtd, J = 66.1, 6.1, 3.8 Hz, 1H), 2.37-2.26 (m, 1H), 1.66 (dtd, J = 23.4, 6.8, 3.8 Hz, 1H), 1.45-1.34 (m, 2H), 1.27-1.11 (m, 3H), 0.99-0.89 (m, 2H), 0.85-0.72 (m, 2H). |

### Example 31: 4-((3-(1-chlorocyclopropyl)pyridin-2-yl)amino)-6-(cyclopropanecarboxamido)-N-(methyl-d3)pyridazine-3-carboxamide (Compound 43)

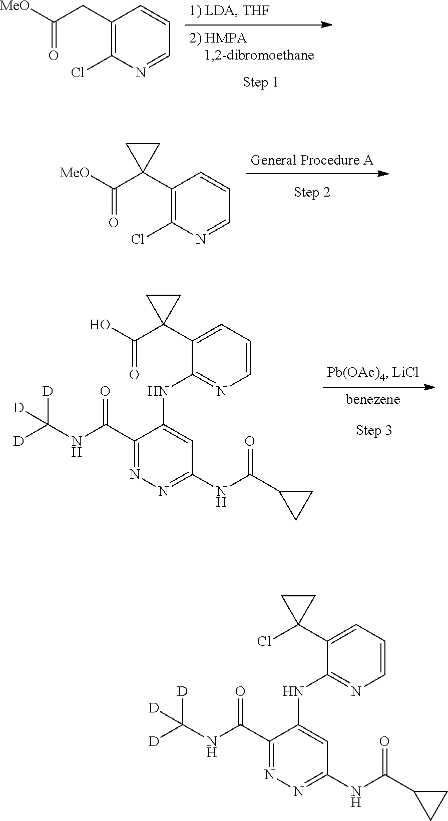 [SMILES: COC(=O)Cc1cccnc1Cl ; COC(=O)C1(c2cccnc2Cl)CC1 ; [2H]C([2H])([2H])NC(=O)c1nnc(NC(=O)C2CC2)cc1Nc1ncccc1C1(C(=O)O)CC1 ; [2H]C([2H])([2H])NC(=O)c1nnc(NC(=O)C2CC2)cc1Nc1ncccc1C1(Cl)CC1]

### Step 1: Synthesis of methyl 1-(2-chloropyridin-3-yl)cyclopropane-1-carboxylate

Butyllithium (12.8 mL, 32.0 mmol) was added to a solution of diisopropylamine (4.81 mL, 34.1 mmol) in THF (20 mL) at −78° C. After 5 min, to this mixture was added THF (87 mL) and Methyl 2-(2-chloropyridin-3-yl)acetate (4.00 g, 21.3 mmol) and the reaction was stirred at −78° C. for 30 min. The resulting mixture was then warmed to 0° C. and hexamethylphosphoramide (HMPA) (7.58 mL, 42.7 mmol) was added slowly. After stirring at 0° C. for 15 mins 1,2-dibromoethane (2.04 mL, 23.5 mmol) was added dropwise to the solution, stirred for 30 min at 0° C. then stirred at room temperature for 18 h. To this mixture was added sat. aq. NH₄Cl and the aqueous portion was extracted with EtOAc. The combined organics were dried over Na₂SO₄, filtered, and concentrated under reduced pressure. The reside was purified by reverse phase flash chromatography on C18 column using ACN in 10 mM AmB buffer (5%-100%). Fractions containing product were extracted with EtOAc and the combined organics were dried over anh. Na₂SO₄, filtered and concentrated under vacuum to afford methyl 1-(2-chloropyridin-3-yl)cyclopropane-1-carboxylate (2.62 g, 58%) as a colorless oil. LCMS Method F (m/z): [M+H]⁺=212.1, retention time=0.86 min.

### Step 2: Synthesis of 1-(2-((6-(cyclopropanecarboxamido)-3-((methyl-d₃)carbamoyl)pyridazin-4-yl)amino)pyridin-3-yl)cyclopropane-1-carboxylic acid

4-amino-6-(cyclopropanecarboxamido)-N-(methyl-d₃)pyridazine-3-carboxamide (338 mg, 1.42 mmol), methyl 1-(2-chloropyridin-3-yl)cyclopropane-1-carboxylate (250 mg, 1.18 mmol), Pd₂(dba)₃ (228 mg, 236 umol), dcpf (287 mg, 472 umol), K₃PO₄ (1.02 g, 4.72 mmol) and dioxane (8.44 mL) were added to a pressure tube fitted with a stir bar and nitrogen was bubbled through the mixture for 5 mins. The tube was sealed and placed on a heating block at 120° C. for 5 h. The reaction mixture was extracted with DCM:MeOH (4:1). The aqueous phase contained desired product. This aqueous phase's pH was adjusted to -1 and extracted with DCM:MeOH (4:1). The combined organic layers were dried over Na₂SO₄, filtered and concentrated under reduced pressure and lyophilized to afford 1-(2-((6-(cyclopropanecarboxamido)-3-((methyl-d₃)carbamoyl)pyridazin-4-yl)amino)pyridin-3-yl)cyclopropane-1-carboxylic acid (97.0 mg, 21%) as a pale yellow solid. This residue was used as it is in the next step without any further purification. LCMS Method E (m/z): [M+H]⁺=400.3, retention time=0.88 min.

### Step 3: Synthesis of 4-((3-(1-chlorocyclopropyl)pyridin-2-yl)amino)-6-(cyclopropanecarboxamido)-N-(methyl-d₃)pyridazine-3-carboxamide

To a mixture of 1-(2-((6-(cyclopropanecarboxamido)-3-((methyl-d₃)carbamoyl)pyridazin-4-yl)amino)pyridin-3-yl)cyclopropane-1-carboxylic acid (25.0 mg, 62.6 umol), lithium chloride (5.63 mg, 131 umol), lead(IV) acetate (70.1 mg, 150 umol) was added benzene (0.500 mL). The reaction mixture was stirred at 100° C. for 6 h. The reaction mixture was quenched with sat. NaHCO₃ and filtered on Celite® and flushed with DCM:MeOH (4:1). This DCM:MeOH mixture was washed with water. The combined organic layers were dried over Na₂SO₄, filtered and concentrated under vacuum. The product was purified over normal phase flash chromatography (dry load) using MeOH in DCM (1%-10%) as an eluent. Fractions containing product were combined and the solvent removed under reduced pressure. The reside was further purified over reverse phase flash chromatography on C18 column using ACN in 10 mM AmB buffer (5% -100%) to afford the title compound (2.30 mg, 9.4%). LCMS Method H (m/z): [M+H]⁺=390.2, retention time=2.24 min. ¹H NMR (500 MHz, DMSO) δ=12.22 (s, 1H), 11.32 (s, 1H), 9.80 (s, 1H), 9.20 (s, 1H), 8.32 (dd, J=4.9, 1.8, 1H), 7.89 (dd, J=7.5, 1.8, 1H), 7.07 (dd, J=7.5, 4.9, 1H), 2.16-2.08 (m, 1H), 1.68-1.61 (m, 2H), 1.43-1.37 (m, 2H), 0.91-0.83 (m, 4H).

### Example 32: 6-(cyclopropanecarboxamido)-4-((3-(1-fluorocyclopropyl)-4-methylpyridin-2-yl)amino)-N-(methyl-d3)pyridazine-3-carboxamide (Compound 50)

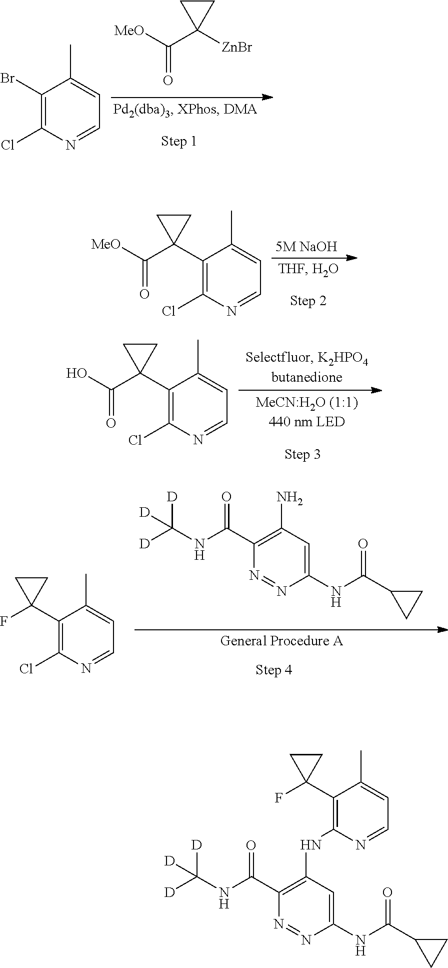

### Step 1: Synthesis of methyl 1-(2-chloro-4-methylpyridin-3-yl)cyclopropane-1-carboxylate

Zinc (7 g) was stirred with 2% (m/m) aqueous hydrochloric acid (25 mL) for 5 min and then filtered. The filter cake was washed with water (2×10 mL), ethanol (2×10 mL), and petroleum ether (2×10 mL). The solid was dried in vacuo to obtain activated Zinc dust.

To a suspension of activated zinc (465 mg, 6.97 mmol) in dry DMA (7 mL) was added 1,2-dibromoethane (106 uL, 1.21 mmol) under a nitrogen atmosphere. The reaction mixture was stirred at 70° C. for 5 min and cooled down to 25° C. This procedure was repeated three times. Then, chlorotrimethylsilane (138 uL, 1.07 mmol) was added dropwise, and the resulting suspension was stirred at 25° C. for 15 min. The mixture was then heated to 65° C. and methyl 1-bromocyclopropanecarboxylate (1.00 mL, 5.36 mmol) in DMA (1 mL) was added dropwise over a period of 30 min. After completion of addition, the reaction mixture was stirred at 65° C. for 1 h and allowed to cool to room temperature. The zinc was allowed to settle, and the supernatant was used further without any analysis. Complete conversion was assumed. The resulting solution was used without any workup in the next step.

A mixture of 3-Bromo-2-chloro-4-picoline (300 mg, 1.44 mmol), Pd₂(dba)₃ (139 mg, 0.144 mmol), and XPhos (105 mg, 0.217 mmol) in DMA (5.78 mL) was degassed by nitrogen bubbling for 2 min. Then, a solution of (1-(methoxycarbonyl)cyclopropyl)zinc(II) bromide (6.57 mL, 3.61 mmol) (0.55 M in DMA) was added rapidly. The reaction mixture was stirred at 80° C. under a nitrogen atmosphere for 1 h. After cooling, the mixture was purified directly by reversed phase flash chromatography (10 mM AmF buffer/acetonitrile). The product containing fraction was concentrated in vacuo to remove acetonitrile. The aqueous solution was extracted with ethyl acetate (3×15 mL). The combined organic layers were dried over anh. Na₂SO₄ and filtered, and the filtrate was concentrated in vacuo (without heat) to give methyl 1-(2-chloro-4-methylpyridin-3-yl)cyclopropane-1-carboxylate (325 mg, 100%), assumed quantitative. LCMS method F [M+H]⁺=226.1 m/z, retention time=0.93 min.

### Step 2: Synthesis of 1-(2-chloro-4-methylpyridin-3-yl)cyclopropane-1-carboxylic acid

To a solution of methyl 1-(2-chloro-4-methylpyridin-3-yl)cyclopropane-1-carboxylate (325 mg, 1.44 mmol) and THF (7.20 mL), MeOH (3.60 mL), and water (3.60 mL) was added aq. 5 M sodium Hydroxide (288 mg, 7.20 mmol). The reaction mixture was stirred at 40° C. for 40 h. 1 M HCl was added and brought pH ~1-3 and extracted with DCM:MeOH (1:4). The combined organic layers were dried over anh. Na₂SO₄, filtered and concentrated under reduced pressure to afford 1-(2-chloro-4-methylpyridin-3-yl)cyclopropane-1-carboxylic acid (150 mg, 49%) as a white solid. The residue was used as it is in the next step without any further purification. LCMS method F [M+H]⁺=212.0 m/z, retention time=0.35 min.

### Step 3: Synthesis of 2-chloro-3-(1-fluorocyclopropyl)-4-methylpyridine

To o a mixture of 1-(2-chloro-4-methylpyridin-3-yl)cyclopropane-1-carboxylic acid (200 mg, 0.945 mmol), Selectfluor® (1.07 g, 3.02 mmol), and Na₂HPO₄ (285 mg, 1.98 mmol) was added MeCN (4.72 mL) and Water (4.72 mL). The reaction mixture was degassed with nitrogen for 5 min. To this mixture was then added 2,3-butanedione (8.47 uL, 0.0945 mmol) and stirred over the weekend (ca. 72 h) under the irradiation of 440 nm LED. The reaction mixture was extracted with DCM and washed with sat. aq. NaHCO₃. The combined organic layers were dried over anh. Na₂SO₄, filtered and concentrated under reduced pressure to afford 2-chloro-3-(1-fluorocyclopropyl)-4-methylpyridine (20.0 mg, 11%) as a pale yellow oil. This residue was used as it is in the next step without any further purification. LCMS Method F (m/z): [M+H]⁺=no ionization, retention time=1.02 min.

### Step 4: Synthesis of 6-(cyclopropanecarboxamido)-4-((3-(1-fluorocyclopropyl)-4-methylpyridin-2-yl)amino)-N-(methyl-d₃)pyridazine-3-carboxamide

To a degassed mixture of 2-chloro-3-(1-fluorocyclopropyl)-4-methylpyridine (20.0 mg, 0.108 mmol), 4-amino-6-(cyclopropanecarboxamido)-N-(methyl-d₃)pyridazine-3-carboxamide (51.3 mg, 0.215 mmol), dcpf (26.2 mg, 0.0431 mmol) and K₃PO₄ (70.0 mg, 0.323 mmol) in degassed dioxane (1.08 mL) was added Pd₂(dba)₃ (19.7 mg, 0.0215 mmol). Nitrogen was immediately bubbled to the mixture for 2 min and the reaction was stirred at 120° C. for 16 h. Upon completion, the mixture was cooled to room temperature, diluted with EtOAc and filtered on Celite®. The Celite® cake was rinsed with EtOAc and the combined organics were concentrated under reduced pressure. The residue was purified by normal phase chromatography (0-10% iPrOH/DCM), followed by reverse phase chromatography (10-100% MeCN/10 mM AmB buffer) to afford 6-(cyclopropanecarboxamido)-4-((3-(1-fluorocyclopropyl)-4-methylpyridin-2-yl)amino)-N-(methyl-d₃)pyridazine-3-carboxamide (11.3 mg, 27%). LCMS Method L (m/z): [M+H]⁺=388.1, retention time=2.55 min. ¹H NMR (500 MHz, DMSO-d₆) δ 12.17 (s, 1H), 11.29 (s, 1H), 9.69 (s, 1H), 9.19 (s, 1H), 8.24 (dd, J=5.0, 1.3 Hz, 1H), 7.00 (d, J=5.1 Hz, 1H), 2.51 (s, 3H), 2.11 (tt, J=7.3, 5.5 Hz, 1H), 1.66 (m, 2H), 1.06 (m, 2H), 0.86 (m, 4H).

### Example 33A: N-(4-((3-(3-fluorooxetan-3-yl)pyrazin-2-yl)amino)-5-propionylpyridin-2-yl)cyclopropanecarboxamide (Compound 102)

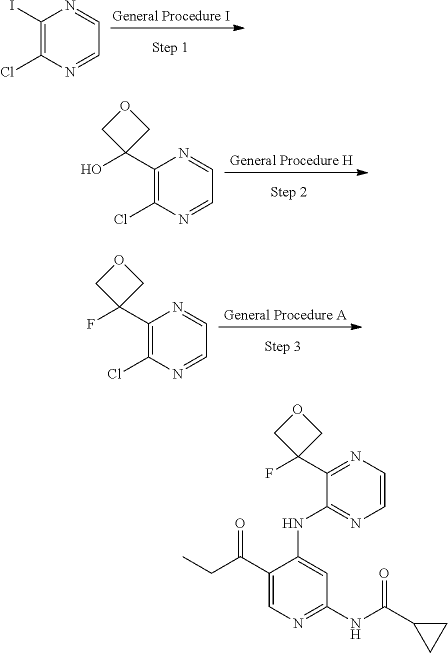

### Step 1: Synthesis of 3-(3-chloropyrazin-2-yl)oxetan-3-ol

The title compound was prepared according to General Procedure I

To a cold (−78° C.) stirring solution of 2-chloro-3-iodopyrazine (1.00 g, 4.03 mmol) in a mixture of heptane (20.0 mL) and toluene (20.0 mL) was added tert-butyllithium 1.7 M in pentane (5.41 mL, 9.20 mmol). The mixture was stirred at −78° C. for 10 min and oxetan-3-one (800 uL, 12.2 mmol) was added. The mixture was stirred at −78° C. for 30 min and NH₄Cl sat. (5 mL) was added. The mixture was allowed to warm to room temperature. The mixture was diluted with EtOAc and washed with water (3×), brine, dried over Na₂SO₄, filtered and concentrated under reduce pressure. The residue was purified by silica gel chromatography (20-60% EtOAc:Hexanes) to provide the title compound (166 mg, 20%) as a clear oil. ¹H NMR (400 MHz, CDCl3) δ 8.51 (d, J=2.5 Hz, 1H), 8.41 (d, J=2.5 Hz, 1H), 5.35 (dd, J=7.5, 1.0 Hz, 2H), 4.91 (dd, J=7.5, 1.0 Hz, 2H). LCMS Method Q (m/z): [M+H]⁺=187.0, retention time=0.45 min.

### Step 2: Synthesis of 2-chloro-3-(3-fluorooxetan-3-yl)pyrazine

The title compound was prepared according to General Procedure H

To a solution of 3-(3-chloropyrazin-2-yl)oxetan-3-ol (166 mg, 801 umol) in DCM (20.0 mL) was added (diethylamino)sulfur trifluoride (500 uL, 3.84 mmol). The solution was stirred at room temperature for 15 min. NaHCO₃ sat. (20 mL) was added and stirred for 1 h. The mixture was extracted with DCM (2×). The combined organics were washed with NaHCO₃ sat. (2×), Na₂S₂O₃ 5% (lx) and brine, dried over anh. Na₂SO₄, filtered and concentrated under reduced pressure to provide the title compound (152 mg, 101%) as an oil. ¹H NMR (400 MHz, CDCl₃) δ 8.57-8.54 (m, 1H), 8.48-8.45 (m, 1H), 5.22 (dddd, J=83.2, 21.5, 8.7, 1.4 Hz, 4H). LCMS Method Q (m/z): [M+H]⁺=174.3, retention time=0.78 min.

### Step 3: Synthesis of N-(4-((3-(3-fluorooxetan-3-yl)pyrazin-2-yl)amino)-5-propionylpyridin-2-yl)cyclopropanecarboxamide

The title compound was prepared according to General Procedure A

To a degassed mixture of N-(4-amino-5-propionylpyridin-2-yl)cyclopropanecarboxamide (110 mg, 472 umol), 2-chloro-3-(3-fluorooxetan-3-yl)pyrazine (152 mg, 806 umol) and K₂CO₃(181 mg, 1.31 mmol) in dioxane (3.00 mL) was added dcpf (103 mg, 170 umol) and Pd₂(dba)₃ (75.8 mg, 81.1 umol). Nitrogen was immediately bubbled in the mixture for 10 min and the mixture was heated at 90° C. for 48 h. The mixture was cooled, diluted with EtOAc and filtered on Celite®. The Celite® cake was rinsed with 10% MeOH in EtOAc and the combined organic was concentrated under reduced pressure. The residue was purified by reverse phase C18 chromatography (5-100% ACN in 10 mM AMF) and normal phase silica gel (0-10% IPA in DCM) to provide 30 mg of material which was then further purified by Prep-HPLC (35-60% ACN in 10 mM AmF) to provide the title compound (15.2 mg, 8.2%). ¹H NMR (400 MHz, DMSO-d₆) δ 11.82 (s, 1H), 11.01 (s, 1H), 9.32 (s, 1H), 8.97 (s, 1H), 8.58-8.49 (m, 1H), 8.35 (d, J=2.5 Hz, 1H), 5.26 (dd, J=23.6, 9.4 Hz, 2H), 5.11 (dd, J=21.2, 8.9 Hz, 2H), 3.16 (q, J=7.1 Hz, 2H), 2.12-2.00 (m, 1H), 1.12 (t, J=7.2 Hz, 3H), 0.90-0.79 (m, 4H). LCMS Method Q (m/z): [M+H]⁺=386.3, retention time=1.04 min.

### Examples 33B-33H

The following examples were prepared in a manner similar to Compound 102 described in Example 33A from their corresponding commercially available ketones:

TABLE 14

| | | | | (m/z) | |
|---|---|---|---|---|---|
| Ex. | | | MW | [M + H]⁺ | |
| No. | Cmpd No. | Structure | (g/mol) | (method) | ¹H NMR |
| 33B | 103 | 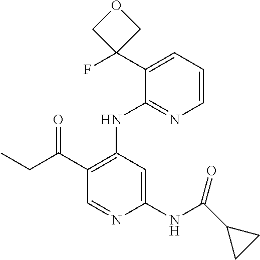 [SMILES: CCC(=O)c1cnc(NC(=O)C2CC2)cc1Nc1ncccc1C1(F)COC1] | 384.41 | 385.3 (S) | ¹H NMR (400 MHZ, DMSO-d₆) δ 11.50 (s, 1H), 10.91 (s, 1H), 9.10 (s, 1H), 8.92 (s, 1H), 8.51- 8.40 (m, 1H), 8.02 (dt, J = 7.6, 2.0 Hz, 1H), 7.22 (dd, J = 6.5, 5.1 Hz, 1H), 5.12 (q, J = 8.2 Hz, 2H), 5.05 (q, J = 8.5 Hz, 2H), 3.13 (q, J = 7.2 Hz, 2H), 2.09- 2.00 (m, 1H), 1.11 (t, J = 7.2 Hz, 3H), 0.81 (d, J = 6.2 Hz, 4H). |
| 33C | 104 | 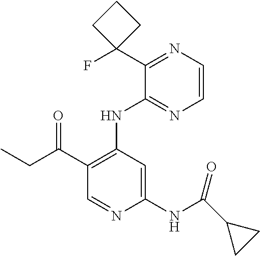 [SMILES: CCC(=O)c1cnc(NC(=O)C2CC2)cc1Nc1nccnc1C1(F)CCC1] | 383.43 | 384.3 (S) | ¹H NMR (400 MHZ, DMSO-d₆) δ 11.86 (s, 1H), 10.97 (s, 1H), 9.34 (s, 1H), 8.96 (s, 1H), 8.44 (s, 1H), 8.30 (d, J = 1.9 Hz, 1H), 3.14 (q, J = 7.1 Hz, 2H), 2.89 (d, J = 9.3 Hz, 2H), 2.71 (td, J = 21.4, 11.0 Hz, 2H), 2.11-1.88 (m, 2H), 1.46 (dt, J = 17.7, 9.0 Hz, 1H), 1.11 (t, J = 7.1 Hz, 3H), 0.83 (d, J = 4.5 Hz, 4H). |
| 33D | 105A | 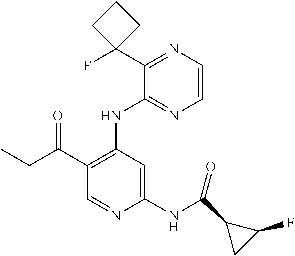 [SMILES: CCC(=O)c1cnc(NC(=O)[C@@H]2C[C@@H]2F)cc1Nc1nccnc1C1(F)CCC1] | 401.42 | 402.3 (S) | ¹H NMR (400 MHZ, DMSO-d₆) δ 11.87 (d, J = 2.9 Hz, 1H), 11.02 (s, 1H), 9.34 (s, 1H), 8.97 (s, 1H), 8.52-8.42 (m, 1H), 8.31 (d, J = 2.6 Hz, 1H), 4.94 (ddd, J = 66.1, 10.0, 6.2 Hz, 1H), 3.15 (q, J = 7.2 Hz, 2H), 2.98-2.80 (m, 2H), 2.80-2.62 (m, 2H), 2.30-2.19 (m, 1H), 2.05-1.90 (m, 1H), 1.72-1.28 (m, 1H), 1.55-1.39 (m, 1H), 1.22-1.09 (m, 1H), 1.11 (t, J = 7.2 Hz, 3H). |
| 33E | 106 | 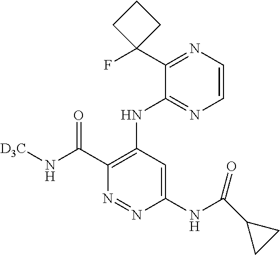 [SMILES: [2H]C([2H])([2H])NC(=O)c1nnc(NC(=O)C2CC2)cc1Nc1nccnc1C1(F)CCC1] | 388.42 | 389.1 (S) | ¹H NMR (400 MHz, DMSO-d₆) δ 12.07 (d, J = 2.3 Hz, 1H), 11.41 (s, 1H), 9.66 (s, 1H), 9.23 (s, 1H), 8.47-8.42 (m, 1H), 8.29 (d, J = 2.6 Hz, 1H), 2.98- 2.84 (m, 2H), 2.80-2.64 (m, 2H), 2.17-2.08 (m, 1H), 2.04- 1.91 (m, 1H), 1.55-1.41 (m, 1H), 0.91-0.82 (m, 4H). |
| 33F | 107 | 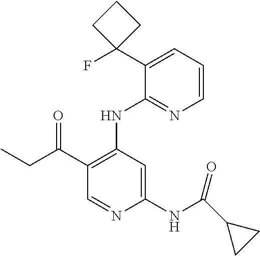 [SMILES: CCC(=O)c1cnc(NC(=O)C2CC2)cc1Nc1ncccc1C1(F)CCC1] | 382.44 | 383.2 (S) | ¹H NMR (400 MHZ, DMSO-d₆) δ 11.56 (s, 1H), 10.87 (s, 1H), 9.14 (s, 1H), 8.91 (s, 1H), 8.38 (d, J = 4.6 Hz, 1H), 7.97 (d, J = 7.6 Hz, 1H), 7.25-7.10 (m, 1H), 3.12 (q, J = 7.1 Hz, 2H), 2.80-2.60 (m, 4H), 2.10-1.92 (m, 2H), 1.61-1.43 (m, 1H), 1.10 (t, J = 7.1 Hz, 3H), 0.80 (d, J = 6.1 Hz, 4H). |
| 33G | 108A | 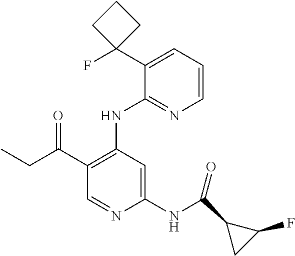 [SMILES: CCC(=O)c1cnc(NC(=O)[C@@H]2C[C@@H]2F)cc1Nc1ncccc1C1(F)CCC1] | 400.43 | 401.3 (S) | ¹H NMR (400 MHZ, DMSO-d₆) δ 11.57 (s, 1H), 10.92 (s, 1H), 9.14 (s, 1H), 8.92 (s, 1H), 8.40 (s, 1H), 7.98 (d, J = 7.1 Hz, 1H), 7.28-7.12 (m, 1H), 4.92 (d, J = 66.4 Hz, 1H), 3.13 (q, J = 6.9 Hz, 2H), 2.77-2.62 (m, 4H), 2.28-2.18 (m, 1H), 2.05-1.92 (m, 1H), 1.71-1.45 (m, 2H), 1.11 (t, J = 7.0 Hz, 4H). |
| 33H | 109 | 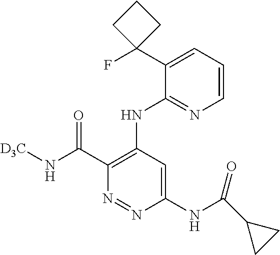 [SMILES: [2H]C([2H])([2H])NC(=O)c1nnc(NC(=O)C2CC2)cc1Nc1ncccc1C1(F)CCC1] | 387.43 | 388.3 (S) | ¹H NMR (400 MHZ, DMSO-d₆) δ 11.67 (d, J = 1.9 Hz, 1H), 11.31 (s, 1H), 9.55 (s, 1H), 9.15 (s, 1H), 8.44-8.33 (m, 1H), 7.96 (d, J = 7.7 Hz, 1H), 7.21- 7.07 (m, 1H), 2.83-2.60 (m, 4H), 2.16-2.07 (m, 1H), 2.08- 1.98 (m, 1H), 1.60-1.45 (m, 1H), 0.85 (d, J = 6.0 Hz, 4H). |

### Example 34: 6-(cyclopropanecarboxamido)-N-ethyl-4-((3-(1-fluorospiro[2.2]pentan-1-yl)pyridin-2-yl)amino)pyridazine-3-carboxamide (Compound 110)

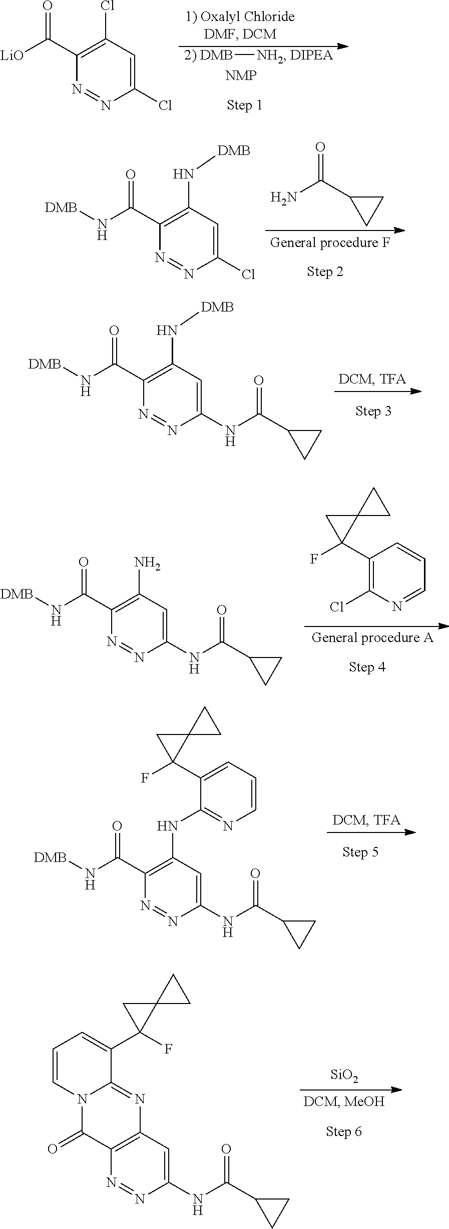

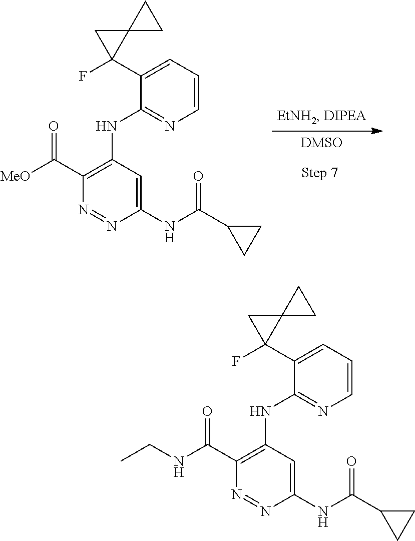 [SMILES: COC(=O)c1nnc(NC(=O)C2CC2)cc1Nc1ncccc1C1(F)CC12CC2 ; CCNC(=O)c1nnc(NC(=O)C2CC2)cc1Nc1ncccc1C1(F)CC12CC2]

### Step 1: Synthesis of 6-chloro-N-(2,4-dimethoxybenzyl)-4-((2,4-dimethoxybenzyl)amino)pyridazine-3-carboxamide

A dry 250 mL round-bottom flask equipped with a Teflon®-coated stir bar and rubber septum was charged with lithium 4,6-dichloropyridazine-3-carboxylate (2.00 g, 9.75 mmol), followed by CH₂Cl₂ (40.0 mL), and DMF (75.7 uL, 975 umol). The suspension was cooled down to 0° C., and oxalyl chloride (5.05 mL, 58.5 mmol) was added dropwise. The reaction mixture was stirred at room temperature for 2 h. The volatiles were removed in vacuo to give a tan powder, which was redissolved in NMP (40.0 mL), and then N,N-diisopropylethylamine (8.58 mL, 48.8 mmol) and 2,4-dimethoxybenzylamine (5.92 mL, 39.0 mmol) were added in quick succession. The flask became warm to the touch. The flask was stirred at room temperature for 10 minutes then heated at 60° C. for 20 minutes.

The reaction mixture was poured into water (100 mL) and extracted with EtOAc (3×50 mL). The combined organic layers were washed with 1 M HCl (2×50 mL) followed by brine (1×50 mL), then dried over Na₂SO₄ and concentrated under vacuum. The material was purified by flash chromatography (wet loaded with small amount of DCM, 0-60% EtOAc in Heptanes) to give the title compound (3.50 g, 76%) as an off-white solid. LCMS Method Q (m/z): [M+H]⁺=473.1, retention time=1.34 min.

### Step 2: Synthesis of 6-(cyclopropanecarboxamido)-N-(2,4-dimethoxybenzyl)-4-((2,4-dimethoxybenzyl)amino)pyridazine-3-carboxamide

The synthesis was performed according to General Procedure F. A dry pressure vessel was charged with 6-chloro-N-(2,4-dimethoxybenzyl)-4-((2,4-dimethoxybenzyl)amino)pyridazine-3-carboxamide (7.00 g, 14.8 mmol) in dioxane (94.3 mL). followed by cyclopropanecarboxamide (3.21 g, 37.0 mmol), XantPhos (1.75 g, 2.96 mmol) and cesium carbonate (12.3 g, 37.0 mmol), The mixture was sparged with nitrogen for 5 min, and then Pd₂(dba)₃ (1.36 g, 1.48 mmol) was added. The mixture was sparged with N₂ for a further 2 minutes and then vessel was sealed. The reaction mixture was heated at 125° C. for 12 h. The solvent was removed in vacuo and the residue was purified by flash column chromatography (dry load, 0-20% MeOH in DCM) to give the title compound (7.60 g, 98%) as an off-white solid. LCMS Method R (m/z): [M+H]⁺=522.1, retention time=1.28 min.

### Step 3: Synthesis of 4-amino-6-(cyclopropanecarboxamido)-N-(2,4-dimethoxybenzyl)pyridazine-3-carboxamide

6-(cyclopropanecarboxamido)-N-(2,4-dimethoxybenzyl)-4-((2,4-dimethoxybenzyl)amino)pyridazine-3-carboxamide (510 mg, 735 umol) was suspended in DCM (4.16 mL), and then trifluoroacetic acid (1.41 mL, 18.4 mmol) in DCM (2.77 mL) was added. A clear solution was obtained. The reaction mixture was stirred at 20° C. for 16 h. The mixture was dried in vacuo and purified directly by reversed phase C18 column (10-50% MeCN in AmB) to give the title compound (114 mg, 42%) as an off-white solid. LCMS Method J (m/z): [M−H]−=370.1, retention time=1.34 min. ¹H NMR (400 MHz, DMSO-d₆) δ 11.07 (s, 1H), 8.92 (t, J=6.2 Hz, 1H), 7.65 (s, 1H), 7.58 (br s, 2H), 7.10 (d, J=8.3 Hz, 1H), 6.56 (d, J=2.3 Hz, 1H), 6.47 (dd, J=8.3, 2.3 Hz, 1H), 4.38 (d, J=6.2 Hz, 2H), 3.82 (s, 3H), 3.74 (s, 3H), 2.07 (p, J=6.1 Hz, 1H), 0.83 (d, J=6.1 Hz, 4H).

### Step 4: Synthesis of 6-(cyclopropanecarboxamido)-N-(2,4-dimethoxybenzyl)-4-((3-(1-fluorospiro[2.2]pentan-1-yl)pyridin-2-yl)amino)pyridazine-3-carboxamide

The synthesis was performed according to General Procedure A. To a degassed mixture of 4-amino-6-(cyclopropanecarboxamido)-N-(2,4-dimethoxybenzyl)pyridazine-3-carboxamide (105 mg, 283 umol), dcpf (68.6 mg, 113 umol) and K₃PO₄ (184 mg, 848 umol) in dioxane (2.83 mL) was added Pd₂(dba)₃ (52.8 mg, 56.5 umol) and 2-chloro-3-(1-fluorospiro[2.2]pentan-1-yl)pyridine (72.6 mg, 368 umol). The mixture was sparged with N₂ for an additional 2 minutes and the reaction was stirred at 120° C. for 1 h. The mixture was cooled, dried in vacuo and purified directly by flash column chromatography (0-20% MeOH in DCM). The fractions containing compound were combined, concentrated, taken up in DMSO, filtered and injected on C18 column for purification (10-100% MeCN in AmB) to give the title compound (124 mg, 82%) as a white solid. LCMS Method R (m/z): [M+H]⁺=533.1, retention time=1.35 min.

### Step 5: Synthesis of N-(6-(1-fluorospiro[2.2]pentan-1-yl)-11-oxo-11H-pyrido[1′,2′:1,2]pyrimido[5,4-c]pyridazin-3-yl)cyclopropanecarboxamide

4-((3-(1,1-difluoroethyl)-5-fluoropyridin-2-yl)amino)-6-(1-fluorocyclopropane-1-carboxamido)-N-(methyl-d₃)nicotinamide (110 mg, 207 umol) was stirred in TFA (5.22 mL) at 40° C. for 16 h. LCMS analysis indicated that complete consumption of the starting material, with the major product being the title compound. The reaction mixture was dried in vacuo to give a solid residue. LCMS Method R (m/z): [M+H]⁺=366.1, retention time=1.06 min.

### Step 6: Synthesis of methyl 6-(cyclopropanecarboxamido)-4-((3-(1-fluorospiro[2.2]pentan-1-yl)pyridin-2-yl)amino)pyridazine-3-carboxylate

The residue from the previous step was purified by flash column chromatography (0-20% MeOH in DCM). The tricyclic product was reacted with MeOH on the column. The fractions were combined to give the title compound (51 mg, 60% purity, 36% yield over two steps) as an off-white solid. LCMS Method Q (m/z): [M+H]⁺=398.1, retention time=1.13 min.

### Step 7: Synthesis of 6-(cyclopropanecarboxamido)-N-ethyl-4-((3-(1-fluorospiro[2.2]pentan-1-yl)pyridin-2-yl)amino)pyridazine-3-carboxamide

Methyl 6-(cyclopropanecarboxamido)-4-((3-(1-fluorospiro[2.2]pentan-1-yl)pyridin-2-yl)amino)pyridazine-3-carboxylate (51.0 mg, 128 umol), DIPEA (294 uL, 1.67 mmol) and ethylamine in water (70% w/w) (124 uL, 1.54 mmol) were stirred in DMSO (1.00 mL) at 110° C. for 16 h, at which time LCMS indicated complete consumption of the starting material. The crude reaction mixture was diluted with DMSO to 2 mL, filtered, and the solution was directly purified by preparative scale HPLC (C18, 10-50% MeCN in AmF) to afford the title compound (3.50 mg, 6.6%). LCMS Method T (m/z): [M+H]⁺=411.3, retention time=2.86 min. ¹H NMR (400 MHz, DMSO-d₆) δ 11.79 (s, 1H), 11.33 (s, 1H), 9.64 (s, 1H), 9.29 (t, J=6.0 Hz, 1H), 8.39 (d, J=4.8 Hz, 1H), 7.95 (d, J=7.5 Hz, 1H), 7.13 (dd, J=7.2, 5.1 Hz, 1H), 3.39-3.34 (m, 2H), 2.18-2.08 (m, 1H), 1.79 (dd, J=13.7, 6.7 Hz, 1H), 1.74-1.67 (m, 1H), 1.56-1.48 (m, 1H), 1.22-1.10 (m, 4H), 0.95-0.74 (m, 6H).

### Example 35: 6-(cyclopropanecarboxamido)-4-((3-(1-methoxyspiro[2.2]pentan-1-yl)pyridin-2-yl)amino)-N-(methyl-d₃)pyridazine-3-carboxamide (Compound 63)

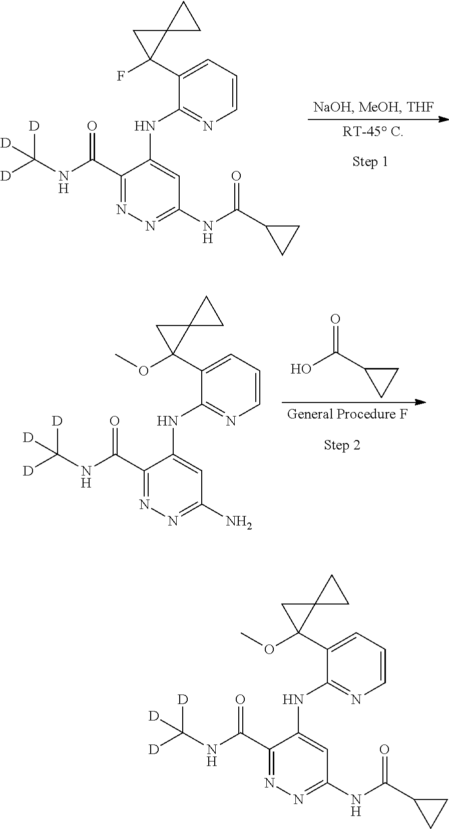 [SMILES: [2H]C([2H])([2H])NC(=O)c1nnc(NC(=O)C2CC2)cc1Nc1ncccc1C1(F)CC12CC2 ; [2H]C([2H])([2H])NC(=O)c1nnc(N)cc1Nc1ncccc1C1(OC)CC12CC2 ; O=C(O)C1CC1 ; [2H]C([2H])([2H])NC(=O)c1nnc(NC(=O)C2CC2)cc1Nc1ncccc1C1(OC)CC12CC2]

### Step 1: Synthesis of 6-amino-4-((3-(1-methoxyspiro[2.2]pentan-1-yl)pyridin-2-yl)amino)-N-(methyl-d₃)pyridazine-3-carboxamide

6-(Cyclopropanecarboxamido)-4-((3-(1-fluorospiro[2.2]pentan-1-yl)pyridin-2-yl)amino)-N-(methyl-d₃)pyridazine-3-carboxamide (187 mg, 469 umol), aq. 4 M sodium hydroxide (1.67 mL, 6.67 mmol), THF (2.78 mL) and MeOH (278 uL) were stirred at room temperature overnight (18 h) The reaction was heated to 45° C. for an additional 24 h at which point it was diluted with EtOAc and washed with water and brine. The organic portion was dried over anh. Na₂SO₄, filtered and adsorbed onto silica gel. Purified by normal phase flash chromatography (Dry Pack) (SiO₂ with DCM/IPA): 0%-10%), the fractions containing products were combined and the solvent removed in vacuo affording the title compound (80.0 mg, 50%) as a pale yellow solid. LCMS Method B (m/z): [M+H]⁺=344.3, retention time=0.89 min.

### Step 2: Synthesis of 6-(cyclopropanecarboxamido)-4-((3-(1-methoxyspiro[2.2]pentan-1-yl)pyridin-2-yl)amino)-N-(methyl-d₃)pyridazine-3-carboxamide

The title compound was prepared according to General Procedure F

6-Amino-4-((3-(1-methoxyspiro[2.2]pentan-1-yl)pyridin-2-yl)amino)-N-(methyl-d₃)pyridazine-3-carboxamide (80.0 mg, 233 umol), 1-methylimidazole (379 uL, 4.66 mmol) and cyclopropanecarboxylic acid (84.4 mg, 932 umol) were stirred in DMF (3.09 mL) and TCFH (674 mg, 2.33 mmol) was added. The reaction was stirred at 50° C. for 3 h then cooled to room temperature and diluted with EtOAc. The organic portion was washed with saturated NH₄Cl and brine, the organic portion was dried over anh. Na₂SO₄, filtered and concentrated under vacuum. The residue was taken up in DMSO and purified by Büchi Pure C850 Benchtop Semi-Prep (C18 silica with 10 mM Ammonium Bicarbonate Buffer pH 10.3/MeCN 5%-95%) to afford the title compound (10.0 mg, 5.2%). LCMS Method C (m/z): [M+H]⁺=412.4 retention time=2.14 min. ¹H NMR (400 MHz, DMSO-d₆) δ11.36 (s, 1H), 11.28 (s, 1H), 9.50 (s, 1H), 9.06 (s, 1H), 8.31 (dd, J=4.8, 1.6 Hz, 1H), 7.82 (dd, J=7.5, 1.7 Hz, 1H), 7.10 (dd, J=7.4, 4.9 Hz, 1H), 3.13 (s, 3H), 2.15-2.06 (m, 1H), 1.50-1.34 (m, 3H), 0.98-0.89 (m, 1H), 0.90-0.79 (m, 4H), 0.63-0.55 (m, 1H), 0.55-0.46 (m, 1H).

### Example 36: 6-((1S,2S)-2-fluorocyclopropane-1-carboxamido)-4-((3-(1-fluorospiro[2.2]pentan-1-yl)pyridin-2-yl)amino)-N-(methyl-d3)pyridazine-3-carboxamide (Compound 64A)

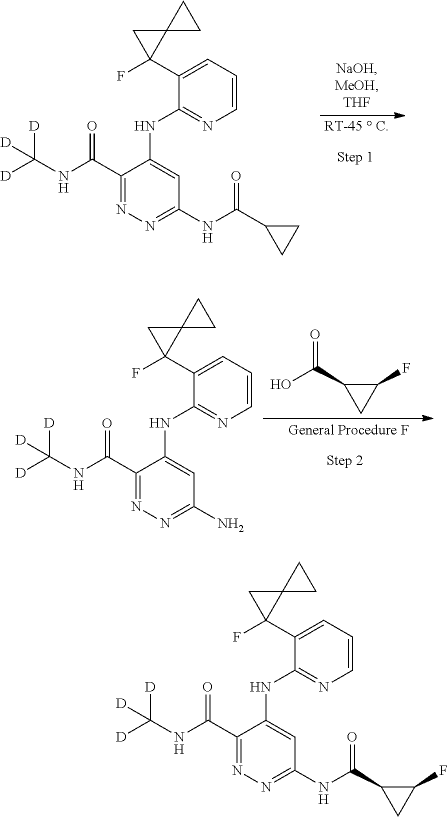

### Step 1: Synthesis of 6-amino-4-((3-(1-fluorospiro[2.2]pentan-1-yl)pyridin-2-yl)amino)-N-(methyl-d₃)pyridazine-3-carboxamide

6-(cyclopropanecarboxamido)-4-((3-(1-fluorospiro[2.2]pentan-1-yl)pyridin-2-yl)amino)-N-(methyl-d₃)pyridazine-3-carboxamide (187 mg, 469 umol), aq. 4 M sodium hydroxide (1.67 mL, 6.67 mmol), THF (2.78 mL) and MeOH (278 uL) were stirred at room temperature overnight (18 h) The reaction was heated to 45° C. for an additional 24 h at which point it was diluted with EtOAc and washed with water and brine. The organic portion was dried over anh. Na₂SO₄, filtered and adsorbed onto silica gel. Purified by normal phase flash chromatography (Dry Pack) (SiO₂ with DCM/IPA): 0%-10%), the fractions containing products were combined and the solvent removed in vacuo affording the title compound (51 mg, 33%) as a pale yellow solid. LCMS Method B (m/z): [M+H]⁺=332.3, retention time=0.97 min.

### Step 2: Synthesis of 6-((1S,2S)-2-fluorocyclopropane-1-carboxamido)-4-((3-(1-fluorospiro[2.2]pentan-1-yl)pyridin-2-yl)amino)-N-(methyl-d₃)pyridazine-3-carboxamide

The title compound was prepared according to General Procedure F

6-Amino-4-((3-(1-fluorospiro[2.2]pentan-1-yl)pyridin-2-yl)amino)-N-(methyl-d₃)pyridazine-3-carboxamide (50.0 mg, 151 umol), (1S,2S)-2-fluorocyclopropanecarboxylic acid (64.8 mg, 604 umol) and TCFH (436 mg, 1.51 mmol) were stirred in DMF (2.00 mL) and 1-methylimidazole (245 uL, 3.02 mmol) was added. The reaction was stirred at 50° C. for 3 h then cooled to room temperature and diluted with EtOAc. The organic portion was washed with saturated NH₄Cl and brine, the organic portion was dried over anh. Na₂SO₄, filtered and concentrated under vacuum. The residue was taken up in DMSO and purified by Büchi Pure C850 Benchtop Semi-Prep (C18 silica with 10 mM Ammonium Bicarbonate Buffer pH 10.3/MeCN 5%-95%) to afford the title compound (12.9 mg, 10%). LCMS Method C (m/z): [M+H]⁺=418.4 retention time=2.22 min. ¹H NMR (400 MHz, DMSO-d₆) δ 11.82 (s, 1H), 11.38 (s, 1H), 9.64 (d, J=5.9 Hz, 1H), 9.21 (s, 1H), 8.41 (d, J=3.7 Hz, 1H), 7.95 (d, J=6.5 Hz, 1H), 7.14 (dd, J=7.4, 5.0 Hz, 1H), 5.13-4.80 (m, 1H), 2.37-2.26 (m, 1H), 1.86-1.75 (m, 1H), 1.75-1.59 (m, 2H), 1.58-1.47 (m, 1H), 1.25-1.10 (m, 2H), 0.95-0.74 (m, 2H).

### Example 37A: 6-(cyclopropanecarboxamido)-4-((5-fluoro-3-(1-fluorospiro[2.2]pentan-1-yl)pyridin-2-yl)amino)-N-(methyl-d3)pyridazine-3-carboxamide (Compound 69)

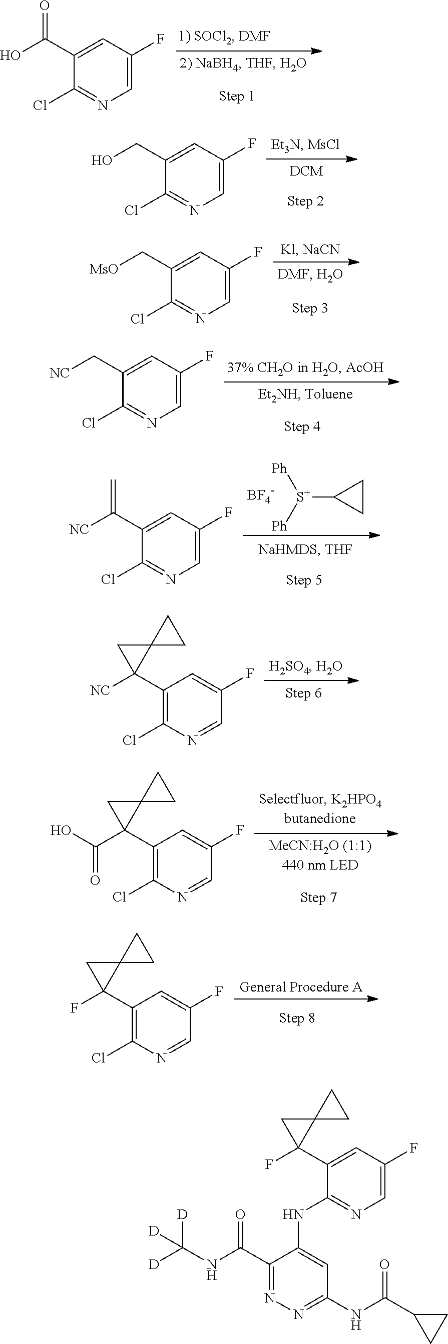

### Step 1: Synthesis of (2-chloro-5-fluoropyridin-3-yl)methanol

A flame-dried 2 L round bottom flask equipped with a Teflon®-coated stir bar and rubber septum was charged with 2-chloro-5-fluoropyridine-3-carboxylic acid (25.0 g, 138 mmol), followed by DCM (460 mL) (no dissolution), and DMF (1.07 mL, 13.8 mmol). The heterogeneous reaction mixture was cooled down to 0° C., at which time SOCl₂ (102 mL, 1.38 mol) was added slowly (white slurry). After complete addition, stirring continued at 0° C. for 20 min. The reaction was warmed up to room temperature, and stirring continued for another 15 min. The reaction mixture was concentrated under reduced pressure. The resulting residue was dissolved in THF (100 mL) and concentrated to dryness. And the resulting tan cake was dried under vacuum.

A 2 L round bottom flask was charged with ice (~230 ml) and water (~350 mL), kept in an ice bath, and was added NaBH₄ (14.5 g, 367 mmol) in portions. To this crushed ice reaction mixture was added dropwise (via addition funnel) a solution of the crude tan intermediate in THF (116 mL) over 30 min. After addition, the stirring continued for 15 min, warned up to room temperature, stirring continued for another 15 min. EtOAc (1 L) was added followed by 5 M aq. NaOH (200 mL). The mixture was stirred for 1 hour, the layers were separated and the aqueous layer was extracted with EtOAc (2×250 mL). The combined organics were dried over anh. MgSO4, filtered, and concentrated under reduced pressure to afford (2-chloro-5-fluoropyridin-3-yl)methanol (7.90 g, 34%) as a white solid. The compound was carried forward to the next step without further purification. LCMS Method B (m/z): [M+H]⁺=162.3, retention time=0.49 min.

### Step 2: Synthesis of (2-chloro-5-fluoropyridin-3-yl)methyl methanesulfonate

A flame-dried 1 L round bottom flask equipped with a Teflon®-coated stir bar and rubber septum was charged with (2-chloro-5-fluoropyridin-3-yl)methanol (7.90 g, 48.9 mmol), followed by DCM (489 mL), and Et₃N (10.3 mL, 73.3 mmol). The reaction mixture was cooled down to 0° C. in an ice bath and methanesulfonyl chloride (4.18 mL, 53.8 mmol) was added dropwise. After addition, stirring continued at the same temperature for 15 min. Saturated aq. NaHCO₃ (300 mL) was added. The reaction was warmed up to room temperature, the layers were separated and the aqueous layers extracted with DCM (200 mL). The combined organics were dried over MgSO₄, filtered, and concentrated under reduced pressure to afford (2-chloro-5-fluoropyridin-3-yl)methyl methanesulfonate as a brown oil which was carried forward to the next step without further purification. LCMS Method B (m/z): [M+H]⁺=240.3, retention time=0.72 min.

### Step 3: Synthesis of 2-(2-chloro-5-fluoropyridin-3-yl)acetonitrile

To a solution of (2-chloro-5-fluoropyridin-3-yl)methyl methanesulfonate (11.7 g, 48.8 mmol) in DMF (55.5 mL) at 0° C., was added KI (973 mg, 5.86 mmol), followed by slow addition of a solution of NaCN (2.86 g, 56.6 mmol) in water (55.5 mL). After addition, the reaction mixture was heated to 60° C., stirring continued for 15 min. The reaction mixture was cooled to room temperature, diluted with EtOAc (300 mL), and, saturated aq. NaHCO₃ was added (300 mL). The mixture was stirred for 5 min, the layers separated, and the organic portion was washed with brine (2×250 mL). The aqueous layers were combined and were back-extracted with EtOAc (250 mL). The combined organics were dried over MgSO₄, filtered, adsorbed on silica gel, and purified by normal phase flash chromatography (0-100% DCM/Heptanes) to afford 2-(2-chloro-5-fluoropyridin-3-yl)acetonitrile (2.50 g, 30%) as a colorless oil. LCMS Method B (m/z): [M−H]−169.1, retention time=0.62 min.

### Step 4: Synthesis of 2-(2-chloro-5-fluoropyridin-3-yl)acrylonitrile

In a 25 mL round-bottomed flask equipped with a magnetic stirring bar, formaldehyde, 37 wt % in H₂O (1.96 mL, 26.4 mmol) and acetic acid (1.02 mL, 17.6 mmol) were combined, and toluene (7.33 mL) was added to facilitate dissolution. Diethylamine (670 uL, 6.45 mmol) was introduced to the solution, which was then stirred at ambient temperature for 1 hour. Subsequently, the corresponding 2-(2-chloro-5-fluoropyridin-3-yl)acetonitrile (1.00 g, 5.86 mmol) was dissolved in toluene and added dropwise to the reaction mixture and allowed to stir at room temperature for 40 h at which point the solvent was removed under reduced pressure. The resulting residue was purified by normal phase chromatography (0-100% Heptanes/DCM) to afford 2-(2-chloro-5-fluoropyridin-3-yl)acrylonitrile (750 mg, 70%) as a white solid. LCMS Method B (m/z): [M+H]⁺=no ionization, retention time=0.81 min.

### Step 5: Synthesis of 1-(2-chloro-5-fluoropyridin-3-yl)spiro[2.2]pentane-1-carbonitrile

2-(2-Chloro-5-fluoropyridin-3-yl)acrylonitrile (750 mg, 4.11 mmol) and cyclopropyldiphenylsulfonium tetrafluoroborate (1.98 g, 6.16 mmol) were suspended in dry THF (41.1 mL) and the flask was purged with nitrogen and sonicated 2 mins. The mixture was cooled in an ice bath with stirring under nitrogen and 1 M NaHMDS (6.16 mL, 6.16 mmol) in THF was added dropwise. After 1 h, the reaction was quenched with saturated NH₄Cl. The mixture was extracted with EtOAc and the combined organics washed with brine, dried over anh. Na₂SO₄ and concentrated under vacuum. The residue was filtered through 30 g of silica gel using Heptanes as eluent, this removed most of the diphenyl sulfide byproduct. The column was flushed with 50/50 Heptanes/DCM to remove the desired product. This filtrate was concentrated under vacuum to afford the title compound 1-(2-chloro-5-fluoropyridin-3-yl)spiro[2.2]pentane-1-carbonitrile (710 mg, 78%) as a pale yellow oil which was carried forward without purification. LCMS Method B (m/z): [M+H]⁺=no ionization, retention time=1.00 min.

### Step 6: Synthesis of 1-(2-chloro-5-fluoropyridin-3-yl)spiro[2.2]pentane-1-carboxylic acid

1-(2-chloro-5-fluoropyridin-3-yl)spiro[2.2]pentane-1-carbonitrile (650 mg, 2.92 mmol) was dissolved in a 1:1 mixture of water (8.59 mL) and H₂SO₄ (8.59 mL). The reaction mixture was heated to 110° C. for 16 h. The reaction mixture was cooled, and ice and EtOAc was added. The pH was adjusted to 9-10 (5 M NaOH), and the phases were separated. The organic phase was discarded. The aqueous phase was reacidified using 6 M HCl to pH 0-1 and extracted with EtOAc (3×). The combined organic layers were dried over Na₂SO₄ and evaporated to dryness to give 1-(2-chloro-5-fluoropyridin-3-yl)spiro[2.2]pentane-1-carboxylic acid (600 mg, 51%) as a crude tan oil. LCMS Method B (m/z): [M−H]⁻=240.2, retention time=0.42 min.

### Step 7: Synthesis of 2-(2-chloro-5-fluoropyridin-3-yl)acrylonitrile

To a mixture of 1-(2-chloro-5-fluoropyridin-3-yl)spiro[2.2]pentane-1-carboxylic acid (600 mg, 1.49 mmol), Selectfluor® (1.69 g, 4.77 mmol) was added MeCN (7.45 mL)/H₂O (7.45 mL). The reaction mixture was degassed with nitrogen for 5 min. To this mixture was then added 2,3-Butanedione (4.65 uL, 0.0520 mmol) and stirred for 1 h under the irradiation of 440 nm LED. The reaction mixture was extracted with EtOAc (3×), washed with sat. NaHCO₃, brine, and dried over anh. Na₂SO₄. The combined organics were filtered and concentrated under vacuum to afford 2-chloro-5-fluoro-3-(1-fluorospiro[2.2]pentan-1-yl)pyridine (75.0 mg, 26%) as a beige oil which was carried forward without purification. LCMS Method B (m/z): [M+H]⁺=no ionization, retention time=1.20 min.

### Step 8: Synthesis of 6-(cyclopropanecarboxamido)-4-((5-fluoro-3-(1-fluorospiro[2.2]pentan-1-yl)pyridin-2-yl)amino)-N-(methyl-d₃)pyridazine-3-carboxamide

The title compound was prepared according to General Procedure A.

To a degassed mixture of 2-chloro-5-fluoro-3-(1-fluorospiro[2.2]pentan-1-yl)pyridine (74.7 mg, 0.346 mmol), 4-amino-6-(cyclopropanecarboxamido)-N-(methyl-d₃)pyridazine-3-carboxamide (75.0 mg, 0.315 mmol), dcpf (76.4 mg, 0.126 mmol), and K₃PO₄ (205 mg, 0.944 mmol) in dioxane (3.15 mL) was added Pd₂(dba)₃ (60.7 mg, 0.0630 mmol). Nitrogen was immediately bubbled to the mixture for 2 min and the reaction was stirred at 120° C. for 1 h. The mixture was cooled, diluted with 50% EtOAc/MeOH and filtered on Celite®. The Celite® cake was rinsed with EtOAc and the combined organic was concentrated under reduced pressure. The residue was purified by normal phase chromatography (0-20% iPrOH/DCM) to afford 6-(cyclopropanecarboxamido)-4-((5-fluoro-3-(1-fluorospiro[2.2]pentan-1-yl)pyridin-2-yl)amino)-N-(methyl-d₃)pyridazine-3-carboxamide (14.0 mg, 11%). LCMS Method L (m/z): [M+H]⁺=418.2, retention time=3.21 min. ¹H NMR (400 MHz, DMSO-d₆) δ 11.80 (s, 1H), 11.35 (s, 1H), 9.52-9.37 (m, 1H), 9.20 (s, 1H), 8.41 (s, 1H), 8.02-7.96 (m, 1H), 2.16-2.08 (m, 1H), 1.84-1.77 (m, 2H), 1.52-1.45 (m, 1H), 1.18-1.11 (m, 1H), 0.89-0.79 (m, 6H).

### Examples 37B-37E

The following examples were prepared in a manner similar to Compound 69 described in Example 37A TTT-250:

TABLE 15

| | | | | (m/z) | |
|---|---|---|---|---|---|
| | | | MW | [M + H]⁺ | |
| Ex. No. | Cmpd No. | Structure | (g/mol) | (method) | ¹H NMR |
| 37B | 73 | 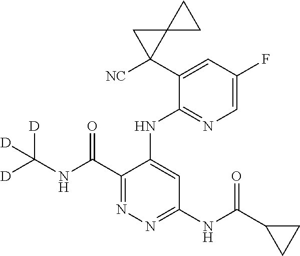 [SMILES: [2H]C([2H])([2H])NC(=O)c1nnc(NC(=O)C2CC2)cc1Nc1ncc(F)cc1C1(C#N)CC12CC2] | | 425.3 (G) | ¹H NMR (400 MHZ, DMSO-d₆) δ 11.88 (s, 1H), 11.34 (s, 1H), 9.41 (s, 1H), 9.25 (s, 1H), 8.37 (d, J = 2.9 Hz, 1H), 7.96 (dd, J = 8.9, 2.9 Hz, 1H), 2.27-2.19 (m, 1H), 2.14-2.02 (m, 2H), 1.72-1.61 (m, 1H), 1.29-1.21 (m, 1H), 1.13-1.03 (m, 1H), 0.99-0.88 (m, 1H), 0.87-0.76 (m, 4H). |
| 37C | 68 | 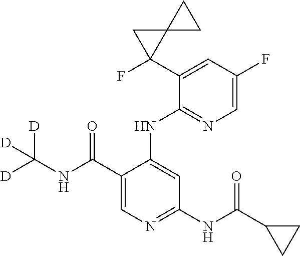 [SMILES: [2H]C([2H])([2H])NC(=O)c1cnc(NC(=O)C2CC2)cc1Nc1ncc(F)cc1C1(F)CC12CC2] | 416.44 | 417.2 (L) | ¹H NMR (400 MHZ, DMSO-d₆) δ 11.20 (s, 1H), 10.75 (s, 1H), 8.97 (s, 1H), 8.62 (s, 1H), 8.54 (s, 1H), 8.41-8.33 (m, 1H), 7.97-7.91 (m, 1H), 2.04-1.96 (m, 1H), 1.81-1.72 (m, 2H), 1.50-1.43 (m, 1H), 1.15-1.09 (m, 1H), 0.83-0.73 (m, 6H). |

### Example 38: 6-(cyclopropanecarboxamido)-4-((3-(1-fluorospiro[2.2]pentan-1-yl)-5-methoxypyridin-2-yl)amino)-N-(methyl-d3)pyridazine-3-carboxamide: (Compound 111)

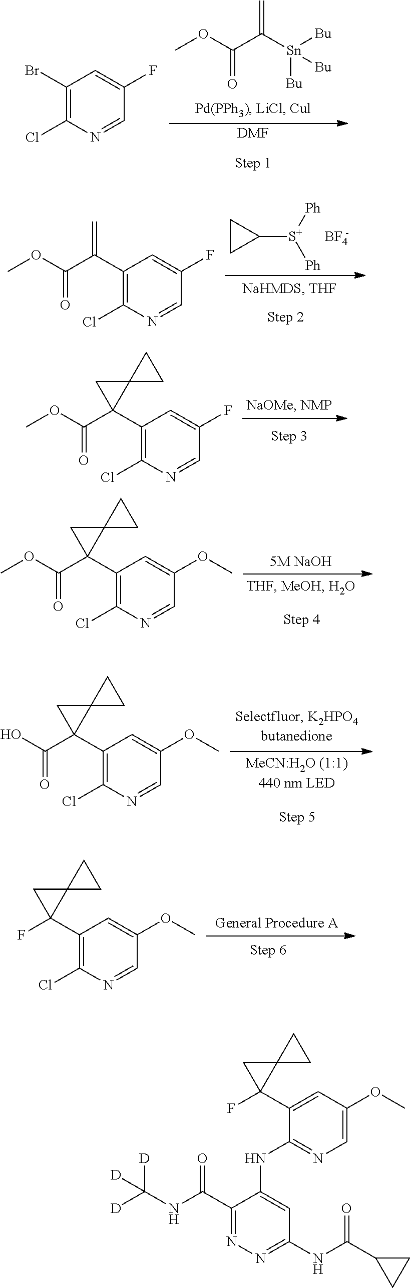

### Step 1: Synthesis of methyl 2-(2-chloro-5-fluoropyridin-3-yl)acrylate

To a solution of 3-bromo-2-chloro-5-fluoropyridine (1.01 g, 4.70 mmol), 2-(tributylstannyl)acrylic acid methyl ester (2.79 g, 7.06 mmol) in DMF (31.4 mL) was added tetrakis(triphenylphosphine)palladium(0) (555 mg, 0.470 mmol), lithium chloride (302 mg, 7.06 mmol), copper(I) iodide (672 mg, 3.53 mmol) and the mixture was degassed with nitrogen for 3 min. The reaction was stirred for 18 h, was quenched with sat. NaHCO₃, then the salts were removed by vacuum filtration. The filtrate was extracted with EtOAc, and the combined organics were dried over anh. Na₂SO₄, filtered and concentrated under vacuum. The brown semi-solid was carried forward without further purification. LCMS Method R (m/z): [M+H]⁺=no ionization, retention time=0.92 min.

### Step 2: Synthesis of methyl 1-(2-chloro-5-fluoropyridin-3-yl)spiro[2.2]pentane-1-carboxylate

Methyl 2-(2-chloro-5-fluoropyridin-3-yl)acrylate (1.01 g, 4.68 mmol) and cyclopropyldiphenylsulfonium tetrafluoroborate (1.86 g, 5.62 mmol) were suspended in dry THF (46.8 mL) and the flask was purged with nitrogen and sonicated for 2 mins. The mixture was cooled in an ice bath with stirring under nitrogen and NaHMDS (5.62 mL, 5.62 mmol) 1 M in THF was added dropwise. The reaction was stirred for 1 h and quenched with saturated NH₄Cl. The aqueous portion was extracted with EtOAc and the combined organics were washed with brine, dried over anh. Na₂SO₄, filtered and adsorbed onto silica gel. The residue was purified by normal phase chromatography 0-100% DCM/Heptanes to afford the title compound (515 mg, 43%) as a pale yellow oil. LCMS Method R (m/z): [M+H]⁺=no ionization, retention time=0.93 min.

### Step 3: Synthesis of methyl 1-(2-chloro-5-methoxypyridin-3-yl)spiro[2.2]pentane-1-carboxylate

To a solution of methyl 1-(2-chloro-5-fluoropyridin-3-yl)spiro[2.2]pentane-1-carboxylate (165 mg, 0.645 mmol) in NMP (6.45 mL) was added 25% sodium methoxide in MeOH (64.5 uL, 6.45 mmol). The reaction was stirred at room temperature overnight. Upon completion, the reaction was poured into water and extracted with EtOAc (3×), washed with brine, dried over anh. Na₂SO₄, filtered, and concentrated under vacuum to afford methyl 1-(2-chloro-5-methoxypyridin-3-yl)spiro[2.2]pentane-1-carboxylate (105 mg, 61%) as a pale yellow oil which was carried forward without purification. LCMS Method R (m/z): [M+H]⁺=268.0, retention time=1.05 min.

### Step 4: Synthesis of 1-(2-chloro-5-methoxypyridin-3-yl)spiro[2.2]pentane-1-carboxylic acid

To a solution of methyl 1-(2-chloro-5-methoxypyridin-3-yl)spiro[2.2]pentane-1-carboxylate (175 mg, 0.654 mmol) in THF (3.27 mL), water (1.63 mL), and MeOH (1.63 mL) was added 5 M aq. NaOH (1.31 mL, 6.54 mmol). The reaction was stirred at room temperature for 16 h. Upon completion, the reaction mixture was poured into a separatory funnel and was washed with DCM (2×). The aqueous layer was acidified with aq. 5 M HCl and extracted with 4:1 DCM:MeOH (×3). The organics were dried over anh. Na₂SO₄, filtered and concentrated under vacuum to afford 1-(2-chloro-5-methoxypyridin-3-yl)spiro[2.2]pentane-1-carboxylic acid (105 mg, 63%) as a brown oil. LCMS Method R (m/z): [M+H]⁺=254.0, retention time=0.58 min.

### Step 5: Synthesis of 2-chloro-3-(1-fluorospiro[2.2]pentan-1-yl)-5-methoxypyridine

To a mixture of 1-(2-chloro-5-methoxypyridin-3-yl)spiro[2.2]pentane-1-carboxylic acid (105 mg, 414.0 umol), Selectfluor® (557 mg, 1.57 mmol) and K₂HPO₄ (184 mg, 1.03 mmol) was added MeCN (3.10 mL)/water (1.03 mL). The reaction mixture was degassed with nitrogen for 5 min. To this mixture was then added 2,3-butanedione (7.35 mg, 0.0828 mmol) and stirred for 2 h under the irradiation of 440 nm LED. The reaction mixture was filtered through a silica and Celite® plug, was extracted with EtOAc 3×, washed with sat. NaHCO₃, brine, and dried over anh. Na₂SO₄. The organics were filtered and concentrated under vacuum to afford 2-chloro-3-(1-fluorospiro[2.2]pentan-1-yl)-5-methoxypyridine (80.0 mg, 85%) as a beige oil which was used without further purification. LCMS Method R (m/z): [M+H]⁺=no ionization, retention time=1.12 min.

### Step 6: Synthesis of 6-(cyclopropanecarboxamido)-4-((3-(1-fluorospiro[2.2]pentan-l-yl)-5-methoxypyridin-2-yl)amino)-N-(methyl-d₃)pyridazine-3-carboxamide

To a degassed mixture of 2-chloro-3-(1-fluorospiro[2.2]pentan-1-yl)-5-methoxypyridine (86.0 mg, 0.378 mmol), 4-amino-6-(cyclopropanecarboxamido)-N-(methyl-d₃)pyridazine-3-carboxamide (60.0 mg, 0.252 mmol), dcpf (61.1 mg, 0.101 mmol), and K₃PO₄ (218 mg, 1.01 mmol) in dioxane (2.52 mL) was added Pd₂(dba)₃ (48.5 mg, 0.0504 mmol). Nitrogen was immediately bubbled into the mixture for 2 mins and the reaction was stirred at 110° C. for 2 h. The mixture was cooled to room temperature, diluted with 50% EtOAc/MeOH and filtered through Celite®. The Celite® cake was rinsed with EtOAc and the filtrate was concentrated under reduced pressure. The residue was purified by Prep-HPLC (10-100% MeCN/10 mM AmB buffer) to afford 6-(cyclopropanecarboxamido)-4-((3-(1-fluorospiro[2.2]pentan-1-yl)-5-methoxypyridin-2-yl)amino)-N-(methyl-d₃)pyridazine-3-carboxamide (6.78 mg, 6.0%). LCMS Method T (m/z): [M+H]⁺=430.2, retention time=2.70 min. ¹H NMR (400 MHz, DMSO-d₆) δ 11.52 (s, 1H), 11.30 (s, 1H), 9.29 (s, 1H), 9.17 (s, 1H), 8.16-8.10 (m, 1H), 7.62-7.56 (m, 1H), 3.88 (s, 3H), 2.14-2.06 (m, 1H), 1.82-1.73 (m, 2H), 1.47-1.39 (m, 1H), 1.15-1.10 (m, 1H), 0.89-0.82 (m, 4H), 0.81-0.75 (m, 1H), 0.75-0.68 (m, 1H).

### Example 39: 6-(cyclopropanecarboxamido)-4-((3-(1-fluorospiro[2.2]pentan-1-yl)-5-methoxypyridin-2-yl)amino)-N-(methyl-d₃)pyridazine-3-carboxamide: (Compound 112)

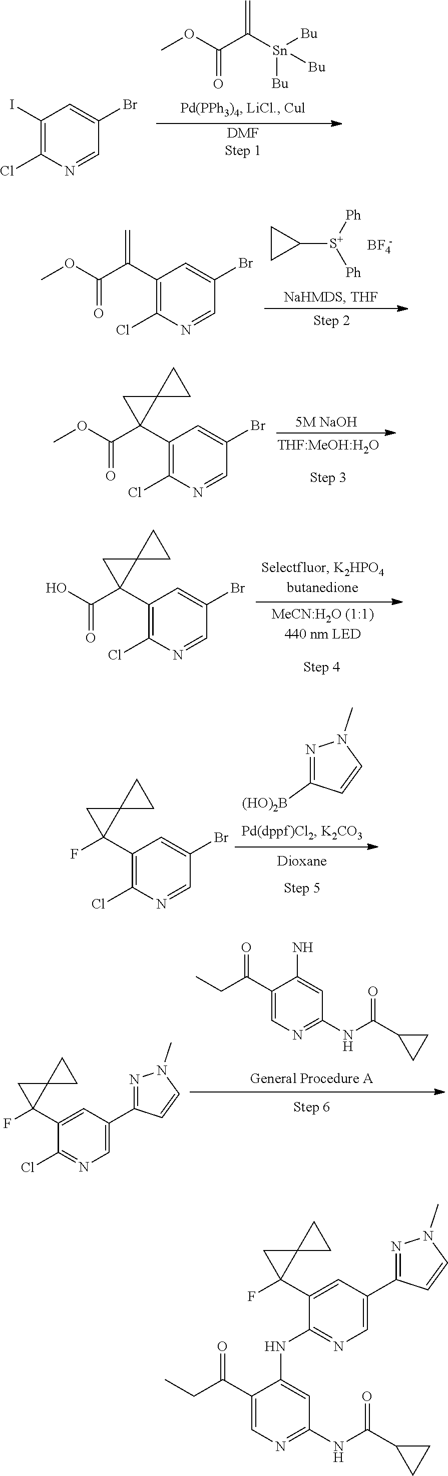

### Step 1: Synthesis of methyl 2-(5-bromo-2-chloropyridin-3-yl)acrylate

To a solution of 5-bromo-2-chloro-3-iodo-pyridine (2.29 mL, 6.16 mmol), 2-(tributylstannyl)acrylic acid methyl ester (3.04 g, 7.70 mmol) in DMF (30.8 mL) was added tetrakis(triphenylphosphine)palladium(0) (726 mg, 0.616 mmol), lithium chloride (395 mg, 9.24 mmol), and copper(I) iodide (879 mg, 4.62 mmol) and the mixture was degassed with nitrogen for 3 min. The reaction was stirred for 18 h at room temperature. The reaction mixture was quenched with sat. NaHCO₃, stirred for 15 minutes, then the salts were filtered off. The filtrate was extracted with EtOAc, washed with brine, dried over anh. Na₂SO₄ and concentrated under vacuum. The residue was dry-loaded onto silica and purified by normal phase chromatography (0-20% EtOAc/Heptanes). Pure fractions were combined and concentrated under vacuum to afford methyl 2-(5-bromo-2-chloropyridin-3-yl)acrylate (1.15 g, 68%) as a clear colourless oil. LCMS Method R (m/z): [M+H]⁺=no ionization, retention time=1.07 min.

### Step 2: Synthesis of methyl 1-(5-bromo-2-chloropyridin-3-yl)spiro[2.2]pentane-1-carboxylate

Methyl 2-(5-bromo-2-chloropyridin-3-yl)acrylate (1.05 g, 3.80 mmol) and cyclopropyldiphenylsulfonium tetrafluoroborate (1.63 g, 4.94 mmol) were suspended in dry THF (38.0 mL) and flask was purged with nitrogen and sonicated 2 mins. The mixture was cooled in an ice bath with stirring under nitrogen and NaHMDS (4.94 mL, 4.94 mmol) 1 M in THF was added dropwise. After 15 min. the reaction was complete and quenched with saturated NH₄Cl. The aqueous portion was extracted with EtOAc and the combined organics washed with brine, dried over anh. Na₂SO₄, filtered and concentrated under vacuum. The residue was taken to the next step without further purification. LCMS Method R (m/z): [M+H]⁺=no ionization, retention time=1.21 min. ¹H NMR (400 MHz, CDCl3) δ 8.36 (d, J=2.4 Hz, 1H), 7.75 (d, J=2.4 Hz, 1H), 3.66 (s, 3H), 2.06 (d, J=4.1 Hz, 1H), 1.70 (d, J=4.2 Hz, 1H), 1.27-1.17 (m, 3H), 1.15-1.10 (m, 1H).

### Step 3: Synthesis of 1-(5-bromo-2-chloropyridin-3-yl)spiro[2.2]pentane-1-carboxylic acid

To a solution of methyl 1-(5-bromo-2-chloropyridin-3-yl)spiro[2.2]pentane-1-carboxylate (1.20 g, 3.79 mmol) in THF (15.2 mL), MeOH (7.58 mL), and water (7.58 mL) was added 5 M aq. NaOH (7.58 mL, 37.9 mmol). The reaction was stirred at 40° C. for 18 h. The reaction mixture was poured into a separatory funnel and was washed with DCM (2×). The aqueous layer was acidified with 5 M HCl and extracted with EtOAc (×3). The organics were dried over anh. Na₂SO₄, filtered and concentrated under vacuum to afford 1-(5-bromo-2-chloropyridin-3-yl)spiro[2.2]pentane-1-carboxylic acid. as a beige oil which was used in the next step without further purification LCMS Method R (m/z): [M+H]⁺=303.9, retention time=0.66 min.

### Step 4: Synthesis of 5-bromo-2-chloro-3-(1-fluorospiro[2.2]pentan-1-yl)pyridine

To a mixture of 1-(5-bromo-2-chloropyridin-3-yl)spiro[2.2]pentane-1-carboxylic acid (1.06 g, 3.50 mmol), Selectfluor® (4.72 g, 13.3 mmol), K₂HPO₄ (1.56 g, 8.76 mmol) was added MeCN (26.3 mL) and water (8.76 mL). The reaction mixture was degassed with nitrogen for 5 min. To this mixture was then added 2,3-butanedione (62.2 mg, 0.701 mmol) and stirred for 1 h under the irradiation of 440 nm LED. The reaction mixture was filtered through a silica and Celite® plug, was extracted with EtOAc 3×, washed with sat. NaHCO₃, brine, and dried over anh. Na₂SO₄. The organics were filtered and concentrated to afford 5-bromo-2-chloro-3-(1-fluorospiro[2.2]pentan-1-yl)pyridine (750 mg, 77%) as a beige oil which was carried forward without purification. LCMS Method R (m/z): [M+H]⁺=no ionization, retention time=1.30 min.

### Step 5: Synthesis of 2-chloro-3-(1-fluorospiro[2.2]pentan-1-yl)-5-(1-methyl-1H-pyrazol-3-yl)pyridine

A degassed mixture of 5-bromo-2-chloro-3-(1-fluorospiro[2.2]pentan-1-yl)pyridine (120 mg, 0.434 mmol), 1-methyl-3-(4,4,5,5-tetramethyl-1,3,2-dioxaborolan-2-yl)-1H-pyrazole (101 mg, 0.477 mmol), K₂CO₃ (180 mg, 1.30 mmol), 1,1′-bis(diphenylphosphino)ferrocene dichloropalladium (II) (32.4 mg, 0.0434 mmol) in dioxane (4.34 mL) was stirred at 70° C. for 3 h. Upon completion, the reaction was filtered over a pad of Celite®, washed with EtOAc, and concentrated under vacuum. The residue was dry-loaded onto silica and purified by normal phase chromatography (0-50% EtOAc/Heptanes) to afford 2-chloro-3-(1-fluorospiro[2.2]pentan-1-yl)-5-(1-methyl-1H-pyrazol-3-yl)pyridine (89.0 mg, 74%) as an off-white solid. LCMS Method R (m/z): [M+H]⁺=278.0, retention time=1.14 min.

### Step 6: Synthesis of N-(4-((3-(1-fluorospiro[2.2]pentan-1-yl)-5-(1-methyl-1H-pyrazol-3-yl)pyridin-2-yl)amino)-5-propionylpyridin-2-yl)cyclopropanecarboxamide

To a degassed mixture of 2-chloro-3-(1-fluorospiro[2.2]pentan-1-yl)-5-(1-methyl-1H-pyrazol-3-yl)pyridine (89.3 mg, 0.322 mmol), N-(4-amino-5-propionylpyridin-2-yl)cyclopropanecarboxamide (60.0 mg, 0.257 mmol), dcpf (62.4 mg, 0.103 mmol), and K₃PO₄ (167 mg, 0.772 mmol) in dioxane (2.57 mL) was added Pd₂(dba)₃ (48.1 mg, 0.0514 mmol). Nitrogen was immediately bubbled through the mixture for 5 min and the reaction was stirred at 110° C. for 3 h. The mixture was cooled, diluted with 75% EtOAc/MeOH and filtered on Celite®. The Celite® cake was rinsed with EtOAc and the combined organics were concentrated under reduced pressure. The residue was purified by normal phase chromatography (0-20% iPrOH/DCM) to afford N-(4-((3-(1-fluorospiro[2.2]pentan-1-yl)-5-(1-methyl-1H-pyrazol-3-yl)pyridin-2-yl)amino)-5-propionylpyridin-2-yl)cyclopropanecarboxamide (35.8 mg, 29%). LCMS Method T (m/z): [M+H]⁺=475.2, retention time=2.99 min. ¹H NMR (400 MHz, DMSO-d₆) δ 11.69 (s, 1H), 10.92 (s, 1H), 9.29-9.22 (m, 1H), 8.95-8.91 (m, 1H), 8.85-8.75 (m, 1H), 8.22 (d, J=1.6 Hz, 1H), 7.78 (d, J=2.0 Hz, 1H), 6.89 (d, J=2.2 Hz, 1H), 3.91 (s, 3H), 3.20-3.07 (m, 2H), 2.09-2.01 (m, 1H), 1.87-1.78 (m, 2H), 1.34-1.28 (m, 1H), 1.20-1.14 (m, 1H), 1.11 (t, J=7.2 Hz, 3H), 0.90-0.78 (m, 5H), 0.78-0.71 (m, 1H).

### Example 40A: 6-(6-chloro-5-(1-fluorospiro[2.2]pentan-1-yl)pyridin-3-yl)-2-oxa-6-azaspiro[3.3]heptane (Compound 113)

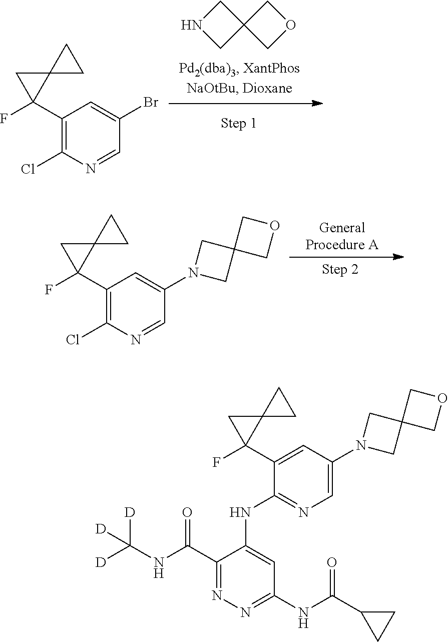 [SMILES: FC1(c2cc(Br)cnc2Cl)CC12CC2 ; C1NCC12COC2 ; FC1(c2cc(N3CC4(COC4)C3)cnc2Cl)CC12CC2 ; [2H]C([2H])([2H])NC(=O)c1nnc(NC(=O)C2CC2)cc1Nc1ncc(N2CC3(COC3)C2)cc1C1(F)CC12CC2]

### Step 1: Synthesis of 6-(6-chloro-5-(1-fluorospiro[2.2]pentan-1-yl)pyridin-3-yl)-2-oxa-6-azaspiro[3.3]heptane

A degassed mixture of 5-bromo-2-chloro-3-(1-fluorospiro[2.2]pentan-1-yl)pyridine (120 mg, 0.434 mmol), 2-oxa-6-azaspiro[3.3]heptane (65.2 mg, 0.651 mmol), XantPhos (51.2 mg, 0.0868 mmol), sodium tert-butoxide (86.0 mg, 0.868 mmol), and Pd₂(dba)₃ (40.5 mg, 0.0434 mmol) in dioxane (4.34 mL) was stirred at 70° C. for 2 h. Upon completion, the reaction was filtered over a pad of Celite®, washed with EtOAc, and concentrated. The residue was dry-loaded onto silica and purified by normal phase chromatography (0-50% EtOAc/Heptanes) to afford 6-(6-chloro-5-(1-fluorospiro[2.2]pentan-1-yl)pyridin-3-yl)-2-oxa-6-azaspiro[3.3]heptane (67.0 mg, 52%) as an off-white solid. LCMS Method R (m/z): [M+H]⁺=295.0, retention time=1.08 min.

### Step 2: Synthesis of 6-(cyclopropanecarboxamido)-4-((3-(1-fluorospiro[2.2]pentan-1-yl)-5-(2-oxa-6-azaspiro[3.3]heptan-6-yl)pyridin-2-yl)amino)-N-(methyl-d₃)pyridazine-3-carboxamide

To a degassed mixture of 6-(6-chloro-5-(1-fluorospiro[2.2]pentan-1-yl)pyridin-3-yl)-2-oxa-6-azaspiro[3.3]heptane (69.6 mg, 0.236 mmol), 4-amino-6-(cyclopropanecarboxamido)-N-(methyl-d₃)pyridazine-3-carboxamide (45.0 mg, 0.189 mmol), dcpf (45.8 mg, 0.0755 mmol), and K₃PO₄ (123 mg, 0.567 mmol) in dioxane (1.89 mL) was added Pd₂(dba)₃ (35.3 mg, 0.0378 mmol). The reaction mixture was degassed for another 5 min, sealed, and allowed to stir at 120° C. for 20 h. The mixture was cooled, diluted with 75% EtOAc/MeOH and filtered on Celite®. The Celite® cake was rinsed with EtOAc and was concentrated under reduced pressure. The residue was purified directly on the Prep-HPLC (15%-100% MeCN/10 mM AmB buffer) to afford 6-(cyclopropanecarboxamido)-4-((3-(1-fluorospiro[2.2]pentan-1-yl)-5-(2-oxa-6-azaspiro[3.3]heptan-6-yl)pyridin-2-yl)amino)-N-(methyl-d₃)pyridazine-3-carboxamide (15.9 mg, 17%). LCMS Method T (m/z): [M+H]⁺=497.2, retention time=2.45 min. ¹H NMR (400 MHz, DMSO-d₆) δ 11.30 (s, 1H), 11.23 (s, 1H), 9.15-9.04 (m, 2H), 7.68-7.62 (m, 1H), 7.09-7.02 (m, 1H), 4.71 (s, 4H), 4.08 (s, 4H), 2.12-2.06 (m, 1H), 1.76-1.68 (m, 2H), 1.43-1.34 (m, 1H), 1.13-1.07 (m, 1H), 0.89-0.79 (m, 4H), 0.78-0.69 (m, 1H), 0.68-0.59 (m, 1H).

### Examples 40B-40C

The following examples were prepared in a manner similar to Compound 113 described in Example 40A:

TABLE 16

| | | | | (m/z) | |
|---|---|---|---|---|---|
| Ex | Cmpd | | MW | [M + H]⁺ | |
| No. | No. | Structure | (g/mol) | (method) | ¹H NMR |
| 40B | 114 | 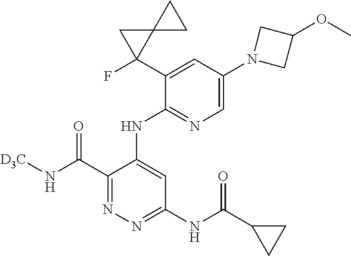 [SMILES: [2H]C([2H])([2H])NC(=O)c1nnc(NC(=O)C2CC2)cc1Nc1ncc(N2CC(OC)C2)cc1C1(F)CC12CC2] | 484.54 | 485.2 (T) | ¹H NMR (400 MHZ, DMSO-d₆) δ 11.31 (s, 1H), 11.22 (s, 1H), 9.13 (s, 1H), 9.10 (s, 1H), 7.68- 7.63 (m, 1H), 7.10-7.05 (m, 1H), 4.37-4.31 (m, 1H), 4.17- 4.10 (m, 2H), 3.75-3.68 (m, 2H), 3.25 (s, 3H), 2.13-2.06 (m, 1H), 1.77-1.67 (m, 2H), 1.43-1.36 (m, 1H), 1.10 (dt, J = 9.2, 4.6 Hz, 1H), 0.89-0.80 (m, 4H), 0.79-0.70 (m, 1H), 0.65 (dt, J = 9.3, 4.7 Hz, 1H). |
| 40C | 115 | 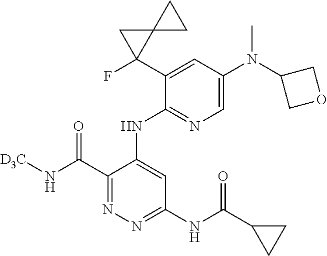 [SMILES: [2H]C([2H])([2H])NC(=O)c1nnc(NC(=O)C2CC2)cc1Nc1ncc(N(C)C2COC2)cc1C1(F)CC12CC2] | 484.54 | 485.2 (T) | ¹H NMR (400 MHZ, DMSO-d₆) δ 11.38 (s, 1H), 11.24 (s, 1H), 9.15 (s, 1H), 9.12 (s, 1H), 7.86- 7.78 (m, 1H), 7.35-7.29 (m, 1H), 4.87-4.74 (m, 3H), 4.64- 4.57 (m, 2H), 2.94 (s, 3H), 2.15- 2.06 (m, 1H), 1.79-1.70 (m, 2H), 1.45-1.37 (m, 1H), 1.14- 1.07 (m, 1H), 0.89-0.79 (m, 4H), 0.79-0.73 (m, 1H), 0.73- 0.64 (m, 1H). |

### Biological Assays:

### Example 41: TYK2 and JAK1 HTRF Competition Binding Assay and Inhibition of IFNα-Induced pSTAT1

### Description of TYK2-JH2 and JAK1-JH2 HTRF Biochemical Assay

A biochemical Homogeneous Time Resolved Fluorescence (HTRF) competition binding assay was performed. The assay measures the ability of a compound to displace the fluorescent signal resulting from a probe (MedChemExpress, HY-102055) binding to the pseudokinase domains (JH2) of TYK2 and JAK1 (Creative Biomart; cat lot: TYK2-703H, JAK1-07H).

For assay preparation, 55.6 nM fluorescein labeled probe (TYK2-JH2 assay, or 18.3 nM for JAK1-JH2 assay), 0.5 nM anti-6×His-terbium labeled antibody (Revvity, 61HI2TLA) plus 0.5 nM protein were prepared in assay buffer (20 mM Hepes pH 7.5, 150 mM NaCl, 10 mM MgCl2, 2 mM DTT, 0.05 mg/mL BSA, and 0.015% Brij 35). Solution mixture was incubated at room temperature in the dark for 30 mins. A series of 8 three-fold serial dilutions were performed using a Tecan D300e Digital Dispenser to give 9 concentrations ranging from 1000 nM down to 0.15 nM in 384 well plate (Revvity, 6008280). Ten microliters (10 uL) of solution mixture were added to the compound plate, with 1% DMSO final. After 1 h at 23° C. in dark, the HTRF signal (ratio of emission 520 nm/emission 495 nm) was measured on an Envision 2 plate reader. Percent inhibition is calculated by comparison to a control without protein (Negative control, NC) and without inhibitor (positive control, PC). Inhibition %=100−(100*(signal compound−signal NC)/(signal PC−signal NC)). TYK2/JAK1 fold selectivity defined as the ratio of IC50 JAK1-JH2/IC50 TYK2-JH2.

Inhibition of IFNα Signaling Through pSTAT1 in THP-1 Cells

The day before the assay, THP-1 cells were diluted at 400,000 cells/mL in RPMI (Multicell 350-000-CL) containing 10% heat-inactivated FBS (Multicell 098-450), 10 mM HEPES (Multicell 330-050-EL) and 0.055 mM 2-Mercaptoethanol (Gibco 21985023). For assay preparation, cells were prepared in HBSS (Gibco 14025092) at 5,000,000 cells/mL and dispensed at 20,000 cells/well with Multidrop Combi+(ThermoFisher Scientific) into ProxiPlate Shallow Well Plus, white, tissue culture treated 384-well plates (Revvity 8313-24345). Cells were allowed to incubate at 37° C. under 5% CO2 conditions for 2 hours. Compounds solubilized in DMSO were serially diluted 1:3 with Tecan D300e Digital Dispenser to produce ten points of 3-x concentrated dilutions starting from 3000 nM, in an intermediate 384-well plate (Greiner 781280), with HBSS containing 0.3% BSA (Millipore Sigma A7906) and normalized to 0.6% DMSO final. Cells were pre-treated with serially diluted compounds for 1 hour at 37° C., then stimulated with human IFNα (25 ng/mL final) for 15 minutes. Stimulation was stopped by the addition of 5-x concentrated alpha-LISA lysis buffer (Revvity Q2074) with cOmplete™, EDTA-free Protease Inhibitor Cocktail (Roche 4093124001) and by snap freezing on dry ice. Cell lysis was allowed to proceed for 10 minutes at room temperature on shaker at 300 rpm. The p-STAT1 (Tyr 701) AlphaLISA reaction was performed following the vendor's recommended protocol (Revvity ALSU-PST1). Alpha-LISA signal was measured using an Envision plate reader (Revvity). Percent inhibition of IFNα-induced p-STAT1 in THP-1 cells was calculated with the following formula using XLFit software (ID Business Solutions Ltd.):

$$Percentage of inhibition = 100 - ( 100 * ( {signal}_{compound} - {signal}_{non - stimulated control} ) / ( {signal}_{stimulated control} - {signal}_{non - stimulated control} ) )$$

Dose response curves from 8 concentration points were generated to determine the concentration of the test compound required to inhibit 50% of cellular response (ICso) as derived by a 4-parameter nonlinear logistic regression with Assay Capture Analysis System (LiveDesign, Schrödinger).

### TRANSIL Procedure:

The TRANSIL Brain Absorption assay is designed to evaluate the affinity of test compounds for brain membranes, thereby predicting the free fraction of these compounds in the brain. In this assay, 1 M of the test compounds was introduced to varying concentrations of lipids immobilized on silica beads. The test compounds and assay controls were prepared as concentrated stock solutions in dimethyl sulfoxide (DMSO), ensuring that the final DMSO concentration in each well was 2%. Each test compound was formulated as an individual stock solution at 16 M in phosphate-buffered saline (PBS) at pH 7.4, containing 32% DMSO. Subsequently, 15 μL of the 16 μM stock solution was added to each well to achieve a final assay concentration of 1 μM.

The samples were incubated on a plate shaker at 1000 rpm for 12 minutes. Following incubation, the plate was centrifuged at 750 g for 10 minutes, and 100 μL of the supernatant was collected. This supernatant was quenched with ice-cold acetonitrile containing 1 μM of internal standard. The quenched supernatants were then diluted with a mixture of acetonitrile:water prior to analysis by LC-MS/MS. Warfarin and fluoxetine were utilized as controls representing moderate and high brain binding, respectively. The free fraction of the drug in the brain (fu brain) was calculated using the following equation:

$${fu}_{brain} = {10}^{{b}_{1} * {logMA}_{brain} + {b}_{2}}$$

Where:
   fᵤ brain=fraction unbound using TRANSIL beads
   MA=The membrane affinity, which is a partitioning coefficient of drug between membrane and buffer.
   b₁ and b₂ are calibration coefficients provided by the manufacturer.

### TRANSIL Quality Index (TQI)

The TRANSIL Quality Index (TQI) is based on five independent measures (1. Intercept & Model fit, 2. Predicted vs measured reference signal, 3. correlation coefficient, 4. number of outliers, 5. data consistency) derived from the data analysis (more information is available in the TRANSIL_XL brain absorption kit user guide −V3.03cox). For each individual measure, a partial quality score on a scale between 0 and 10 is attributed to the estimate. 0 represents lowest quality, while 10 represents highest quality. The final quality index is a weighted average of the partial quality scores. Results with an index greater than 7 are of good quality, results with an index between 5-7 are compromised, but may be reasonable accurate, while results with an index below 5 are poor and should be reported with caution.

TABLE 17
TYK2 and JAK1 HTRF Competition Binding, Inhibition of IFNa-Induced
pSTAT1, Unbound brain fraction, and IFNa-Induced pSTAT1 IC50 adjusted for brain %
free

| | | | IFNa- | | |
|---|---|---|---|---|---|
| | | | Induced | | |
| | TYK2- | | pSTAT1 | | IC₅₀ adj. for |
| | JH2 | | IC₅₀ | Fu, brain | brain % free |
| Cmpd # | IC₅₀ (nM) | fold selectivity vs JAK1(JH2) | (nM) | (TRANSIL) | (nM)¹ |
| Deucravacitinib | 0.37 | 7 | 150 | 0.29 | 7.2 |
| 1 | 0.56 | 700 | 15 | 0.013 | 1100 |
| 7 | 0.53 | 740 | 9.2 | 0.11 | 83 |
| 10 | 0.68 | 29 | 26 | 0.048 | 520 |
| 11 | 0.44 | 63 | 8 | 0.12 | 68 |
| 2 | 0.86 | >1200 | 36 | 0.031 | 1100 |
| 6 | 0.64 | 380 | 15 | 0.14 | 110 |
| 3 | 2.80 | >360 | 200 | 0.021 | 9000 |
| 13 | 10.20 | 45 | 830 | | |
| 9 | 3.60 | >280 | 200 | 0.029 | 6600 |
| 5 | 0.80 | 630 | 20 | 0.008 | 2400 |
| 4 | 1.40 | >720 | 65 | 0.014 | 4400 |
| 8 | 1.20 | 29 | 80 | 0.011 | 6800 |
| 12 | 0.61 | 70 | 14 | 0.032 | 430 |
| 14 | 4.60 | 190 | 190 | 0.087 | 2100 |
| 15 | 5.90 | >170 | 200 | 0.16 | 1200 |
| 18 | 5.90 | >170 | 310 | 0.030 | 9800 |
| 26 | 1.20 | >480 | 33 | 0.057 | 570 |
| 17 | 0.44 | 20 | 1.9 | 0.10 | 18 |
| 16 | 2.30 | 100 | 61 | 0.066 | 910 |
| 22 | 1.10 | >890 | 31 | 0.090 | 340 |
| 27 | 1.00 | 590 | 34 | 0.055 | 610 |
| 28 | 0.70 | 75 | 17 | 0.014 | 1200 |
| 19 | 0.39 | 55 | 3.21 | 0.041 | 73 |
| 29 | 40 | >25 | >1000 | 0.010 | 9800 |
| 30 | 36 | >28 | >510 | 0.26 | 2000 |
| 31 | 2.5 | 150 | 34 | 0.015 | 2100 |
| 32 | 0.89 | 370 | 17 | 0.026 | 620 |
| 33 | 1.10 | 290 | 34 | 0.078 | 420 |
| 34 | 0.47 | 130 | 8 | 0.073 | 110 |
| 35 | 0.44 | 73 | 5.6 | 0.014 | 380 |
| 36A | 0.37 | 94 | 4.8 | 0.057 | 84 |
| 37 | 0.75 | 220 | 21 | 0.014 | 1400 |
| 38 | 1.2 | 280 | 79 | 0.035 | 2200 |
| 23 | 0.44 | 28 | 2 | 0.038 | 50 |
| 39 | 1.5 | 23 | 120 | 0.012 | 9600 |
| 40 | 0.39 | 32 | 5.2 | 0.017 | 290 |
| 96 | 0.47 | 130 | 3 | 0.052 | 56 |
| 41A | 0.81 | 290 | 20 | 0.017 | 1100 |
| 42A | 0.96 | 79 | 22 | 0.014 | 1500 |
| 97A | 0.48 | 260 | 6.5 | 0.027 | 240 |
| 97B | 0.54 | 82 | 11 | 0.026 | 390 |
| 20 | 0.34 | 70 | 3.1 | 0.017 | 170 |
| 43 | 0.62 | 370 | 14 | 0.018 | 750 |
| 44 | 2.3 | >440 | 120 | 0.033 | 3600 |
| 88 | 0.58 | 46 | 8.5 | 0.028 | 290 |
| 45 | 0.35 | 65 | 4.6 | 0.003 | 1100 |
| 46A | 0.39 | 78 | 4.3 | | |
| 47 | 7.5 | >130 | 530 | 0.031 | 16000 |
| 48 | 12 | >81 | 860 | 0.033 | 25000 |
| 49 | 0.46 | 62 | 9.9 | 0.030 | 300 |
| 50 | 0.92 | 50 | 16 | 0.015 | 980 |
| 51 | 0.94 | 180 | 37 | 0.033 | 1100 |
| 52 | 1.1 | 35 | 26 | 0.014 | 1700 |
| 53 | 0.59 | 88 | 9.9 | 0.021 | 440 |
| 54 | 3.2 | 120 | 110 | | |
| 55A | 0.96 | 100 | 22 | 0.045 | 490 |
| 21A | 0.38 | 81 | 2.8 | 0.025 | 110 |
| 56 | 0.55 | 69 | 8.1 | 0.032 | 250 |
| 57 | 18 | >56 | >1000 | | |
| 58 | 0.74 | 510 | 20 | 0.039 | 490 |
| 59 | 1.6 | 220 | 41 | 0.068 | 600 |
| 60B | 0.62 | 83 | 12 | 0.047 | 260 |
| 60A | 0.62 | 36 | 8.9 | 0.040 | 220 |
| 61 | 1.3 | 99 | 52 | 0.024 | 2100 |
| 85 | 0.40 | 88 | 7.6 | 0.002 | 2300 |
| 84 | 2.8 | >360 | | 0.15 | |
| 83 | 0.34 | 38 | 3.1 | 0.045 | 67 |
| 82 | 1.40 | 93 | 58 | 0.013 | 4300 |
| 81 | 0.46 | 21 | 0.9 | 0.02 | 42 |
| 80 | 2.2 | 270 | | 0.025 | |
| 79 | 0.34 | 400 | 3.7 | 0.070 | 53 |
| 78 | 1.3 | 120 | | 0.016 | |
| 77 | 0.83 | 300 | 11 | 0.072 | 150 |
| 76A | 0.60 | 370 | 9.7 | 0.022 | 430 |
| 75 | 0.30 | 490 | 2.9 | 0.007 | 370 |
| 73 | 2.5 | 120 | 58 | 0.050 | 1100 |
| 72 | 0.36 | 58 | 2.6 | 0.015 | 170 |
| 71A | 1.7 | 59 | 45 | 0.023 | 1900 |
| 70 | 7.4 | 95 | | 0.047 | |
| 98A | 0.78 | 110 | 12 | 0.051 | 240 |
| 69 | 0.51 | 34 | 6.6 | 0.012 | 520 |
| 68 | 0.38 | 150 | 12 | 0.030 | 380 |
| 86 | 0.76 | 140 | 44 | 0.030 | 1400 |
| 67A | 1.2 | 43 | | 0.028 | |
| 66 | 350 | >3 | | 0.066 | |
| 65 | 8.6 | 96 | >780 | 0.029 | 26000 |
| 64A | 0.37 | 38 | 1.9 | 0.033 | 56 |
| 63 | 16 | >36 | 180 | 0.14 | 1300 |
| 62 | 0.68 | 140 | 11 | 0.029 | 380 |
| 89 | 0.56 | 96 | 12 | 0.074 | 160 |
| 90 | 0.92 | 260 | 20 | 0.017 | 1100 |
| 91 | 2.3 | >240 | 77 | 0.058 | 1300 |
| 92 | 2.6 | >380 | 260 | | |
| 93 | 6.1 | >160 | | 0.018 | |
| 87 | 6.6 | 44 | | | |
| 111 | 0.51 | 39 | 5.1 | 0.022 | 220 |
| 102 | 20 | >50 | | 0.15 | |
| 103 | 20 | >49 | | 0.21 | |
| 112 | 0.76 | 190 | 11 | 0.004 | 2500 |
| 113 | 0.53 | 93 | 5.3 | 0.15 | 36 |
| 114 | 0.56 | 33 | 4.8 | 0.052 | 92 |
| 115 | 0.48 | 36 | 6.9 | 0.15 | 45 |
| 104 | 2.6 | 260 | | 0.011 | |
| 105A | 2.2 | >430 | | 0.011 | |
| 100A | 0.44 | 9 | 2.9 | 0.021 | 130 |
| 106 | 0.93 | 120 | 21 | 0.039 | 530 |
| 110 | 3.3 | >310 | | 0.023 | |
| 107 | 3.8 | >240 | >1000 | 0.033 | 29000 |
| 108A | 1.4 | 560 | 1100 | 0.031 | 36000 |
| 109 | 0.49 | 140 | 72 | 0.094 | 830 |
| 94A | 0.68 | 260 | 20 | 0.053 | 360 |
| 95 | 6.0 | 71 | 6 | | |
| 99 | 0.51 | 10 | 7.7 | 0.017 | 430 |
| 101 | 1.4 | | | 0.12 | |

${}^{1} Adj IC 50 = ( \frac{IFNa IC 50}{Fu , brain} ) \div ( serum \% ( \frac{1}{Fu , brain} - 1 ) + 1 ) ; serum \% = 0.1 \% = 0.001$

### Example 42: Mice Pharmacokinetic Study

The pharmacokinetic (PK) protocol was reviewed and assessed by the Animal Care Committee of the AdMare Innovation Center, part of AdMare Bioinnovations in Montreal, Quebec, Canada. Female C57Bl/6 mice (6-8 weeks old) were ordered from Charles River Laboratories (Raleigh, North Carolina, USA). Following arrival, all animals were group-housed in ventilated cages. Mice were provided with ad libitum access to food and water.

### Formulations:

Intravenous cassette formulation: 5% DMAc, 76% PEG-400, 19% water. All (4-5) compounds from the cassette were pre-weighed in one glass vial. PEG-400 and N—N-Dimethylacetamide (DMAc) were added and sonicated until clear. Water was added slowly under constant agitation. The resulting formulations were clear solutions.

Intravenous discrete formulations: 10% DMSO, 10% Solutol HS-15, 30% PEG-400 and 50% water. Compounds were pre-weighed in glass vials. DMSO was added and sonicated for 3 to 5 min followed by Solutol HS-15 and PEG-400. Finally, water was added slowly while vortexing, and the solution was sonicated for 5 to 30 minutes. The resulting formulations were solutions.

Oral formulations: 5% EtOH, 90% PEG-300, 5% Vit E TPGS. Excipients are premixed in advance. Compounds were pre-weighed in glass vials and the premix was added. The formulations were then sonicated for 5-30 minutes, vortexing at regular intervals. Other formulations were evaluated such as 1% NMP, 0.3% Tween-80 and 98.7% of 0.5% methylcellulose in water or amorphous suspensions.

### PK Procedure Summary:

On the morning of the PK study, all animals were weighed and the dosing formulation volumes to be delivered were calculated accordingly.

Intravenous administrations of cassette were carried at 5 mL/kg over a period of 40 sec. into the right jugular vein surgically exposed for dosing in animals under anesthesia (N=3). At selected time points 0.083, 0.25, 0.5, 1, 2, 5 and 8 hrs, the animals (n=3) were bled from the tail using heparinized capillaries (Drummond Plasticrit). Plasma was generated by centrifugation at 6,000 g×5 min at 4° C. and stored at −80° C. until analysis.

Oral administrations were performed by intragastric gavage at lOmL/kg. At selected time points 0.25, 0.5, 1, 2, 5 and 8 hrs, the animals (n=3) were bled from the tail using heparinized capillaries (Drummond Plasticrit). Plasma was generated by centrifugation at 6,000 g×5 min at 4° C. and stored at −80° C. until analysis.

Satellite groups of N=2 animals were also dosed for a terminal bleed and tissue collection at specific timepoints.

For terminal collections requiring tissue collections, animals were anesthetized with isoflurane in order to perform a cardiac puncture, followed by whole body perfusion with phosphate buffered saline (PBS, pH 7.4) to wash out any remaining blood from the organs. The brains were then harvested, rinsed in PBS, frozen on dry ice in Omni bead vials and stored at −80° C. until homogenization using an Omni Bead Ruptor (dilution 5 times in 25% Isopropyl alcohol/75% water). Plasma was prepared by centrifugation at 6,000 g, 4C, 5 min stored at −80° C. until LC-MS/MS analysis along with brain homogenates.

Bioanalysis: Samples were extracted using protein precipitation and were analyzed by reversed-phase LC-MS/MS analysis.

Non-compartmental analysis (NCA) was performed using WinNonlin 8.4 or 8.5.

Kp values are calculated: Brain concentration/plasma concentration in mouse (at specific timepoints)

TABLE 18
Ratio of exposure in brain to plasma

| | Ratio of exposure in brain to plasma |
|---|---|
| Compd No. | (6 h, 10 mpk, po) |
| 11 | 0.46 |
| 12 | 0.83 |
| 22 | 0.31 |
| 19 | 0.22 |
| 23 | 0.46 |
| 20 | 0.26 |
| 21A | 0.11 |
| 60A | 0.14 |
| 77 | 0.08 |
| 75 | 0.22 |
| 72 | 0.11 |
| 62 | 0.91 |

### Example 43: Rat Pharmacokinetic Study

The pharmacokinetic (PK) protocol was reviewed and assessed by the Animal Care Committee of the AdMare Innovation Center, part of AdMare Bioinnovations in Montreal, Qeuebec, Canada. Male Sprague-Dawley Rats from CRL were ordered from Charles River Laboratories (Raleigh, North Carolina, USA). Following arrival, all animals were group-housed in ventilated cages. Rats were provided with ad libitum access to food and water.

### Formulations:

Intravenous formulation: 10% DMSO, 10% Solutol HS-15, 30% PEG-400 and 50% water. Compounds were pre-weighed in glass vials. DMSO was added and sonicated for 3 to 5 min followed by Solutol HS-15 and PEG-400. Finally, water was added slowly while vortexing, and the solution was sonicated for 5 to 30 minutes. The resulting formulations were solutions.

Oral formulation: 5% EtOH, 90% PEG-300, 5% Vit E TPGS. Excipients are premixed in advance. Compounds were pre-weighed in glass vials and the premix vehicle was added. The formulations were then sonicated for 5-30 minutes, vortexing at regular intervals. Other formulations were evaluated such as using combinations with cyclodextrins, Tween-80, NMP as well as amorphous suspensions.

Oral formulation: 0.5% Methocel A4M/1% Soluplus in water. The Compound was formulated as a spray-dried amorphous dispersion was pre-weighed in a glass vial. A small amount of a premix 0.5% Methocel A4M/1% Soluplus in water was added and the mixture was stirred to form a paste. The remaining volume was slowly added and left stirring for an additional 10 min. The resulting formulations were uniform suspensions.

### PK Procedure Summary:

On the morning of the PK study, all animals were weighed and the dosing formulation volumes to be delivered were calculated accordingly.

Intravenous administrations were carried at 2 mL/kg into the tail vein. At selected time points 0.083, 0.25, 0.5, 1, 2, 5, 8 and 24 hrs, the animals (n=3) were bled by jugular puncture and plasma was generated by centrifugation at 6,000 g×5 min at 4° C. and stored at −80° C. until analysis.

Oral administrations were performed by intragastric gavage at 10 mL/kg. At selected time points 0.25, 0.5, 1, 2, 5 and 8 hrs, the animals (n=3) were bled by jugular puncture and at 24 hrs, by terminal cardiac puncture. Plasma was generated by centrifugation at 6,000 g×5 min at 4° C. and stored at −80° C. until analysis.

Satellite groups of N=2 animals were also dosed for a terminal bleed and tissue collection at specific timepoints.

For terminal collections requiring tissue collections, animals were anesthetized with isoflurane in order to perform a cardiac puncture, followed by whole body perfusion with phosphate buffered saline (PBS, pH 7.4) to wash out any remaining blood from the organs. The brains were then harvested, rinsed in PBS, frozen on dry ice in Omni bead vials and stored at −80° C. until homogenization (dilution 5 times in 25% Isopropyl alcohol/75% water). Plasma was prepared by centrifugation at 6,000 g, 4C, 5 min stored at −80° C. until LC-MS/MS analysis along with brain homogenates.

Bioanalysis: Samples were extracted using protein precipitation and were analyzed by reversed-phase LC-MS/MS analysis.

Non-compartmental analysis (NCA) was performed s using WinNonlin 8.5.

Kp values are calculated: Brain concentration/plasma concentration in rat (at specific timepoints).

TABLE 19
Ratio of exposure in brain to plasma

| | Ratio of exposure in |
|---|---|
| | brain to plasma |
| Compound No. | (6 h, 10 mpk, po) |
| Deucravacitinib | 0.02 |
| 11 | 0.25 |
| 12 | 0.64 |
| 22 | 0.39 |
| 19 | 0.14 |
| 23 | 0.26 |
| 20 | 0.16 |
| 76A | 1.2 |

### PD Assay

Compounds are resuspended in formulation (5% EtOH, 90% PEG300, 5% Vit E TPGS) and administered orally by gavage to non-fasted, 6-9-weeks old female C57BL/6J mice (The Jackson Laboratory). Two (2) hours after administration, mice are injected with 10 ng/g (10,000 U in 100 μL) of IFNα-2 (BioLegend 752804) by intravenous tail vein. Control group will receive intravenous vehicle (endotoxin-free certified PBS, EMD Millipore TMS-012-A).

Blood is collected by cardiac puncture and put in K₃ EDTA tubes (Sarstedt 20.1345.100). 200 μL is added to 200 μL of DNA/RNA Shield (Zymo Research R¹²⁰⁰) and frozen at −80° C. Brains (left hemisphere) are put in RNALater (Invitrogen 2896148) for 24 h before being transferred in a 7 mL bead tube (ESBE Scientific INC BER-P000935LYSKOAO) and frozen at -80° C.

Blood:RNALater samples are thawed on ice, then RNA is extracted using Quick-RNA Whole Blood RNA extraction kit (Zymo Research R1201(ZY)), as per manufacturer's instruction. RNA elution is done in 50 μL.

Brain homogenization is performed in 4 mL of RL Buffer+1% β-mercaptoethanol using Precellys 24 from Bertin Technologies for 30 sec. RNA purification is performed on 15 mg (Animal Tissue RNA Purification Kit NorgenBiotek corp. 25700), and according to manufacturer's instructions. RNA elution is done in 30 μL.

All RNAs are quantified using QuantiFluor RNA System (Promega E3310). Reverse Transcription is performed using 500 ng of RNA with High-Capacity cDNA Reverse Transcription kit (Life Technologies 4368814). Subsequently, qPCR is performed on 25 ng of cDNA using TaqMan Gene Expression Assays, listed in the table below (ThermoFisher Scientific) and Taqman Fast Advanced Master Mix (ThermoFisher Scientific 4444557), in a total reaction volume of 10 μL. Each gene analyzed is performed in duplex with the housekeeping gene Gusb.

TABLE 20
List of Taqman Assays from ThermoFisher Scientific.

| Gene | Taqman Assay | Dye | Amplicon |
|---|---|---|---|
| Cxcl10 | Mm00445235_m1 | FAM | 59 |
| Mx1 | Mm00487796_m1 | FAM | 121 |
| Usp18 | Mm01188805_m1 | FAM | 65 |
| Gusb | Mm01197698_m1 | VIC | 71 |

Delta-delta-Ct method is used to analyze qPCR data and determine fold-change of each individual compared to vehicle group. % of control is defined as the level of gene expression of each individual compared to gene expression in IFNα condition (normalized at 100%). Graphs were generated by GraphPad Prism 10 and statistical significance was determined by 2-way ANOVA with Tukey's correction for multiple comparison.

### Results

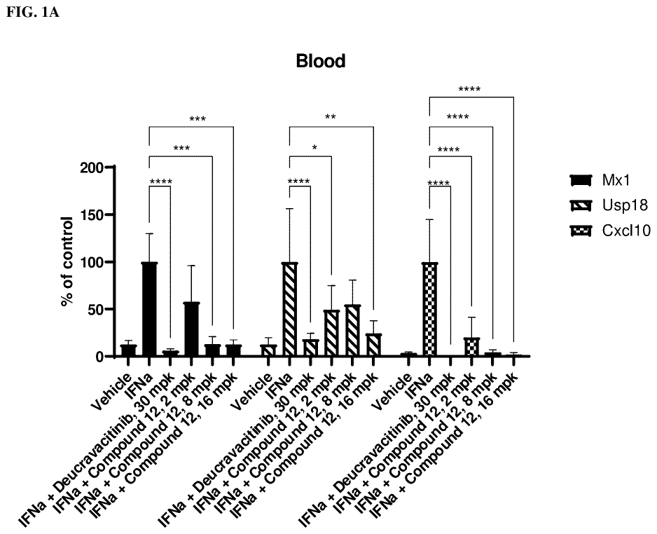

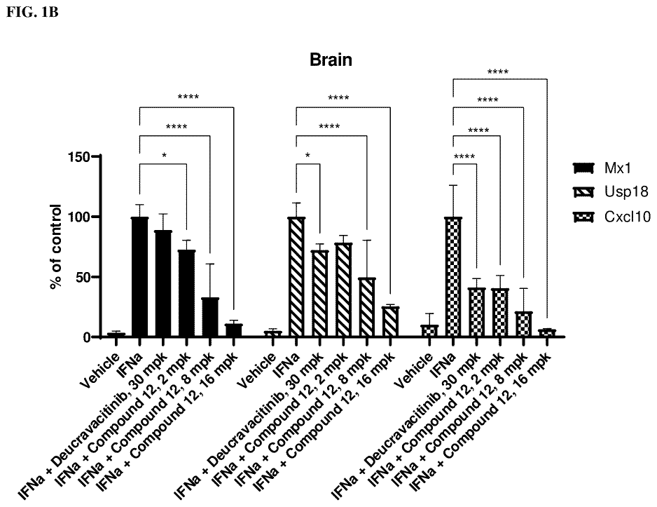

As shown in FIGS. 1A-1B peripherally restricted inhibitor (deucravacitinib) provides little-to-no inhibition of IFNα-stimulated ISG production in the brain following a peripheral IFNα stimulus, even with complete suppression of IFN-response in the blood. The brain-penetrant Compound 12, provides inhibition in both the brain and the blood consistent with the exposure profile.

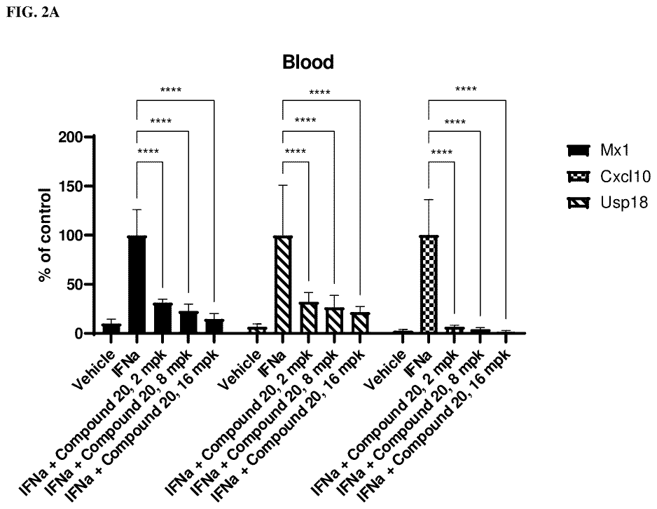

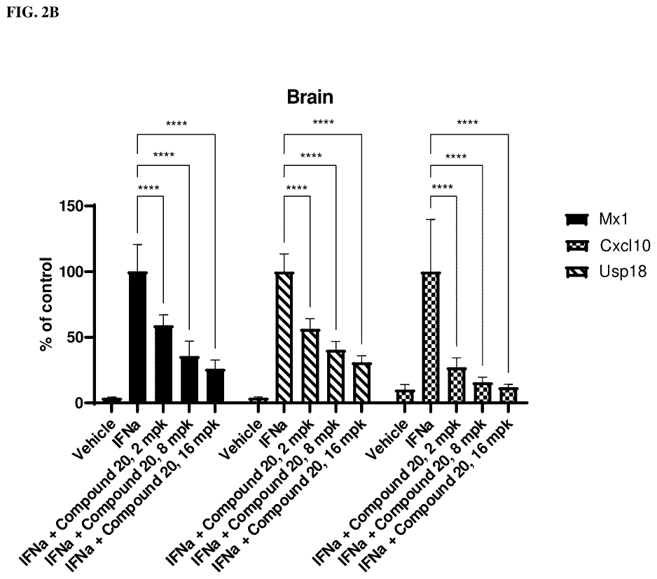

As shown in FIGS. 2A-2B The brain-penetrant Compound 20, provides inhibition in both the brain and the blood consistent with the exposure profile in the respective compartments.

1-63. (canceled)

64. A compound of Formula (IIB): 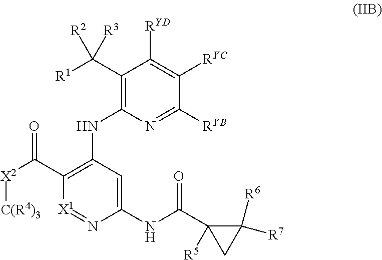
or a pharmaceutically acceptable salt, deuterated form, or stereoisomer thereof; wherein:
R¹ is —H, halo, —CN, —C₁₋₆ alkyl, —C₁₋₆ alkenyl, —C₁₋₆ alkynyl, —C₃₋₆ cycloalkyl, —OH, —OC₁₋₆ alkyl, —OC₃₋₆ cycloalkyl, —Oheterocyclyl, —NH₂, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl); wherein the —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, —Oheterocyclyl, —NH(C₁₋₆ alkyl), —N(C₁₋₆ alkyl)₂, —NH(C₃₋₆ cycloalkyl), or —N(C₁₋₆ alkyl)(C₃₋₆ cycloalkyl) are each independently optionally substituted with 1 or more halo, —CN, —OC₁₋₆ alkyl, —OC₁₋₆ haloalkyl, or -CO₂alkyl;
R² is —H, halo, —CN, —C₁₋₆ alkyl, —OC₁₋₆ alkyl, —C₁₋₆ alkenyl, —C₁₋₆ alkynyl, —C₃₋₆ cycloalkyl, or heterocyclyl; wherein the —C₁₋₆ alkyl, —OC₁₋₆ alkyl, —C₁₋₆ alkenyl, —C₁₋₆ alkynyl, —C₃₋₆ cycloalkyl, or heterocyclyl are each independently optionally substituted with 1 or more halo, —CN, —OC₁₋₆ alkyl, —OC₁₋₆ haloalkyl, or —CO₂alkyl;
or R¹ and R² are taken together to form a carbocyclyl or saturated or partially unsaturated heterocyclyl, wherein the carbocyclyl or saturated or partially unsaturated heterocyclyl are each independently optionally substituted with 1 or more halo, —OC₁₋₆ alkyl, —C₁₋₆ alkyl, —C₃₋₆ cycloalkyl, or —CN; wherein the —C₁₋₆ alkyl or —C₃₋₆ cycloalkyl, are each independently optionally substituted with 1 or more —CN, —OC₁₋₆ alkyl, —OC₁₋₆ haloalkyl, or —CO₂alkyl;
R³ is —H, halo, —C₁₋₆ alkyl, —O—C₁₋₆ alkyl, or —CN; wherein the —C₁₋₆ alkyl or —OC₁₋₆ alkyl, are each independently optionally substituted with 1 or more halo, —CN, —OC₁₋₆ alkyl, —OC₁₋₆ haloalkyl, or —CO₂alkyl;
each R⁴ is independently —H or -D;
X¹ is CH or N;
X² is C(R⁸)₂ or NH;
each R⁸ is independently H, halo, —C₁₋₆ alkyl, —C₁₋₆ haloalkyl, —O—C₁₋₆ alkyl, or —CN;
R^{YB}, R^{YC}, and R^{YD} are each independently —H, —CN, halo, —NR^{A}R^{B}, —C₁₋₆ alkyl, —OC₁₋₆ alkyl, —C₁₋₆ alkenyl, —C₁₋₆ alkynyl, —C₃₋₆ cycloalkyl, heterocyclyl, or aryl, wherein the, —C₁₋₆ alkyl, —OC₁₋₆ alkyl, —C₁₋₆ alkenyl, —C₁₋₆ alkynyl, —C₃₋₆ cycloalkyl, heterocyclyl, or aryl are each optionally substituted with halo, —C₁₋₆ alkyl, —C₁₋₆ haloalkyl, —OC₁₋₆ alkyl, —OC₃₋₆ cycloalkyl, or —CN;
R^{A} and R^{B} are each independently —H, —C₁-C₆ alkyl, cycloalkyl, or heterocyclyl; and
R⁵, R⁶ and R⁷ are each independently selected from the group consisting of —H, —CN, halo, —C₁₋₆ alkyl, —C₁₋₆ haloalkyl, —OC₁₋₆ alkyl, or —OC₃₋₆ cycloalkyl, or R⁶ and R⁷ are taken together to form a carbocyclyl.

65. The compound of claim 64, wherein:
R¹ is —H, halo, —OH, —CN, —C₁₋₆ alkyl, —C₁₋₆ alkenyl, —C₁₋₆ alkynyl, —C₃₋₆ cycloalkyl, —OC₁₋₆ alkyl, —OC₃₋₆ cycloalkyl, or —O-saturated heterocyclyl;
R² is —H, halo, —C₁₋₆ alkyl, —C₁₋₆ alkenyl, —C₁₋₆ alkynyl, —C₃₋₆ cycloalkyl, or saturated heterocyclyl; or
R¹ and R² are taken together to form a carbocyclyl or saturated heterocyclyl.

66. The compound of claim 64, wherein:
R¹ is —C₁₋₆ alkyl or —C₃₋₆ cycloalkyl optionally substituted with 1 or more halo;
R² is —H, —C₁₋₆ alkyl, or halo; or
R¹ and R² are taken together to form a carbocyclyl or saturated heterocyclyl, wherein the carbocyclyl or saturated heterocyclyl are optionally substituted with 1 or more halo.

67. The compound of claim 64, wherein R¹ is C₁₋₆ alkyl.

68. The compound of claim 67, wherein R¹ is —CH₃.

69. The compound of claim 64, wherein R¹ and R² are taken together to form a carbocyclyl.

70. The compound of claim 69, wherein R¹ and R² are taken together to form a —C₃₋₆ cycloalkyl.

71. The compound of claim 64, wherein R¹ and R² are taken together to form a cyclopropyl optionally substituted with 1 or 2 —F, oxetanyl, cyclobutyl,

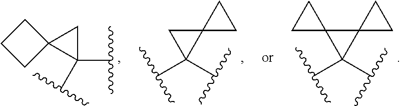 [SMILES: *C1(*)CC12CCC2 ; *C1(*)CC12CC2 ; *C1(*)C2(CC2)C12CC2]

72. The compound of claim 71, wherein R¹ and R² are taken together to form a cyclopropyl or

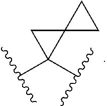 [SMILES: *C1(*)CC12CC2]

73. The compound of claim 64, wherein R² is —H, —C₁₋₆ alkyl or halo.

74. The compound of claim 73, wherein R² is —C₁₋₆ alkyl or halo.

75. The compound of claim 73, wherein R² is —CH₃.

76. The compound of claim 73, wherein R² is F.

77. The compound of claim 65, wherein R³ is —H, halo, —C₁₋₆ alkyl, —C₁₋₆ haloalkyl, —O—C₁₋₆ alkyl, or CN, wherein the —C₁₋₆ alkyl is optionally substituted with 1 or more OC₁₋₆ alkyl.

78. The compound of claim 77, wherein R³ is halo, —C₁₋₆ alkyl, —C₁₋₆ haloalkyl or CN.

79. The compound of claim 78, wherein R³ is —H, —F, —Cl, —CH₃, —CH₂F, —CHF₂, —CF₃, —OCH₃, —OCH₂CH₃, —CH₂CH₃, —CH₂OCH₃ or CN.

80. The compound of claim 64, wherein R³ is F.

81. The compound of claim 64, wherein R⁵ is —H, —C₁₋₆ alkyl, —CN, or halo.

82. The compound of claim 64, wherein R⁶ and R⁷ are each independently selected from the group consisting of —H, halo, —CN, —C₁₋₆ alkyl, —C₁₋₆ haloalkyl, or —OC₁₋₆ alkyl, or R⁶ and R⁷ are taken together to form a C₃₋₆cycloalkyl.

83. The compound of claim 64, wherein R⁵, R⁶ and R⁷ are H.

84. The compound of claim 64, wherein R^{YA}, R^{YB}, and R^{YC} are each independently —H, —CN, halo, —N(C₁₋₆ alkyl)(saturated heterocyclyl), —C₁₋₆ alkyl, —C₁₋₆ alkenyl, —C₁₋₆ alkynyl, —OC₁₋₆ alkyl, —C₃₋₆ cycloalkyl, saturated heterocyclyl, aryl, or heteroaryl, wherein the —C₁₋₆ alkyl, —C₁₋₆ alkenyl, —C₁₋₆ alkynyl, —OC₁₋₆ alkyl, —C₃₋₆ cycloalkyl, saturated heterocyclyl, aryl, or heteroaryl are optionally substituted with —F, —C₁₋₆ alkyl, or —OC₁₋₆ alkyl and R^{YD} is —H, —CN, halo, —C₁₋₆ alkyl, —C₁₋₆ alkenyl, —C₁₋₆ alkynyl, —C₃₋₆ cycloalkyl, a saturated heterocyclyl, or aryl.

85. The compound of claim 64, wherein R^{YB}, R^{YC}, and R^{YD} are each independently —H, —N(C₁₋₆ alkyl)(saturated heterocyclyl), —C₁₋₆ alkyl, —OC₁₋₆ alkyl, —CN, halo, saturated heterocyclyl, or heteroaryl, wherein the —C₁₋₆ alkyl, —OC₁₋₆ alkyl, saturated heterocyclyl, or heteroaryl are optionally substituted with —F, —C₁₋₆ alkyl, or —OC₁₋₆ alkyl.

86. The compound of claim 85, wherein two or three of R^{YB}, R^{YC}, and R^{YD} are —H.

87. The compound of claim 64, wherein X² is NH.

88. The compound of claim 64, wherein X² is CH₂.

89. The compound of claim 64, wherein each R⁴ is —H.

90. The compound of claim 64, wherein each R⁴ is -D.

91. The compound of claim 64, wherein X¹ is CH or N.

92. The compound of claim 64, wherein the compound is selected from the group consisting of: 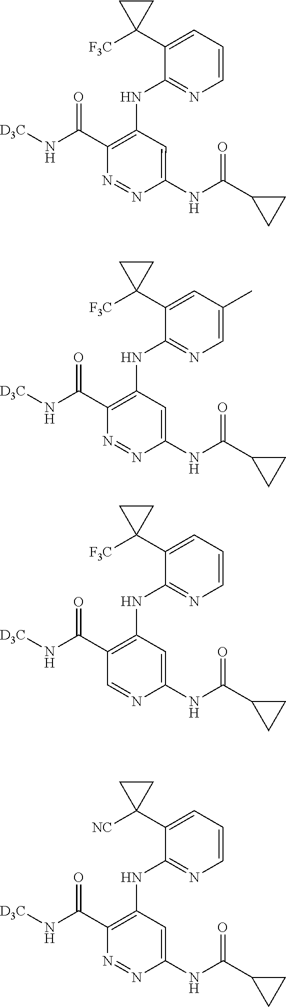 [SMILES: [2H]C([2H])([2H])NC(=O)c1nnc(NC(=O)C2CC2)cc1Nc1ncccc1C1(C(F)(F)F)CC1 ; [2H]C([2H])([2H])NC(=O)c1nnc(NC(=O)C2CC2)cc1Nc1ncc(C)cc1C1(C(F)(F)F)CC1 ; [2H]C([2H])([2H])NC(=O)c1cnc(NC(=O)C2CC2)cc1Nc1ncccc1C1(C(F)(F)F)CC1 ; [2H]C([2H])([2H])NC(=O)c1nnc(NC(=O)C2CC2)cc1Nc1ncccc1C1(C#N)CC1] 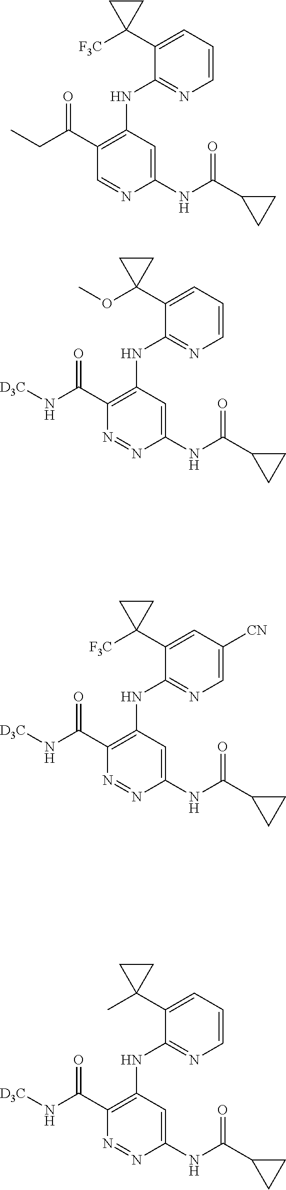 [SMILES: CCC(=O)c1cnc(NC(=O)C2CC2)cc1Nc1ncccc1C1(C(F)(F)F)CC1 ; [2H]C([2H])([2H])NC(=O)c1nnc(NC(=O)C2CC2)cc1Nc1ncccc1C1(OC)CC1 ; [2H]C([2H])([2H])NC(=O)c1nnc(NC(=O)C2CC2)cc1Nc1ncc(C#N)cc1C1(C(F)(F)F)CC1 ; [2H]C([2H])([2H])NC(=O)c1nnc(NC(=O)C2CC2)cc1Nc1ncccc1C1(C)CC1] 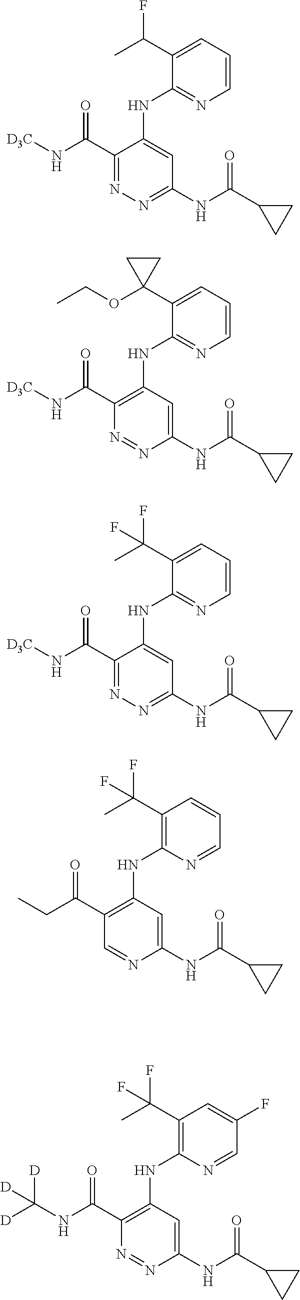 [SMILES: [2H]C([2H])([2H])NC(=O)c1nnc(NC(=O)C2CC2)cc1Nc1ncccc1C(C)F ; [2H]C([2H])([2H])NC(=O)c1nnc(NC(=O)C2CC2)cc1Nc1ncccc1C1(OCC)CC1 ; [2H]C([2H])([2H])NC(=O)c1nnc(NC(=O)C2CC2)cc1Nc1ncccc1C(C)(F)F ; CCC(=O)c1cnc(NC(=O)C2CC2)cc1Nc1ncccc1C(C)(F)F ; [2H]C([2H])([2H])NC(=O)c1nnc(NC(=O)C2CC2)cc1Nc1ncc(F)cc1C(C)(F)F] 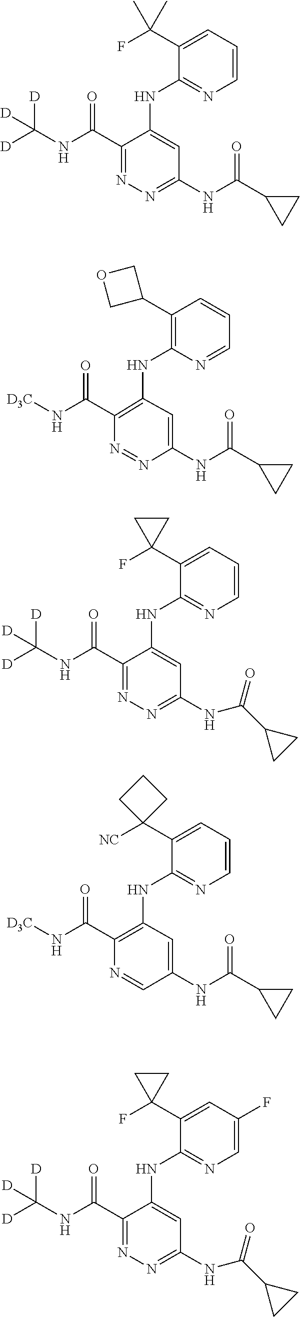 [SMILES: [2H]C([2H])([2H])NC(=O)c1nnc(NC(=O)C2CC2)cc1Nc1ncccc1C(C)(C)F ; [2H]C([2H])([2H])NC(=O)c1nnc(NC(=O)C2CC2)cc1Nc1ncccc1C1COC1 ; [2H]C([2H])([2H])NC(=O)c1nnc(NC(=O)C2CC2)cc1Nc1ncccc1C1(F)CC1 ; [2H]C([2H])([2H])NC(=O)c1ncc(NC(=O)C2CC2)cc1Nc1ncccc1C1(C#N)CCC1 ; [2H]C([2H])([2H])NC(=O)c1nnc(NC(=O)C2CC2)cc1Nc1ncc(F)cc1C1(F)CC1] 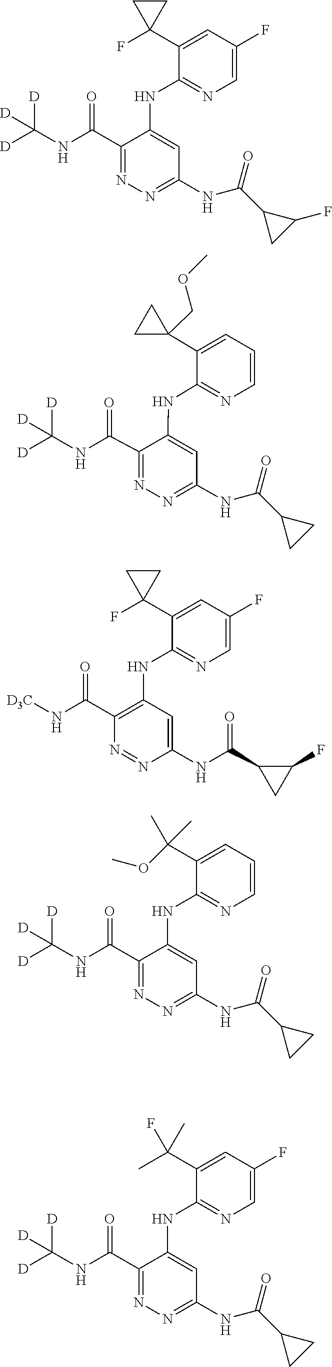 [SMILES: [2H]C([2H])([2H])NC(=O)c1nnc(NC(=O)C2CC2F)cc1Nc1ncc(F)cc1C1(F)CC1 ; [2H]C([2H])([2H])NC(=O)c1nnc(NC(=O)C2CC2)cc1Nc1ncccc1C1(COC)CC1 ; [2H]C([2H])([2H])NC(=O)c1nnc(NC(=O)[C@@H]2C[C@@H]2F)cc1Nc1ncc(F)cc1C1(F)CC1 ; [2H]C([2H])([2H])NC(=O)c1nnc(NC(=O)C2CC2)cc1Nc1ncccc1C(C)(C)OC ; [2H]C([2H])([2H])NC(=O)c1nnc(NC(=O)C2CC2)cc1Nc1ncc(F)cc1C(C)(C)F] 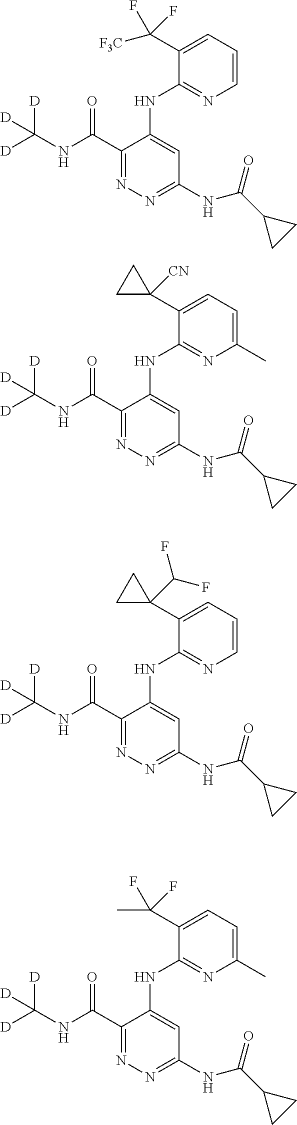 [SMILES: [2H]C([2H])([2H])NC(=O)c1nnc(NC(=O)C2CC2)cc1Nc1ncccc1C(F)(F)C(F)(F)F ; [2H]C([2H])([2H])NC(=O)c1nnc(NC(=O)C2CC2)cc1Nc1nc(C)ccc1C1(C#N)CC1 ; [2H]C([2H])([2H])NC(=O)c1nnc(NC(=O)C2CC2)cc1Nc1ncccc1C1(C(F)F)CC1 ; [2H]C([2H])([2H])NC(=O)c1nnc(NC(=O)C2CC2)cc1Nc1nc(C)ccc1C(C)(F)F] 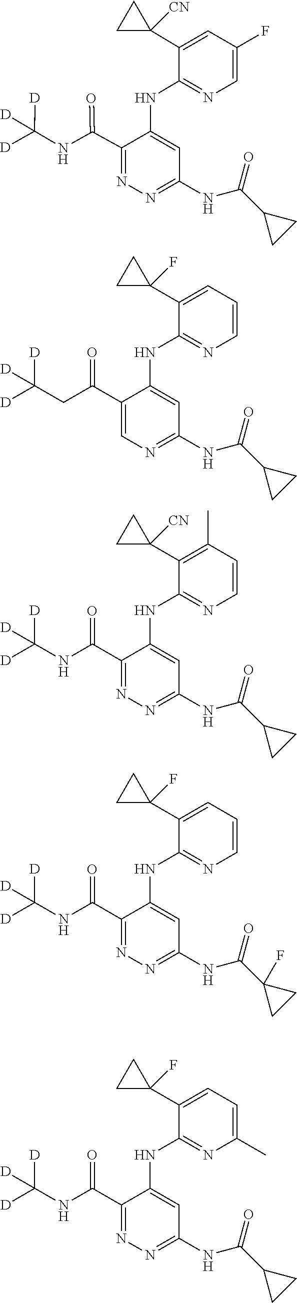 [SMILES: [2H]C([2H])([2H])NC(=O)c1nnc(NC(=O)C2CC2)cc1Nc1ncc(F)cc1C1(C#N)CC1 ; [2H]C([2H])([2H])CC(=O)c1cnc(NC(=O)C2CC2)cc1Nc1ncccc1C1(F)CC1 ; [2H]C([2H])([2H])NC(=O)c1nnc(NC(=O)C2CC2)cc1Nc1nccc(C)c1C1(C#N)CC1 ; [2H]C([2H])([2H])NC(=O)c1nnc(NC(=O)C2(F)CC2)cc1Nc1ncccc1C1(F)CC1 ; [2H]C([2H])([2H])NC(=O)c1nnc(NC(=O)C2CC2)cc1Nc1nc(C)ccc1C1(F)CC1] 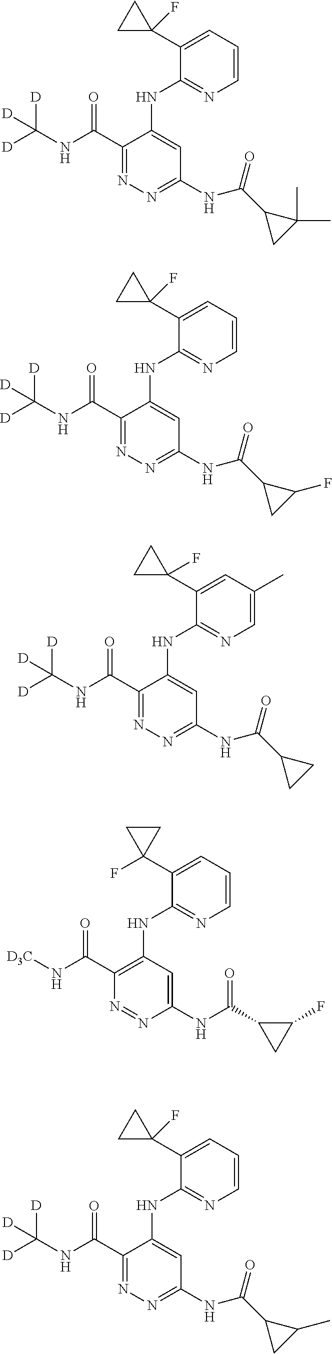 [SMILES: [2H]C([2H])([2H])NC(=O)c1nnc(NC(=O)C2CC2(C)C)cc1Nc1ncccc1C1(F)CC1 ; [2H]C([2H])([2H])NC(=O)c1nnc(NC(=O)C2CC2F)cc1Nc1ncccc1C1(F)CC1 ; [2H]C([2H])([2H])NC(=O)c1nnc(NC(=O)C2CC2)cc1Nc1ncc(C)cc1C1(F)CC1 ; [2H]C([2H])([2H])NC(=O)c1nnc(NC(=O)[C@H]2C[C@H]2F)cc1Nc1ncccc1C1(F)CC1 ; [2H]C([2H])([2H])NC(=O)c1nnc(NC(=O)C2CC2C)cc1Nc1ncccc1C1(F)CC1] 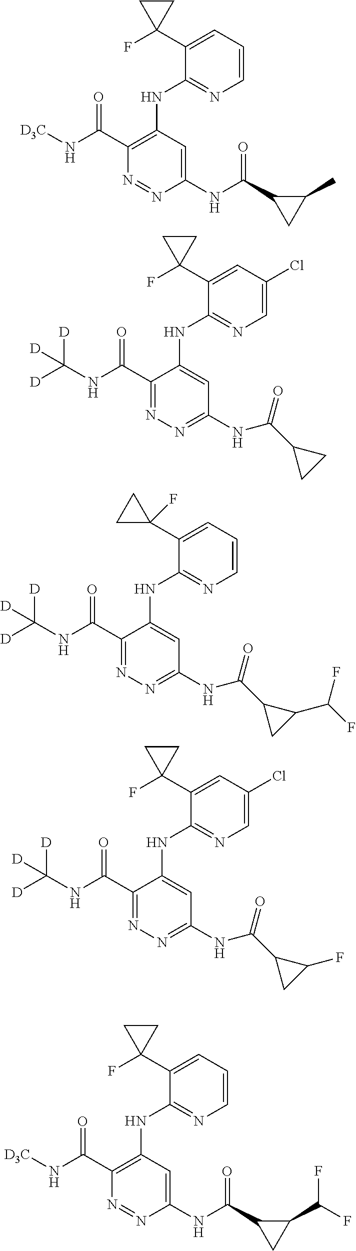 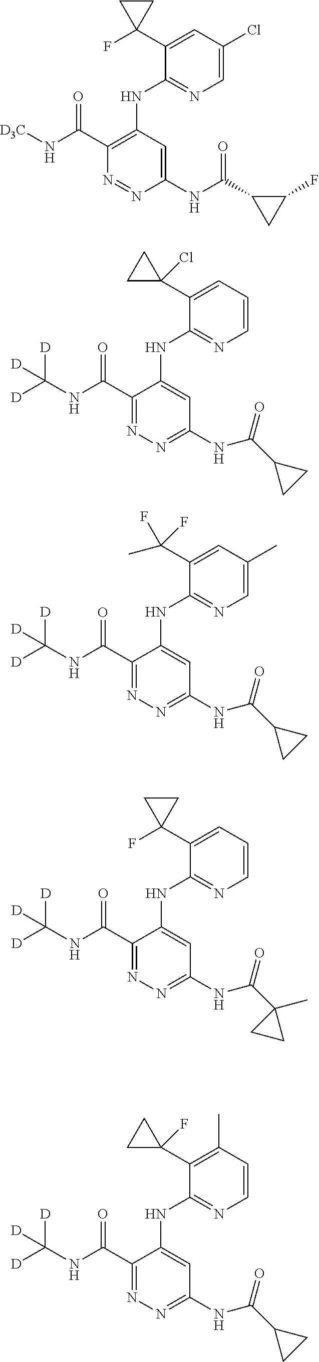 [SMILES: [2H]C([2H])([2H])NC(=O)c1nnc(NC(=O)[C@H]2C[C@H]2F)cc1Nc1ncc(Cl)cc1C1(F)CC1 ; [2H]C([2H])([2H])NC(=O)c1nnc(NC(=O)C2CC2)cc1Nc1ncccc1C1(Cl)CC1 ; [2H]C([2H])([2H])NC(=O)c1nnc(NC(=O)C2CC2)cc1Nc1ncc(C)cc1C(C)(F)F ; [2H]C([2H])([2H])NC(=O)c1nnc(NC(=O)C2(C)CC2)cc1Nc1ncccc1C1(F)CC1 ; [2H]C([2H])([2H])NC(=O)c1nnc(NC(=O)C2CC2)cc1Nc1nccc(C)c1C1(F)CC1] 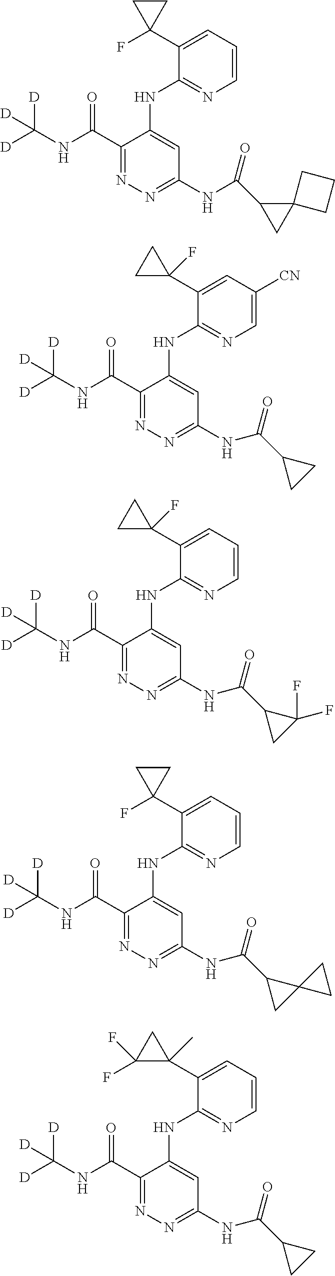 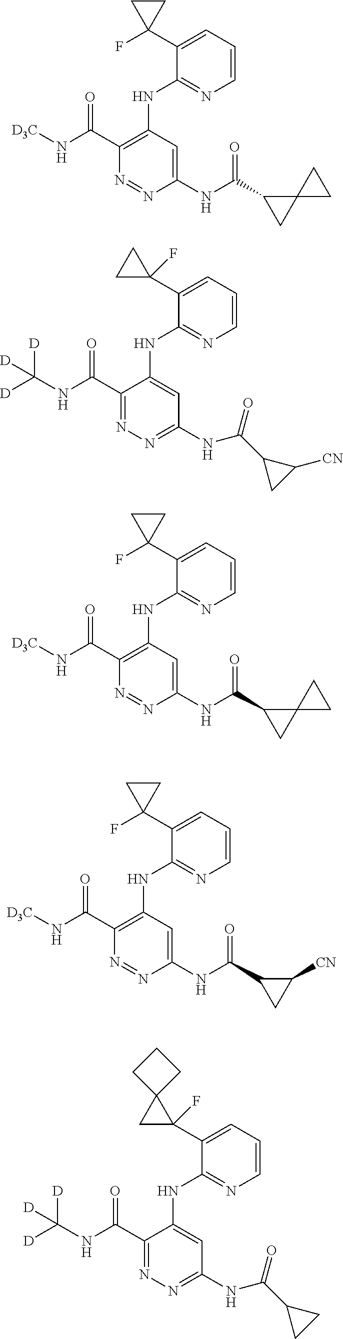 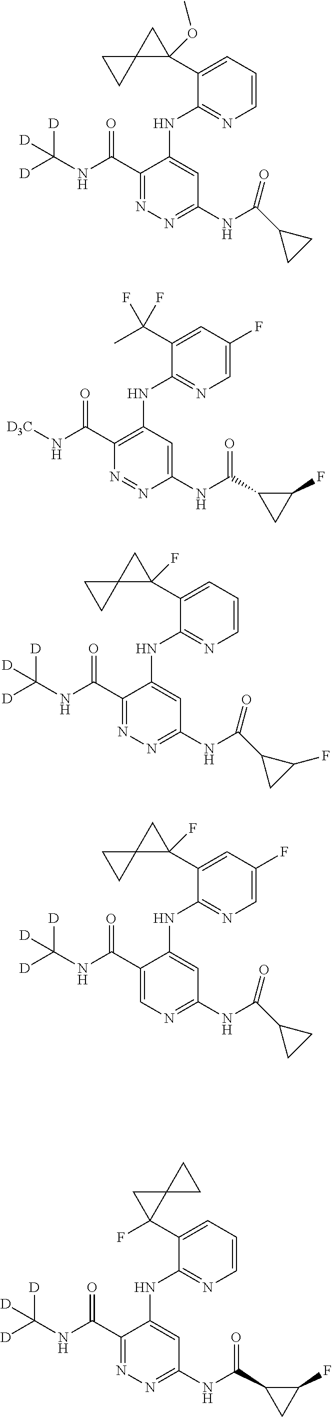 [SMILES: [2H]C([2H])([2H])NC(=O)c1nnc(NC(=O)C2CC2)cc1Nc1ncccc1C1(OC)CC12CC2 ; [2H]C([2H])([2H])NC(=O)c1nnc(NC(=O)[C@H]2C[C@@H]2F)cc1Nc1ncc(F)cc1C(C)(F)F ; [2H]C([2H])([2H])NC(=O)c1nnc(NC(=O)C2CC2F)cc1Nc1ncccc1C1(F)CC12CC2 ; [2H]C([2H])([2H])NC(=O)c1cnc(NC(=O)C2CC2)cc1Nc1ncc(F)cc1C1(F)CC12CC2 ; [2H]C([2H])([2H])NC(=O)c1nnc(NC(=O)[C@@H]2C[C@@H]2F)cc1Nc1ncccc1C1(F)CC12CC2] 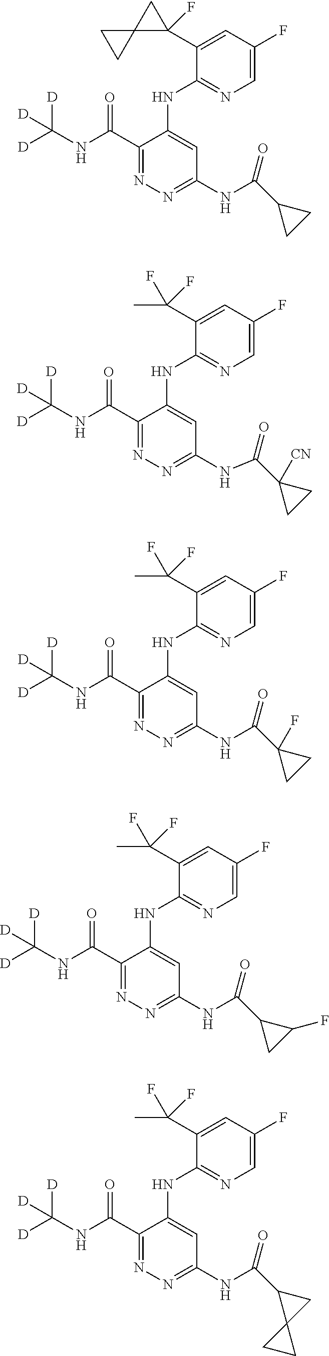 [SMILES: [2H]C([2H])([2H])NC(=O)c1nnc(NC(=O)C2CC2)cc1Nc1ncc(F)cc1C1(F)CC12CC2 ; [2H]C([2H])([2H])NC(=O)c1nnc(NC(=O)C2(C#N)CC2)cc1Nc1ncc(F)cc1C(C)(F)F ; [2H]C([2H])([2H])NC(=O)c1nnc(NC(=O)C2(F)CC2)cc1Nc1ncc(F)cc1C(C)(F)F ; [2H]C([2H])([2H])NC(=O)c1nnc(NC(=O)C2CC2F)cc1Nc1ncc(F)cc1C(C)(F)F ; [2H]C([2H])([2H])NC(=O)c1nnc(NC(=O)C2CC23CC3)cc1Nc1ncc(F)cc1C(C)(F)F] 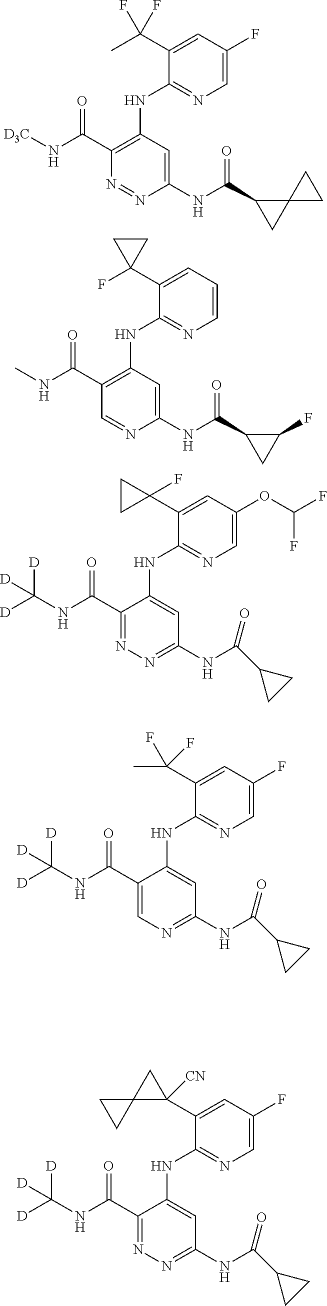 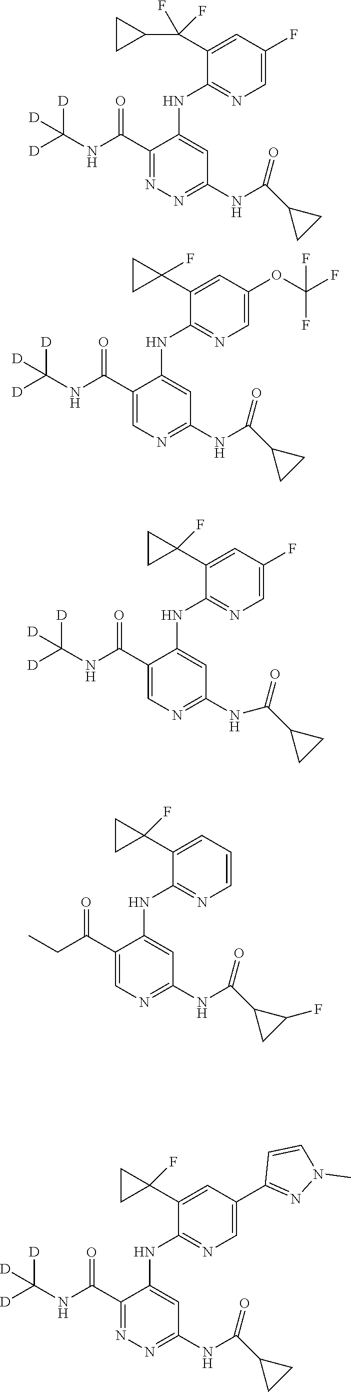 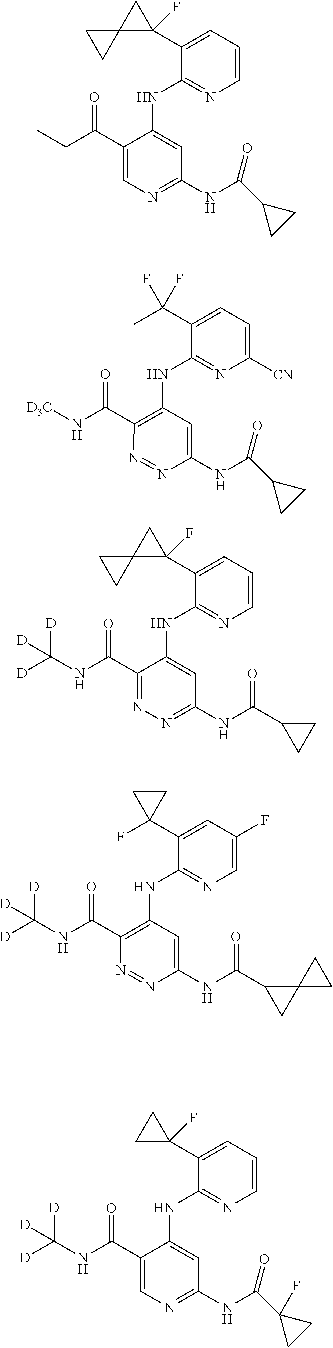 [SMILES: CCC(=O)c1cnc(NC(=O)C2CC2)cc1Nc1ncccc1C1(F)CC12CC2 ; [2H]C([2H])([2H])NC(=O)c1nnc(NC(=O)C2CC2)cc1Nc1nc(C#N)ccc1C(C)(F)F ; [2H]C([2H])([2H])NC(=O)c1nnc(NC(=O)C2CC2)cc1Nc1ncccc1C1(F)CC12CC2 ; [2H]C([2H])([2H])NC(=O)c1nnc(NC(=O)C2CC23CC3)cc1Nc1ncc(F)cc1C1(F)CC1 ; [2H]C([2H])([2H])NC(=O)c1cnc(NC(=O)C2(F)CC2)cc1Nc1ncccc1C1(F)CC1] 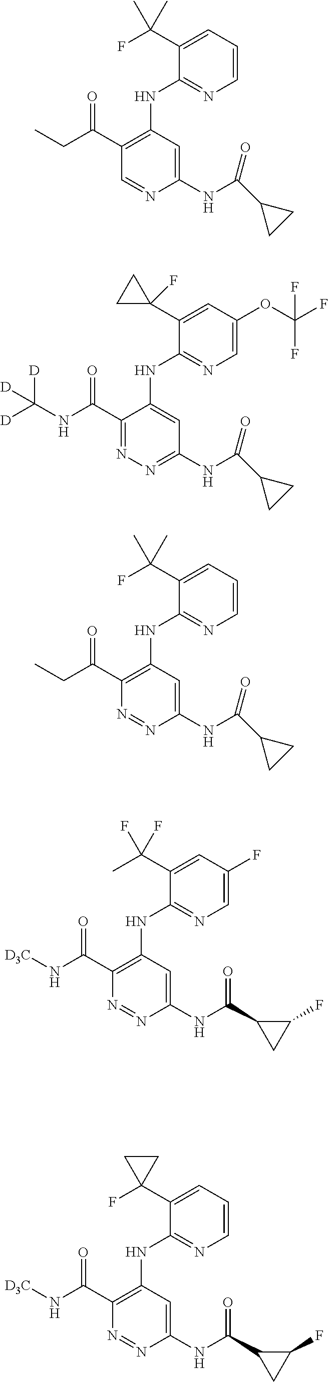 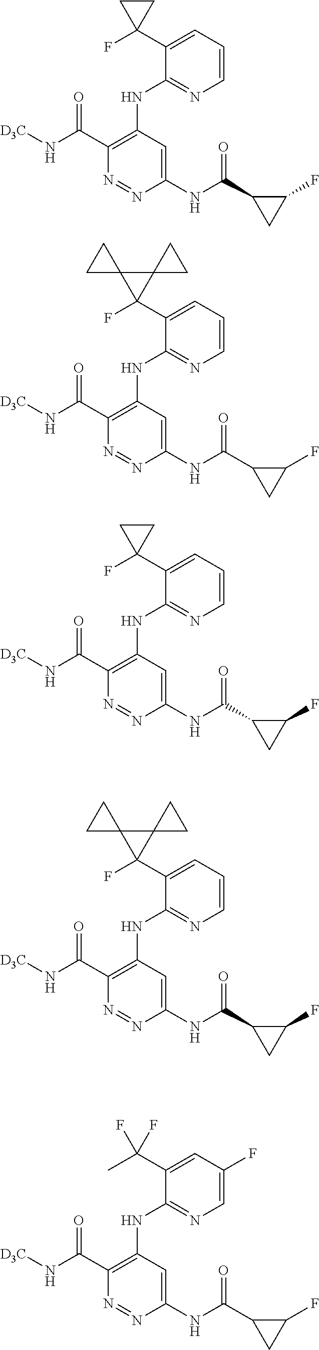 [SMILES: [2H]C([2H])([2H])NC(=O)c1nnc(NC(=O)[C@@H]2C[C@H]2F)cc1Nc1ncccc1C1(F)CC1 ; [2H]C([2H])([2H])NC(=O)c1nnc(NC(=O)C2CC2F)cc1Nc1ncccc1C1(F)C2(CC2)C12CC2 ; [2H]C([2H])([2H])NC(=O)c1nnc(NC(=O)[C@H]2C[C@@H]2F)cc1Nc1ncccc1C1(F)CC1 ; [2H]C([2H])([2H])NC(=O)c1nnc(NC(=O)[C@@H]2C[C@@H]2F)cc1Nc1ncccc1C1(F)C2(CC2)C12CC2 ; [2H]C([2H])([2H])NC(=O)c1nnc(NC(=O)C2CC2F)cc1Nc1ncc(F)cc1C(C)(F)F] 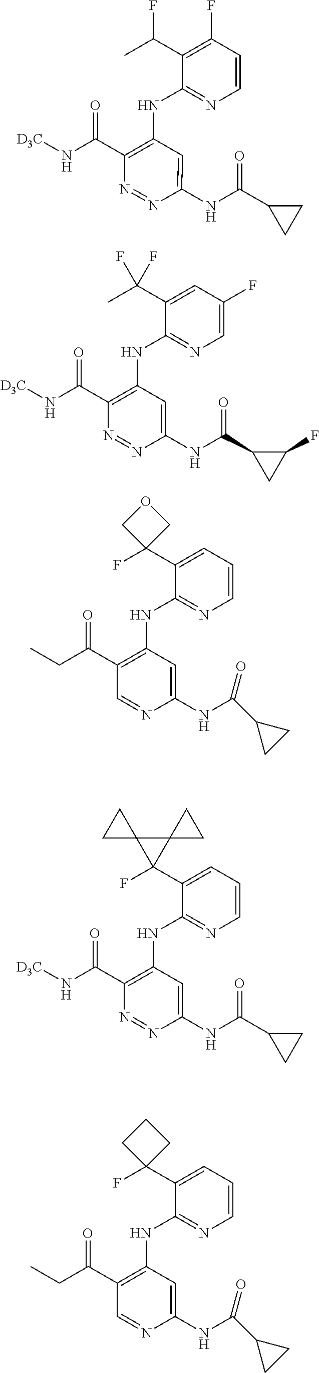 [SMILES: [2H]C([2H])([2H])NC(=O)c1nnc(NC(=O)C2CC2)cc1Nc1nccc(F)c1C(C)F ; [2H]C([2H])([2H])NC(=O)c1nnc(NC(=O)[C@@H]2C[C@@H]2F)cc1Nc1ncc(F)cc1C(C)(F)F ; CCC(=O)c1cnc(NC(=O)C2CC2)cc1Nc1ncccc1C1(F)COC1 ; [2H]C([2H])([2H])NC(=O)c1nnc(NC(=O)C2CC2)cc1Nc1ncccc1C1(F)C2(CC2)C12CC2 ; CCC(=O)c1cnc(NC(=O)C2CC2)cc1Nc1ncccc1C1(F)CCC1] 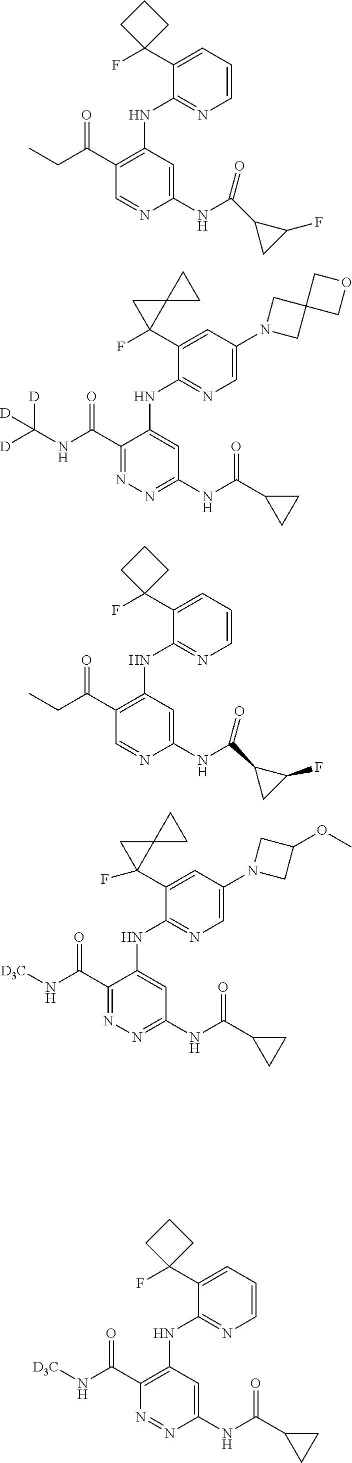 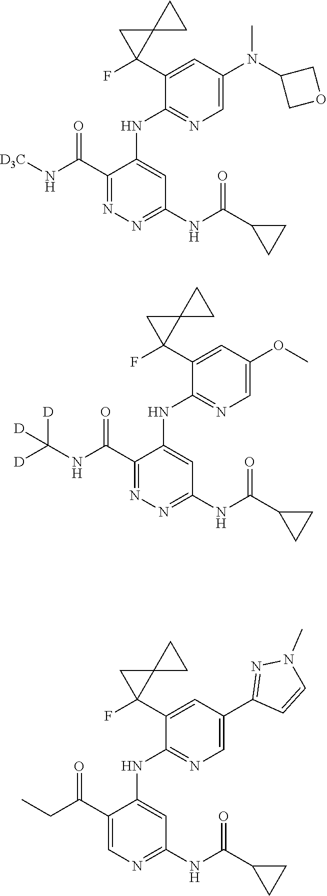 [SMILES: [2H]C([2H])([2H])NC(=O)c1nnc(NC(=O)C2CC2)cc1Nc1ncc(N(C)C2COC2)cc1C1(F)CC12CC2 ; [2H]C([2H])([2H])NC(=O)c1nnc(NC(=O)C2CC2)cc1Nc1ncc(OC)cc1C1(F)CC12CC2 ; CCC(=O)c1cnc(NC(=O)C2CC2)cc1Nc1ncc(-c2ccn(C)n2)cc1C1(F)CC12CC2]
or a pharmaceutically acceptable salt, deuterated form, or stereoisomer thereof.

93. A pharmaceutical composition comprising a compound of claim 64, or a pharmaceutically acceptable salt, deuterated form, or stereoisomer thereof, and a pharmaceutically acceptable carrier.
